SEQUENCE LISTING (3 sequences)

SEQ ID NO: 1 - DNA, 22 bp, synthetic construct
  source 1..22: mol_type = other DNA
catccagcaaatacaccatcca

SEQ ID NO: 2 - DNA, 28 bp, synthetic construct
  source 1..28: mol_type = other DNA
ttctgcacatcataattaggagtatcaa

SEQ ID NO: 3 - DNA, 15 bp, synthetic construct
  source 1..15: mol_type = other DNA
cggagcacaggagat

(19) United States
(12) Patent Application Publication
(10) Pub. No.: US 2023/0382940 A1
(43) Pub. Date: Nov. 30, 2023

(54) ANTIVIRAL COMPOUNDS AND METHODS OF MAKING AND USING THE SAME

(71) Applicant: Gilead Sciences, Inc., Foster City, CA (US)

(72) Inventors: Byoung-Kwon Chun, Pleasanton, CA (US); Michael O. Clarke, Redwood City, CA (US); Deeba Ensan, Foster City, CA (US); Rao V. Kalla, Foster City, CA (US); Richard L. Mackman, Millbrae, CA (US); Devan Naduthambi, San Bruno, CA (US); Dustin S. Siegel, Half Moon Bay, CA (US)

(21) Appl. No.: 18/176,864

(22) Filed: Mar. 1, 2023

Related U.S. Application Data
(60) Provisional application No. 63/316,231, filed on Mar. 3, 2022.

(51) Int. Cl. C07H 7/06; A61P 31/16

(52) U.S. Cl. CPC C07H 7/06; A61P 31/16

(57) ABSTRACT

Antiviral compounds and methods of using the same, singly or in combination with additional agents, and pharmaceutical compositions of said compounds for the treatment of viral infections are disclosed.

### CROSS-REFERENCE TO RELATED APPLICATIONS

This application claims priority to U.S. Provisional Application No. 63/316,231, filed Mar. 3, 2022, which is incorporated herein in its entireties for all purposes.

### SEQUENCE LISTING

The instant application contains a Sequence Listing which has been submitted electronically in .XML file format and is hereby incorporated by reference in its entirety. Said .XML copy, created on Feb. 9, 2023, is named 1429-US-NP.xml and is 3,489 bytes in size.

### BACKGROUND

There is a need for compounds, pharmaceutical compositions, and methods for treating viral infections, for example, Paramyxoviridae, Pneumoviridae, Picornaviridae, Flaviviridae, Filoviridae, and Orthomyxovirus infections. Embodiments of the present disclosure can address these and other needs.

### SUMMARY

Disclosed herein are, among other things, compounds of Formula I:

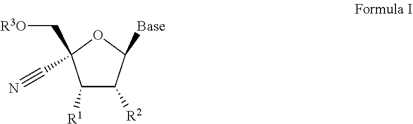

or a pharmaceutically acceptable salt thereof, wherein:
R¹ is OH, OC(O)R⁴, or OC(O)OR⁴;
R² is OH, OC(O)R⁵. Or OC(O)OR⁵; or
R¹ and R² are taken together to form —OC(O)O— or —OCR⁶O—, wherein
   R⁶ is H, C₁-C₆ alkyl, C₁-C₆ alkoxy, or C₆-C₁₀ aryl;
R³ is H or C(O)OR⁷, or
R¹ and R³ are taken together to form —OC(O)—;
R⁴, R⁵, and R⁷ are each independently C₁-C₈ alkyl, C₂-C₈ alkenyl, C₂-C₈ alkynyl, C₃-C₈ carbocyclyl, C₆-C₁₀ aryl, 4 to 6 membered heterocyclyl containing 1, 2, or 3 O, or 5 to 6 membered heteroaryl containing 1, 2, or 3 heteroatoms selected from N, O, and S;
wherein R⁴, R⁵, and R⁷ are each, independently, optionally substituted with one, two or three substituents independently selected from the group consisting of halo, oxo, cyano, —N₃, —OR⁸, C₁-C₈ alkyl, —NR⁹R¹⁰, C₃-C₈ carbocyclyl, and phenyl optionally substituted with one, two, or three substituents independently selected from halo, cyano, and C₁-C₆ alkyl; and
   each R⁸ is independently H, C₁-C₆ alkyl, C₁-C₆ haloalkyl, and C₃-C₆ cycloalkyl;
   each R⁹ is independently H, C₁-C₆ alkyl, C₁-C₆ haloalkyl, and C₃-C₆ cycloalkyl;
   each R¹⁰ is independently H, C₁-C₆ alkyl, C₁-C₆ haloalkyl, and C₃-C₆ cycloalkyl; and
Base is

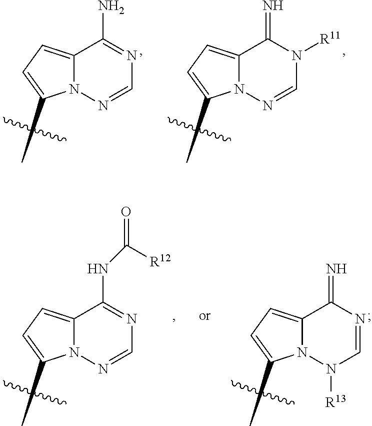

wherein
   R¹¹ is C₁-C₆ alkyl substituted with —OP(O)(OH)₂ or —OC(O)R¹²;
   R¹² is C₁-C₈ alkyl or O—C₁-C₈ alkyl; and
   R¹³ is C₁-C₆ alkyl substituted with —OP(O)(OH)₂ or —OC(O)R¹²;
   wherein the compound is not

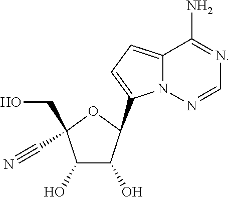 [SMILES: N#C[C@]1(CO)O[C@@H](c2ccc3c(N)ncnn23)[C@H](O)[C@@H]1O]

Also disclosed herein are compounds and pharmaceutically acceptable salts thereof of sub-formulas of Formula I, such as Formula II.

Disclosed herein are pharmaceutical compositions comprising a pharmaceutically effective amount of a compound disclosed herein, or a pharmaceutically acceptable salt thereof, and a pharmaceutically acceptable carrier or excipient.

Also disclosed herein are methods of treating or preventing a viral infection in a subject in need thereof, wherein the method comprises administering to the subject a compound disclosed herein, or a pharmaceutically acceptable salt thereof.

Also disclosed herein are methods of treating or preventing a viral infection in a human in need thereof, wherein the method comprises administering to the human a compound disclosed herein, or a pharmaceutically acceptable salt thereof.

The present disclosure provides a method for manufacturing a medicament for treating or preventing a viral infection in a subject in need thereof, characterized in that a compound disclosed herein, or a pharmaceutically acceptable salt thereof, is used.

The present disclosure provides a method for manufacturing a medicament for treating or preventing a viral infection in a human in need thereof, characterized in that a compound disclosed herein, or a pharmaceutically acceptable salt thereof, is used.

The present disclosure provides use of a compound disclosed herein, or a pharmaceutically acceptable salt thereof, for the manufacture of a medicament for the treatment or prevention of a viral infection in a human in need thereof.

The present disclosure provides use of a compound disclosed herein, or a pharmaceutically acceptable salt thereof, for the manufacture of a medicament for the treatment or prevention of a viral infection in a human in need thereof.

### DETAILED DESCRIPTION

### I. General

The disclosure relates generally to methods and compounds for treating or preventing viral infections, for example Paramyxoviridae, Pneumoviridae, Picornaviridae, Flaviviridae, Filoviridae, and Orthomyxovirus infections. The following description sets forth exemplary methods, parameters and the like. It should be recognized, however, that such description is not intended as a limitation on the scope of the present disclosure but is instead provided as a description of exemplary embodiments.

### II. Definitions

As used in the present specification, the following words, phrases and symbols are generally intended to have the meanings as set forth below, except to the extent that the context in which they are used indicates otherwise.

A dash (“-”) that is not between two letters or symbols is used to indicate a point of attachment for a substituent. For example, —CONH₂ is attached through the carbon atom. A dash at the front or end of a chemical group is a matter of convenience; chemical groups can be depicted with or without one or more dashes without losing their ordinary meaning. A wavy line drawn through a line in a structure indicates a point of attachment of a group. Unless chemically or structurally required, no directionality is indicated or implied by the order in which a chemical group is written or named.

A squiggly line on a chemical group as shown below, for example,

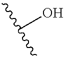 [SMILES: *O]

indicates a point of attachment, i.e., it shows the broken bond by which the group is connected to another described group.

As used herein, “a compound of the disclosure” can mean a compound of any of the Formulas I-II or a pharmaceutically acceptable salt thereof Similarly, the phrase “a compound of Formula (number)” means a compound of that formula and pharmaceutically acceptable salts thereof.

The prefix “Cᵤ-Cᵥ” indicates that the following group has from u to v carbon atoms. For example, “C₁-C₈ alkyl” indicates that the alkyl group has from 1 to 8 carbon atoms.

“Alkyl” refers to an unbranched or branched saturated hydrocarbon chain. For example, an alkyl group can have 1 to 20 carbon atoms (i.e., C₁-C₂₀ alkyl), 1 to 8 carbon atoms (i.e., C₁-C₈ alkyl), 1 to 6 carbon atoms (i.e., C₁-C₆ alkyl), or 1 to 3 carbon atoms (i.e., C₁-C₃ alkyl). Examples of suitable alkyl groups include, but are not limited to, methyl (Me, —CH₃), ethyl (Et, —CH₂CH₃), 1-propyl (n-Pr, n-propyl, —CH₂CH₂CH₃), 2-propyl (i-Pr, i-propyl, —CH(CH₃)₂), 1-butyl (n-Bu, n-butyl, —CH₂CH₂CH₂CH₃), 2-methyl-1-propyl (i-Bu, i-butyl, —CH₂CH(CH₃)₂), 2-butyl (s-Bu, s-butyl, —CH(CH₃)CH₂CH₃), 2-methyl-2-propyl (t-Bu, t-butyl, —C(CH₃)₃), 1-pentyl (n-pentyl, —CH₂CH₂CH₂CH₂CH₃), 2-pentyl (—CH(CH₃)CH₂CH₂CH₃), 3-pentyl (—CH(CH₂CH₃)₂), 2-methyl-2-butyl (—C(CH₃)₂CH₂CH₃), 3-methyl-2-butyl (—CH(CH₃)CH(CH₃)₂), 3-methyl-1-butyl (—CH₂CH₂CH(CH₃)₂), 2-methyl-1-butyl (—CH₂CH(CH₃)CH₂CH₃), 1-hexyl (—CH₂CH₂CH₂CH₂CH₂CH₃), 2-hexyl (—CH(CH₃)CH₂CH₂CH₂CH₃), 3-hexyl (—CH(CH₂CH₃)(CH₂CH₂CH₃)), 2-methyl-2-pentyl (—C(CH₃)₂CH₂CH₂CH₃), 3-methyl-2-pentyl (—CH(CH₃)CH(CH₃)CH₂CH₃), 4-methyl-2-pentyl (—CH(CH₃)CH₂CH(CH₃)₂), 3-methyl-3-pentyl (—C(CH₃)(CH₂CH₃)₂), 2-methyl-3-pentyl (—CH(CH₂CH₃)CH(CH₃)₂), 2,3-dimethyl-2-butyl (—C(CH₃)₂CH(CH₃)₂), and 3,3-dimethyl-2-butyl (—CH(CH₃)C(CH₃)₃. Other alkyl groups include, but are not limited to, heptyl, octyl, nonyl, decyl, undecyl, dodecyl, pentadecyl, hexadecyl, heptadecyl and octadecyl.

“Alkenyl” refers to an unbranched or branched hydrocarbon chain containing at least two carbon atoms and at least one carbon-carbon double bond. As used herein, alkenyl can have from 2 to 20 carbon atoms (i.e., C₂₋₂₀ alkenyl), 2 to 8 carbon atoms (i.e., C₂₋ₛ alkenyl), 2 to 6 carbon atoms (i.e., C₂₋₆ alkenyl), or 2 to 4 carbon atoms (i.e., C₂₋₄ alkenyl). Alkenyl can include any number of carbons, such as C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, or any range therein. Alkenyl groups can have any suitable number of double bonds, including, but not limited to, 1, 2, 3, 4, 5 or more. Examples of alkenyl groups include, but are not limited to, vinyl (ethenyl), propenyl, isopropenyl, 1-butenyl, 2-butenyl, isobutenyl, butadienyl, 1-pentenyl, 2-pentenyl, isopentenyl, 1,3-pentadienyl, 1,4-pentadienyl, 1-hexenyl, 2-hexenyl, 3-hexenyl, 1,3-hexadienyl, 1,4-hexadienyl, 1,5-hexadienyl, 2,4-hexadienyl, or 1,3,5-hexatrienyl.

“Alkoxy” means a group having the formula —O-alkyl, in which an alkyl group, as defined above, is attached to the parent molecule via an oxygen atom. The alkyl portion of an alkoxy group can have 1 to 20 carbon atoms (i.e., C₁-C₂₀ alkoxy), 1 to 12 carbon atoms (i.e., C₁-C₁₂ alkoxy), 1 to 8 carbon atoms (i.e., C₁-C₈ alkoxy), 1 to 6 carbon atoms (i.e., C₁-C₆ alkoxy) or 1 to 3 carbon atoms (i.e., C₁-C₃ alkoxy). Examples of suitable alkoxy groups include, but are not limited to, methoxy (—O—CH₃ or —OMe), ethoxy (—OCH₂CH₃ or -OEt), isopropoxy (—O—CH(CH₃)₂), t-butoxy (—O—C(CH₃)₃ or -OtBu) and the like. Other examples of suitable alkoxy groups include, but are not limited to, sec-butoxy, tert-butoxy, pentoxy, hexoxy, and the like.

“Alkynyl” refers to an unbranched or branched hydrocarbon chain containing at least one carbon-carbon triple bond. For example, an alkynyl group can have from 2 to 20 carbon atoms (i.e., C₂₋₂₀ alkynyl), 2 to 8 carbon atoms (i.e., C₂₋₈ alkynyl), 2 to 6 carbon atoms (i.e., C₂₋₆ alkynyl), or 2 to 4 carbon atoms (i.e., C₂₋₄ alkynyl). The term “alkynyl” also includes those groups having one triple bond and one double bond. Examples of C₂₋₆alkynyl include, but are not limited to, ethynyl, prop-1-ynyl, but-1-ynyl, pent-1-ynyl, pent-4-ynyl and penta-1,4-diynyl.

“Aryl” means an aromatic hydrocarbon radical derived by the removal of one hydrogen atom from a single carbon atom of a parent aromatic ring system. For example, an aryl group can have 6 to 20 carbon atoms, 6 to 14 carbon atoms, or 6 to 10 carbon atoms. Exemplary aryl groups include, but are not limited to, radicals derived from benzene (e.g., phenyl), naphthalene, anthracene, biphenyl, and the like.

“Carbocyclyl” or “carbocyclic ring” refers to a non-aromatic hydrocarbon ring consisting of carbon and hydrogen atoms, having from three to twenty carbon atoms, in certain embodiments having from three to fifteen carbon atoms, in certain embodiments having from three to ten carbon atoms, from three to eight carbon atoms, from three to seven carbon atoms, or from 3 to 6 carbon atoms and which is saturated or partially unsaturated and attached to the rest of the molecule by a single bond. Carbocyclic rings include, for example, cyclopropane, cyclobutane, cyclopentane, cyclopentene, cyclohexane, cyclohexene, 1,3-cyclohexadiene, 1,4-cyclohexadiene, cycloheptane, cycloheptene, and cyclooctane.

“Cycloalkyl” refers to a saturated or partially saturated cyclic alkyl group having a single ring or multiple rings including fused, bridged, and spiro ring systems. As used herein, cycloalkyl has from 3 to 20 ring carbon atoms (i.e., C₃₋₂₀ cycloalkyl), 3 to 12 ring carbon atoms (i.e., C₃₋₁₂ cycloalkyl), 3 to 10 ring carbon atoms (i.e., C₃₋₁₀ cycloalkyl), 3 to 8 ring carbon atoms (i.e., C₃₋₈ cycloalkyl), or 3 to 6 ring carbon atoms (i.e., C₃₋₆ cycloalkyl). Examples of cycloalkyl groups include, but are not limited to, cyclopropyl, cyclobutyl, cyclopentyl, cyclohexyl, cycloheptyl, and cyclooctyl. Cycloalkyl groups also include partially unsaturated ring systems containing one or more double bonds, including fused ring systems with one aromatic ring and one non-aromatic ring, but not fully aromatic ring systems.

“Cyano” or “carbonitrile” refers to the group CN.

“Halo” or “halogen” as used herein refers to fluoro (—F), chloro (—Cl), bromo (—Br) and iodo (—I).

“Haloalkyl” is an alkyl group, as defined above, in which one or more hydrogen atoms of the alkyl group is replaced with a halogen atom. The alkyl portion of a haloalkyl group can have 1 to 20 carbon atoms (i.e., C₁-C₂₀ haloalkyl), 1 to 12 carbon atoms (i.e., C₁-C₁₂ haloalkyl), 1 to 8 carbon atoms (i.e., C₁-C₈ haloalkyl), 1 to 6 carbon atoms (i.e., C₁-C₆ alkyl) or 1 to 3 carbon atoms (i.e., C₁-C₃ alkyl). Examples of suitable haloalkyl groups include, but are not limited to, —CF₃, —CHF₂, —CFH₂, —CH₂CF₃, fluorochloromethyl, difluorochloromethyl, 1,1,1-trifluoroethyl and pentafluoroethyl.

“Heteroaryl” refers to an aromatic group, including groups having an aromatic tautomer or resonance structure, having a single ring, multiple rings, or multiple fused rings, with at least one heteroatom in the ring, i.e., one or more ring heteroatoms independently selected from nitrogen, oxygen, and sulfur, wherein the nitrogen or sulfur can be oxidized. Thus, the term includes rings having one or more annular O, N, S, S(O), S(O)₂, and N-oxide groups. The term includes rings having one or more annular C(O) groups. As used herein, heteroaryl include 5 to 20 ring atoms (i.e., 5- to 20-membered heteroaryl), 5 to 12 ring atoms (i.e., 5- to 12-membered heteroaryl), or 5 to 10 ring atoms (i.e., 5- to 10-membered heteroaryl), and 1 to 5 heteroatoms independently selected from nitrogen, oxygen, and sulfur, and oxidized forms of the heteroatoms. Examples of heteroaryl groups include, but are not limited to, pyridin-2(1H)-one, pyridazin-3(2H)-one, pyrimidin-4(3H)-one, quinolin-2(1H)-one, pyrimidinyl, purinyl, pyridyl, pyridazinyl, benzothiazolyl, and pyrazolyl. Heteroaryl does not encompass or overlap with aryl as defined above.

“Heterocycle” or “heterocyclyl” refer to a saturated or unsaturated cyclic alkyl group, with one or more ring heteroatoms independently selected from nitrogen, oxygen and sulfur. A heterocyclyl can be a single ring or multiple rings wherein the multiple rings can be fused, bridged, or spiro. As used herein, heterocyclyl has 3 to 20 ring atoms (i.e., 3 to 20 membered heterocyclyl), 3 to 12 ring atoms (i.e., 3 to 12 membered heterocyclyl), 3 to 10 ring atoms (i.e., 3 to 10 membered heterocyclyl), 3 to 8 ring atoms (i.e., 3 to 8 membered heterocyclyl), 4 to 12 ring carbon atoms (i.e., 4 to 12 membered heterocyclyl), 4 to 8 ring atoms (i.e., 4 to 8 membered heterocyclyl), or 4 to 6 ring atoms (i.e., 4 to 6 membered heterocyclyl). Examples of heterocyclyl groups include pyrrolidinyl, piperidinyl, piperazinyl, oxetanyl, dioxolanyl, azetidinyl, and morpholinyl.

The term “optionally substituted” in reference to a particular moiety of the compound disclosed herein (e.g., an optionally substituted aryl group) refers to a moiety wherein all substituents are hydrogen or wherein one or more of the hydrogens of the moiety can be replaced by the listed substituents.

Oxo″ refers to the group (═O) or (O).

Provided are also pharmaceutically acceptable salts, hydrates, solvates, tautomeric forms, polymorphs, and prodrugs of the compounds described herein. “Pharmaceutically acceptable” or “physiologically acceptable” refer to compounds, salts, formulations, dosage forms and other materials which are useful in preparing a pharmaceutical composition that is suitable for veterinary or human pharmaceutical use.

The compounds described herein can be prepared and/or formulated as pharmaceutically acceptable salts or when appropriate as a free base. Pharmaceutically acceptable salts are non-toxic salts of a free base form of a compound that possess the desired pharmacological activity of the free base. These salts can be derived from inorganic or organic acids or bases. For example, a compound that contains a basic nitrogen can be prepared as a pharmaceutically acceptable salt by contacting the compound with an inorganic or organic acid. Non-limiting examples of pharmaceutically acceptable salts include sulfates, pyrosulfates, bisulfates, sulfites, bisulfites, phosphates, monohydrogen-phosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, methylsulfonates, propylsulfonates, besylates, xylenesulfonates, naphthalene-1-sulfonates, naphthalene-2-sulfonates, phenylacetates, phenylpropionates, phenylbutyrates, citrates, lactates, γ-hydroxybutyrates, glycolates, tartrates, and mandelates. Lists of other suitable pharmaceutically acceptable salts are found in REMINGTON: THE SCIENCE AND PRACTICE OF PHARMACY, 21^{st} Edition, Lippincott Wiliams and Wilkins, Philadelphia, Pa., 2006.

Examples of “pharmaceutically acceptable salts” of the compounds disclosed herein also include salts derived from an appropriate base, such as an alkali metal (for example, sodium, potassium), an alkaline earth metal (for example, magnesium), ammonium and NX₄⁺ (wherein X is C₁-C₄ alkyl). Also included are base addition salts, such as sodium or potassium salts.

Provided are also compounds described herein or pharmaceutically acceptable salts, isomers, or a mixture thereof, in which from 1 to n hydrogen atoms attached to a carbon atom can be replaced by a deuterium atom or D, in which n is the number of hydrogen atoms in the molecule. As known in the art, the deuterium atom is a non-radioactive isotope of the hydrogen atom. Such compounds can increase resistance to metabolism, and thus can be useful for increasing the half-life of the compounds described herein or pharmaceutically acceptable salts, isomer, or a mixture thereof when administered to a mammal. See, e.g., Foster, “Deuterium Isotope Effects in Studies of Drug Metabolism,” TRENDS PHARMACOL. SCI., 5(12):524-527 (1984). Such compounds are synthesized by means well known in the art, for example by employing starting materials in which one or more hydrogen atoms have been replaced by deuterium. The compounds disclosed herein can be deuterated at various positions, including (but not limited to), the following positions:

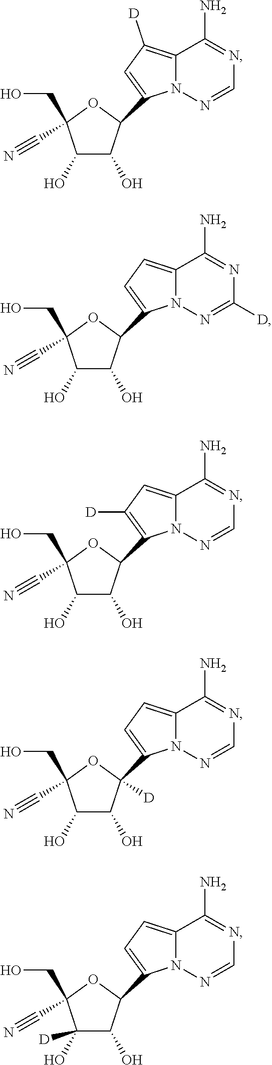

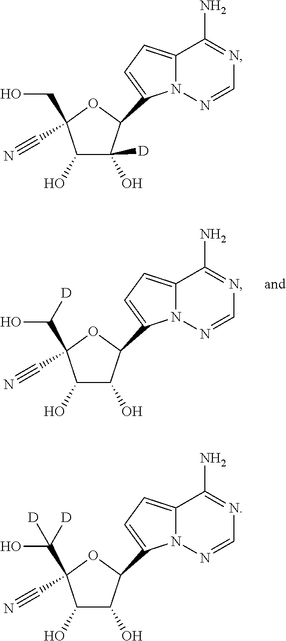 [SMILES: [2H][C@]1(O)[C@H](c2ccc3c(N)ncnn23)O[C@](C#N)(CO)[C@H]1O ; [2H]C(O)[C@@]1(C#N)O[C@@H](c2ccc3c(N)ncnn23)[C@H](O)[C@@H]1O ; "and" ; [2H]C([2H])(O)[C@@]1(C#N)O[C@@H](c2ccc3c(N)ncnn23)[C@H](O)[C@@H]1O]

Examples of isotopes that can be incorporated into the disclosed compounds also include isotopes of hydrogen, carbon, nitrogen, oxygen, phosphorous, fluorine, chlorine, and iodine, such as ²H, ³H, ¹¹C, ¹³C, ¹⁵C, ¹³N, ¹⁵N, ¹⁵O, ¹⁷O, ¹⁸O, ³¹P, ³²P, ³⁵S, ¹⁸F, ³⁶Cl, ¹²³I, and ¹²⁵I respectively. Substitution with positron emitting isotopes, such as ¹¹C, ¹⁸F, ¹⁵O and ¹³N, can be useful in Positron Emission Topography (PET) studies for examining substrate receptor occupancy. Isotopically-labeled compounds of Formula I-II, can generally be prepared by conventional techniques known to those skilled in the art or by processes analogous to those described in the Examples as set out below using an appropriate isotopically-labeled reagent in place of the non-labeled reagent previously employed.

The compounds of the embodiments disclosed herein, or their pharmaceutically acceptable salts can contain one or more asymmetric centers and can thus give rise to enantiomers, diastereomers, and other stereoisomeric forms that can be defined, in terms of absolute stereochemistry, as (R)- or (S)- or, as (D)- or (L)- for amino acids. The present disclosure is meant to include all such possible isomers, as well as their racemic and optically pure forms. Optically active (+) and (−), (R)- and (S)-, or (D)- and (L)-isomers can be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high-pressure liquid chromatography (HPLC). When the compounds described herein contain olefinic double bonds or other centers of geometric asymmetry, and unless specified otherwise, it is intended that the compounds include both E and Z geometric isomers. Likewise, all tautomeric forms are also intended to be included. Where compounds are represented in their chiral form, it is understood that the embodiment encompasses, but is not limited to, the specific diastereomerically or enantiomerically enriched form. Where chirality is not specified but is present, it is understood that the embodiment is directed to either the specific diastereomerically or enantiomerically enriched form; or a racemic or scalemic mixture of such compound(s). As used herein, “scalemic mixture” is a mixture of stereoisomers at a ratio other than 1:1.

The terms “prevention” or “preventing” means any treatment of a disease or condition that causes the clinical symptoms of the disease or condition not to develop. The term “prevention” or “preventing” also encompasses the administration of a compound or composition according to the embodiments disclosed herein post-exposure of the subject to the virus but before the appearance of symptoms of the disease, and/or prior to the detection of the virus in the blood, to prevent the appearance of symptoms of the disease and/or to prevent the virus from reaching detectible levels in the blood, and the administration of a compound or composition according to the embodiments disclosed herein to prevent perinatal transmission of viral infection from mother to baby, by administration to the mother before giving birth and to the child within the first days of life.

“Racemates” refers to a mixture of enantiomers. The mixture can comprise equal or unequal amounts of each enantiomer.

“Stereoisomer” and “stereoisomers” refer to compounds that differ in the chirality of one or more stereocenters. Stereoisomers include enantiomers and diastereomers. The compounds can exist in stereoisomeric form if they possess one or more asymmetric centers or a double bond with asymmetric substitution and, therefore, can be produced as individual stereoisomers or as mixtures. Unless otherwise indicated, the description is intended to include individual stereoisomers as well as mixtures. The methods for the determination of stereochemistry and the separation of stereoisomers are well-known in the art (see, e.g., Chapter 4 of ADVANCED ORGANIC CHEMISTRY, 4th ed., J. March, John Wiley & Sons, New York, 1992).

A “subject” or “patient” is meant to describe a human or vertebrate animal including a dog, cat, pocket pet, marmoset, horse, cow, pig, sheep, goat, elephant, giraffe, chicken, lion, monkey, owl, rat, squirrel, slender loris, and mouse. A “pocket pet” refers to a group of vertebrate animals capable of fitting into a commodious coat pocket such as, for example, hamsters, chinchillas, ferrets, rats, guinea pigs, gerbils, rabbits and sugar gliders.

Unless defined otherwise, all technical and scientific terms used herein have the same meaning as commonly understood by one of ordinary skill in the art. A dash at the front or end of a chemical group is a matter of convenience; chemical groups can be depicted with or without one or more dashes without losing their ordinary meaning. A wavy line drawn through a line in a structure indicates a point of attachment of a group. A dashed line indicates an optional bond.

Unless chemically or structurally required, no directionality is indicated or implied by the order in which a chemical group is written or the point at which it is attached to the remainder of the molecule. For instance, the group “—SO₂CH₂—” is equivalent to “—CH₂SO₂—” and both can be connected in either direction. Similarly, an “arylalkyl” group, for example, can be attached to the remainder of the molecule at either an aryl or an alkyl portion of the group. A prefix such as “Cᵤ-Cᵥ” or “(Cᵤ-Cᵥ)” indicates that the following group has from u to v carbon atoms. For example, “C₁₋₆ alkyl” and “C₁-C₆ alkyl” both indicate that the alkyl group has from 1 to 6 carbon atoms.

Unless otherwise specified, the carbon atoms of the compounds of Formulas I-II are intended to have a valence of four. If in some chemical structure representations, carbon atoms do not have a sufficient number of variables attached to produce a valence of four, the remaining carbon substituents needed to provide a valence of four should be assumed to be hydrogen.

The terms “treating” and “treatment” as used herein are intended to mean the administration of a compound or composition according to the embodiments disclosed herein to alleviate or eliminate symptoms of a viral infection and/or to reduce viral load in a subject.

The term “therapeutically effective amount,” as used herein, is the amount of compound disclosed herein present in a formulation described herein that is needed to provide a desired level of drug in the secretions and tissues of the airways and lungs, or alternatively, in the bloodstream of a subject to be treated to give an anticipated physiological response or desired biological effect when such a formulation is administered by the chosen route of administration. The precise amount will depend upon numerous factors, for example the particular compound disclosed herein, the specific activity of the formulation, the delivery device employed, the physical characteristics of the formulation, its intended use, as well as subject considerations such as severity of the disease state, subject cooperation, etc., and can readily be determined by one skilled in the art based upon the information provided herein. The term “therapeutically effective amount” or “effective amount” also means amounts that eliminate or reduce the subject's viral burden and/or viral reservoir.

The term “adjacent carbons” as used herein refers to consecutive carbons atoms that are directly attached to each other. For example,

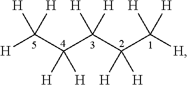

C₁ and C₂ are adjacent carbons, C₂ and C₃ are adjacent carbons, C₃ and C₄ are adjacent carbons, and C₄ and C₅ are adjacent carbons. Similarly, in

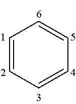

C₁ and C₂ are adjacent carbons, C₂ and C₃ are adjacent carbons, C₃ and C₄ are adjacent carbons, and C₄ and C₅ are adjacent carbons, C₅ and C₆ are adjacent carbons and C₆ and C₁ are adjacent carbons.

“Solvate” as used herein refers to the result of the interaction of a solvent and a compound. Solvates of salts of the compounds described herein are also provided. Hydrates of the compounds described herein are also provided.

“Prodrug” as used herein refers to a derivative of a drug that upon administration to the human body is converted to the parent drug according to some chemical or enzymatic pathway.

As used herein, “pharmaceutically acceptable carrier” or “pharmaceutically acceptable excipient” includes, but is not limited to, any and all solvents, dispersion media, coatings, antibacterial and antifungal agents, isotonic and absorption delaying agents, and combinations thereof. The use of pharmaceutically acceptable carriers and pharmaceutically acceptable excipients for pharmaceutically active substances is well known in the art. Except insofar as any conventional media or agent is incompatible with the active ingredient, its use in the therapeutic formulations is contemplated. Supplementary active ingredients can also be incorporated into the formulations. The carrier(s) must be “acceptable” in the sense of being compatible with the other ingredients of the formulation and physiologically innocuous to the recipient thereof.

### III. Compounds

Disclosed herein are, among other things, compounds of Formula I:

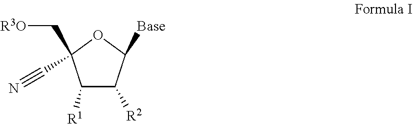

or a pharmaceutically acceptable salt thereof, wherein:
R¹ is OH, OC(O)R⁴, or OC(O)OR⁴, and
R² is OH, OC(O)R⁵, or OC(O)OR⁵; or
R¹ and R² are taken together to form —OC(O)O— or —OCHR⁶O—, wherein
   R⁶ is H, C₁-C₆ alkyl, C₁-C₆ alkoxy, or C₆-C₁₀ aryl;
R³ is H or C(O)OR⁷, or
R¹ and R³ are taken together to form —OC(O)—;
R⁴, R⁵, and R⁷ are each independently C₁-C₈ alkyl, C₂-C₈ alkenyl, C₂-C₈ alkynyl, C₃-C₈ carbocyclyl, C₆-C₁₀ aryl, 4 to 6 membered heterocyclyl containing 1, 2, or 3 O, or 5 to 6 membered heteroaryl containing 1, 2, or 3 heteroatoms selected from N, O, and S;
wherein R⁴, R⁵, and R⁷ are each, independently, optionally substituted with one, two or three substituents independently selected from the group consisting of halo, oxo, cyano, —N₃, —OR⁸, C₁-C₈ alkyl, —NR⁹R¹⁰, C₃-C₈ carbocyclyl, and phenyl optionally substituted with one, two, or three substituents independently selected from halo, cyano, and C₁-C₆ alkyl; and
   each R⁸ is independently H, C₁-C₆ alkyl, C₁-C₆ haloalkyl, and C₃-C₆ cycloalkyl;
   each R⁹ is independently H, C₁-C₆ alkyl, C₁-C₆ haloalkyl, and C₃-C₆ cycloalkyl;
   each R¹⁰ is independently H, C₁-C₆ alkyl, C₁-C₆ haloalkyl, and C₃-C₆ cycloalkyl; and
Base is

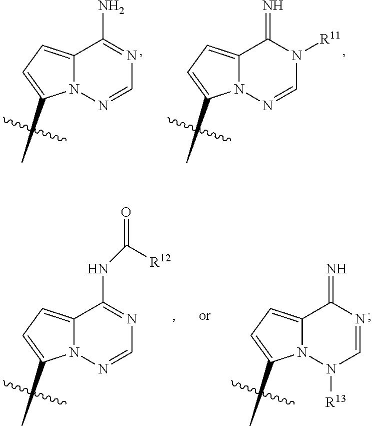

wherein
   R¹¹ is C₁-C₆ alkyl substituted with —OP(O)(OH)₂ or —OC(O)R¹²;
   R¹² is C₁-C₈ alkyl or C₁-C₈ alkoxy; and
   R¹³ is C₁-C₆ alkyl substituted with —OP(O)(OH)₂ or —OC(O)R¹²;
   wherein the compound is not

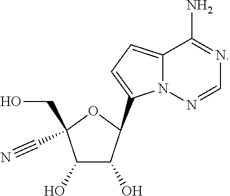 [SMILES: N#C[C@]1(CO)O[C@@H](c2ccc3c(N)ncnn23)[C@H](O)[C@@H]1O]

In some embodiments, the compound of Formula I has a Formula II.

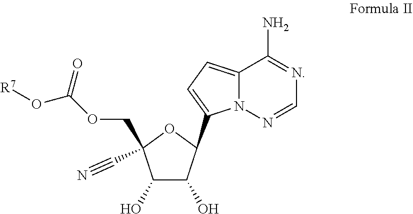

R¹ can be OH, OC(O)R⁴, or OC(O)OR⁴. In some embodiments, R¹ is OH. In some embodiments, R¹ is OC(O)R⁴. In some embodiments, R¹ is OC(O)OR⁴.

R² can be OH, OC(O)R⁵, or OC(O)OR⁵. In some embodiments, R² is OH. In some embodiments, R² is OC(O)R⁵. In some embodiments, R² is OC(O)OR⁵.

In some embodiments, R¹ is OH and R² is OH. In some embodiments, R¹ and R² are taken together to form —OC(O)O—.

In some embodiments, R¹ and R² are taken together to form —OCH₂O—. In some embodiments, R¹ and R² are taken together to form —OCR⁶O—.

R³ can be H or C(O)OR⁷. In some embodiments, R³ is H. In some embodiments, R³ is C(O)OR⁷. In some embodiments, R³ is selected from

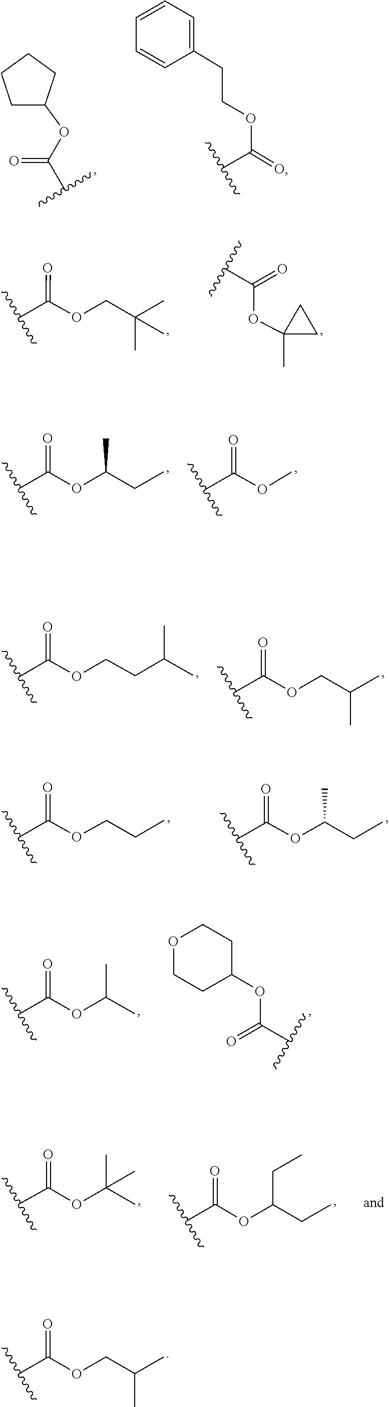

In some embodiments, R³ is selected from

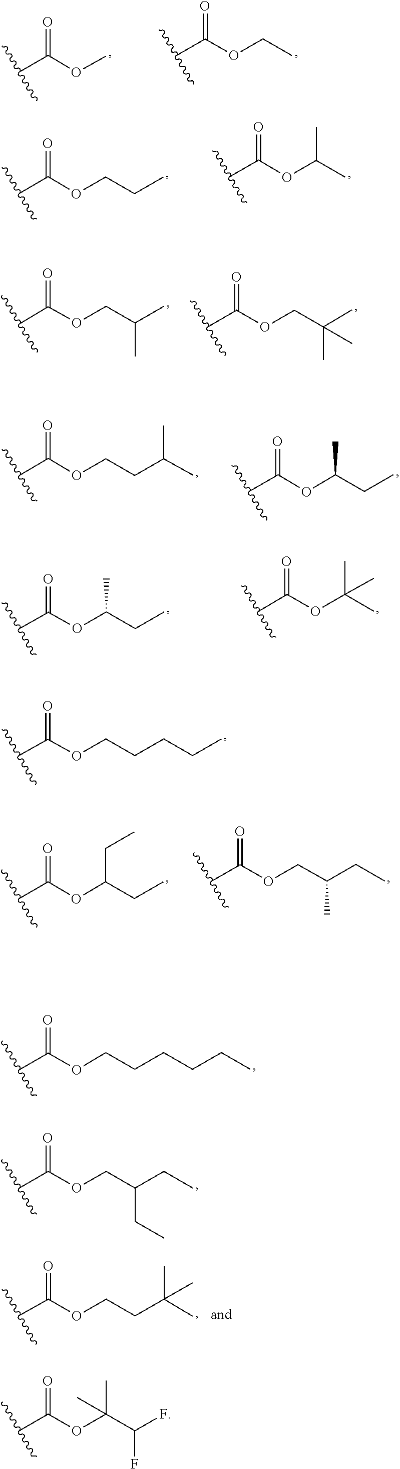

In some embodiments, R³ is selected from

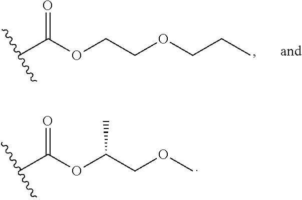

In some embodiments, R³ is selected from

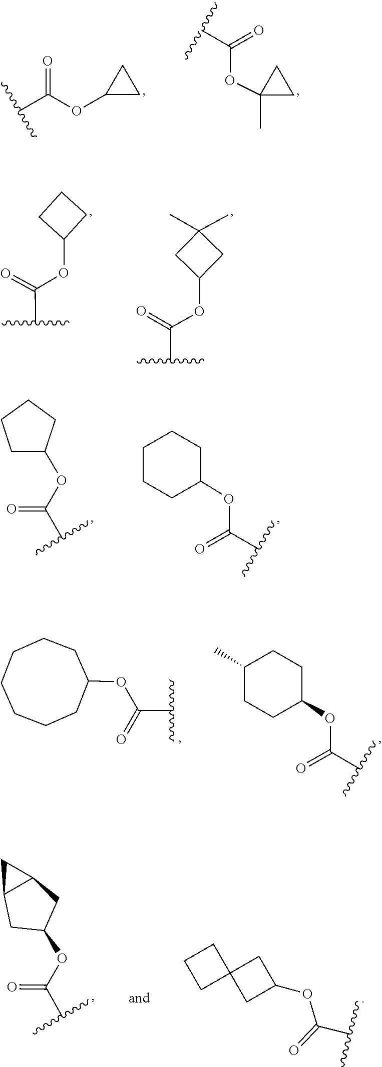

In some embodiments, R³ is selected from

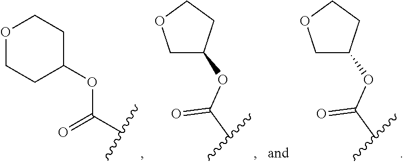 [SMILES: *C(=O)OC1CCOCC1 ; *C(=O)O[C@@H]1CCOC1 ; *C(=O)O[C@H]1CCOC1]

In some embodiments, R³ is selected from

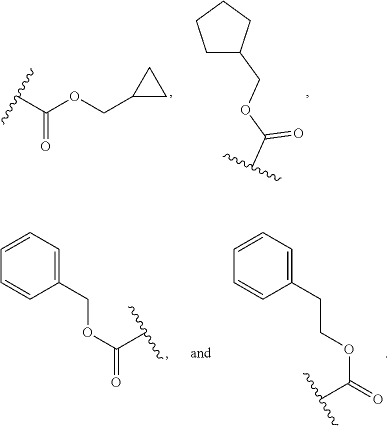 [SMILES: *C(=O)OCC1CC1 ; *C(=O)OCC1CCCC1 ; *C(=O)OCc1ccccc1 ; *C(=O)OCCc1ccccc1]

In some embodiments, R¹ and R³ are taken together to form —OC(O)—.

R⁴ can be C₁-C₈ alkyl, C₂-C₈ alkenyl, C₂-C₈ alkynyl, C₃-C₈ carbocyclyl, C₆-C₁₀ aryl, 4 to 6 membered heterocyclyl containing 1, 2, or 3 O, or 5 to 6 membered heteroaryl containing 1, 2, or 3 heteroatoms selected from N, O, and S; wherein the alkyl, alkenyl, alkynyl, carbocyclyl, aryl, heterocyclyl or heteroaryl of R⁴ can be optionally substituted with one, two or three substituents independently selected from the group consisting of halo, oxo, cyano, —N₃, —OR⁸, C₁-C₈ alkyl, —NR⁹R¹⁰, C₃-C₈ carbocyclyl, and phenyl optionally substituted with one, two, or three substituents independently selected from halo, cyano, and C₁-C₆ alkyl; and R⁸ can be H, C₁-C₆ alkyl, C₁-C₆ haloalkyl, and C₃-C₆ cycloalkyl; R⁹ can be H, C₁-C₆ alkyl, C₁-C₆ haloalkyl, and C₃-C₆ cycloalkyl; and R¹⁰ can be H, C₁-C₆ alkyl, C₁-C₆ haloalkyl, and C₃-C₆ cycloalkyl.

In some embodiments, R⁴ is a C₁-C₆ (e.g., C₁, C₂, C₃, C₄, C₅, or C₆) alkyl. In some embodiments, the C₁-C₆ alkyl is methyl, ethyl, -n-propyl, isopropyl, -n-butyl, isobutyl, -s-butyl, -t-butyl, -n-pentyl, -2-pentyl, -3-pentyl, -2-methyl-2-butyl, -3-methyl-2-butyl, -3-methyl-1-butyl, -2-methyl-1-butyl, -1-hexyl, -2-hexyl, -3-hexyl, -2-methyl-2-pentyl, -3-methyl-2-pentyl, -4-methyl-2-pentyl, -3-methyl-3-pentyl, -2-methyl-3-pentyl, -2,3-dimethyl-2-butyl, or -3,3-dimethyl-2-butyl. In some embodiments, the C₁-C₆ alkyl is C₁-C₃ alkyl. In some embodiments, the C₁-C₆ alkyl is C₂-C₅ alkyl. In some embodiments, the C₁-C₆ alkyl is C₄-C₆ alkyl. In some embodiments, the C₁-C₆ alkyl is a branched C₁-C₆ alkyl. In some embodiments, the C₁-C₆ alkyl is an unbranched C₁-C₆ alkyl. In some embodiments, the C₁-C₆ alkyl is an unsubstituted C₁-C₆ alkyl. In some embodiments, the C₁-C₆ alkyl is a substituted C₁-C₆ alkyl. In some embodiments, the C₁-C₆ alkyl is a branched, unsubstituted C₁-C₆ alkyl. In some embodiments, the C₁-C₆ alkyl is a branched, substituted C₁-C₆ alkyl. In some embodiments, the C₁-C₆ alkyl is an unbranched, unsubstituted C₁-C₆ alkyl. In some embodiments, R⁴ is C₃-C₈ alkyl. In some embodiments, R⁴ is tert-butyl or isobutyl.

In some embodiments, R⁴ is C₂-C₈ (e.g., C₂, C₃, C₄, C₅, C₆, C₇, or C₈) alkenyl. In some embodiments, the C₂-C₅ alkenyl has at least one (e.g., at least 2, at least 3, at least 3, at least 4, or at least 5) double bond. In some embodiments, the C₂-C₅ alkenyl is an unsubstituted C₂-C₅ alkenyl. In some embodiments, the C₂-C₅ alkenyl is a substituted C₂-C₅ alkenyl. Examples of the C₂-C₅ alkenyl include, but are not limited to, vinyl (ethenyl), propenyl, isopropenyl, 1-butenyl, 2-butenyl, isobutenyl, butadienyl, 1-pentenyl, 2-pentenyl, isopentenyl, 1,3-pentadienyl, 1,4-pentadienyl, 1-hexenyl, 2-hexenyl, 3-hexenyl, 1,3-hexadienyl, 1,4-hexadienyl, 1,5-hexadienyl, 2,4-hexadienyl, or 1,3,5-hexatrienyl. In some embodiments, R⁴ is unsubstituted C₂-C₈ alkenyl. In some embodiments, R⁴ is substituted C₂-C₈ alkenyl.

In some embodiments, R⁴ is C₂-C₈ (e.g., C₂, C₃, C₄, C₅, C₆, C₇, or C₈) alkynyl. In some embodiments, the C₂-C₈ alkynyl has at least one triple bond. In some embodiments, the C₂-C₈ alkynyl has at least one triple bond and at least one double bond. Exemplary C₂-C₈ alkynyl include, but are not limited to, ethynyl, prop-1-ynyl, but-1-ynyl, pent-1-ynyl, pent-4-ynyl and penta-1,4-diynyl. In some embodiments, the C₂-C₅ alkynyl is unsubstituted C₂-C₅ alkynyl. In some embodiments, the C₂-C₈ alkynyl is substituted C₂-C₈ alkynyl. In some embodiments, the C₂-C₈ alkynyl is branched C₂-C₈ alkynyl. In some embodiments, the C₂-C₈ alkynyl is unbranched C₂-C₈ alkynyl.

In some embodiments, R⁴ is C₃-C₈ (e.g., C₃, C₄, C₅, C₆, C₇, or C₈) carbocyclyl. In some embodiments, the C₃-C₈ carbocyclyl is saturated. In some embodiments, the C₃-C₈ carbocyclyl is partially unsaturated. Exemplary C₃-C₈ carbocyclic rings include, but are not limited to, cyclopropane, cyclobutane, cyclopentane, cyclopentene, cyclohexane, cyclohexene, 1,3-cyclohexadiene, 1,4-cyclohexadiene, cycloheptane, cycloheptene, and cyclooctane. In some embodiments, the C₃-C₈ carbocyclyl is unsubstituted C₃-C₈ carbocyclyl. In some embodiments, the C₃-C₈ carbocyclyl is substituted C₃-C₈ carbocyclyl. In some embodiments, the C₃-C₈ carbocyclyl is unbranched C₃-C₈ carbocyclyl. In some embodiments, the C₃-C₈ carbocyclyl is branched C₃-C₈ carbocyclyl.

In some embodiments, R⁴ is C₆-C₁₀ aryl. In some embodiments, the C₆-C₁₀ aryl is C₆ aryl (e.g., phenyl) or C₁₀ aryl (e.g., naphthyl). In some embodiments, the C₆-C₁₀ aryl is unsubstituted C₆-C₁₀ aryl. In some embodiments, the C₆-C₁₀ aryl is substituted C₆-C₁₀ aryl. In some embodiments, the C₆-C₁₀ aryl is phenyl. In some embodiments, the C₆-C₁₀ aryl is naphthyl.

In some embodiments, R⁴ is 4- to 6- (e.g., 4-, 5-, 6-) membered heterocyclyl containing one, two, or three O. In some embodiments, the 4- to 6-membered heterocyclyl is 4-membered heterocyclyl containing one heteroatom selected from O. In some embodiments, the 4- to 6-membered heterocyclyl is 4-membered heterocyclyl containing two O. In some embodiments, the 4- to 6-membered heterocyclyl is 4-membered heterocyclyl containing three O. In some embodiments, the 4- to 6-membered heterocyclyl is 5-membered heterocyclyl containing one O. In some embodiments, the 4- to 6-membered heterocyclyl is 5-membered heterocyclyl containing two O. In some embodiments, R⁴ is 5-membered heterocyclyl containing three O. In some embodiments, the 4- to 6-membered heterocyclyl is 6-membered heterocyclyl containing one m O. In some embodiments, the 4- to 6-membered heterocyclyl is 6-membered heterocyclyl containing two O. In some embodiments, the 4- to 6-membered heterocyclyl is 6-membered heterocyclyl containing three O. In some embodiments, the 4- to 6-membered heterocyclyl is an unsubstituted 4- to 6-membered heterocyclyl. In some embodiments, the 4- to 6-membered heterocyclyl is a substituted 4- to 6-membered heterocyclyl.

In some embodiments, R⁴ is 5- to 6-membered heteroaryl containing 1, 2, or 3 heteroatoms selected from N, O, and S. In some embodiments, the 5- to 6-membered heteroaryl is 5-membered heteroaryl containing one heteroatom selected from N, O, and S. In some embodiments, the 5- to 6-membered heteroaryl is 5-membered heteroaryl containing two heteroatoms selected from N, O, and S. In some embodiments, the 5- to 6-membered heteroaryl is 5-membered heteroaryl containing three heteroatoms selected from N, O, and S. In some embodiments, the 5- to 6-membered heteroaryl is 6-membered heteroaryl containing one heteroatom selected from N, O, and S. In some embodiments, the 5- to 6-membered heteroaryl is 6-membered heteroaryl containing two heteroatoms selected from N, O, and S. In some embodiments, the 5- to 6-membered heteroaryl is 6-membered heteroaryl containing three heteroatoms selected from N, O, and S. In some embodiments, the 5- to 6-membered heteroaryl is pyridinyl. In some embodiments, the 5- to 6-membered heteroaryl is pyrimidinyl. In some embodiments, the 5- to 6-membered heteroaryl is an unsubstituted 5- to 6-membered heteroaryl. In some embodiments, the 5- to 6-membered heteroaryl is a substituted 5- to 6-membered heteroaryl.

In some embodiments, R⁴ is unsubstituted. In some embodiments, R⁴ is substituted with one substituent. In some embodiments, R⁴ is substituted with two substituents. In some embodiments, R⁴ is substituted three substituents. In some embodiments, R⁴ is substituted with one, two or three substituents wherein at least one substituent is halo. In some embodiments, R⁴ is substituted with one, two or three substituents wherein at least one substituent is oxo. In some embodiments, R⁴ is substituted with one, two or three substituents wherein at least one substituent is cyano. In some embodiments, R⁴ is substituted with one, two or three substituents wherein at least one substituent is N₃. In some embodiments, R⁴ is substituted with one, two or three substituents wherein at least one substituent is OR⁸.

In some embodiments, R⁴ is substituted with one, two or three substituents wherein at least one substituent is a C₁-C₈ (e.g., C₁, C₂, C₃, C₄, C₅, C₆, C₇, or C₈) alkyl. In some embodiments, the C₁-C₈ alkyl is methyl, ethyl, -n-propyl, isopropyl, -n-butyl, isobutyl, -s-butyl, -t-butyl, -n-pentyl, -2-pentyl, -3-pentyl, -2-methyl-2-butyl, -3-methyl-2-butyl, -3-methyl-1-butyl, -2-methyl-1-butyl, -1-hexyl, -2-hexyl, -3-hexyl, -2-methyl-2-pentyl, -3-methyl-2-pentyl, -4-methyl-2-pentyl, -3-methyl-3-pentyl, -2-methyl-3-pentyl, -2,3-dimethyl-2-butyl, or -3,3-dimethyl-2-butyl. In some embodiments, the C₁-C₈ alkyl is C₁-C₆ alkyl. In some embodiments, the C₁-C₈ alkyl is C₁-C₃ alkyl. In some embodiments, the C₁-C₈ alkyl is C₂-C₅ alkyl. In some embodiments, the C₁-C₈ alkyl is C₄-C₈ alkyl. In some embodiments, the C₁-C₈ alkyl is a branched C₁-C₈ alkyl. In some embodiments, the C₁-C₈ alkyl is an unbranched C₁-C₈ alkyl. In some embodiments, the C₁-C₈ alkyl is an unsubstituted C₁-C₈ alkyl. In some embodiments, the C₁-C₈ alkyl is a branched, unsubstituted C₁-C₈ alkyl. In some embodiments, the C₁-C₈ alkyl is an unbranched, unsubstituted C₁-C₈ alkyl.

In some embodiments, R⁴ is substituted with one, two or three substituents wherein at least one substituent is NR⁹R¹⁰. In some embodiments, R⁴ is substituted with one, two or three substituents wherein at least one substituent is NH₂.

In some embodiments, R⁴ is substituted with one, two or three substituents wherein at least one substituent is C₃-C₈ (e.g., C₃, C₄, C₅, C₆, C₇, or C₈) carbocyclyl. In some embodiments, the C₃-C₈ carbocyclyl is saturated. In some embodiments, the C₃-C₈ carbocyclyl is partially unsaturated. Exemplary C₃-C₈ carbocyclic rings include, but are not limited to, cyclopropane, cyclobutane, cyclopentane, cyclopentene, cyclohexane, cyclohexene, 1,3-cyclohexadiene, 1,4-cyclohexadiene, cycloheptane, cycloheptene, and cyclooctane. In some embodiments, the C₃-C₈ carbocyclyl is unsubstituted C₃-C₈ carbocyclyl. In some embodiments, the C₃-C₈ carbocyclyl is substituted C₃-C₈ carbocyclyl. In some embodiments, the C₃-C₈ carbocyclyl is unbranched C₃-C₈ carbocyclyl. In some embodiments, the C₃-C₈ carbocyclyl is branched C₃-C₈ carbocyclyl.

In some embodiments, R⁴ is substituted with one, two or three substituents wherein at least one substituent is phenyl. In some embodiments, R⁴ is substituted with one, two or three substituents wherein at least one substituent is phenyl substituted with one, two, or three substituents independently selected from halo, cyano, and C₁-C₆ alkyl. In some embodiments, R⁴ is substituted with one, two or three substituents wherein at least one substituent is phenyl substituted with one, two, or three substituents wherein at least one substituent is halo (e.g., fluoro, chloro, iodo, bromo). In some embodiments, R⁴ is substituted with one, two or three substituents wherein at least one substituent is phenyl substituted with one, two, or three substituents wherein at least one substituent is cyano. In some embodiments, R⁴ is substituted with at least one halo and at least one oxo. In some embodiments, R⁴ is substituted with at least one halo and at least one cyano. In some embodiments, R⁴ is substituted with at least one halo and at least one N₃. In some embodiments, R⁴ is substituted with at least one halo and at least one OR⁸. In some embodiments, R⁴ is substituted with at least one halo and at least one NR⁹R¹⁰. In some embodiments, R⁴ is substituted with at least one halo and at least one C₃-C₈ carbocyclyl. In some embodiments, R⁴ is substituted with at least one halo and at least one unsubstituted phenyl. In some embodiments, R⁴ is substituted with at least one halo and at least one phenyl substituted with one, two, or three substituents independently selected from halo, cyano, and C₁-C₆ alkyl. In some embodiments, R⁴ is substituted with at least one oxo and at least one cyano. In some embodiments, R⁴ is substituted with at least one oxo and at least one N3. In some embodiments, R⁴ is substituted with at least one oxo and at least one OR⁸. In some embodiments, R⁴ is substituted with at least one oxo and at least one C₁-C₈ alkyl. In some embodiments, R⁴ is substituted with at least one oxo and at least one NR⁹R¹⁰. In some embodiments, R⁴ is substituted with at least one oxo and at least one C₃-C₈ carbocyclyl. In some embodiments, R⁴ is substituted with at least one oxo and at least one unsubstituted phenyl. In some embodiments, R⁴ is substituted with at least one oxo and at least one phenyl substituted with one, two, or three substituents independently selected from halo, cyano, and C₁-C₆ alkyl. In some embodiments, R⁴ is substituted with at least one cyano and at least one N₃. In some embodiments, R⁴ is substituted with at least one N₃ and at least one OR⁸. In some embodiments, R⁴ is substituted with at least one cyano and at least one C₁-C₈ alkyl. In some embodiments, R⁴ is substituted with at least one cyano and at least one NR⁹R¹⁰. In some embodiments, R⁴ is substituted with at least one cyano and at least one C₃-C₈ carbocyclyl. In some embodiments, R⁴ is substituted with at least one cyano and at least one unsubstituted phenyl. In some embodiments, R⁴ is substituted with at least one cyano and at least one phenyl substituted with one, two, or three substituents independently selected from halo, cyano, and C₁-C₆ alkyl. In some embodiments, R⁴ is substituted with at least one N₃ and at least one OR⁸. In some embodiments, R⁴ is substituted with at least one N₃ and at least one C₁-C₈ alkyl. In some embodiments, R⁴ is substituted with at least one N₃ and at least one NR⁹R¹⁰. In some embodiments, R⁴ is substituted with at least one N₃ and at least one C₃-C₈ carbocyclyl. In some embodiments, R⁴ is substituted with at least one N₃ and at least one unsubstituted phenyl. In some embodiments, R⁴ is substituted with at least one N₃ and at least one phenyl substituted with one, two, or three substituents independently selected from halo, cyano, and C₁-C₆ alkyl. In some embodiments, R⁴ is substituted with at least one OR⁸ and at least one C₁-C₈ alkyl. In some embodiments, R⁴ is substituted with at least one OR⁸ and at least one NR⁹R¹⁰. In some embodiments, R⁴ is substituted with at least one OR⁸ and at least one C₃-C₈ carbocyclyl. In some embodiments, R⁴ is substituted with at least one OR⁸ and at least one unsubstituted phenyl. In some embodiments, R⁴ is substituted with at least one OR⁸ and at least one phenyl substituted with one, two, or three substituents independently selected from halo, cyano, and C₁-C₆ alkyl. In some embodiments, R⁴ is substituted with at least one C₁-C₈ alkyl and at least one NR⁹R¹⁰. In some embodiments, R⁴ is substituted with at least one C₁-C₈ alkyl and at least one C₃-C₈ carbocyclyl. In some embodiments, R⁴ is substituted with at least one C₁-C₈ alkyl and at least one unsubstituted phenyl. In some embodiments, R⁴ is substituted with at least one C₁-C₈ alkyl and at least one phenyl substituted with one, two, or three substituents independently selected from halo, cyano, and C₁-C₆ alkyl. In some embodiments, R⁴ is substituted with at least one C₃-C₈ carbocyclyl and at least one unsubstituted phenyl. In some embodiments, R⁴ is substituted with at least one C₃-C₈ carbocyclyl and at least one phenyl substituted with one, two, or three substituents independently selected from halo, cyano, and C₁-C₆ alkyl.

R⁵ can be C₁-C₈ alkyl, C₂-C₈ alkenyl, C₂-C₈ alkynyl, C₃-C₈ carbocyclyl, C₆-C₁₀ aryl, 4 to 6 membered heterocyclyl containing 1, 2, or 3 O, or 5 to 6 membered heteroaryl containing 1, 2, or 3 heteroatoms selected from N, O, and S; wherein R⁵ can be optionally substituted with one, two or three substituents independently selected from the group consisting of halo, oxo, cyano, —N₃, —OR⁸, C₁-C₈ alkyl, —NR⁹R¹⁰, C₃-C₈ carbocyclyl, and phenyl optionally substituted with one, two, or three substituents independently selected from halo, cyano, and C₁-C₆ alkyl; and R⁸ can be H, C₁-C₆ alkyl, C₁-C₆ haloalkyl, and C₃-C₆ cycloalkyl; R⁹ can be H, C₁-C₆ alkyl, C₁-C₆ haloalkyl, and C₃-C₆ cycloalkyl; and R¹⁰ can be H, C₁-C₆ alkyl, C₁-C₆ haloalkyl, and C₃-C₆ cycloalkyl.

In some embodiments, R⁵ is a C₁-C₆ (e.g., C₁, C₂, C₃, C₄, C₅, or C₆) alkyl. In some embodiments, the C₁-C₆ alkyl is methyl, ethyl, -n-propyl, isopropyl, -n-butyl, isobutyl, -s-butyl, -t-butyl, -n-pentyl, -2-pentyl, -3-pentyl, -2-methyl-2-butyl, -3-methyl-2-butyl, -3-methyl-1-butyl, -2-methyl-1-butyl, -1-hexyl, -2-hexyl, -3-hexyl, -2-methyl-2-pentyl, -3-methyl-2-pentyl, -4-methyl-2-pentyl, -3-methyl-3-pentyl, -2-methyl-3-pentyl, -2,3-dimethyl-2-butyl, or -3,3-dimethyl-2-butyl. In some embodiments, the C₁-C₆ alkyl is C₁-C₃ alkyl. In some embodiments, the C₁-C₆ alkyl is C₂-C₅ alkyl. In some embodiments, the C₁-C₆ alkyl is C₄-C₆ alkyl. In some embodiments, the C₁-C₆ alkyl is a branched C₁-C₆ alkyl. In some embodiments, the C₁-C₆ alkyl is an unbranched C₁-C₆ alkyl. In some embodiments, the C₁-C₆ alkyl is an unsubstituted C₁-C₆ alkyl. In some embodiments, the C₁-C₆ alkyl is a substituted C₁-C₆ alkyl. In some embodiments, the C₁-C₆ alkyl is a branched, unsubstituted C₁-C₆ alkyl. In some embodiments, the C₁-C₆ alkyl is a branched, substituted C₁-C₆ alkyl. In some embodiments, the C₁-C₆ alkyl is an unbranched, unsubstituted C₁-C₆ alkyl.

In some embodiments, R⁵ is C₂-C₈ (e.g., C₂, C₃, C₄, C₅, C₆, C₇, or C₈) alkenyl. In some embodiments, the C₂-C₈ alkenyl has at least one (e.g., at least 2, at least 3, at least 3, at least 4, or at least 5) double bond. In some embodiments, the C₂-C₈ alkenyl is an unsubstituted C₂-C₈ alkenyl.

In some embodiments, the C₂-C₅ alkenyl is a substituted C₂-C₈ alkenyl. Examples of the C₂-C₈ alkenyl include, but are not limited to, vinyl (ethenyl), propenyl, isopropenyl, 1-butenyl, 2-butenyl, isobutenyl, butadienyl, 1-pentenyl, 2-pentenyl, isopentenyl, 1,3-pentadienyl, 1,4-pentadienyl, 1-hexenyl, 2-hexenyl, 3-hexenyl, 1,3-hexadienyl, 1,4-hexadienyl, 1,5-hexadienyl, 2,4-hexadienyl, or 1,3,5-hexatrienyl. In some embodiments, R⁵ is unsubstituted C₂-C₈ alkenyl. In some embodiments, R⁵ is substituted C₂-C₈ alkenyl.

In some embodiments, R⁵ is C₂-C₈ (e.g., C₂, C₃, C₄, C₅, C₆, C₇, or C₈) alkynyl. In some embodiments, the C₂-C₈ alkynyl has at least one triple bond. In some embodiments, the C₂-C₈ alkynyl has at least one triple bond and at least one double bond. Exemplary C₂-C₈ alkynyl include, but are not limited to, ethynyl, prop-1-ynyl, but-1-ynyl, pent-1-ynyl, pent-4-ynyl and penta-1,4-diynyl. In some embodiments, the C₂-C₅ alkynyl is unsubstituted C₂-C₈ alkynyl. In some embodiments, the C₂-C₅ alkynyl is substituted C₂-C₅ alkynyl. In some embodiments, the C₂-C₅ alkynyl is branched C₂-C₈ alkynyl. In some embodiments, the C₂-C₈ alkynyl is unbranched C₂-C₈ alkynyl.

In some embodiments, R⁵ is C₃-C₈ (e.g., C₃, C₄, C₅, C₆, C₇, or C₈) carbocyclyl. In some embodiments, the C₃-C₈ carbocyclyl is saturated. In some embodiments, the C₃-C₈ carbocyclyl is partially unsaturated. Exemplary C₃-C₈ carbocyclic rings include, but are not limited to, cyclopropane, cyclobutane, cyclopentane, cyclopentene, cyclohexane, cyclohexene, 1,3-cyclohexadiene, 1,4-cyclohexadiene, cycloheptane, cycloheptene, and cyclooctane. In some embodiments, the C₃-C₈ carbocyclyl is unsubstituted C₃-C₈ carbocyclyl. In some embodiments, the C₃-C₈ carbocyclyl is substituted C₃-C₈ carbocyclyl. In some embodiments, the C₃-C₈ carbocyclyl is unbranched C₃-C₈ carbocyclyl. In some embodiments, the C₃-C₈ carbocyclyl is branched C₃-C₈ carbocyclyl.

In some embodiments, R⁵ is C₆-C₁₀ aryl. In some embodiments, the C₆-C₁₀ aryl is C₆ aryl (e.g., phenyl) or C₁₀ aryl (e.g., naphthyl). In some embodiments, the C₆-C₁₀ aryl is unsubstituted C₆-C₁₀ aryl. In some embodiments, the C₆-C₁₀ aryl is substituted C₆-C₁₀ aryl. In some embodiments, the C₆-C₁₀ aryl is phenyl. In some embodiments, the C₆-C₁₀ aryl is naphthyl.

In some embodiments, R⁵ is 4- to 6- (e.g., 4-, 5-, 6-) membered heterocyclyl containing one, two, or three O. In some embodiments, the 4- to 6-membered heterocyclyl is 4-membered heterocyclyl containing one heteroatom selected from O. In some embodiments, the 4- to 6-membered heterocyclyl is 4-membered heterocyclyl containing two O. In some embodiments, the 4- to 6-membered heterocyclyl is 4-membered heterocyclyl containing three O. In some embodiments, the 4- to 6-membered heterocyclyl is 5-membered heterocyclyl containing one O. In some embodiments, the 4- to 6-membered heterocyclyl is 5-membered heterocyclyl containing two O. In some embodiments, the 4- to 6-membered heterocyclyl is 5-membered heterocyclyl containing three O. In some embodiments, the 4- to 6-membered heterocyclyl is 6-membered heterocyclyl containing one O. In some embodiments, the 4- to 6-membered heterocyclyl is 6-membered heterocyclyl containing two O. In some embodiments, the 4- to 6-membered heterocyclyl is 6-membered heterocyclyl containing three O. In some embodiments, the 4- to 6-membered heterocyclyl is an unsubstituted 4- to 6-membered heterocyclyl. In some embodiments, the 4- to 6-membered heterocyclyl is a substituted 4- to 6-membered heterocyclyl.

In some embodiments, R⁵ is 5- to 6-membered heteroaryl containing 1, 2, or 3 heteroatoms selected from N, O, and S. In some embodiments, the 5- to 6-membered heteroaryl is 5-membered heteroaryl containing one heteroatom selected from N, O, and S. In some embodiments, the 5- to 6-membered heteroaryl is 5-membered heteroaryl containing two heteroatoms selected from N, O, and S. In some embodiments, the 5- to 6-membered heteroaryl is 5-membered heteroaryl containing three heteroatoms selected from N, O, and S. In some embodiments, the 5- to 6-membered heteroaryl is 6-membered heteroaryl containing one heteroatom selected from N, O, and S. In some embodiments, the 5- to 6-membered heteroaryl is 6-membered heteroaryl containing two heteroatoms selected from N, O, and S. In some embodiments, the 5- to 6-membered heteroaryl is 6-membered heteroaryl containing three heteroatoms selected from N, O, and S. In some embodiments, the 5- to 6-membered heteroaryl is pyridinyl. In some embodiments, the 5- to 6-membered heteroaryl is pyrimidinyl. In some embodiments, the 5- to 6-membered heteroaryl is an unsubstituted 5- to 6-membered heteroaryl. In some embodiments, the 5- to 6-membered heteroaryl is a substituted 5- to 6-membered heteroaryl.

In some embodiments, R⁵ is unsubstituted. In some embodiments, R⁵ is substituted with one substituent. In some embodiments, R⁵ is substituted with two substituents. In some embodiments, R⁵ is substituted three substituents. In some embodiments, R⁵ is substituted with one, two or three substituents wherein at least one substituent is halo. In some embodiments, R⁵ is substituted with one, two or three substituents wherein at least one substituent is oxo. In some embodiments, R⁵ is substituted with one, two or three substituents wherein at least one substituent is cyano. In some embodiments, R⁵ is substituted with one, two or three substituents wherein at least one substituent is N₃. In some embodiments, R⁵ is substituted with one, two or three substituents wherein at least one substituent is OR⁸.

In some embodiments, R⁵ is substituted with one, two or three substituents wherein at least one substituent is a C₁-C₈ (e.g., C₁, C₂, C₃, C₄, C₅, C₆, C₇, or C₈) alkyl. In some embodiments, the C₁-C₈ alkyl is methyl, ethyl, -n-propyl, isopropyl, -n-butyl, isobutyl, -s-butyl, -t-butyl, -n-pentyl, -2-pentyl, -3-pentyl, -2-methyl-2-butyl, -3-methyl-2-butyl, -3-methyl-1-butyl, -2-methyl-1-butyl, -1-hexyl, -2-hexyl, -3-hexyl, -2-methyl-2-pentyl, -3-methyl-2-pentyl, -4-methyl-2-pentyl, -3-methyl-3-pentyl, -2-methyl-3-pentyl, -2,3-dimethyl-2-butyl, or -3,3-dimethyl-2-butyl.

In some embodiments, the C₁-C₈ alkyl is C₁-C₆ alkyl. In some embodiments, the C₁-C₈ alkyl is C₁-C₃ alkyl. In some embodiments, the C₁-C₈ alkyl is C₂-C₅ alkyl. In some embodiments, the C₁-C₈ alkyl is C₄-C₅ alkyl. In some embodiments, the C₁-C₈ alkyl is a branched C₁-C₈ alkyl. In some embodiments, the C₁-C₈ alkyl is an unbranched C₁-C₈ alkyl. In some embodiments, the C₁-C₈ alkyl is an unsubstituted C₁-C₈ alkyl. In some embodiments, the C₁-C₈ alkyl is a branched, unsubstituted C₁-C₈ alkyl. In some embodiments, the C₁-C₈ alkyl is an unbranched, unsubstituted C₁-C₈ alkyl.

In some embodiments, R⁵ is substituted with one, two or three substituents wherein at least one substituent is NR⁹R¹⁰. In some embodiments, R⁵ is substituted with one, two or three substituents wherein at least one substituent is NH₂.

In some embodiments, R⁵ is substituted with one, two or three substituents wherein at least one substituent is C₃-C₈ (e.g., C₃, C₄, C₅, C₆, C₇, or C₈) carbocyclyl. In some embodiments, the C₃-C₈ carbocyclyl is saturated. In some embodiments, the C₃-C₈ carbocyclyl is partially unsaturated. Exemplary C₃-C₈ carbocyclic rings include, but are not limited to, cyclopropane, cyclobutane, cyclopentane, cyclopentene, cyclohexane, cyclohexene, 1,3-cyclohexadiene, 1,4-cyclohexadiene, cycloheptane, cycloheptene, and cyclooctane. In some embodiments, the C₃-C₈ carbocyclyl is unsubstituted C₃-C₈ carbocyclyl. In some embodiments, the C₃-C₈ carbocyclyl is substituted C₃-C₈ carbocyclyl. In some embodiments, the C₃-C₈ carbocyclyl is unbranched C₃-C₈ carbocyclyl. In some embodiments, the C₃-C₈ carbocyclyl is branched C₃-C₈ carbocyclyl.

In some embodiments, R⁵ is substituted with one, two or three substituents wherein at least one substituent is phenyl. In some embodiments, R⁵ is substituted with one, two or three substituents wherein at least one substituent is phenyl substituted with one, two, or three substituents independently selected from halo, cyano, and C₁-C₆ alkyl. In some embodiments, R⁵ is substituted with one, two or three substituents wherein at least one substituent is phenyl substituted with one, two, or three substituents wherein at least one substituent is halo (e.g., fluoro, chloro, iodo, bromo). In some embodiments, R⁵ is substituted with one, two or three substituents wherein at least one substituent is phenyl substituted with one, two, or three substituents wherein at least one substituent is cyano. In some embodiments, R⁵ is substituted with at least one halo and at least one oxo. In some embodiments, R⁵ is substituted with at least one halo and at least one cyano. In some embodiments, R⁵ is substituted with at least one halo and at least one N₃. In some embodiments, R⁵ is substituted with at least one halo and at least one OR⁸. In some embodiments, R⁵ is substituted with at least one halo and at least one NR⁹R¹⁰. In some embodiments, R⁵ is substituted with at least one halo and at least one C₃-C₈ carbocyclyl. In some embodiments, R⁵ is substituted with at least one halo and at least one unsubstituted phenyl. In some embodiments, R⁵ is substituted with at least one halo and at least one phenyl substituted with one, two, or three substituents independently selected from halo, cyano, and C₁-C₆ alkyl. In some embodiments, R⁵ is substituted with at least one oxo and at least one cyano. In some embodiments, R⁵ is substituted with at least one oxo and at least one N3. In some embodiments, R⁵ is substituted with at least one oxo and at least one OR⁸. In some embodiments, R⁵ is substituted with at least one oxo and at least one C₁-C₈ alkyl. In some embodiments, R⁵ is substituted with at least one oxo and at least one NR⁹R¹⁰. In some embodiments, R⁵ is substituted with at least one oxo and at least one C₃-C₈ carbocyclyl. In some embodiments, R⁵ is substituted with at least one oxo and at least one unsubstituted phenyl. In some embodiments, R⁵ is substituted with at least one oxo and at least one phenyl substituted with one, two, or three substituents independently selected from halo, cyano, and C₁-C₆ alkyl. In some embodiments, R⁵ is substituted with at least one cyano and at least one N₃. In some embodiments, R⁵ is substituted with at least one N₃ and at least one OR⁸. In some embodiments, R⁵ is substituted with at least one cyano and at least one C₁-C₈ alkyl. In some embodiments, R⁵ is substituted with at least one cyano and at least one NR⁹R¹⁰. In some embodiments, R⁵ is substituted with at least one cyano and at least one C₃-C₈ carbocyclyl. In some embodiments, R⁵ is substituted with at least one cyano and at least one unsubstituted phenyl. In some embodiments, R⁵ is substituted with at least one cyano and at least one phenyl substituted with one, two, or three substituents independently selected from halo, cyano, and C₁-C₆ alkyl. In some embodiments, R⁵ is substituted with at least one N₃ and at least one OR₈. In some embodiments, R⁵ is substituted with at least one N₃ and at least one C₁-C₈ alkyl. In some embodiments, R⁵ is substituted with at least one N₃ and at least one NR⁹R¹⁰. In some embodiments, R⁵ is substituted with at least one N₃ and at least one C₃-C₈ carbocyclyl. In some embodiments, R⁵ is substituted with at least one N₃ and at least one unsubstituted phenyl. In some embodiments, R⁵ is substituted with at least one N₃ and at least one phenyl substituted with one, two, or three substituents independently selected from halo, cyano, and C₁-C₆ alkyl. In some embodiments, R⁵ is substituted with at least one OR⁸ and at least one C₁-C₈ alkyl. In some embodiments, R⁵ is substituted with at least one OR⁸ and at least one NR⁹R¹⁰. In some embodiments, R⁵ is substituted with at least one OR⁸ and at least one C₃-C₈ carbocyclyl. In some embodiments, R⁵ is substituted with at least one OR⁸ and at least one unsubstituted phenyl. In some embodiments, R⁵ is substituted with at least one OR⁸ and at least one phenyl substituted with one, two, or three substituents independently selected from halo, cyano, and C₁-C₆ alkyl. In some embodiments, R⁵ is substituted with at least one C₁-C₈ alkyl and at least one NR⁹R¹⁰. In some embodiments, R⁵ is substituted with at least one C₁-C₈ alkyl and at least one C₃-C₈ carbocyclyl. In some embodiments, R⁵ is substituted with at least one C₁-C₈ alkyl and at least one unsubstituted phenyl. In some embodiments, R⁵ is substituted with at least one C₁-C₈ alkyl and at least one phenyl substituted with one, two, or three substituents independently selected from halo, cyano, and C₁-C₆ alkyl. In some embodiments, R⁵ is substituted with at least one C₃-C₈ carbocyclyl and at least one unsubstituted phenyl. In some embodiments, R⁵ is substituted with at least one C₃-C₈ carbocyclyl and at least one phenyl substituted with one, two, or three substituents independently selected from halo, cyano, and C₁-C₆ alkyl.

R⁶ can be C₁-C₆ alkyl, C₁-C₆ alkoxy, or C₆-C₁₀ aryl. In some embodiments, R⁶ is C₁-C₆ (e.g., C₁, C₂, C₃, C₄, C₅, or C₆) alkyl. In some embodiments, the C₁-C₆ alkyl is methyl, ethyl, -n-propyl, isopropyl, -n-butyl, isobutyl, -s-butyl, -t-butyl, -n-pentyl, -2-pentyl, -3-pentyl, -2-methyl-2-butyl, -3-methyl-2-butyl, -3-methyl-1-butyl, -2-methyl-1-butyl, -1-hexyl, -2-hexyl, -3-hexyl, -2-methyl-2-pentyl, -3-methyl-2-pentyl, -4-methyl-2-pentyl, -3-methyl-3-pentyl, -2-methyl-3-pentyl, -2,3-dimethyl-2-butyl, or -3,3-dimethyl-2-butyl. In some embodiments, the C₁-C₆ alkyl is C₁-C₃ alkyl. In some embodiments, the C₁-C₆ alkyl is C₂-C₅ alkyl. In some embodiments, the C₁-C₆ alkyl is C₄-C₆ alkyl. In some embodiments, the C₁-C₆ alkyl is a branched C₁-C₆ alkyl. In some embodiments, the C₁-C₆ alkyl is an unbranched C₁-C₆ alkyl. In some embodiments, the C₁-C₆ alkyl is an unsubstituted C₁-C₆ alkyl. In some embodiments, the C₁-C₆ alkyl is a branched, unsubstituted C₁-C₆ alkyl. In some embodiments, the C₁-C₆ alkyl is an unbranched, unsubstituted C₁-C₆ alkyl.

In some embodiments, R⁶ is C₁-C₆ (e.g., C₁, C₂, C₃, C₄, C₅, or C₆) alkoxy. The alkyl portion of an alkoxy group can have 1, 2, 3, 4, 5, or 6 carbon atoms (i.e., C₁-C₆ alkoxy) or 1, 2, or 3 carbon atoms (i.e., C₁-C₃ alkoxy). Examples of suitable alkoxy groups include, but are not limited to, methoxy (—O—CH₃ or —OMe), ethoxy (—OCH₂CH₃ or -OEt), isopropoxy (—O—CH(CH₃)₂), t-butoxy (—O—C(CH₃)₃ or -OtBu) and the like. Other examples of suitable alkoxy groups include, but are not limited to, sec-butoxy, tert-butoxy, pentoxy, and hexoxy.

In some embodiments, R⁶ is C₆-C₁₀ aryl (e.g., C₆-C₇ aryl, C₆-C₅ aryl, C₆-C₉ aryl, C₇-C₈ aryl, C₇-C₉ aryl, C₇-C₁₀ aryl, C₈-C₉ aryl, C₅-C₁₀ aryl, or C₉-C₁₀ aryl). In some embodiments, the C₆-C₁₀ aryl is C₆ aryl (e.g., phenyl), C₇ aryl, C₈ aryl, C₉ aryl, or C₁₀ aryl (e.g., naphthyl). In some embodiments, the C₆-C₁₀ aryl is unsubstituted C₆-C₁₀ aryl. In some embodiments, the C₆-C₁₀ aryl is phenyl. In some embodiments, the C₆-C₁₀ aryl is naphthyl.

R⁷ can be C₁-C₈ alkyl, C₂-C₅ alkenyl, C₂-C₈ alkynyl, C₃-C₈ carbocyclyl, C₆-C₁₀ aryl, 4 to 6 membered heterocyclyl containing 1, 2, or 3 O, or 5 to 6 membered heteroaryl containing 1, 2, or 3 heteroatoms selected from N, O, and S; wherein the alkyl, alkenyl, alkynyl, carbocyclyl, aryl, heterocyclyl or heteroaryl of R⁷ can be optionally substituted with one, two or three substituents independently selected from the group consisting of halo, oxo, cyano, —N₃, —OR⁸, C₁-C₈ alkyl, —NR⁹R¹⁰, C₃-C₈ carbocyclyl, and phenyl optionally substituted with one, two, or three substituents independently selected from halo, cyano, and C₁-C₆ alkyl; and R⁸ can be H, C₁-C₆ alkyl, C₁-C₆ haloalkyl, and C₃-C₆ cycloalkyl; R⁹ can be H, C₁-C₆ alkyl, C₁-C₆ haloalkyl, and C₃-C₆ cycloalkyl; and R¹⁰ can be H, C₁-C₆ alkyl, C₁-C₆ haloalkyl, and C₃-C₆ cycloalkyl.

In some embodiments, R⁷ is a C₁-C₆ (e.g., C₁, C₂, C₃, C₄, C₅, or C₆) alkyl. In some embodiments, the C₁-C₆ alkyl is methyl, ethyl, -n-propyl, isopropyl, -n-butyl, isobutyl, -s-butyl, -t-butyl, -n-pentyl, -2-pentyl, -3-pentyl, -2-methyl-2-butyl, -3-methyl-2-butyl, -3-methyl-1-butyl, -2-methyl-1-butyl, -1-hexyl, -2-hexyl, -3-hexyl, -2-methyl-2-pentyl, -3-methyl-2-pentyl, -4-methyl-2-pentyl, -3-methyl-3-pentyl, -2-methyl-3-pentyl, -2,3-dimethyl-2-butyl, or -3,3-dimethyl-2-butyl. In some embodiments, the C₁-C₆ alkyl is C₁-C₃ alkyl. In some embodiments, the C₁-C₆ alkyl is C₂-C₅ alkyl. In some embodiments, the C₁-C₆ alkyl is C₄-C₆ alkyl. In some embodiments, the C₁-C₆ alkyl is a branched C₁-C₆ alkyl. In some embodiments, the C₁-C₆ alkyl is an unbranched C₁-C₆ alkyl. In some embodiments, the C₁-C₆ alkyl is an unsubstituted C₁-C₆ alkyl. In some embodiments, the C₁-C₆ alkyl is a substituted C₁-C₆ alkyl. In some embodiments, the C₁-C₆ alkyl is a branched, unsubstituted C₁-C₆ alkyl. In some embodiments, the C₁-C₆ alkyl is a branched, substituted C₁-C₆ alkyl. In some embodiments, the C₁-C₆ alkyl is an unbranched, unsubstituted C₁-C₆ alkyl. In some embodiments, R⁷ is C₁-C₆ alkyl optionally substituted with one, two or three substituents independently selected from halo, oxo, cyano, N₃, OR⁸, C₁-C₈ alkyl, NR⁹R¹⁰, C₃-C₈ carbocyclyl, and phenyl optionally substituted with one, two, or three substituents independently selected from halo, cyano, and C₁-C₆ alkyl. In some embodiments, R⁷ is C₁-C₆ alkyl optionally substituted with one, two or three substituents independently selected from halo, OR⁸, C₃-C₈ carbocyclyl, and phenyl, wherein R⁸ is C₁-C₆ alkyl. In some embodiments, R⁷ is C₁-C₆ alkyl substituted with one, two or three F. In some embodiments, R⁷ is C₁-C₆ alkyl substituted with C₃-C₈ carbocyclyl. In some embodiments, R⁷ is C₁-C₆ alkyl substituted with phenyl. In some embodiments, R⁷ is C₂-C₈ (e.g., C₂, C₃, C₄, C₅, C₆, C₇, or C₈) alkenyl. In some embodiments, the C₂-C₈ alkenyl has at least one (e.g., at least 2, at least 3, at least 3, at least 4, or at least 5) double bond. In some embodiments, the C₂-C₈ alkenyl is an unsubstituted C₂-C₈ alkenyl.

In some embodiments, the C₂-C₈ alkenyl is a substituted C₂-C₈ alkenyl. Examples of the C₂-C₈ alkenyl include, but are not limited to, vinyl (ethenyl), propenyl, isopropenyl, 1-butenyl, 2-butenyl, isobutenyl, butadienyl, 1-pentenyl, 2-pentenyl, isopentenyl, 1,3-pentadienyl, 1,4-pentadienyl, 1-hexenyl, 2-hexenyl, 3-hexenyl, 1,3-hexadienyl, 1,4-hexadienyl, 1,5-hexadienyl, 2,4-hexadienyl, or 1,3,5-hexatrienyl. In some embodiments, R⁷ is unsubstituted C₂-C₈ alkenyl. In some embodiments, R⁷ is substituted C₂-C₈ alkenyl.

In some embodiments, R⁷ is C₂-C₈ (e.g., C₂, C₃, C₄, C₅, C₆, C₇, or C₈) alkynyl. In some embodiments, the C₂-C₈ alkynyl has at least one triple bond. In some embodiments, the C₂-C₈ alkynyl has at least one triple bond and at least one double bond. Exemplary C₂-C₈ alkynyl include, but are not limited to, ethynyl, prop-1-ynyl, but-1-ynyl, pent-1-ynyl, pent-4-ynyl and penta-1,4-diynyl. In some embodiments, the C₂-C₈ alkynyl is unsubstituted C₂-C₈ alkynyl. In some embodiments, the C₂-C₈ alkynyl is substituted C₂-C₈ alkynyl. In some embodiments, the C₂-C₈ alkynyl is branched C₂-C₈ alkynyl. In some embodiments, the C₂-C₈ alkynyl is unbranched C₂-C₈ alkynyl.

In some embodiments, R⁷ is C₃-C₈ (e.g., C₃, C₄, C₅, C₆, C₇, or C₈) carbocyclyl. In some embodiments, the C₃-C₈ carbocyclyl is saturated. In some embodiments, the C₃-C₈ carbocyclyl is partially unsaturated. Exemplary C₃-C₈ carbocyclic rings include, but are not limited to, cyclopropane, cyclobutane, cyclopentane, cyclopentene, cyclohexane, cyclohexene, 1,3-cyclohexadiene, 1,4-cyclohexadiene, cycloheptane, cycloheptene, and cyclooctane. In some embodiments, the C₃-C₈ carbocyclyl is unsubstituted C₃-C₈ carbocyclyl. In some embodiments, the C₃-C₈ carbocyclyl is substituted C₃-C₈ carbocyclyl. In some embodiments, the C₃-C₈ carbocyclyl is unbranched C₃-C₈ carbocyclyl. In some embodiments, the C₃-C₈ carbocyclyl is branched C₃-C₈ carbocyclyl. In some embodiments, R⁷ is C₃-C₈ carbocyclyl optionally substituted with one, two, or three substituents independently selected from halo and C₁-C₆ alkyl.

In some embodiments, R⁷ is C₆-C₁₀ aryl. In some embodiments, the C₆-C₁₀ aryl is C₆ aryl (e.g., phenyl) or C₁₀ aryl (e.g., naphthyl). In some embodiments, the C₆-C₁₀ aryl is unsubstituted C₆-C₁₀ aryl. In some embodiments, the C₆-C₁₀ aryl is substituted C₆-C₁₀ aryl. In some embodiments, the C₆-C₁₀ aryl is phenyl. In some embodiments, the C₆-C₁₀ aryl is naphthyl.

In some embodiments, R⁷ is 4- to 6- (e.g., 4-, 5-, 6-) membered heterocyclyl containing one, two, or three O. In some embodiments, the 4- to 6-membered heterocyclyl is 4-membered heterocyclyl containing one O. In some embodiments, the 4- to 6-membered heterocyclyl is 4-membered heterocyclyl containing two O. In some embodiments, the 4- to 6-membered heterocyclyl is 4-membered heterocyclyl containing three O. In some embodiments, the 4- to 6-membered heterocyclyl is 5-membered heterocyclyl containing one O. In some embodiments, the 4- to 6-membered heterocyclyl is 5-membered heterocyclyl containing two O. In some embodiments, the 4- to 6-membered heterocyclyl is 5-membered heterocyclyl containing three O. In some embodiments, the 4- to 6-membered heterocyclyl is 6-membered heterocyclyl containing one heteroatom selected from O. In some embodiments, the 4- to 6-membered heterocyclyl is 6-membered heterocyclyl containing two O. In some embodiments, the 4- to 6-membered heterocyclyl is 6-membered heterocyclyl containing three O. In some embodiments, the 4- to 6-membered heterocyclyl is an unsubstituted 4- to 6-membered heterocyclyl. In some embodiments, the 4- to 6-membered heterocyclyl is a substituted 4- to 6-membered heterocyclyl.

In some embodiments, R⁷ is 5- to 6-membered heteroaryl containing 1, 2, or 3 heteroatoms selected from N, O, and S. In some embodiments, the 5- to 6-membered heteroaryl is 5-membered heteroaryl containing one heteroatom selected from N, O, and S. In some embodiments, the 5- to 6-membered heteroaryl is 5-membered heteroaryl containing two heteroatoms selected from N, O, and S. In some embodiments, the 5- to 6-membered heteroaryl is 5-membered heteroaryl containing three heteroatoms selected from N, O, and S. In some embodiments, the 5- to 6-membered heteroaryl is 6-membered heteroaryl containing one heteroatom selected from N, O, and S. In some embodiments, the 5- to 6-membered heteroaryl is 6-membered heteroaryl containing two heteroatoms selected from N, O, and S. In some embodiments, the 5- to 6-membered heteroaryl is 6-membered heteroaryl containing three heteroatoms selected from N, O, and S. In some embodiments, the 5- to 6-membered heteroaryl is pyridinyl. In some embodiments, the 5- to 6-membered heteroaryl is pyrimidinyl. In some embodiments, the 5- to 6-membered heteroaryl is an unsubstituted 5- to 6-membered heteroaryl. In some embodiments, the 5- to 6-membered heteroaryl is a substituted 5- to 6-membered heteroaryl.

In some embodiments, R⁷ is unsubstituted. In some embodiments, R⁷ is substituted with one substituent. In some embodiments, R⁷ is substituted with two substituents. In some embodiments, R⁷ is substituted three substituents. In some embodiments, R⁷ is substituted with one, two or three substituents wherein at least one substituent is halo. In some embodiments, R⁷ is substituted with one, two or three substituents wherein at least one substituent is oxo. In some embodiments, R⁷ is substituted with one, two or three substituents wherein at least one substituent is cyano. In some embodiments, R⁷ is substituted with one, two or three substituents wherein at least one substituent is N₃. In some embodiments, R⁷ is substituted with one, two or three substituents wherein at least one substituent is OR⁸.

In some embodiments, R⁷ is substituted with one, two or three substituents wherein at least one substituent is a C₁-C₈ (e.g., C₁, C₂, C₃, C₄, C₅, C₆, C₇, or C₈) alkyl. In some embodiments, the C₁-C₈ alkyl is methyl, ethyl, -n-propyl, isopropyl, -n-butyl, isobutyl, -s-butyl, -t-butyl, -n-pentyl, -2-pentyl, -3-pentyl, -2-methyl-2-butyl, -3-methyl-2-butyl, -3-methyl-1-butyl, -2-methyl-1-butyl, -1-hexyl, -2-hexyl, -3-hexyl, -2-methyl-2-pentyl, -3-methyl-2-pentyl, -4-methyl-2-pentyl, -3-methyl-3-pentyl, -2-methyl-3-pentyl, -2,3-dimethyl-2-butyl, or -3,3-dimethyl-2-butyl.

In some embodiments, the C₁-C₈ alkyl is C₁-C₆ alkyl. In some embodiments, the C₁-C₈ alkyl is C₁-C₃ alkyl. In some embodiments, the C₁-C₈ alkyl is C₂-C₅ alkyl. In some embodiments, the C₁-C₈ alkyl is C₄-C₅ alkyl. In some embodiments, the C₁-C₈ alkyl is a branched C₁-C₈ alkyl. In some embodiments, the C₁-C₈ alkyl is an unbranched C₁-C₈ alkyl. In some embodiments, the C₁-C₈ alkyl is an unsubstituted C₁-C₈ alkyl. In some embodiments, the C₁-C₈ alkyl is a branched, unsubstituted C₁-C₈ alkyl. In some embodiments, the C₁-C₈ alkyl is a branched, substituted C₁-C₈ alkyl. In some embodiments, the C₁-C₈ alkyl is an unbranched, unsubstituted C₁-C₈ alkyl.

In some embodiments, R⁷ is substituted with one, two or three substituents wherein at least one substituent is NR⁹R¹⁰. In some embodiments, R⁷ is substituted with one, two or three substituents wherein at least one substituent is NH₂.

In some embodiments, R⁷ is substituted with one, two or three substituents wherein at least one substituent is C₃-C₈ (e.g., C₃, C₄, C₅, C₆, C₇, or C₈) carbocyclyl. In some embodiments, the C₃-C₈ carbocyclyl is saturated. In some embodiments, the C₃-C₈ carbocyclyl is partially unsaturated. Exemplary C₃-C₈ carbocyclic rings include, but are not limited to, cyclopropane, cyclobutane, cyclopentane, cyclopentene, cyclohexane, cyclohexene, 1,3-cyclohexadiene, 1,4-cyclohexadiene, cycloheptane, cycloheptene, and cyclooctane. In some embodiments, the C₃-C₈ carbocyclyl is unsubstituted C₃-C₈ carbocyclyl. In some embodiments, the C₃-C₈ carbocyclyl is substituted C₃-C₈ carbocyclyl. In some embodiments, the C₃-C₈ carbocyclyl is unbranched C₃-C₈ carbocyclyl. In some embodiments, the C₃-C₈ carbocyclyl is branched C₃-C₈ carbocyclyl.

In some embodiments, R⁷ is substituted with one, two or three substituents wherein at least one substituent is phenyl. In some embodiments, R⁷ is substituted with one, two or three substituents wherein at least one substituent is phenyl substituted with one, two, or three substituents independently selected from halo, cyano, and C₁-C₆ alkyl. In some embodiments, R⁷ is substituted with one, two or three substituents wherein at least one substituent is phenyl substituted with one, two, or three substituents wherein at least one substituent is halo (e.g., fluoro, chloro, iodo, bromo). In some embodiments, R⁷ is substituted with one, two or three substituents wherein at least one substituent is phenyl substituted with one, two, or three substituents wherein at least one substituent is cyano. In some embodiments, R⁷ is substituted with at least one halo and at least one oxo. In some embodiments, R⁷ is substituted with at least one halo and at least one cyano. In some embodiments, R⁷ is substituted with at least one halo and at least one N₃. In some embodiments, R⁷ is substituted with at least one halo and at least one OR⁸. In some embodiments, R⁷ is substituted with at least one halo and at least one NR⁹R¹⁰. In some embodiments, R⁷ is substituted with at least one halo and at least one C₃-C₈ carbocyclyl. In some embodiments, R⁷ is substituted with at least one halo and at least one unsubstituted phenyl. In some embodiments, R⁷ is substituted with at least one halo and at least one phenyl substituted with one, two, or three substituents independently selected from halo, cyano, and C₁-C₆ alkyl. In some embodiments, R⁷ is substituted with at least one oxo and at least one cyano. In some embodiments, R⁷ is substituted with at least one oxo and at least one N₃. In some embodiments, R⁷ is substituted with at least one oxo and at least one OR⁸. In some embodiments, R⁷ is substituted with at least one oxo and at least one C₁-C₈ alkyl. In some embodiments, R⁷ is substituted with at least one oxo and at least one NR⁹R¹⁰. In some embodiments, R⁷ is substituted with at least one oxo and at least one C₃-C₈ carbocyclyl. In some embodiments, R⁷ is substituted with at least one oxo and at least one unsubstituted phenyl. In some embodiments, R⁷ is substituted with at least one oxo and at least one phenyl substituted with one, two, or three substituents independently selected from halo, cyano, and C₁-C₆ alkyl. In some embodiments, R⁷ is substituted with at least one cyano and at least one N₃. In some embodiments, R⁷ is substituted with at least one N₃ and at least one OR⁸. In some embodiments, R⁷ is substituted with at least one cyano and at least one C₁-C₈ alkyl. In some embodiments, R⁷ is substituted with at least one cyano and at least one NR⁹R¹⁰. In some embodiments, R⁷ is substituted with at least one cyano and at least one C₃-C₈ carbocyclyl. In some embodiments, R⁷ is substituted with at least one cyano and at least one unsubstituted phenyl. In some embodiments, R⁷ is substituted with at least one cyano and at least one phenyl substituted with one, two, or three substituents independently selected from halo, cyano, and C₁-C₆ alkyl. In some embodiments, R⁷ is substituted with at least one N₃ and at least one OR⁸. In some embodiments, R⁷ is substituted with at least one N₃ and at least one C₁-C₈ alkyl. In some embodiments, R⁷ is substituted with at least one N₃ and at least one NR⁹R¹⁰. In some embodiments, R⁷ is substituted with at least one N₃ and at least one C₃-C₈ carbocyclyl. In some embodiments, R⁷ is substituted with at least one N₃ and at least one unsubstituted phenyl. In some embodiments, R⁷ is substituted with at least one N₃ and at least one phenyl substituted with one, two, or three substituents independently selected from halo, cyano, and C₁-C₆ alkyl. In some embodiments, R⁷ is substituted with at least one OR⁸ and at least one C₁-C₈ alkyl. In some embodiments, R⁷ is substituted with at least one OR⁸ and at least one NR⁹R¹⁰. In some embodiments, R⁷ is substituted with at least one OR⁸ and at least one C₃-C₈ carbocyclyl. In some embodiments, R⁷ is substituted with at least one OR⁸ and at least one unsubstituted phenyl. In some embodiments, R⁷ is substituted with at least one OR⁸ and at least one phenyl substituted with one, two, or three substituents independently selected from halo, cyano, and C₁-C₆ alkyl. In some embodiments, R⁷ is substituted with at least one C₁-C₈ alkyl and at least one NR⁹R¹⁰. In some embodiments, R⁷ is substituted with at least one C₁-C₈ alkyl and at least one C₃-C₈ carbocyclyl. In some embodiments, R⁷ is substituted with at least one C₁-C₈ alkyl and at least one unsubstituted phenyl. In some embodiments, R⁷ is substituted with at least one C₁-C₈ alkyl and at least one phenyl substituted with one, two, or three substituents independently selected from halo, cyano, and C₁-C₆ alkyl. In some embodiments, R⁷ is substituted with at least one C₃-C₈ carbocyclyl and at least one unsubstituted phenyl. In some embodiments, R⁷ is substituted with at least one C₃-C₈ carbocyclyl and at least one phenyl substituted with one, two, or three substituents independently selected from halo, cyano, and C₁-C₆ alkyl.

Each R⁸ can be independently H, C₁-C₆ alkyl, C₁-C₆ haloalkyl, and C₃-C₆ cycloalkyl. In some embodiments, at least one R⁵ is H. In some embodiments, at least one R⁸ is C₁-C₆ (e.g., C₁, C₂, C₃, C₄, C₅, or C₆) alkyl, for instance, methyl, ethyl, -n-propyl, isopropyl, -n-butyl, isobutyl, -s-butyl, -t-butyl, -n-pentyl, -2-pentyl, -3-pentyl, -2-methyl-2-butyl, -3-methyl-2-butyl, -3-methyl-1-butyl, -2-methyl-1-butyl, -1-hexyl, -2-hexyl, -3-hexyl, -2-methyl-2-pentyl, -3-methyl-2-pentyl, -4-methyl-2-pentyl, -3-methyl-3-pentyl, -2-methyl-3-pentyl, -2,3-dimethyl-2-butyl, or -3,3-dimethyl-2-butyl. In some embodiments, at least one R⁸ is C₁-C₃ alkyl. In some embodiments, at least one R⁸ is C₂-C₅ alkyl. In some embodiments, at least one R⁸ is C₄-C₆ alkyl. In some embodiments, at least one R¹ is a branched C₁-C₆ alkyl. In some embodiments, R⁸ is an unbranched C₁-C₆ alkyl. In some embodiments, at least one R¹ is an unsubstituted C₁-C₆ alkyl. In some embodiments, at least one R⁸ is a branched, unsubstituted C₁-C₆ alkyl. In some embodiments, at least one R⁸ is an unbranched, unsubstituted C₁-C₆ alkyl.

In some embodiments, at least one R¹ is C₁-C₆ (e.g., C₁, C₂, C₃, C₄, C₅, or C₆) haloalkyl, wherein a C₁-C₆ alkyl is substituted with at least one halo (e.g., fluoro, iodo, chloro, or bromo). Exemplary C₁-C₆ haloalkyl include halomethyl, haloethyl, halo-n-propyl, haloisopropyl, halo-n-butyl, halo-isobutyl, halo-s-butyl, halo-t-butyl, halo-n-pentyl, halo-2-pentyl, halo-3-pentyl, halo-2-methyl-2-butyl, halo-3-methyl-2-butyl, halo-3-methyl-1-butyl, halo-2-methyl-1-butyl, halo-1-hexyl, halo-2-hexyl, halo-3-hexyl, halo-2-methyl-2-pentyl, halo-3-methyl-2-pentyl, halo-4-methyl-2-pentyl, halo-3-methyl-3-pentyl, halo-2-methyl-3-pentyl, halo-2,3-dimethyl-2-butyl, or halo-3,3-dimethyl-2-butyl. In some embodiments, the C₁-C₆ haloalkyl contains at least one (e.g., one, two, three, four, or five) halo. In some embodiments, the C₁-C₆ haloalkyl contains at least one fluoro. In some embodiments, the C₁-C₆ haloalkyl contains at least one chloro. In some embodiments, the C₁-C₆ haloalkyl contains at least one iodo. In some embodiments, the C₁-C₆ haloalkyl contains at least one bromo. In some embodiments, the C₁-C₆ haloalkyl contains at least one fluoro and at least one chloro. In some embodiments, the C₁-C₆ haloalkyl contains at least one fluoro and at least one iodo. In some embodiments, the C₁-C₆ haloalkyl contains at least one fluoro and at least one bromo. In some embodiments, the C₁-C₆ haloalkyl contains at least one chloro and at least one iodo. In some embodiments, the C₁-C₆ haloalkyl contains at least one chloro and at least one bromo. In some embodiments, the C₁-C₆ haloalkyl contains at least one iodo and at least one bromo. Examples of suitable C₁-C₆ haloalkyl groups include, but are not limited to, —CF₃, —CHF₂, —CFH₂, —CH₂CF₃, fluorochloromethyl, difluorochloromethyl, 1,1,1-trifluoroethyl and pentafluoroethyl.

In some embodiments, at least one R⁸ is C₃-C₆ (e.g., C₃, C₄, C₅, or C₆) cycloalkyl. In some embodiments, R⁸ is a C₃ cycloalkyl, C₄ cycloalkyl, C₅ cycloalkyl, or C₆ cycloalkyl. In some embodiments, the C₃-C₁₀ cycloalkyl is saturated. In some embodiments, the C₃-C₆ cycloalkyl is partially saturated. In some embodiments, the C₃-C₆ cycloalkyl includes partially unsaturated ring systems containing at least (e.g., one, two) double bonds. In some embodiments, R⁸ is cyclopropyl, cyclobutyl, cyclopentyl, or cyclohexyl.

Each R⁹ can be independently H, C₁-C₆ alkyl, C₁-C₆ haloalkyl, and C₃-C₆ cycloalkyl. In some embodiments, at least one R⁹ is H. In some embodiments, at least one R⁹ is C₁-C₆ (e.g., C₁, C₂, C₃, C₄, C₅, or C₆) alkyl, for instance, methyl, ethyl, -n-propyl, isopropyl, -n-butyl, isobutyl, -s-butyl, -t-butyl, -n-pentyl, -2-pentyl, -3-pentyl, -2-methyl-2-butyl, -3-methyl-2-butyl, -3-methyl-1-butyl, -2-methyl-1-butyl, -1-hexyl, -2-hexyl, -3-hexyl, -2-methyl-2-pentyl, -3-methyl-2-pentyl, -4-methyl-2-pentyl, -3-methyl-3-pentyl, -2-methyl-3-pentyl, -2,3-dimethyl-2-butyl, or -3,3-dimethyl-2-butyl. In some embodiments, at least one R⁹ is C₁-C₃ alkyl. In some embodiments, at least one R⁹ is C₂-C₅ alkyl. In some embodiments, at least one R⁹ is C₄-C₆ alkyl. In some embodiments, at least one R⁹ is a branched C₁-C₆ alkyl. In some embodiments, R⁹ is an unbranched C₁-C₆ alkyl. In some embodiments, at least one R⁹ is an unsubstituted C₁-C₆ alkyl. In some embodiments, at least one R⁹ is a branched, unsubstituted C₁-C₆ alkyl. In some embodiments, at least one R⁹ is an unbranched, unsubstituted C₁-C₆ alkyl.

In some embodiments, at least one R⁹ is C₁-C₆ (e.g., C₁, C₂, C₃, C₄, C₅, or C₆) haloalkyl, wherein a C₁-C₆ alkyl is substituted with at least one halo (e.g., fluoro, iodo, chloro, or bromo). Exemplary C₁-C₆ haloalkyl include halomethyl, haloethyl, halo-n-propyl, haloisopropyl, halo-n-butyl, halo-isobutyl, halo-s-butyl, halo-t-butyl, halo-n-pentyl, halo-2-pentyl, halo-3-pentyl, halo-2-methyl-2-butyl, halo-3-methyl-2-butyl, halo-3-methyl-1-butyl, halo-2-methyl-1-butyl, halo-1-hexyl, halo-2-hexyl, halo-3-hexyl, halo-2-methyl-2-pentyl, halo-3-methyl-2-pentyl, halo-4-methyl-2-pentyl, halo-3-methyl-3-pentyl, halo-2-methyl-3-pentyl, halo-2,3-dimethyl-2-butyl, or halo-3,3-dimethyl-2-butyl. In some embodiments, the C₁-C₆ haloalkyl contains at least one (e.g., one, two, three, four, or five) halo. In some embodiments, the C₁-C₆ haloalkyl contains at least one fluoro. In some embodiments, the C₁-C₆ haloalkyl contains at least one chloro. In some embodiments, the C₁-C₆ haloalkyl contains at least one iodo. In some embodiments, the C₁-C₆ haloalkyl contains at least one bromo. In some embodiments, the C₁-C₆ haloalkyl contains at least one fluoro and at least one chloro. In some embodiments, the C₁-C₆ haloalkyl contains at least one fluoro and at least one iodo. In some embodiments, the C₁-C₆ haloalkyl contains at least one fluoro and at least one bromo. In some embodiments, the C₁-C₆ haloalkyl contains at least one chloro and at least one iodo. In some embodiments, the C₁-C₆ haloalkyl contains at least one chloro and at least one bromo. In some embodiments, the C₁-C₆ haloalkyl contains at least one iodo and at least one bromo. Examples of suitable C₁-C₆ haloalkyl groups include, but are not limited to, —CF₃, —CHF₂, —CFH₂, —CH₂CF₃, fluorochloromethyl, difluorochloromethyl, 1,1,1-trifluoroethyl and pentafluoroethyl.

In some embodiments, at least one R⁹ is C₃-C₆ (e.g., C₃, C₄, C₅, or C₆) cycloalkyl. In some embodiments, R⁹ is a C₃ cycloalkyl, C₄ cycloalkyl, C₅ cycloalkyl, or C₆ cycloalkyl. In some embodiments, the C₃-C₁₀ cycloalkyl is saturated. In some embodiments, the C₃-C₆ cycloalkyl is partially saturated. In some embodiments, the C₃-C₆ cycloalkyl includes partially unsaturated ring systems containing at least (e.g., one, two) double bonds. In some embodiments, R⁹ is cyclopropyl, cyclobutyl, cyclopentyl, or cyclohexyl.

Each R¹⁰ can be independently H, C₁-C₆ alkyl, C₁-C₆ haloalkyl, and C₃-C₆ cycloalkyl. In some embodiments, at least one R¹⁰ is H. In some embodiments, at least one R¹⁰ is C₁-C₆ (e.g., C₁, C₂, C₃, C₄, C₅, or C₆) alkyl, for instance, methyl, ethyl, -n-propyl, isopropyl, -n-butyl, isobutyl, -s-butyl, -t-butyl, -n-pentyl, -2-pentyl, -3-pentyl, -2-methyl-2-butyl, -3-methyl-2-butyl, -3-methyl-1-butyl, -2-methyl-1-butyl, -1-hexyl, -2-hexyl, -3-hexyl, -2-methyl-2-pentyl, -3-methyl-2-pentyl, -4-methyl-2-pentyl, -3-methyl-3-pentyl, -2-methyl-3-pentyl, -2,3-dimethyl-2-butyl, or -3,3-dimethyl-2-butyl. In some embodiments, at least one R¹⁰ is C₁-C₃ alkyl. In some embodiments, at least one R¹⁰ is C₂-C₅ alkyl. In some embodiments, at least one R¹⁰ is C₄-C₆ alkyl. In some embodiments, at least one R¹⁰ is a branched C₁-C₆ alkyl. In some embodiments, R¹⁰ is an unbranched C₁-C₆ alkyl. In some embodiments, at least one R¹⁰ is an unsubstituted C₁-C₆ alkyl. In some embodiments, at least one R¹⁰ is a branched, unsubstituted C₁-C₆ alkyl. In some embodiments, at least one R¹⁰ is an unbranched, unsubstituted C₁-C₆ alkyl.

In some embodiments, at least one R¹⁰ is C₁-C₆ (e.g., C₁, C₂, C₃, C₄, C₅, or C₆) haloalkyl, wherein a C₁-C₆ alkyl is substituted with at least one halo (e.g., fluoro, iodo, chloro, or bromo). Exemplary C₁-C₆ haloalkyl include halomethyl, haloethyl, halo-n-propyl, haloisopropyl, halo-n-butyl, halo-isobutyl, halo-s-butyl, halo-t-butyl, halo-n-pentyl, halo-2-pentyl, halo-3-pentyl, halo-2-methyl-2-butyl, halo-3-methyl-2-butyl, halo-3-methyl-1-butyl, halo-2-methyl-1-butyl, halo-1-hexyl, halo-2-hexyl, halo-3-hexyl, halo-2-methyl-2-pentyl, halo-3-methyl-2-pentyl, halo-4-methyl-2-pentyl, halo-3-methyl-3-pentyl, halo-2-methyl-3-pentyl, halo-2,3-dimethyl-2-butyl, or halo-3,3-dimethyl-2-butyl. In some embodiments, the C₁-C₆ haloalkyl contains at least one (e.g., one, two, three, four, or five) halo. In some embodiments, the C₁-C₆ haloalkyl contains at least one fluoro. In some embodiments, the C₁-C₆ haloalkyl contains at least one chloro. In some embodiments, the C₁-C₆ haloalkyl contains at least one iodo. In some embodiments, the C₁-C₆ haloalkyl contains at least one bromo. In some embodiments, the C₁-C₆ haloalkyl contains at least one fluoro and at least one chloro. In some embodiments, the C₁-C₆ haloalkyl contains at least one fluoro and at least one iodo. In some embodiments, the C₁-C₆ haloalkyl contains at least one fluoro and at least one bromo. In some embodiments, the C₁-C₆ haloalkyl contains at least one chloro and at least one iodo. In some embodiments, the C₁-C₆ haloalkyl contains at least one chloro and at least one bromo. In some embodiments, the C₁-C₆ haloalkyl contains at least one iodo and at least one bromo. Examples of suitable C₁-C₆ haloalkyl groups include, but are not limited to, —CF₃, —CHF₂, —CFH₂, —CH₂CF₃, fluorochloromethyl, difluorochloromethyl, 1,1,1-trifluoroethyl and pentafluoroethyl.

In some embodiments, at least one R¹⁰ is C₃-C₆ (e.g., C₃, C₄, C₅, or C₆) cycloalkyl. In some embodiments, R¹⁰ is a C₃ cycloalkyl, C₄ cycloalkyl, C₅ cycloalkyl, or C₆ cycloalkyl. In some embodiments, the C₃-C₁₀ cycloalkyl is saturated. In some embodiments, the C₃-C₆ cycloalkyl is partially saturated. In some embodiments, the C₃-C₆ cycloalkyl includes partially unsaturated ring Systems containing at least (e.g., one, two) double bonds. In some embodiments, R¹⁰ is cyclopropyl, cyclobutyl, cyclopentyl, or cyclohexyl.

Base can be

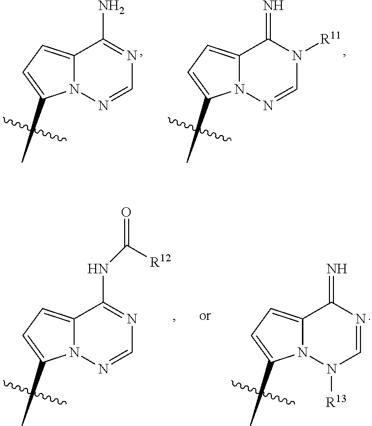

In some embodiments Base is

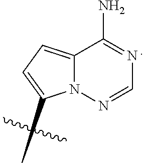

In some embodiments, Base is

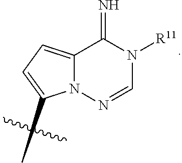

In some embodiments, Base is

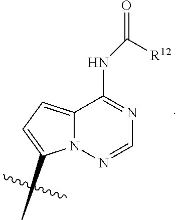

In some embodiments, Base is

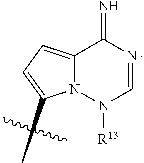

R¹¹ can be C₁-C₆ (e.g., C₁, C₂, C₃, C₄, C₅, or C₆) alkyl substituted with —OP(O)(OH)₂ or —OC(O)R¹². In some embodiments, the C₁-C₆ alkyl is methyl, ethyl, -n-propyl, isopropyl, -n-butyl, isobutyl, -s-butyl, -t-butyl, -n-pentyl, -2-pentyl, -3-pentyl, -2-methyl-2-butyl, -3-methyl-2-butyl, -3-methyl-1-butyl, -2-methyl-1-butyl, -1-hexyl, -2-hexyl, -3-hexyl, -2-methyl-2-pentyl, -3-methyl-2-pentyl, -4-methyl-2-pentyl, -3-methyl-3-pentyl, -2-methyl-3-pentyl, -2,3-dimethyl-2-butyl, or -3,3-dimethyl-2-butyl. In some embodiments, the C₁-C₆ alkyl is C₁-C₃ alkyl. In some embodiments, the C₁-C₆ alkyl is C₂-C₅ alkyl. In some embodiments, the C₁-C₆ alkyl is C₄-C₆ alkyl. In some embodiments, the C₁-C₆ alkyl is a branched C₁-C₆ alkyl. In some embodiments, the C₁-C₆ alkyl is an unbranched C₁-C₆ alkyl. In some embodiments, the C₁-C₆ alkyl is a substituted C₁-C₆ alkyl. In some embodiments, the C₁-C₆ alkyl is a branched, substituted C₁-C₆ alkyl. In some embodiments, the C₁-C₆ alkyl is an unbranched, substituted C₁-C₆ alkyl. In some embodiments, the tC₁-C₆ alkyl is substituted with —OP(O)(OH)₂. In some embodiments, the C₁-C₆ alkyl is substituted with —OC(O)R¹².

R¹² can be C₁-C₈ alkyl or C₁-C₈ alkoxy. In some embodiments, R¹² is C₁-C₈ (e.g., C₁, C₂, C₃, C₄, C₅, C₆, C₇, or C₈) alkyl. In some embodiments, the C₁-C₈ alkyl is methyl, ethyl, -n-propyl, isopropyl, -n-butyl, isobutyl, -s-butyl, -t-butyl, -n-pentyl, -2-pentyl, -3-pentyl, -2-methyl-2-butyl, -3-methyl-2-butyl, -3-methyl-1-butyl, -2-methyl-1-butyl, -1-hexyl, -2-hexyl, -3-hexyl, -2-methyl-2-pentyl, -3-methyl-2-pentyl, -4-methyl-2-pentyl, -3-methyl-3-pentyl, -2-methyl-3-pentyl, -2,3-dimethyl-2-butyl, or -3,3-dimethyl-2-butyl. In some embodiments, the C₁-C₈ alkyl is C₁-C₆ alkyl. In some embodiments, the C₁-C₈ alkyl is C₁-C₃ alkyl. In some embodiments, the C₁-C₈ alkyl is C₂-C₅ alkyl. In some embodiments, the C₁-C₈ alkyl is C₄-C₅ alkyl. In some embodiments, the C₁-C₈ alkyl is a branched C₁-C₈ alkyl. In some embodiments, the C₁-C₈ alkyl is an unbranched C₁-C₈ alkyl. In some embodiments, the C₁-C₈ alkyl is an unsubstituted C₁-C₈ alkyl. In some embodiments, the C₁-C₈ alkyl is a branched, unsubstituted C₁-C₈ alkyl. In some embodiments, the C₁-C₈ alkyl is an unbranched, unsubstituted C₁-C₈ alkyl.

In some embodiments, R¹² is C₁-C₈ (e.g., C₁, C₂, C₃, C₄, C₅, C₆, C₇, or C₈) alkoxy. The alkyl portion of an alkoxy group can have 1, 2, 3, 4, 5, or 6 carbon atoms (i.e., C₁-C₆ alkoxy) or 1, 2, or 3 carbon atoms (i.e., C₁-C₃ alkoxy). Examples of suitable alkoxy groups include, but are not limited to, methoxy (—O—CH₃ or —OMe), ethoxy (—OCH₂CH₃ or -OEt), isopropoxy (—O—CH(CH₃)₂), t-butoxy (—O—C(CH₃)₃ or -OtBu) and the like. Other examples of suitable alkoxy groups include, but are not limited to, sec-butoxy, tert-butoxy, pentoxy, and hexoxy.

R¹³ can be C₁-C₆ (e.g., C₁, C₂, C₃, C₄, C₅, or C₆) alkyl substituted with —OP(O)(OH)₂ or —OC(O)R¹². In some embodiments, the C₁-C₆ alkyl is methyl, ethyl, -n-propyl, isopropyl, -n-butyl, isobutyl, -s-butyl, -t-butyl, -n-pentyl, -2-pentyl, -3-pentyl, -2-methyl-2-butyl, -3-methyl-2-butyl, -3-methyl-1-butyl, -2-methyl-1-butyl, -1-hexyl, -2-hexyl, -3-hexyl, -2-methyl-2-pentyl, -3-methyl-2-pentyl, -4-methyl-2-pentyl, -3-methyl-3-pentyl, -2-methyl-3-pentyl, -2,3-dimethyl-2-butyl, or -3,3-dimethyl-2-butyl. In some embodiments, the C₁-C₆ alkyl is C₁-C₃ alkyl. In some embodiments, the C₁-C₆ alkyl is C₂-C₅ alkyl. In some embodiments, the C₁-C₆ alkyl is C₄-C₆ alkyl. In some embodiments, the C₁-C₆ alkyl is a branched C₁-C₆ alkyl. In some embodiments, the C₁-C₆ alkyl is an unbranched C₁-C₆ alkyl. In some embodiments, the C₁-C₆ alkyl is a substituted C₁-C₆ alkyl. In some embodiments, the C₁-C₆ alkyl is a branched, substituted C₁-C₆ alkyl. In some embodiments, the C₁-C₆ alkyl is an unbranched, substituted C₁-C₆ alkyl. In some embodiments, the C₁-C₆ alkyl is substituted with —OP(O)(OH)₂. In some embodiments, the C₁-C₆ alkyl is substituted with —OC(O)R¹².

The compound disclosed here is not

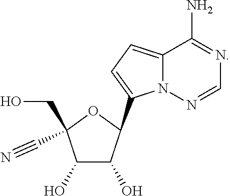 [SMILES: N#C[C@]1(CO)O[C@@H](c2ccc3c(N)ncnn23)[C@H](O)[C@@H]1O]

In some embodiments, when R³ is H, Base is not

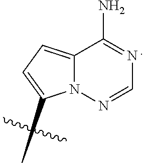

In some embodiments, when R³ is H, R¹ is not OH. In some embodiments, when R³ is H, R² is not OH. In some embodiments, when R³ is H, R¹ is not OH, R² is not OH, and Base is not

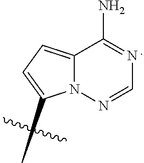

In some embodiments, R¹ is OH and R² is OH. In some embodiments, R¹ is OH and R³ is C(O)OR⁷, wherein R⁷ is C₃-C₈ carbocycle (e.g., cyclopentyl). In some embodiments, R¹ is OH and R³ is C(O)OR⁷, wherein R⁷ is C₁-C₈ alkyl (e.g., methyl, propyl, butyl, pentyl). In some embodiments, R¹ is OH and R³ is C(O)OR⁷, wherein R⁷ is C₃-C₈ carbocycle (e.g., cyclopropyl) substituted with one C₁-C₈ alkyl (e.g., methyl). In some embodiments, R¹ is OH and R³ is C(O)OR⁷, wherein R⁷ is C₁-C₈ alkyl (e.g., ethyl) substituted with phenyl. In some embodiments, R¹ is OH and R³ is C(O)OR⁷, wherein R⁷ is a 4- to 6-membered (e.g., 6-membered) heterocyclyl containing 1, 2, or 3 (e.g., one) O. In some embodiments, R² is OH and R³ is C(O)OR⁷, wherein R⁷ is C₃-C₈ carbocycle (e.g., cyclopentyl). In some embodiments, R² is OH and R³ is C(O)OR⁷, wherein R⁷ is C₁-C₈ alkyl (e.g., methyl, propyl, butyl, pentyl). In some embodiments, R² is OH and R³ is C(O)OR⁷, wherein R⁷ is C₃-C₈ carbocycle (e.g., cyclopropyl) substituted with one C₁-C₈ alkyl (e.g., methyl). In some embodiments, R² is OH and R³ is C(O)OR⁷, wherein R⁷ is C₁-C₈ alkyl (e.g., ethyl) substituted with phenyl. In some embodiments, R² is OH and R³ is C(O)OR⁷, wherein R⁷ is a 4- to 6-membered (e.g., 6-membered) heterocyclyl containing 1, 2, or 3 (e.g., one) O.

In some embodiments, R¹ is OH, R² is OH, and R³ is C(O)OR⁷, wherein R⁷ is C₃-C₈ carbocycle (e.g., cyclopentyl). In some embodiments, R¹ is OH, R² is OH, and R³ is C(O)OR⁷, wherein R⁷ is C₁-C₈ alkyl (e.g., methyl, propyl, butyl, pentyl). In some embodiments, R¹ is OH, R² is OH, and R³ is C(O)OR⁷, wherein R⁷ is C₃-C₈ carbocycle (e.g., cyclopropyl) substituted with one C₁-C₈ alkyl (e.g., methyl). In some embodiments, R¹ is OH, R² is OH, and R³ is C(O)OR⁷, wherein R⁷ is C₁-C₈ alkyl (e.g., ethyl) substituted with phenyl. In some embodiments, R¹ is OH, R² is OH, and R³ is C(O)OR⁷, wherein R⁷ is a 4- to 6-membered (e.g., 6-membered) heterocyclyl containing 1, 2, or 3 (e.g., one) O. In some embodiments, R¹ is OH, R² is OH, and R³ is C(O)OR⁷, wherein R⁷ is C₃-C₈ alkyl. In some embodiments, R¹ is OH, R² is OH, and R³ is C(O)OR⁷, wherein R⁷ is isobutyl. In some embodiments, R¹ is OH, R² is OH, and R³ is C(O)OR⁷, wherein R⁷ is tert-butyl.

In some embodiments, R¹ and R² are taken together to form —OC(O)O— and R³ is C(O)OR⁷, wherein R⁷ is C₁-C₈ alkyl (e.g., butyl). In some embodiments, R¹ is OH and R³ is H. In some embodiments, R¹ is OH and Base is

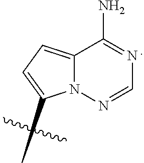

In some embodiments, R² is OH and Base is

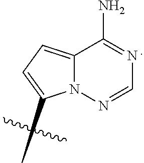

In some embodiments, R¹ is OH and Base is

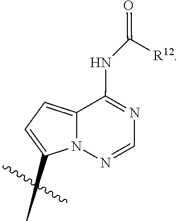

In some embodiments, R¹ is OH, Base is

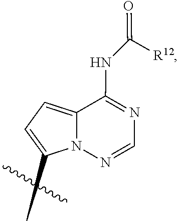

and R¹² is C₁-C₈ alkyl (e.g., butyl). In some embodiments, R¹ is OH and Base is

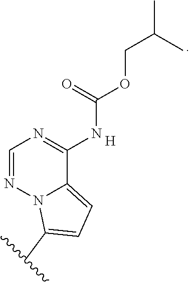

In some embodiments, R² is OH and Base is

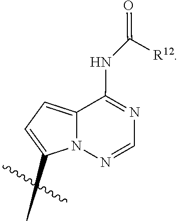

In some embodiments, R² is OH, Base is

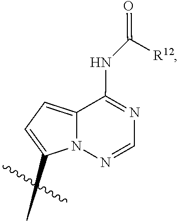

and R¹² is C₁-C₈ alkyl (e.g., butyl). In some embodiments, R¹ is OH, R² is OH, and Base is

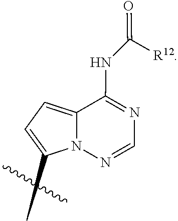

In some embodiments, R¹ is OH, R² is OH, and Base is

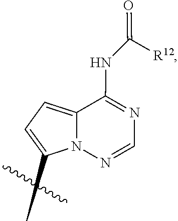

and R¹² is C₁-C₈ alkyl (e.g., butyl). In some embodiments, R¹ is OH, R² is OH, and Base is

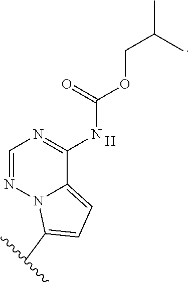

In some embodiments, R³ is H and Base is

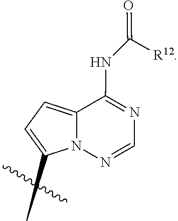

In some embodiments, R³ is H and Base is

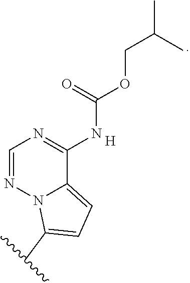

In some embodiments, R³ is C(O)OR⁷, R⁷ is C₃-C₈ carbocyclyl, and Base is

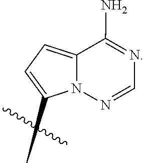

In some embodiments, R³ is C(O)OR⁷, R⁷ is C₃-C₈ carbocyclyl substituted with C₁-C₈ alkyl, and Base is

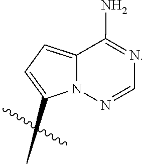

In some embodiments, R³ is C(O)OR⁷, R⁷ is C₁-C₈ alkyl, and Base is

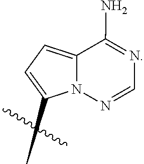

In some embodiments, R³ is C(O)OR⁷, R⁷ is C₁-C₈ alkyl substituted with phenyl, and Base is

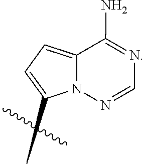

In some embodiments, R³ is C(O)OR⁷, R⁷ is 4- to 6-membered heterocyclyl containing 1, 2, or 3 O, and Base is

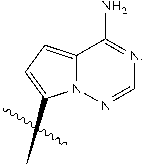

One of skill in the art is aware that each and every embodiment of a group (e.g., R¹) disclosed herein may be combined with any other embodiment of each of the remaining groups (e.g., R¹, R³, etc.) to generate a complete compound of Formula (I) or (II), or any Formula described herein or a pharmaceutically acceptable salt, stereoisomer, mixture of stereoisomers, or tautomer thereof, each of which is deemed within the ambit of the present disclosure.

In some embodiments, the compounds and pharmaceutically acceptable salts of Formula I include the compounds in Table 1 and the pharmaceutically acceptable salts thereof In some embodiments, the compounds disclosed herein include compounds in Table 1.

TABLE 1
Some Compounds disclosed herein

| COMPOUND # | STRUCTURE |
|---|---|
| 1 | 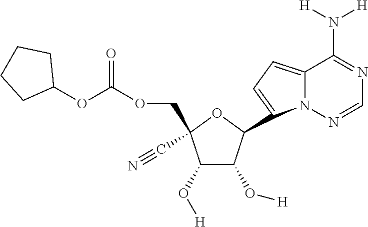 [SMILES: N#C[C@]1(COC(=O)OC2CCCC2)O[C@@H](c2ccc3c(N)ncnn23)[C@H](O)[C@@H]1O] |
| 2 | 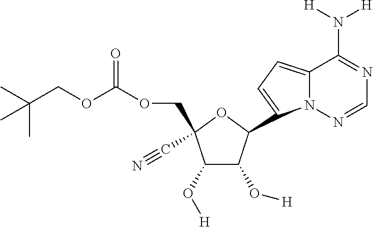 [SMILES: CC(C)(C)COC(=O)OC[C@@]1(C#N)O[C@@H](c2ccc3c(N)ncnn23)[C@H](O)[C@@H]1O] |
| 3 | 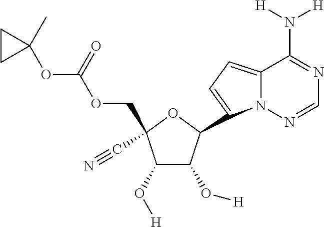 [SMILES: CC1(OC(=O)OC[C@@]2(C#N)O[C@@H](c3ccc4c(N)ncnn34)[C@H](O)[C@@H]2O)CC1] |
| 4 | 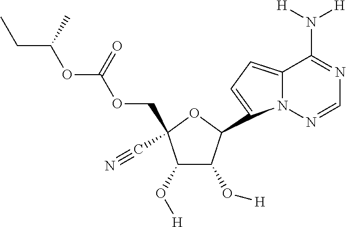 [SMILES: CC[C@H](C)OC(=O)OC[C@@]1(C#N)O[C@@H](c2ccc3c(N)ncnn23)[C@H](O)[C@@H]1O] |
| 5 | 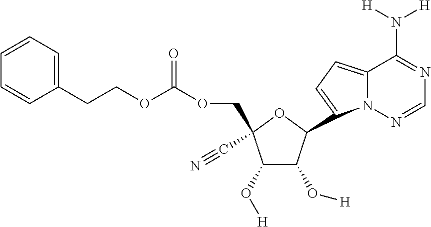 [SMILES: N#C[C@]1(COC(=O)OCCc2ccccc2)O[C@@H](c2ccc3c(N)ncnn23)[C@H](O)[C@@H]1O] |
| 6 | 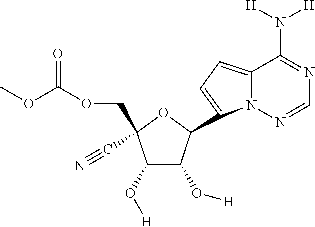 [SMILES: COC(=O)OC[C@@]1(C#N)O[C@@H](c2ccc3c(N)ncnn23)[C@H](O)[C@@H]1O] |
| 7 | 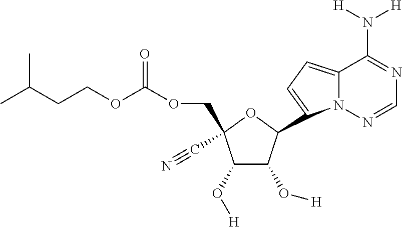 [SMILES: CC(C)CCOC(=O)OC[C@@]1(C#N)O[C@@H](c2ccc3c(N)ncnn23)[C@H](O)[C@@H]1O] |
| 8 | 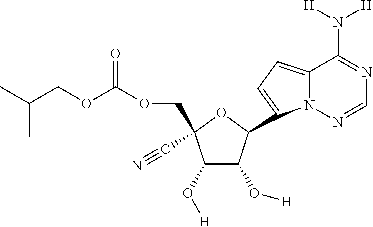 [SMILES: CC(C)COC(=O)OC[C@@]1(C#N)O[C@@H](c2ccc3c(N)ncnn23)[C@H](O)[C@@H]1O] |
| 9 | 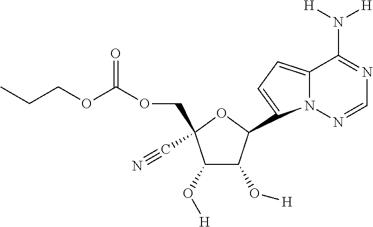 [SMILES: CCCOC(=O)OC[C@@]1(C#N)O[C@@H](c2ccc3c(N)ncnn23)[C@H](O)[C@@H]1O] |
| 10 | 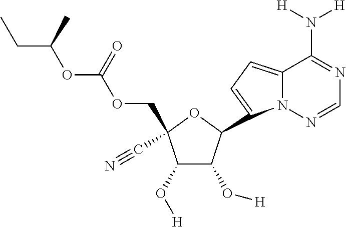 [SMILES: CC[C@@H](C)OC(=O)OC[C@@]1(C#N)O[C@@H](c2ccc3c(N)ncnn23)[C@H](O)[C@@H]1O] |
| 11 | 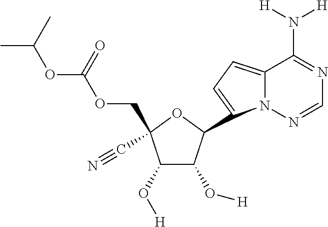 [SMILES: CC(C)OC(=O)OC[C@@]1(C#N)O[C@@H](c2ccc3c(N)ncnn23)[C@H](O)[C@@H]1O] |
| 12 | 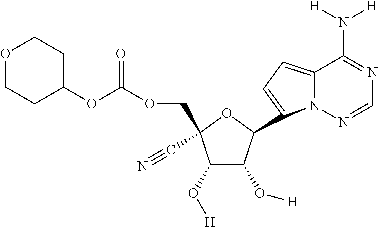 [SMILES: N#C[C@]1(COC(=O)OC2CCOCC2)O[C@@H](c2ccc3c(N)ncnn23)[C@H](O)[C@@H]1O] |
| 13 | 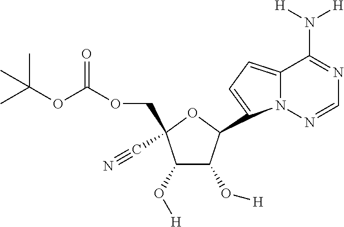 [SMILES: CC(C)(C)OC(=O)OC[C@@]1(C#N)O[C@@H](c2ccc3c(N)ncnn23)[C@H](O)[C@@H]1O] |
| 14 | 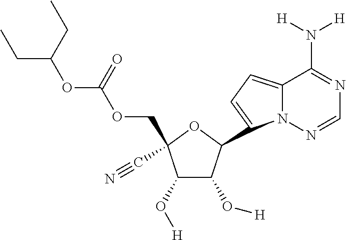 [SMILES: CCC(CC)OC(=O)OC[C@@]1(C#N)O[C@@H](c2ccc3c(N)ncnn23)[C@H](O)[C@@H]1O] |
| 15 | 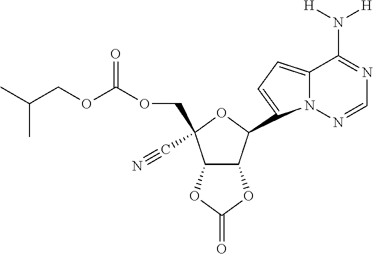 [SMILES: CC(C)COC(=O)OC[C@@]1(C#N)O[C@@H](c2ccc3c(N)ncnn23)[C@@H]2OC(=O)O[C@@H]21] |
| 16 | 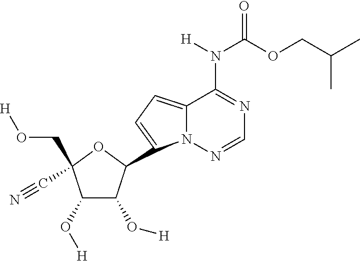 [SMILES: CC(C)COC(=O)Nc1ncnn2c([C@@H]3O[C@](C#N)(CO)[C@@H](O)[C@H]3O)ccc12] |
| 17 | 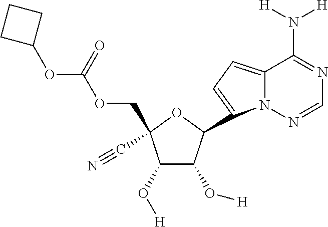 [SMILES: N#C[C@]1(COC(=O)OC2CCC2)O[C@@H](c2ccc3c(N)ncnn23)[C@H](O)[C@@H]1O] |
| 18 | 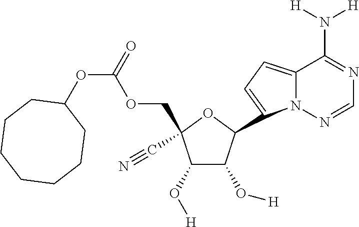 [SMILES: N#C[C@]1(COC(=O)OC2CCCCCCC2)O[C@@H](c2ccc3c(N)ncnn23)[C@H](O)[C@@H]1O] |
| 19 | 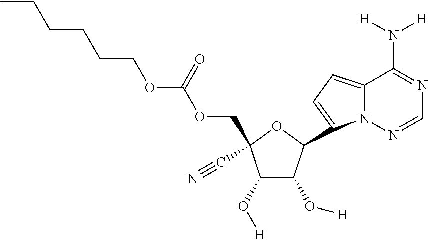 [SMILES: CCCCCCOC(=O)OC[C@@]1(C#N)O[C@@H](c2ccc3c(N)ncnn23)[C@H](O)[C@@H]1O] |
| 20 | 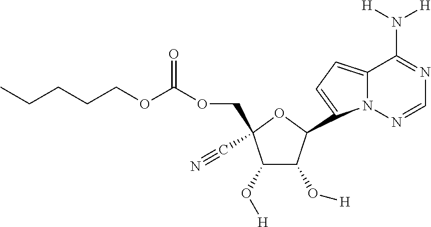 [SMILES: CCCCCOC(=O)OC[C@@]1(C#N)O[C@@H](c2ccc3c(N)ncnn23)[C@H](O)[C@@H]1O] |
| 21 | 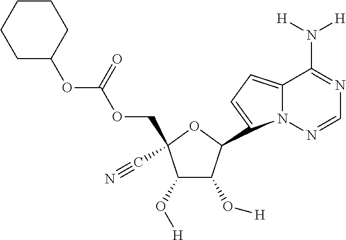 [SMILES: N#C[C@]1(COC(=O)OC2CCCCC2)O[C@@H](c2ccc3c(N)ncnn23)[C@H](O)[C@@H]1O] |
| 22 | 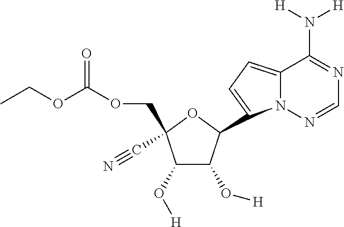 [SMILES: CCOC(=O)OC[C@@]1(C#N)O[C@@H](c2ccc3c(N)ncnn23)[C@H](O)[C@@H]1O] |
| 23 | 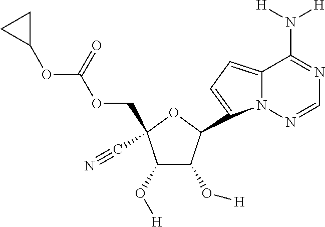 [SMILES: N#C[C@]1(COC(=O)OC2CC2)O[C@@H](c2ccc3c(N)ncnn23)[C@H](O)[C@@H]1O] |
| 24 | 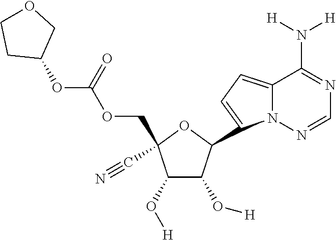 [SMILES: N#C[C@]1(COC(=O)O[C@@H]2CCOC2)O[C@@H](c2ccc3c(N)ncnn23)[C@H](O)[C@@H]1O] |
| 25 | 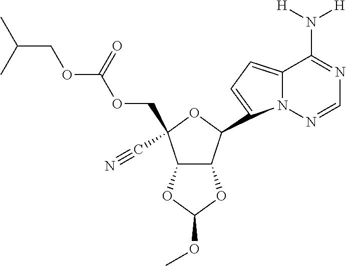 [SMILES: CO[C@@H]1O[C@H]2[C@H](c3ccc4c(N)ncnn34)O[C@](C#N)(COC(=O)OCC(C)C)[C@H]2O1] |
| 26 | 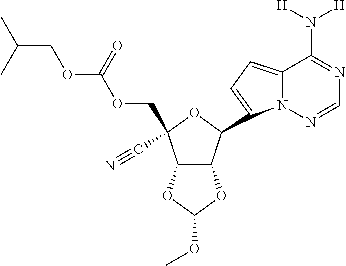 [SMILES: CO[C@H]1O[C@H]2[C@H](c3ccc4c(N)ncnn34)O[C@](C#N)(COC(=O)OCC(C)C)[C@H]2O1] |
| 27 | 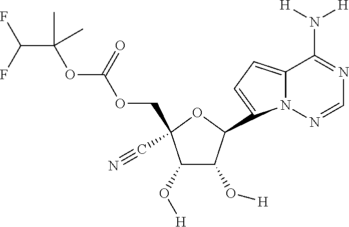 [SMILES: CC(C)(OC(=O)OC[C@@]1(C#N)O[C@@H](c2ccc3c(N)ncnn23)[C@H](O)[C@@H]1O)C(F)F] |
| 28 | 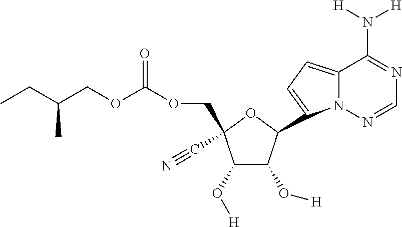 [SMILES: CC[C@H](C)COC(=O)OC[C@@]1(C#N)O[C@@H](c2ccc3c(N)ncnn23)[C@H](O)[C@@H]1O] |
| 29 | 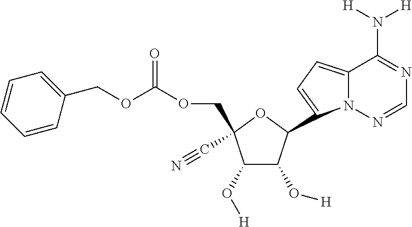 [SMILES: N#C[C@]1(COC(=O)OCc2ccccc2)O[C@@H](c2ccc3c(N)ncnn23)[C@H](O)[C@@H]1O] |
| 30 | 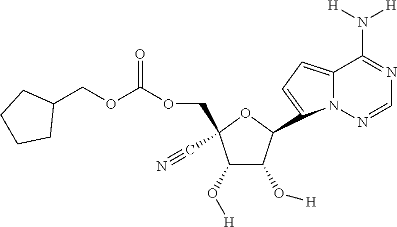 [SMILES: N#C[C@]1(COC(=O)OCC2CCCC2)O[C@@H](c2ccc3c(N)ncnn23)[C@H](O)[C@@H]1O] |
| 31 | 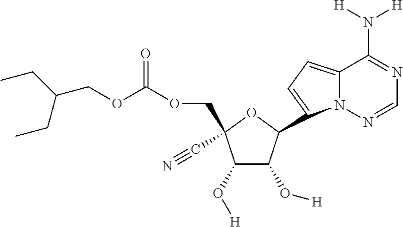 [SMILES: CCC(CC)COC(=O)OC[C@@]1(C#N)O[C@@H](c2ccc3c(N)ncnn23)[C@H](O)[C@@H]1O] |
| 32 | 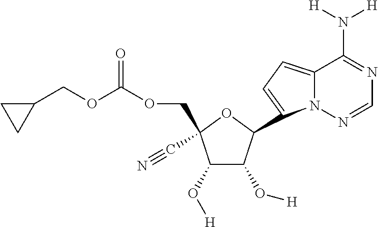 [SMILES: N#C[C@]1(COC(=O)OCC2CC2)O[C@@H](c2ccc3c(N)ncnn23)[C@H](O)[C@@H]1O] |
| 33 | 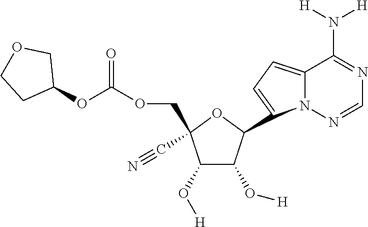 [SMILES: N#C[C@]1(COC(=O)O[C@H]2CCOC2)O[C@@H](c2ccc3c(N)ncnn23)[C@H](O)[C@@H]1O] |
| 34 | 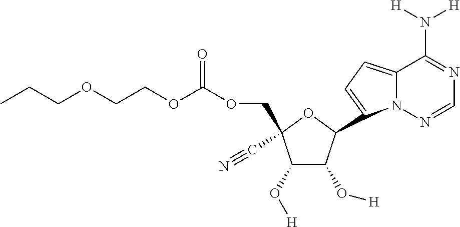 [SMILES: CCCOCCOC(=O)OC[C@@]1(C#N)O[C@@H](c2ccc3c(N)ncnn23)[C@H](O)[C@@H]1O] |
| 35 | 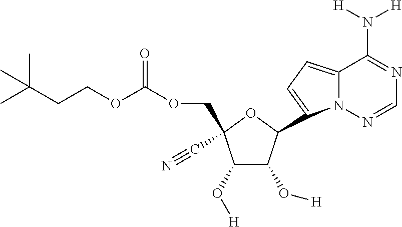 [SMILES: CC(C)(C)CCOC(=O)OC[C@@]1(C#N)O[C@@H](c2ccc3c(N)ncnn23)[C@H](O)[C@@H]1O] |
| 36 | 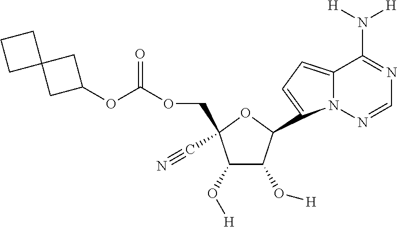 [SMILES: N#C[C@]1(COC(=O)OC2CC3(CCC3)C2)O[C@@H](c2ccc3c(N)ncnn23)[C@H](O)[C@@H]1O] |
| 37 | 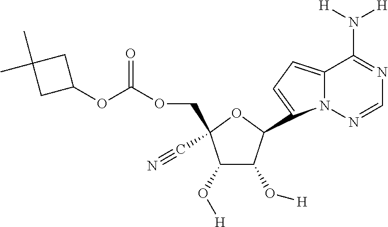 [SMILES: CC1(C)CC(OC(=O)OC[C@@]2(C#N)O[C@@H](c3ccc4c(N)ncnn34)[C@H](O)[C@@H]2O)C1] |
| 38 | 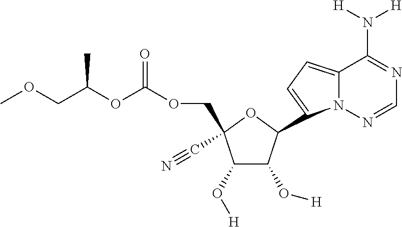 [SMILES: COC[C@@H](C)OC(=O)OC[C@@]1(C#N)O[C@@H](c2ccc3c(N)ncnn23)[C@H](O)[C@@H]1O] |
| 39 | 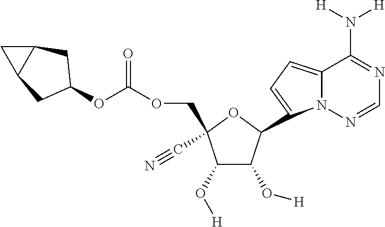 [SMILES: N#C[C@]1(COC(=O)O[C@@H]2C[C@@H]3C[C@@H]3C2)O[C@@H](c2ccc3c(N)ncnn23)[C@H](O)[C@@H]1O] |
| 40 | 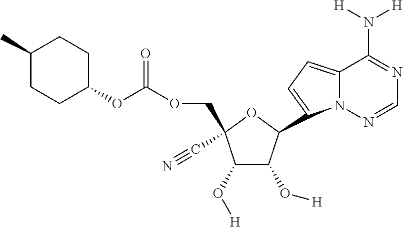 [SMILES: C[C@H]1CC[C@H](OC(=O)OC[C@@]2(C#N)O[C@@H](c3ccc4c(N)ncnn34)[C@H](O)[C@@H]2O)CC1] |
| 42 | 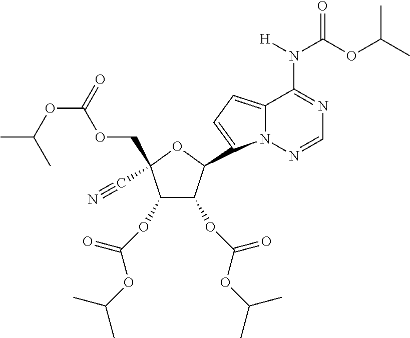 [SMILES: CC(C)OC(=O)Nc1ncnn2c([C@@H]3O[C@](C#N)(COC(=O)OC(C)C)[C@@H](OC(=O)OC(C)C)[C@H]3OC(=O)OC(C)C)ccc12] |
| 43 | 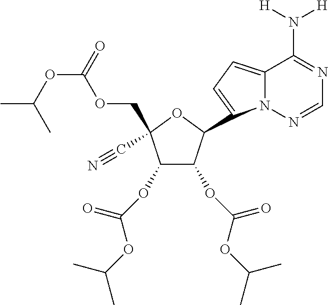 [SMILES: CC(C)OC(=O)OC[C@@]1(C#N)O[C@@H](c2ccc3c(N)ncnn23)[C@H](OC(=O)OC(C)C)[C@@H]1OC(=O)OC(C)C] |
| 44 | 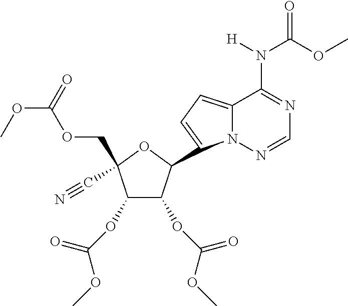 |
| 45 | 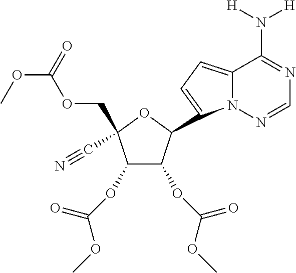 [SMILES: COC(=O)OC[C@@]1(C#N)O[C@@H](c2ccc3c(N)ncnn23)[C@H](OC(=O)OC)[C@@H]1OC(=O)OC] |

Also falling within the scope herein are the in vivo metabolic products of the compounds described herein. Such products may result for example from the oxidation, reduction, hydrolysis, amidation, esterification and the like of the administered compound, primarily due to enzymatic processes. Accordingly, included are novel and unobvious compounds produced by a process comprising contacting a compound with a mammal for a period of time sufficient to yield a metabolic product thereof. Such products typically are identified by preparing a radiolabelled (e.g., ¹⁴C or ³H) compound, administering it parenterally in a detectable dose (e.g., greater than about 0.5 mg/kg) to an animal such as rat, mouse, guinea pig, monkey, or to man, allowing sufficient time for metabolism to occur (typically about 30 seconds to 30 hours) and isolating its conversion products from the urine, blood or other biological samples. These products are easily isolated since they are labeled (others are isolated by the use of antibodies capable of binding epitopes surviving in the metabolite). The metabolite structures are determined in conventional fashion, e.g., by MS or NMR analysis. In general, analysis of metabolites is done in the same way as conventional drug metabolism studies. The conversion products, so long as they are not otherwise found in vivo, are useful in diagnostic assays for therapeutic dosing of the compounds even if they possess no HSV antiviral activity of their own.

Recipes and methods for determining stability of compounds in surrogate gastrointestinal secretions are known. Compounds are defined herein as stable in the gastrointestinal tract where less than about 50 mole percent of the protected groups are deprotected in surrogate intestinal or gastric juice upon incubation for 1 hour at 37° C. Simply because the compounds are stable to the gastrointestinal tract does not mean that they cannot be hydrolyzed in vivo. The prodrugs typically will be stable in the digestive system but may be substantially hydrolyzed to the parental drug in the digestive lumen, liver, lung or other metabolic organ, or within cells in general. As used herein, a prodrug is understood to be a compound that is chemically designed to efficiently liberate the parent drug (i.e., Compound 0 below) after overcoming biological barriers to oral delivery.

### IV. Pharmaceutical Compositions

Also disclosed herein are pharmaceutical compositions comprising a pharmaceutically effective amount of a compound of the present disclosure (e.g., a compound of Formulas I to Formula II), or a pharmaceutically acceptable salt thereof, and a pharmaceutically acceptable carrier or excipient. Also provided herein is a pharmaceutical composition comprising a pharmaceutically effective amount of a compound of the present disclosure, or a pharmaceutically acceptable salt thereof, and a pharmaceutically acceptable carrier or excipient.

The compounds disclosed herein can be formulated with conventional carriers and excipients. Tablets can contain, for instance, excipients, glidants, fillers, binders, or a combination thereof. Aqueous formulations are prepared in sterile form, and when intended for delivery by other than oral administration generally will be isotonic. Exemplary excipients include, but are not limited to, those set forth in the “HANDBOOK OF PHARMACEUTICAL EXCIPIENTS” (1986). Excipients can include, for example, ascorbic acid and other antioxidants, chelating agents such as EDTA, carbohydrates such as dextran, hydroxyalkylcellulose, hydroxyalkylmethylcellulose, stearic acid, and combinations thereof. In some embodiments, the formulation is basic. In some embodiments, the formulation is acidic. In some embodiments, the formulation has a neutral pH. In some embodiments, the pH of the formulations is from 2 to 11 (e.g., 2, 3, 4, 5, 6, 7, 8, 9, 10, 11, 2-3, 2-4, 2-5, 2-6, 2-7, 2-8, 2-9, 2-10, 3-4, 3-5, 3-6, 3-7, 3-8, 3-9, 3-10, 4-5, 4-6, 4-7, 4- 8, 4-9, 4-10, 4-11, 5-6, 5-7, 5-8, 5-9, 5-10, 5-11, 6-7, 6-8, 6-9, 6-10, 6-11, 7-8, 7-9, 7-10, 7-11, 8-9, 8-10, 8-11, 9-10, or 9-11).

In some embodiments, the compounds disclosed herein have pharmacokinetic properties (e.g., oral bioavailability) suitable for oral administration of the compounds. Formulations suitable for oral administration can, for instance, be presented as discrete units such as capsules, cachets or tablets, each containing a predetermined amount of the active ingredient; as a powder or granules; as a solution or a suspension in an aqueous or non-aqueous liquid; or as an oil-in-water liquid emulsion or a water-in-oil liquid emulsion. The active ingredient can also be administered, for instance, as a bolus, electuary, or paste.

A tablet can be made by compression or molding, optionally with at least accessory ingredients. Compressed tablets can be prepared by compressing in a suitable machine the active ingredient in a free-flowing form such as, for instance, a powder or granules, optionally mixed with a binder, lubricant, inert diluent, preservative, surface active, dispersing agent, or a combination thereof. Molded tablets can be made by molding in a suitable machine a mixture of the powdered active ingredient moistened with an inert liquid diluent. The tablets can optionally be coated or scored and optionally are formulated so as to provide slow or controlled release of the active ingredient therefrom.

For infections of the eye or other external tissues (e.g., mouth and skin), the formulations can be applied as a topical ointment or cream containing the active ingredient(s) in an amount of, for example, 0.075 to 20% w/w (including active ingredient(s) in a range from 0.1% to 20% in increments of 0.1% w/w such as 0.6% w/w, 0.7% w/w, etc.), from 0.2% to 15% w/w, or from 0.5% to 10% w/w. When formulated in an ointment, the active ingredients can be employed in some embodiments with either a paraffinic or a water-miscible ointment base. Alternatively, the active ingredients can be formulated in a cream with an oil-in-water cream base.

In some embodiments, the aqueous phase of the cream base can include, for example, from 30% to 90% (e.g., 35%, 40%, 45%, 50%, 55%, 60%, 65%, 70%, 75%, 80%, 85%) w/w of a polyhydric alcohol, i.e., an alcohol having two or more hydroxyl groups such as propylene glycol, butane 1,3-diol, mannitol, sorbitol, glycerol and polyethylene glycol (including PEG 400) and mixtures thereof. In some embodiments, the cream base can include, for instance, a compound that enhances absorption or penetration of the active ingredient through the skin or other affected areas. Examples of such dermal penetration enhancers include, but are not limited to, dimethyl sulfoxide and related analogs. In some embodiments, the cream or emulsion does not include water.

The oily phase of the emulsions can be constituted from known ingredients in a known manner. In some embodiments, the phase comprises merely an emulsifier (otherwise known as an emulgent). In some embodiments, the phase comprises a mixture of at least one emulsifier with a fat, an oil, or a combination thereof. In some embodiments, a hydrophilic emulsifier is included together with a lipophilic emulsifier that acts as a stabilizer. Together, the emulsifier(s) with or without stabilizer(s) can make up the so-called emulsifying wax, and the wax together with the oil and fat make up the so-called emulsifying ointment base that can form the oily dispersed phase of the cream formulations.

Emulgents and emulsion stabilizers suitable for use in the formulation can include, but are not limited to, TWEEN® 60, TWEEN® 80, SPAN® 80, cetostearyl alcohol, benzyl alcohol, myristyl alcohol, glyceryl mono-stearate, sodium lauryl sulfate, and combinations thereof.

The choice of suitable oils or fats for the formulation can be based on achieving the desired cosmetic properties. In some embodiments, the cream can be a non-greasy, non-staining, and washable product with suitable consistency to avoid leakage from tubes or other containers. In some embodiments, esters can be included, such as, for example, straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate, a blend of branched chain esters known as CRODAMOL® CAP, or a combination thereof. In some embodiments, high melting point lipids such as white soft paraffin and/or liquid paraffin or other mineral oils can be included.

In some embodiments, the compounds disclosed herein are administered alone. In some embodiments, the compounds disclosed herein are administered in pharmaceutical compositions. In some embodiments, the pharmaceutical compositions are for veterinary use. In some embodiments, the pharmaceutical compositions are for human use. In some embodiments, the pharmaceutical compositions disclosed herein include at least one additional therapeutic agent.

Pharmaceutical compositions disclosed herein can be in any form suitable for the intended method of administration. The pharmaceutical compositions disclosed herein can be presented in unit dosage form and can be prepared by any of the methods well known in the art of pharmacy. Exemplary techniques and formulations can be found, for instance, in REMINGTON'S PHARMACEUTICAL SCIENCES (Mack Publishing Co., Easton, PA). Such methods can include the step of bringing into association a compound disclosed herein with the carrier that constitutes at least accessory ingredients. In general, the formulations can be prepared by uniformly and intimately bringing into association the active ingredient with liquid carriers or finely divided solid carriers or both, and then, if necessary, shaping the product.

When used for oral use for example, tablets, troches, lozenges, aqueous or oil suspensions, dispersible powders or granules, emulsions, hard or soft capsules, solutions, syrups or elixirs can be prepared. Formulations intended for oral use can be prepared according to any method known to the art for the manufacture of pharmaceutical compositions and such formulations can contain at least agents including sweetening agents, flavoring agents, coloring agents and preserving agents, in order to provide a palatable preparation. Tablets containing the active ingredient in admixture with non-toxic pharmaceutically acceptable excipient which are suitable for manufacture of tablets are acceptable. These excipients can be, for example, inert diluents, such as calcium or sodium carbonate, lactose, calcium or sodium phosphate; granulating and disintegrating agents, such as maize starch, or alginic acid; binding agents, such as starch, gelatin or acacia; and lubricating agents, such as magnesium stearate, stearic acid or talc. Tablets can be uncoated or can be coated by known techniques including microencapsulation to delay disintegration and adsorption in the gastrointestinal tract and thereby provide a sustained action over a longer period. For example, a time delay material such as glyceryl monostearate or glyceryl distearate alone or with a wax can be employed.

Formulations for oral use can be also presented as hard gelatin capsules where the active ingredient is mixed with an inert solid diluent, for example calcium phosphate or kaolin, or as soft gelatin capsules wherein the active ingredient is mixed with water or an oil medium, such as peanut oil, liquid paraffin, or olive oil.

Aqueous suspensions can contain the active materials in admixture with excipients suitable for the manufacture of aqueous suspensions. Such excipients can include, for instance, a suspending agent, such as sodium carboxymethylcellulose, methylcellulose, hydroxypropyl methylcelluose, sodium alginate, polyvinylpyrrolidone, gum tragacanth and gum acacia, and dispersing or wetting agents such as a naturally-occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycetanol), a condensation product of ethylene oxide with a partial ester derived from a fatty acid and a hexitol anhydride (e.g., polyoxyethylene sorbitan monooleate). The aqueous suspension can also contain, for example, at least preservatives such as ethyl or n-propyl p-hydroxy-benzoate, one or more coloring agents, one or more flavoring agents, one or more sweetening agents (such as sucrose or saccharin), or combinations thereof. Further non-limiting examples of suspending agents include cyclodextrin. In some embodiments, the suspending agent is sulfobutyl ether beta-cyclodextrin (SEB-beta-CD), for example CAPTISOL©.

Oil suspensions can be formulated by suspending the active ingredient in a vegetable oil (e.g., arachis oil, olive oil, sesame oil, coconut oil, or a combination thereof), a mineral oil such as liquid paraffin, or a combination thereof. The oral suspensions can contain, for instance, a thickening agent, such as beeswax, hard paraffin, cetyl alcohol, or a combination thereof. In some embodiments, sweetening agents, such as those set forth above, and/or flavoring agents, are added to provide a palatable oral preparation. In some embodiments, the formulations disclosed herein are preserved by the addition of an antioxidant such as ascorbic acid.

Dispersible powders and granules suitable for preparation of an aqueous suspension by the addition of water can provide the active ingredient in admixture with a dispersing or wetting agent, a suspending agent, a preservative, and combinations thereof. Suitable dispersing or wetting agents and suspending agents are exemplified by those disclosed above. Additional excipients, for example sweetening, flavoring and coloring agents, can also be present.

The pharmaceutical compositions can also be in the form of oil-in-water emulsions. The oily phase can be a vegetable oil, such as olive oil or arachis oil, a mineral oil, such as liquid paraffin, or a mixture of these. Suitable emulsifying agents include naturally-occurring gums, such as gum acacia and gum tragacanth, naturally-occurring phosphatides, such as soybean lecithin, esters or partial esters derived from fatty acids and hexitol anhydrides, such as sorbitan monooleate, and condensation products of these partial esters with ethylene oxide, such as polyoxyethylene sorbitan monooleate. The emulsion can also contain sweetening and flavoring agents. Syrups and elixirs can be formulated with sweetening agents, such as for instance, glycerol, sorbitol or sucrose. Such formulations can also contain, for instance, a demulcent, a preservative, a flavoring, a coloring agent, or a combination thereof.

The pharmaceutical compositions can be in the form of a sterile injectable or intravenous preparation, such as a sterile injectable aqueous or oleaginous suspension. This suspension can be formulated according to the known art using those suitable dispersing or wetting agents and suspending agents which have been mentioned above. The sterile injectable or intravenous preparation can also be a sterile injectable solution or suspension in a non-toxic parenterally acceptable diluent or solvent, such as a solution in 1,3-butane-diol or prepared as a lyophilized powder. Among the acceptable vehicles and solvents that can be employed are water, Ringer's solution and isotonic sodium chloride solution. In addition, sterile fixed oils can be employed as a solvent or suspending medium. For this purpose, any bland fixed oil can be employed including synthetic mono- or diglycerides. In addition, fatty acids such as oleic acid can likewise be used in the preparation of injectables. Among the acceptable vehicles and solvents that can be employed include, but are not limited to, water, Ringer's solution isotonic sodium chloride solution, and hypertonic sodium chloride solution.

The amount of active ingredient that can be combined with the carrier material to produce a single dosage form will vary depending upon the host treated and the particular mode of administration. For example, a time-release formulation intended for oral administration to humans can contain approximately 1 mg to 2000 mg of active material compounded with an appropriate and convenient amount of carrier material, which can vary from 5% to 95% of the total formulations (weight:weight). For example, a time-release formulation intended for oral administration to humans can contain approximately 1 mg to 1000 mg of active material compounded with an appropriate and convenient amount of carrier material, which can vary from 5% to 95% of the total formulations (weight:weight). The pharmaceutical composition can be prepared to provide easily measurable amounts for administration. For example, an aqueous solution intended for intravenous infusion can contain from 3 μg to 500 μg of the active ingredient per milliliter of solution in order that infusion of a suitable volume at a rate of 30 mL/hr can occur.

Formulations suitable for topical administration to the eye also include eye drops wherein the active ingredient is dissolved or suspended in a suitable carrier, especially an aqueous solvent for the active ingredient. In some embodiments, the compounds disclosed herein are included in the pharmaceutical compositions disclosed herein in a concentration of 0.5% to 20% (e.g., 0.5% to 10%, 1.5% w/w).

Formulations suitable for topical administration in the mouth include lozenges can comprise an active ingredient (i.e., a compound disclosed herein and/or additional therapeutic agents) in a flavored basis, usually sucrose and acacia or tragacanth; pastilles comprising the active ingredient in an inert basis such as gelatin and glycerin, or sucrose and acacia; and mouthwashes comprising the active ingredient in a suitable liquid carrier.

Formulations for rectal administration can be presented as a suppository with a suitable base comprising for example cocoa butter or a salicylate.

Formulations suitable for vaginal administration can be presented as pessaries, tampons, creams, gels, pastes, foams or spray formulations containing in addition to the active ingredient such carriers as are known in the art to be appropriate.

Formulations suitable for parenteral administration include aqueous and non-aqueous sterile injection solutions that can contain anti-oxidants, buffers, bacteriostats and solutes that render the formulation isotonic with the blood of the intended recipient; and aqueous and non-aqueous sterile suspensions that can include suspending agents and thickening agents.

The formulations can be presented in unit-dose or multi-dose containers, for example, sealed ampoules and vials, and can be stored in a freeze-dried (lyophilized) condition requiring only the addition of the sterile liquid carrier, for example water for injection, immediately before use. Extemporaneous injection solutions and suspensions are prepared from sterile powders, granules and tablets of the kind previously described. Preferred unit-dosage formulations are those containing a daily dose or unit daily sub-dose, as herein above recited, or an appropriate fraction thereof, of the active ingredient.

It should be understood that in addition to the ingredients particularly mentioned above the formulations can include other agents conventional in the art having regard to the type of formulation in question, for example those suitable for oral administration can include flavoring agents.

Further provided are veterinary formulations comprising a compound disclosed herein together with a veterinary carrier therefor.

Veterinary carriers are materials useful for the purpose of administering the formulation and can be solid, liquid or gaseous materials which are otherwise inert or acceptable in the veterinary art and are compatible with the active ingredient. These veterinary formulations can be administered orally, parenterally, or by any other desired route.

Compounds herein are used to provide controlled release pharmaceutical compositions containing as active ingredient one or more of the compounds (“controlled release formulations”) in which the release of the active ingredient can be controlled and regulated to allow less frequency dosing or to improve the pharmacokinetic or toxicity profile of a given active ingredient.

Effective dose of active ingredient depends at least on the nature of the condition being treated, toxicity, whether the compound is being used prophylactically (lower doses) or against an active viral infection, the method of delivery, and the pharmaceutical composition, and will be determined by the clinician using conventional dose escalation studies. In some embodiments, the effective dose is from 0.0001 to 100 mg/kg body weight per day; for instance, from 10 to 30 mg/kg body weight per day; from 15 to 25 mg/kg body weight per day; from 10 to 15 mg/kg body weight per day; or from 20 to 30 mg/kg body weight per day. For example, the daily candidate dose for an adult human of approximately 70 kg body weight can range from 1 mg to 2000 mg (e.g., from 5 mg to 500 mg, from 500 mg to 1000 mg, from 1000 mg to 1500 mg, from 1500 mg to 2000 mg), and can take the form of single or multiple doses. For example, the daily candidate dose for an adult human of approximately 70 kg body weight can range from 1 mg to 1000 mg (e.g., from 5 mg to 500 mg), and can take the form of single or multiple doses.

### V. Kits

Also provided herein are kits that includes a compound disclosed herein or a pharmaceutically acceptable salt thereof. In some embodiments the kits described herein can comprise a label and/or instructions for use of the compound in the treatment of a disease or condition in a subject (e.g., human) in need thereof. In some embodiments, the disease or condition is viral infection.

In some embodiments, the kit can also comprise one or more additional therapeutic agents and/or instructions for use of additional therapeutic agents in combination with the compound disclosed herein in the treatment of the disease or condition in a subject (e.g., human) in need thereof.

In some embodiments, the kits provided herein comprise individual dose units of a compound as described herein, or a pharmaceutically acceptable salt, racemate, enantiomer, diastereomer, tautomer, polymorph, pseudopolymorph, amorphous form, hydrate or solvate thereof. Examples of individual dosage units can include pills, tablets, capsules, prefilled syringes or syringe cartridges, IV bags, inhalers, nebulizers etc., each comprising a therapeutically effective amount of the compound in question, or a pharmaceutically acceptable salt, racemate, enantiomer, diastereomer, tautomer, polymorph, pseudopolymorph, amorphous form, hydrate or solvate thereof. In some embodiments, the kit can contain a single dosage unit and in others multiple dosage units are present, such as the number of dosage units required for a specified regimen or period.

Also provided are articles of manufacture that include a compound disclosed herein, or a pharmaceutically acceptable salt, stereoisomer, mixture of stereoisomers or tautomer thereof, and a container. In some embodiments, the container of the article of manufacture is a vial, jar, ampoule, preloaded syringe, blister package, tin, can, bottle, box, an intravenous bag, an inhaler, or a nebulizer.

### VI. Administration

One or more compounds of the disclosure are administered by any route appropriate to the condition to be treated. Suitable routes include oral, rectal, inhalation, pulmonary, topical (including buccal and sublingual), vaginal and parenteral (including subcutaneous, intramuscular, intravenous, intradermal, intrathecal and epidural), and the like. In some embodiments, the compounds disclosed herein are administered by inhalation or intravenously. It will be appreciated that the route can vary with for example the condition of the recipient.

In the methods of the present disclosure for the treatment of a viral infection, the compounds of the present disclosure can be administered at any time to a subject who can come into contact with the virus or is already suffering from the viral infection. In some embodiments, the compounds of the present disclosure can be administered prophylactically to subjects coming into contact with subjects suffering from the viral infection or at risk of coming into contact with humans suffering from the viral infection, e.g., healthcare providers. In some embodiments, administration of the compounds of the present disclosure can be to subjects testing positive for the viral infection but not yet showing symptoms of the viral infection. In the methods of the present disclosure for the treatment of a viral infection, the compounds of the present disclosure can be administered at any time to a human who can come into contact with the virus or is already suffering from the viral infection. In some embodiments, the compounds of the present disclosure can be administered prophylactically to humans coming into contact with humans suffering from the viral infection or at risk of coming into contact with humans suffering from the viral infection, e.g., healthcare providers. In some embodiments, administration of the compounds of the present disclosure can be to humans testing positive for the viral infection but not yet showing symptoms of the viral infection. In some embodiments, administration of the compounds of the present disclosure can be to humans upon commencement of symptoms of the viral infection.

In some embodiments, the methods disclosed herein comprise event driven administration of the compound disclosed herein, or a pharmaceutically acceptable salt thereof, to the subject.

As used herein, the terms “event driven” or “event driven administration” refer to administration of a compound of Formula I-II, or a pharmaceutically acceptable salt thereof, (1) before an event (e.g., 2 hours, 1 day, 2 days, 5 day, or 7 or more days before the event) that would expose the subject to the virus (or that would otherwise increase the subject's risk of acquiring the viral infection); and/or (2) during an event (or more than one recurring event) that would expose the subject to the virus (or that would otherwise increase the subject's risk of acquiring the viral infection); and/or (3) after an event (or after the final event in a series of recurring events) that would expose the subject to the virus (or that would otherwise increase the subject's risk of acquiring the viral infection). In some embodiments, the event driven administration is performed pre-exposure of the subject to the virus. In some embodiments, the event driven administration is performed post-exposure of the subject to the virus. In some embodiments, the event driven administration is performed pre-exposure of the subject to the virus and post-exposure of the subject to the virus.

In certain embodiments, the methods disclosed herein involve administration prior to and/or after an event that would expose the subject to the virus or that would otherwise increase the subject's risk of acquiring the viral infection, e.g., as pre-exposure prophylaxis (PrEP) and/or as post-exposure prophylaxis (PEP). In some embodiments, the methods disclosed herein comprise pre-exposure prophylaxis (PrEP). In some embodiments, methods disclosed herein comprise post-exposure prophylaxis (PEP).

In some embodiments, a compound disclosed herein, or a pharmaceutically acceptable salt thereof, is administered before exposure of the subject to the virus.

In some embodiments, a compound disclosed herein, or a pharmaceutically acceptable salt thereof, is administered before and after exposure of the subject to the virus.

In some embodiments, a compound disclosed herein, or a pharmaceutically acceptable salt thereof, is administered after exposure of the subject to the virus.

An example of event driven dosing regimen includes administration of a compound disclosed herein, or a pharmaceutically acceptable salt thereof, within 24 to 2 hours before the virus, followed by administration of a compound disclosed herein, or a pharmaceutically acceptable salt, every 24 hours during the period of exposure, followed by a further administration of a compound disclosed herein, or a pharmaceutically acceptable salt thereof, after the last exposure, and one last administration of a compound disclosed herein, or a pharmaceutically acceptable salt thereof, 24 hours later.

A further example of an event driven dosing regimen includes administration of the compound of any one of Formulas I-II, or a pharmaceutically acceptable salt thereof, within 24 hours before the viral exposure, then daily administration during the period of exposure, followed by a last administration approximately 24 hours later after the last exposure (which can be an increased dose, such as a double dose).

Effective dose of active ingredient depends at least on the nature of the condition being treated, toxicity, whether the compound is being used prophylactically or against an active viral infection, the method of delivery, and the pharmaceutical composition, and will be determined by the clinician using conventional dose escalation studies. It can be expected to be from 0.0001 mg/kg to 100 mg/kg body weight per day (e.g., from 0.01 mg/kg to 10 mg/kg body weight per day; from 0.01 mg/kg to 5 mg/kg body weight per day; from 0.05 mg/kg to 0.5 mg/kg body weight per day). In some embodiments, the daily candidate dose for an adult human of approximately 70 kg body weight is from 1 mg to 4000 mg (e.g., 5 mg to 500 mg, 500 mg to 1000 mg, 1000 mg to 1500 mg, 1500 mg to 2000 mg, 2000 mg to 3000 mg, 3000 mg to 4000 mg) and can take the form of single or multiple doses (e.g., 2 doses per day, 3 doses per day). For example, the daily candidate dose for an adult human of approximately 70 kg body weight can range from 1 mg to 1000 mg (e.g., from 5 mg to 500 mg) and can take the form of single or multiple doses.

Any suitable period of time for administration of the compounds of the present disclosure is contemplated. For example, administration can be for from 1 day to 100 days, including 2, 3, 4, 5, 6, 7, 8, 9, 10, 15, 20, 25, 30, 40, 50, 60, 70, 80, or 90 days. The administration can also be for from 1 week to 15 weeks, including 1, 2, 3, 4, 5, 6, 7, 8, 9, 10, 11, 12, 13, 14, or 15 weeks. Longer periods of administration are also contemplated.

In some embodiments, the compounds disclosed herein are administered once daily. In some embodiments, the compounds disclosed herein are administered twice daily. In some embodiments, the compounds disclosed herein are administered once every alternate day. In some embodiments, the compounds disclosed herein are administered once a week. In some embodiments, the compounds disclosed herein are administered twice a week.

In some embodiments, one or more compounds disclosed herein are administered once daily. The once daily dose can be administered for as long as required, for example for up to 5 days, up to 7 days, up to 10 days, up to 15 days, up to 20 days, up to 25 days, up to a month or longer. In some embodiments, the once daily dose is administered for up to 20 days, up to 15 days, up to 14 days, up to 13 days, up to 12 days, up to 10 days, up to 8 days, up to 6 days, up to 4 days, up to 3 days, up to 2 days, or for one day.

In some embodiments, the one or more compounds disclosed herein are dosed once daily, for 6 to 12 days, for example for 8-10 days. In some embodiments, the one or more compounds are administered once daily for 9 days. In some embodiments, the one or more compounds are administered once daily for 10 days. In some embodiments 50-150 mg of one or more compounds disclosed herein is administered once daily for 5 to 12 days, for e.g., for 5 days, 6 days, 7 days, 8 days, 9 days, 10 days, 11 days, or 12 days. In some embodiments 100 mg of one or more compounds disclosed herein is administered once daily for 5 to 12 days, for e.g., for 5 days, 6 days, 7 days, 8 days, 9 days, 10 days, 11 days, or 12 days. In some embodiments 500-2000 mg (e.g., 500-1000 mg, 1000-1500 mg) of one or more compounds disclosed herein is administered once daily for 5 to 12 days, for e.g., for 5 days, 6 days, 7 days, 8 days, 9 days, 10 days, 11 days, or 12 days.

In some embodiments, one or more compounds disclosed herein are administered twice daily. The twice daily dose can be administered for as long as required, for example for up to 5 days, up to 7 days, up to 10 days, up to 15 days, up to 20 days, up to 25 days, up to a month or longer. In some embodiments, the twice daily dose is administered for up to 20 days, up to 15 days, up to 14 days, up to 13 days, up to 12 days, up to 10 days, up to 8 days, up to 6 days, up to 4 days, up to 3 days, up to 2 days, or for one day.

In some embodiments, the one or more compounds disclosed herein are dosed twice daily, for 6 to 12 days, for example for 8-10 days. In some embodiments, the one or more compounds are administered twice daily for 9 days. In some embodiments, the one or more compounds are administered twice daily for 10 days. In some embodiments 1-2000 mg of one or more compounds disclosed herein is administered twice daily for 5 to 12 days, for e.g., for 5 days, 6 days, 7 days, 8 days, 9 days, 10 days, 11 days, or 12 days. In some embodiments 500-2000 mg (e.g., 500-1000 mg, 1000-1500 mg, 1500-2000 mg) of one or more compounds disclosed herein is administered twice daily for 5 to 12 days, for e.g., for 5 days, 6 days, 7 days, 8 days, 9 days, 10 days, 11 days, or 12 days.

### VII. Methods of Use

The present disclosure also provides a method of treating or preventing a viral infection in a subject (e.g., human) in need thereof, the method comprising administering to the subject a compound described herein.

In some embodiments, the present disclosure provides a method of treating a viral infection in a subject (e.g., human) in need thereof, the method comprising administering to a subject in need thereof a compound described herein.

In some embodiments, the present disclosure provides for methods of treating or preventing a viral infection in a subject (e.g., human) in need thereof, the method comprising administering to the subject a compound disclosed herein and at least one additional active therapeutic or prophylactic agent.

In some embodiments, the present disclosure provides for methods of treating a viral infection in a subject (e.g., human) in need thereof, the method comprising administering to the subject a compound disclosed herein, and at least one additional active therapeutic agent.

In some embodiments, the present disclosure provides for methods of inhibiting a viral polymerase in a cell, the methods comprising contacting the cell infected a virus with a compound disclosed herein, whereby the viral polymerase is inhibited.

In some embodiments, the present disclosure provides for methods of inhibiting a viral polymerase in a cell, the methods comprising contacting the cell infected a virus with a compound disclosed herein, and at least one additional active therapeutic agent, whereby the viral polymerase is inhibited.

Also provided here are the uses of the compounds disclosed herein for use in treating or preventing a viral infection in a subject in need thereof. For example, provided herein are uses of the compounds disclosed herein for use in treating a viral infection in a subject in need thereof.

### A. Paramyxoviridae

In some embodiments, the viral infection is a Paramyxoviridae virus infection. As such, in some embodiments, the present disclosure provides methods for treating a Paramyxoviridae infection in a subject (e.g., a human) in need thereof, the method comprising administering to the subject a compound disclosed herein. In some embodiments, the Paramyxoviridae virus includes a BSL4 pathogen. Paramyxoviridae viruses include, but are not limited to, Nipah virus, Hendra virus, measles, mumps, and parainfluenza virus.

In some embodiments, the present disclosure provides a method for manufacturing a medicament for treating a Paramyxoviridae virus infection in a subject (e.g., human) in need thereof, characterized in that the compound of the present disclosure, or a pharmaceutically acceptable salt thereof, is used. In some embodiments, the present disclosure provides use of a compound of the present disclosure, or a pharmaceutically acceptable salt thereof, for the manufacture of a medicament for the treatment in a subject (e.g., human) of Paramyxoviridae virus infection.

In some embodiments, the present disclosure provides a compound of the present disclosure, or a pharmaceutically acceptable salt thereof, for use in the treatment of a Paramyxoviridae virus infection in a subject (e.g., human) in need thereof.

### B. Pneumoviridae

In some embodiments, the viral infection is a Pneumoviridae virus infection. In some embodiments, the present disclosure provides a method of treating a Pneumoviridae virus infection in a subject (e.g., human) in need thereof, the method comprising administering to the human a therapeutically effective amount of a compound of the present disclosure, or a pharmaceutically acceptable salt thereof. Pneumoviridae viruses include, but are not limited to, respiratory syncytial virus (RSV) and human metapneumovirus. In some embodiments, the Pneumoviridae virus infection is a respiratory syncytial virus (RSV) infection. In some embodiments, the Pneumoviridae virus infection is human metapneumovirus infection.

In some embodiments, the present disclosure provides a method for manufacturing a medicament for treating a Pneumoviridae virus infection in a subject (e.g., human) in need thereof, characterized in that the compound of the present disclosure, or a pharmaceutically acceptable salt thereof, is used. In some embodiments, the present disclosure provides use of a compound of the present disclosure, or a pharmaceutically acceptable salt thereof, for the manufacture of a medicament for the treatment in a subject (e.g., human) of a Pneumoviridae virus infection. In some embodiments, the Pneumoviridae virus infection is a respiratory syncytial virus infection.

In some embodiments, the Pneumoviridae virus infection is human metapneumovirus infection.

In some embodiments, the present disclosure provides a method for manufacturing a medicament for treating a Pneumoviridae virus infection in a human in need thereof, characterized in that the compound of the present disclosure, or a pharmaceutically acceptable salt thereof, is used. In some embodiments, the present disclosure provides use of a compound of the present disclosure, or a pharmaceutically acceptable salt thereof, for the manufacture of a medicament for the treatment in a human of a Pneumoviridae virus infection. In some embodiments, the Pneumoviridae virus infection is a respiratory syncytial virus infection. In some embodiments, the Pneumoviridae virus infection is human metapneumovirus infection.

In some embodiments, the present disclosure provides a compound of the present disclosure, or a pharmaceutically acceptable salt thereof, for use in the treatment of a Pneumoviridae virus infection in a human in need thereof. In some embodiments, the Pneumoviridae virus infection is a respiratory syncytial virus (RSV) infection. In some embodiments, the Pneumoviridae virus infection is human metapneumovirus infection.

In certain embodiments, the present disclosure provides methods for treating an RSV infection, comprising administering to a subject (e.g., a human) infected with respiratory syncytial virus a therapeutically effective amount a compound of the present disclosure or a pharmaceutically acceptable salt thereof. In some embodiments, the human is suffering from a chronic respiratory syncytial viral infection. In some embodiments, the human is acutely infected with RSV.

In certain embodiments, a method of inhibiting RSV replication is provided, comprising administering a compound of the present disclosure, or a pharmaceutically acceptable salt thereof, to a subject (e.g., a human).

In certain embodiments, the present disclosure provides a method for reducing the viral load associated with RSV infection, wherein the method comprises administering to a subjectl (e.g., a human) infected with RSV a therapeutically effective amount of a compound of the present disclosure, or a pharmaceutically acceptable salt thereof, wherein the therapeutically effective amount is sufficient to reduce the RSV viral load in the subject.

As described more fully herein, compounds of the present disclosure can be administered with one or more additional therapeutic agent(s) to a subject (e.g., a human) infected with RSV.

The additional therapeutic agent(s) can be administered to the infected subject (e.g., a human) at the same time as a compound of the present disclosure or before or after administration of a compound of the present disclosure.

In certain embodiments, a compound of the present disclosure, or a pharmaceutically acceptable salt thereof, for use in treating or preventing an RSV infection is provided. In certain embodiments, a compound of the present disclosure (e.g., a compound of Formula I through Formula II), or a pharmaceutically acceptable salt thereof, for the manufacture of a medicament for treating or preventing an RSV infection is provided.

In some embodiments, a method of inhibiting RSV replication is provided, wherein the method comprises administering to a subject (e.g., human) in need thereof, a compound disclosed herein, wherein the administration is by inhalation.

In some embodiments, the present disclosure provides a method for reducing the viral load associated with RSV infection, wherein the method comprises administering to a human infected with RSV a compound disclosed herein.

### C. Picornaviridae

In some embodiments, the viral infection is a Picornaviridae virus infection. In some embodiments, the present disclosure provides a method of treating a Picornaviridae virus infection in a human in need thereof, the method comprising administering to the subject (e.g., human) a therapeutically effective amount of a compound of the present disclosure, or a pharmaceutically acceptable salt thereof. Picornaviridae viruses are enteroviruses causing a heterogeneous group of infections including herpangina, aseptic meningitis, a common-cold-like syndrome (human rhinovirus infection), a non-paralytic poliomyelitis-like syndrome, epidemic pleurodynia (an acute, febrile, infectious disease generally occurring in epidemics), hand-foot-mouth syndrome, pediatric and adult pancreatitis and serious myocarditis. In some embodiments, the Picornaviridae virus infection is human rhinovirus infection. In some embodiments, the Picornaviridae virus infection is enterovirus infection. In some embodiments, the Picornaviridae virus infection is selected from the group consisting of Coxsackie A virus infection, Coxsackie A virus infection, enterovirus D68 infection, enterovirus B69 infection, enterovirus D70 infection, enterovirus A71 infection, and poliovirus infection. In some embodiments, the Picornaviridae virus is foot and mouth disease virus (FMDV).

In some embodiments, the present disclosure provides a method for manufacturing a medicament for treating a Picornaviridae virus infection in a subject (e.g., human) in need thereof, characterized in that the compound of the present disclosure, or a pharmaceutically acceptable salt thereof, is used. In some embodiments, the present disclosure provides use of a compound of the present disclosure, or a pharmaceutically acceptable salt thereof, for the manufacture of a medicament for the treatment in a subject (e.g., human) of a Picornaviridae virus infection. In some embodiments, the Picornaviridae virus infection is human rhinovirus infection.

In some embodiments, the present disclosure provides a compound of the present disclosure, or a pharmaceutically acceptable salt thereof, for use in the treatment of a Picornaviridae virus infection in a subject (e.g., human) in need thereof. In some embodiments, the Picornaviridae virus infection is human rhinovirus infection.

### D. Flaviviridae

In some embodiments, the viral infection is a Flaviviridae virus infection. In some embodiments, the present disclosure provides a method of treating a Flaviviridae virus infection in a subject (e.g., human) in need thereof, the method comprising administering to the subject (e.g., human) a therapeutically effective amount of a compound of the present disclosure, or a pharmaceutically acceptable salt thereof. Representative Flaviviridae viruses include, but are not limited to, dengue, Yellow fever, West Nile, Zika, Japanese encephalitis virus, tick-borne encephalitis virus (TBEV), and Hepatitis C (HCV). In some embodiments, the Flaviviridae virus infection is a dengue virus infection. In some embodiments, the Flaviviridae virus infection is a Yellow fever virus infection. In some embodiments, the Flaviviridae virus infection is a West Nile virus infection. In some embodiments, the Flaviviridae virus infection is a Zika virus infection. In some embodiments, the Flaviviridae virus infection is a Japanese encephalitis virus infection. In some embodiments, the Flaviviridae virus infection is a tick-borne encephalitis virus (TBEV) infection. In some embodiments, the Flaviviridae virus infection is a Hepatitis C virus infection. In some embodiments, the Flaviviridae virus infection is bovine viral diarrhea virus (BVDV). In some embodiments, the Flaviviridae virus infection is swine fever virus (SFV).

In some embodiments, the present disclosure provides a method for manufacturing a medicament for treating a Flaviviridae virus infection in a subject (e.g., human) in need thereof, characterized in that the compound of the present disclosure, or a pharmaceutically acceptable salt thereof, is used. In some embodiments, the present disclosure provides use of a compound of the present disclosure, or a pharmaceutically acceptable salt thereof, for the manufacture of a medicament for the treatment in a subject (e.g., human) of a Flaviviridae virus infection. In some embodiments, the Flaviviridae virus infection is a dengue virus infection. In some embodiments, the Flaviviridae virus infection is a Yellow fever virus infection. In some embodiments, the Flaviviridae virus infection is a West Nile virus infection. In some embodiments, the Flaviviridae virus infection is a Zika virus infection. In some embodiments, the Flaviviridae virus infection is a Hepatitis C virus infection.

In some embodiments, the present disclosure provides a compound of the present disclosure, or a pharmaceutically acceptable salt thereof, for use in the treatment of a Flaviviridae virus infection in a human in need thereof. In some embodiments, the Flaviviridae virus infection is a dengue virus infection. In some embodiments, the Flaviviridae virus infection is a Yellow fever virus infection. In some embodiments, the Flaviviridae virus infection is a West Nile virus infection. In some embodiments, the Flaviviridae virus infection is a Zika virus infection. In some embodiments, the Flaviviridae virus infection is a Hepatitis C virus infection.

### E. Filoviridae

In some embodiments, the viral infection is a Filoviridae virus infection. In some embodiments, the present disclosure provides a method of treating a Filoviridae virus infection in a subject (e.g., human) in need thereof, the method comprising administering to the subject (e.g., human) a therapeutically effective amount of a compound of the present disclosure, or a pharmaceutically acceptable salt thereof. Representative Filoviridae viruses include, but are not limited to, ebola (variants Zaire, Bundibugio, Sudan, Tai forest, or Reston) and marburg. In some embodiments, the Filoviridae virus infection is an ebola virus infection. In some embodiments, the Filoviridae virus infection is a marburg virus infection.

In some embodiments, the present disclosure provides a method for manufacturing a medicament for treating a Filoviridae virus infection in a human in need thereof, characterized in that the compound of the present disclosure, or a pharmaceutically acceptable salt thereof, is used. In some embodiments, the present disclosure provides use of a compound of the present disclosure, or a pharmaceutically acceptable salt thereof, for the manufacture of a medicament for the treatment in a human of a Filoviridae virus infection. In some embodiments, the Filoviridae virus infection is an ebola virus infection.

In some embodiments, the present disclosure provides a compound of the present disclosure, or a pharmaceutically acceptable salt thereof, for use in the treatment of a Filoviridae virus infection in a subject (e.g., human) in need thereof. In some embodiments, the Filoviridae virus infection is an ebola virus infection. In some embodiments, the Filoviridae virus infection is a marburg virus infection.

### VIII. Combination Therapy

The compounds described herein can also be used in combination with one or more additional therapeutic agents or prophylactic agents. As such, also provided herein are methods for treatment of viral infections in a subject in need thereof, wherein the methods comprise administering to the subject a compound disclosed herein and a therapeutically effective amount of one or more additional therapeutic or prophylactic agents. In some embodiments, the methods comprise administering to the subject a compound disclosed herein and a therapeutically effective amount of one or more additional therapeutic agents. In some embodiments, the compounds disclosed herein are combined with at least one other active therapeutic agent, wherein the combination is used for treating a viral infection in a subject in need thereof. In some embodiments, the combination can be used to treat multiple separate viral infections (e.g., RSV and HIV) in one subject. In some embodiments, the compounds disclosed herein are combined with at least one other active therapeutic agent to cover a broader spectrum of respiratory viruses in one treatment without need for a diagnostic.

In some embodiments, the combination can be used to treat the same virus (e.g., RSV) in one subject. Active therapeutic agents include, but are not limited to, approved drugs, therapeutic agents currently in clinical trials, therapeutic agents that have shown efficacy in an animal model, therapeutic agents that have shown potency in in vitro assays, or any of the above.

In some embodiments, the additional therapeutic agent is an antiviral agent. Any suitable antiviral agent can be used in the methods described herein. In some embodiments, the antiviral agent is selected from the group consisting of 5-substituted 2′-deoxyuridine analogues, nucleoside analogues, pyrophosphate analogues, nucleoside reverse transcriptase inhibitors, non-nucleoside reverse transcriptase inhibitors, protease inhibitors, integrase inhibitors, entry inhibitors, acyclic guanosine analogues, acyclic nucleoside phosphonate analogues, HCV NS5A/NS5B inhibitors, influenza virus inhibitors, interferons, immunostimulators, oligonucleotides, antimitotic inhibitors, and combinations thereof.

In some embodiments, the additional therapeutic agent is a 5-substituted 2′-deoxyuridine analogue. For example, in some embodiments, the additional therapeutic agent is selected from the group consisting of idoxuridine, trifluridine, brivudine [BVDU], and combinations thereof.

In some embodiments, the additional therapeutic agent is a nucleoside analogue. For example, in some embodiments, the additional therapeutic agent is selected from the group consisting of vidarabine, entecavir (ETV), telbivudine, lamivudine, adefovir dipivoxil, tenofovir disoproxil fumarate (TDF) and combinations thereof. In some embodiments, the additional therapeutic agent is favipiravir, ribavirin, galidesivir, p-D-N4-hydroxycytidine or a combination thereof.

In some embodiments, the additional therapeutic agent is a pyrophosphate analogue. For example, in some embodiments, the additional therapeutic agent is foscarnet or phosphonoacetic acid. In some embodiments, the additional therapeutic agent is foscarnet.

In some embodiments, the additional therapeutic agent is nucleoside reverse transcriptase inhibitor. In some embodiments, the antiviral agent is zidovudine, didanosine, zalcitabine, stavudine, lamivudine, abacavir, emtricitabine, and combinations thereof.

In some embodiments, the additional therapeutic agent is a non-nucleoside reverse transcriptase inhibitor. In some embodiments, the antiviral agent is selected from the group consisting of nevirapine, delavirdine, efavirenz, etravirine, rilpivirine, and combinations thereof.

In some embodiments, the additional therapeutic agent is a protease inhibitor. In some embodiments, the protease inhibitor is a HIV protease inhibitor. For example, in some embodiments, the antiviral agent is selected from the group consisting of saquinavir, ritonavir, indinavir, nelfinavir, amprenavir, lopinavir, atazanavir, fosamprenavir, darunavir, tipranavir, cobicistat, and combinations thereof. In some embodiments, the antiviral agent is selected from the group consisting of saquinavir, ritonavir, indinavir, nelfinavir, amprenavir, lopinavir, atazanavir, fosamprenavir, darunavir, tipranavir, and combinations thereof. In some embodiments, the protease inhibitor is an HCV NS3/4A protease inhibitor. For example, in some embodiments, the additional therapeutic agent is selected from the group consisting of voxilaprevir, asunaprevir, boceprevir, paritaprevir, simeprevir, telaprevir, vaniprevir, grazoprevir, ribavirin, danoprevir, faldaprevir, vedroprevir, sovaprevir, deldeprevir, narlaprevir and combinations thereof. In some embodiments, the additional therapeutic agent is selected from the group consisting of voxilaprevir, asunaprevir, boceprevir, paritaprevir, simeprevir, telaprevir, vaniprevir, grazoprevir, and combinations thereof.

In some embodiments, the additional therapeutic agent is an integrase inhibitor. For example, in some embodiments, the additional therapeutic agent is selected from the group consisting of raltegravir, dolutegravir, elvitegravir, abacavir, lamivudine, and combinations thereof. In some embodiments, the additional therapeutic agent is selected from the group consisting of bictegravir, raltegravir, dolutegravir, cabotegravir, elvitegravir, and combinations thereof. In some embodiments, the additional therapeutic agent is selected from the group consisting of bictegravir, dolutegravir, and cabotegravir, and combinations thereof. In some embodiments, the additional therapeutic agent is bictegravir.

In some embodiments, the additional therapeutic agent is an entry inhibitor. For example, in some embodiments, the additional therapeutic agent is selected from the group consisting of docosanol, enfuvirtide, maraviroc, ibalizumab, fostemsavir, leronlimab, ibalizumab, fostemsavir, leronlimab, palivizumab, respiratory syncytial virus immune globulin, intravenous [RSV-IGIV], varicella-zoster immunoglobulin [VariZIG], varicella-zoster immune globulin [VZIG]), and combinations thereof.

In some embodiments, the additional therapeutic agent is an acyclic guanosine analogue. For example, in some embodiments, the additional therapeutic agent is selected from the group consisting of acyclovir, ganciclovir, valacyclovir (also known as valaciclovir), valganciclovir, penciclovir, famciclovir, and combinations thereof.

In some embodiments, the additional therapeutic agent is an acyclic nucleoside phosphonate analogues. For example, in some embodiments, the additional therapeutic agent is selected from a group consisting of cidofovir, adefovir, adefovir dipivoxil, tenofovir, TDF, emtricitabine, efavirenz, rilpivirine, elvitegravir, and combinations thereof. In some embodiments, the additional therapeutic agent is selected from the group consisting of cidofovir, adefovir, adefovir dipivoxil, tenofovir, TDF, and combinations thereof. In some embodiments, the additional therapeutic agent is selected from the group consisting of cidofovir, adefovir dipivoxil, TDF, and combinations thereof.

In some embodiments, the additional therapeutic agent is an HCV NS5A/NS5B inhibitor. In some embodiments, the additional therapeutic agent is a NS3/4A protease inhibitor. In some embodiments, the additional therapeutic agent is a NS5A protein inhibitor. In some embodiments, the additional therapeutic agent is a NS5B polymerase inhibitor of the nucleoside/nucleotide type. In some embodiments, the additional therapeutic agent is a NS5B polymerase inhibitor of the nonnucleoside type. In some embodiments, the additional therapeutic agent is selected from the group consisting of daclatasvir, ledipasvir, velpatasvir, ombitasvir, elbasvir, sofosbuvir, dasabuvir, ribavirin, asunaprevir, simeprevir, paritaprevir, ritonavir, elbasvir, grazoprevir, and combinations thereof. In some embodiments, the additional therapeutic agent is selected from the group consisting of daclatasvir, ledipasvir, velpatasvir, ombitasvir, elbasvir, sofosbuvir, dasabuvir, and combinations thereof.

In some embodiments, the additional therapeutic agent is an influenza virus inhibitor. In some embodiments, the additional therapeutic agent is a matrix 2 inhibitor. For example, in some embodiments, the additional therapeutic agent is selected from the group consisting of amantadine, rimantadine, and combinations thereof. In some embodiments, the additional therapeutic agent is a neuraminidase inhibitor. For example, in some embodiments, the additional therapeutic agent is selected from the group consisting of zanamivir, oseltamivir, peramivir, laninamivir octanoate, and combinations thereof. In some embodiments, the additional therapeutic agent is a polymerase inhibitor. For example, in some embodiments, the additional therapeutic agent is selected from the group consisting of ribavirin, favipiravir, and combinations thereof. In some embodiments, the additional therapeutic agent is selected from the group consisting of amantadine, rimantadine, arbidol (umifenovir), baloxavir marboxil, oseltamivir, peramivir, ingavirin, laninamivir octanoate, zanamivir, favipiravir, ribavirin, and combinations thereof. In some embodiments, the additional therapeutic agent is selected from the group consisting of amantadine, rimantadine, zanamivir, oseltamivir, peramivir, laninamivir octanoate, ribavirin, favipiravir, and combinations thereof.

In some embodiments, the additional therapeutic agent is an interferon. In some embodiments, the additional therapeutic agent is selected from the group consisting of interferon alfacon 1, interferon alfa 1b, interferon alfa 2a, interferon alfa 2b, pegylated interferon alfacon 1, pegylated interferon alfa 1b, pegylated interferon alfa 2a (PegIFNα-2a), and PegIFNα-2b. e embodiments, the additional therapeutic agent is selected from the group consisting of interferon alfacon 1, interferon alfa 1b, interferon alfa 2a, interferon alfa 2b, pegylated interferon alfa 2a (PegIFNα-2a), and PegIFNα-2b. In some embodiments, the additional therapeutic agent is selected from the group consisting of interferon alfacon 1, pegylated interferon alfa 2a (PegIFNα-2a), PegIFNα-2b, and ribavirin. In some embodiments, the additional therapeutic agent is pegylated interferon alfa-2a, pegylated interferon alfa-2b, or a combination thereof.

In some embodiments, the additional therapeutic agent is an immunostimulatory agent. In some embodiments, the additional therapeutic agent is an oligonucleotide. In some embodiments, the additional therapeutic agent is an antimitotic inhibitor. For example, in some embodiments, the additional therapeutic agent is selected from the group consisting of fomivirsen, podofilox, imiquimod, sinecatechins, and combinations thereof.

In some embodiments, the additional therapeutic agent is selected from the group consisting of besifovir, nitazoxanide, REGN2222, doravirine, sofosbuvir, velpatasvir, daclatasvir, asunaprevir, beclabuvir, FV100, and letermovir, and combinations thereof.

In some embodiments, the additional therapeutic agent is an agent for treatment of RSV. For example, in some embodiments, the antiviral agent is ribavirin, ALS-8112 or presatovir. For example, in some embodiments, the antiviral agent is ALS-8112 or presatovir.

In some embodiments, the additional therapeutic agent is an agent for treatment of picornavirus. In some embodiments, the additional therapeutic agent is selected from the group consisting of hydantoin, guanidine hydrochloride, L-buthionine sulfoximine, Py-11, and combinations thereof. In some embodiments, the additional therapeutic agent is a picornavirus polymerase inhibitor. In some embodiments, the additional therapeutic agent is rupintrivir.

In some embodiments, the additional therapeutic agent is an agent for treatment of malaria. In some embodiments, the additional therapeutic agent is chloroquine.

In some embodiments, the additional therapeutic agent is selected from the group consisting of hydroxychloroquine, chloroquine, artemether, lumefantrine, atovaquone, proguanil, tafenoquine, pyronaridine, artesunate, artenimol, piperaquine, artesunate, amodiaquine, pyronaridine, artesunate, halofantrine, quinine sulfate, mefloquine, solithromycin, pyrimethamine, MMV-390048, ferroquine, artefenomel mesylate, ganaplacide, DSM-265, cipargamin, artemisone, and combinations thereof.

In some embodiments, the additional therapeutic agent is an agent for treatment of coronavirus. In some embodiments, the additional therapeutic agent is an agent for treatment of COVID-19 (coronavirus disease 2019, a disease caused by a virus named SARS-CoV-2). In some embodiments, the additional therapeutic agent is selected from a group consisting of IFX-1, FM-201, CYNK-001, DPP4-Fc, ranpirnase, nafamostat, LB-2, AM-1, anti-viroporins, remdesivir, VV116, GS-441524, GS-5245, and combinations thereof.

In some embodiments, the additional therapeutic agent is an agent for treatment of ebola virus. For example, in some embodiments, the additional therapeutic agent is selected from the group consisting of ribavirin, palivizumab, motavizumab, RSV-IGIV (RespiGam©), MEDI-557, A-60444, MDT-637, BMS-433771, amiodarone, dronedarone, verapamil, Ebola Convalescent Plasma (ECP), TKM-100201, BCX4430 ((2S,3S,4R,5R)-2-(4-amino-5H-pyrrolo[3,2-d]pyrimidin-7-yl)-5-(hydroxymethyl)pyrrolidine-3,4-diol), favipiravir (also known as T-705 or Avigan), T-705 monophosphate, T-705 diphosphate, T-705 triphosphate, FGI-106 (1-N,7-N-bis[3-(dimethylamino)propyl]-3,9-dimethylquinolino[8,7-h]quinolone-1,7-diamine), JK-05, TKM-Ebola, ZMapp, rNAPc2, VRC-EBOADC076-00-VP, OS-2966, MVA-BN filo, brincidofovir, Vaxart adenovirus vector 5-based ebola vaccine, Ad26-ZEBOV, FiloVax vaccine, GOVX-E301, GOVX-E302, ebola virus entry inhibitors (NPC1 inhibitors), rVSV-EBOV, and combinations thereof. In some embodiments, the additional therapeutic agent is ZMapp, mAB 114, REGEN-EB3, and combinations thereof.

In some embodiments, the additional therapeutic agent is an agent for treatment of HCV. In some embodiments, the additional therapeutic agent is a HCV polymerase inhibitor. For example, in some embodiments, the additional therapeutic agent is selected from the group consisting of sofosbuvir, GS-6620, PSI-938, ribavirin, tegobuvir, radalbuvir, MK-0608, and combinations thereof. In some embodiments, the additional therapeutic agent is a HCV protease inhibitor. For example, in some embodiments, the additional therapeutic agent is selected from the group consisting of such as GS-9256, vedroprevir, voxilaprevir, and combinations thereof.

In some embodiments, the additional therapeutic agent is a NS5A inhibitor. For example, in some embodiments, the additional therapeutic agent is selected from the group consisting of ledipasvir, velpatasvir, and combinations thereof.

In some embodiments, the additional therapeutic agent is an anti HBV agent. For example, in some embodiments, the additional therapeutic agent is tenofovir disoproxil fumarate and emtricitabine, or a combination thereof. Examples of additional anti HBV agents include but are not limited to alpha-hydroxytropolones, amdoxovir, antroquinonol, beta-hydroxycytosine nucleosides, ARB-199, CCC-0975, ccc-R08, elvucitabine, ezetimibe, cyclosporin A, gentiopicrin (gentiopicroside), HH-003, hepalatide, JNJ-56136379, nitazoxanide, birinapant, NJK14047, NOV-205 (molixan, BAM-205), oligotide, mivotilate, feron, GST-HG-131, levamisole, Ka Shu Ning, alloferon, WS-007, Y-101 (Ti Fen Tai), rSIFN-co, PEG-IIFNm, KW-3, BP-Inter-014, oleanolic acid, HepB-nRNA, cTP-5 (rTP-5), HSK-II-2, HEISCO-106-1, HEISCO-106, Hepbarna, IBPB-006IA, Hepuyinfen, DasKloster 0014-01, ISA-204, Jiangantai (Ganxikang), MIV-210, OB-AI-004, PF-06, picroside, DasKloster-0039, hepulantai, IMB-2613, TCM-800B, reduced glutathione, RO-6864018, RG-7834, QL-007sofosbuvir, ledipasvir, UB-551, and ZH-2N, and the compounds disclosed in US20150210682, (Roche), US 2016/0122344 (Roche), WO2015173164, WO2016023877, US2015252057A (Roche), WO16128335A1 (Roche), WO16120186A1 (Roche), US2016237090A (Roche), WO16107833A1 (Roche), WO16107832A1 (Roche), US2016176899A (Roche), WO16102438A1 (Roche), WO16012470A1 (Roche), US2016220586A (Roche), and US2015031687A (Roche). In some embodiments, the additional therapeutic agent is a HBV polymerase inhibitor. Examples of HBV DNA polymerase inhibitors include, but are not limited to, adefovir (HEPSERA®), emtricitabine (EMTRIVA®), tenofovir disoproxil fumarate (VIREAD®), tenofovir alafenamide, tenofovir, tenofovir disoproxil, tenofovir alafenamide fumarate, tenofovir alafenamide hemifumarate, tenofovir dipivoxil, tenofovir dipivoxil fumarate, tenofovir octadecyloxyethyl ester, CMX-157, tenofovir exalidex, besifovir, entecavir (BARACLUDE®), entecavir maleate, telbivudine (TYZEKA®), filocilovir, pradefovir, clevudine, ribavirin, lamivudine (EPIVIR-HBV®), phosphazide, famciclovir, fusolin, metacavir, SNC-019754, FMCA, AGX-1009, AR-II-04-26, HIP-1302, tenofovir disoproxil aspartate, tenofovir disoproxil orotate, and HS-10234. In some embodiments, the additional therapeutic agent is an HBV capsid inhibitor.

In some embodiments, the additional therapeutic agent is an agent for treatment of HIV. In some embodiments, the additional therapeutic agent is selected from the group consisting of HIV protease inhibitors, HIV integrase inhibitors, entry inhibitors, HIV nucleoside reverse transcriptase inhibitors, HIV nonnucleoside reverse transcriptase inhibitors, acyclic nucleoside phosphonate analogues, and combinations thereof.

In some embodiments, the additional therapeutic agent is selected from the group consisting of HIV protease inhibitors, HIV non-nucleoside or non-nucleotide inhibitors of reverse transcriptase, HIV nucleoside or nucleotide inhibitors of reverse transcriptase, HIV integrase inhibitors, HIV non-catalytic site (or allosteric) integrase inhibitors, HIV entry inhibitors, HIV maturation inhibitors, immunomodulators, immunotherapeutic agents, antibody-drug conjugates, gene modifiers, gene editors (such as CRISPR/Cas9, zinc finger nucleases, homing nucleases, synthetic nucleases, TALENs), and cell therapies (such as chimeric antigen receptor T-cell, CAR-T, and engineered T cell receptors, TCR-T, autologous T cell therapies).

In some embodiments, the additional therapeutic agent is selected from the group consisting of combination drugs for HIV, other drugs for treating HIV, HIV protease inhibitors, HIV reverse transcriptase inhibitors, HIV integrase inhibitors, HIV non-catalytic site (or allosteric) integrase inhibitors, HIV entry (fusion) inhibitors, HIV maturation inhibitors, latency reversing agents, capsid inhibitors, immune-based therapies, PI3K inhibitors, HIV antibodies, and bispecific antibodies, and “antibody-like” therapeutic proteins, and combinations thereof.

In some embodiments, the additional therapeutic agent is a HIV combination drug. Examples of the HIV combination drugs include, but are not limited to ATRIPLA© (efavirenz, tenofovir disoproxil fumarate, and emtricitabine); BIKTARVY® (bictegravir, emtricitabine, and tenofovir alafenamide); COMPLERA® (EVIPLERA®; rilpivirine, tenofovir disoproxil fumarate, and emtricitabine); STRIBILD® (elvitegravir, cobicistat, tenofovir disoproxil fumarate, and emtricitabine); TRUVADA® (tenofovir disoproxil fumarate and emtricitabine; TDF+FTC); DESCOVY® (tenofovir alafenamide and emtricitabine); ODEFSEY® (tenofovir alafenamide, emtricitabine, and rilpivirine); GENVOYA® (tenofovir alafenamide, emtricitabine, cobicistat, and elvitegravir); SYMTUZA® (darunavir, tenofovir alafenamide hemifumarate, emtricitabine, and cobicistat); SYMFI™ (efavirenz, lamivudine, and tenofovir disoproxil fumarate); CIMDU™ (lamivudine and tenofovir disoproxil fumarate); tenofovir and lamivudine; tenofovir alafenamide and emtricitabine; tenofovir alafenamide hemifumarate and emtricitabine; tenofovir alafenamide hemifumarate, emtricitabine, and rilpivirine; tenofovir alafenamide hemifumarate, emtricitabine, cobicistat, and elvitegravir; COMBIVIR® (zidovudine and lamivudine; AZT+3TC); EPZICOM® (LIVEXA®; abacavir sulfate and lamivudine; ABC+3TC); KALETRA® (ALUVIA®; lopinavir and ritonavir); TRIUMEQ® (dolutegravir, abacavir, and lamivudine); TRIZIVIR® (abacavir sulfate, zidovudine, and lamivudine; ABC+AZT+3TC); atazanavir and cobicistat; atazanavir sulfate and cobicistat; atazanavir sulfate and ritonavir; darunavir and cobicistat; dolutegravir and rilpivirine; dolutegravir and rilpivirine hydrochloride; dolutegravir, abacavir sulfate, and lamivudine; lamivudine, nevirapine, and zidovudine; raltegravir and lamivudine; doravirine, lamivudine, and tenofovir disoproxil fumarate; doravirine, lamivudine, and tenofovir disoproxil; dapivirine+levonorgestrel, dolutegravir+lamivudine, dolutegravir+emtricitabine+tenofovir alafenamide, elsulfavirine+emtricitabine+tenofovir disoproxil, lamivudine+abacavir+zidovudine, lamivudine+abacavir, lamivudine+tenofovir disoproxil fumarate, lamivudine+zidovudine+nevirapine, lopinavir+ritonavir, lopinavir+ritonavir+abacavir+lamivudine, lopinavir+ritonavir+zidovudine+lamivudine, tenofovir+lamivudine, and tenofovir disoproxil fumarate+emtricitabine+rilpivirine hydrochloride, lopinavir, ritonavir, zidovudine and lamivudine.

In some embodiments, the additional therapeutic agent is a HIV capsid inhibitor (e.g., lenacapavir).

In some embodiments, the additional therapeutic agent is a HIV protease inhibitor. For example, in some embodiments the additional therapeutic agent is selected from the group consisting of saquinavir, ritonavir, indinavir, nelfinavir, amprenavir, lopinavir, atazanavir, fosamprenavir, darunavir, tipranavir, cobicistat, ASC-09, AEBL-2, MK-8718, GS-9500, GS-1156, and combinations thereof. For example, in some embodiments the additional therapeutic agent is selected from the group consisting of saquinavir, ritonavir, indinavir, nelfinavir, amprenavir, lopinavir, atazanavir, fosamprenavir, darunavir, tipranavir, cobicistat. In some embodiments, the additional therapeutic agent is selected from the group consisting of amprenavir, atazanavir, brecanavir, darunavir, fosamprenavir, fosamprenavir calcium, indinavir, indinavir sulfate, lopinavir, nelfinavir, nelfinavir mesylate, ritonavir, saquinavir, saquinavir mesylate, tipranavir, DG-17, TMB-657 (PPL-100), T-169, BL-008, MK-8122, TMB-607, TMC-310911, and combinations thereof.

In some embodiments, the additional therapeutic agent is a HIV integrase inhibitor. For example, in some embodiments, the additional therapeutic agent is selected from the group consisting of raltegravir, elvitegravir, dolutegravir, abacavir, lamivudine, bictegravir and combinations thereof. In some embodiments, the additional therapeutic agent is bictegravir. In some embodiments, the additional therapeutic agent is selected from a group consisting of bictegravir, elvitegravir, curcumin, derivatives of curcumin, chicoric acid, derivatives of chicoric acid, 3,5-dicaffeoylquinic acid, derivatives of 3,5-dicaffeoylquinic acid, aurintricarboxylic acid, derivatives of aurintricarboxylic acid, caffeic acid phenethyl ester, derivatives of caffeic acid phenethyl ester, tyrphostin, derivatives of tyrphostin, quercetin, derivatives of quercetin, raltegravir, dolutegravir, JTK-351, bictegravir, AVX-15567, BMS-986197, cabotegravir (long-acting injectable), diketo quinolin-4-1 derivatives, integrase-LEDGF inhibitor, ledgins, M-522, M-532, NSC-310217, NSC-371056, NSC-48240, NSC-642710, NSC-699171, NSC-699172, NSC-699173, NSC-699174, stilbenedisulfonic acid, T-169, VM-3500, cabotegravir, and combinations thereof.

In some embodiments, the additional therapeutic agent is a HIV entry inhibitor. For example, in some embodiments, the additional therapeutic agent is selected from the group consisting of enfuvirtide, maraviroc, and combinations thereof. Further examples of HIV entry inhibitors include, but are not limited to, cenicriviroc, CCR5 inhibitors, gp41 inhibitors, CD4 attachment inhibitors, DS-003 (BMS-599793), gp120 inhibitors, and CXCR4 inhibitors. Examples of CCR5 inhibitors include aplaviroc, vicriviroc, maraviroc, cenicriviroc, leronlimab (PRO-140), adaptavir (RAP-101), nifeviroc (TD-0232), anti-GP120/CD4 or CCR5 bispecific antibodies, B-07, MB-66, polypeptide C₂₅P, TD-0680, and vMIP (Haimipu). Examples of CXCR4 inhibitors include plerixafor, ALT-1188, N15 peptide, and vMIP (Haimipu).

In some embodiments, the additional therapeutic agent is a HIV nucleoside reverse transcriptase inhibitors. In some embodiments, the additional therapeutic agent is a HIV nonnucleoside reverse transcriptase inhibitors. In some embodiments, the additional therapeutic agent is an acyclic nucleoside phosphonate analogue. In some embodiments, the additional therapeutic agent is a HIV capsid inhibitor.

In some embodiments, the additional therapeutic agent is a HIV nucleoside or nucleotide inhibitor of reverse transcriptase. For example, the additional therapeutic agent is selected from the group consisting of adefovir, adefovir dipivoxil, azvudine, emtricitabine, tenofovir, tenofovir alafenamide, tenofovir alafenamide fumarate, tenofovir alafenamide hemifumarate, tenofovir disoproxil, tenofovir disoproxil fumarate, tenofovir disoproxil hemifumarate, VIDEX® and VIDEX EC® (didanosine, ddl), abacavir, abacavir sulfate, alovudine, apricitabine, censavudine, didanosine, elvucitabine, festinavir, fosalvudine tidoxil, CMX-157, dapivirine, doravirine, etravirine, OCR-5753, tenofovir disoproxil orotate, fozivudine tidoxil, islatravir, lamivudine, phosphazid, stavudine, zalcitabine, zidovudine, rovafovir etalafenamide (GS-9131), GS-9148, MK-8504, MK-8591, MK-858, VM-2500, KP-1461, and combinations thereof.

In some embodiments, the additional therapeutic agent is a HIV non-nucleoside or non-nucleotide inhibitor of reverse transcriptase. For example, the additional agent is selected from the group consisting of dapivirine, delavirdine, delavirdine mesylate, doravirine, efavirenz, etravirine, lentinan, MK-8583, nevirapine, rilpivirine, TMC-278LA, ACC-007, AIC-292, KM-023, PC-1005, elsulfavirine rilp (VM-1500), combinations thereof.

In some embodiments, the additional therapeutic agents are selected from ATRIPLA® (efavirenz, tenofovir disoproxil fumarate, and emtricitabine); COMPLERA® (EVIPLERA®; rilpivirine, tenofovir disoproxil fumarate, and emtricitabine); STRIBILD® (elvitegravir, cobicistat, tenofovir disoproxil fumarate, and emtricitabine); TRUVADA® (tenofovir disoproxil fumarate and emtricitabine; TDF+FTC); DESCOVY® (tenofovir alafenamide and emtricitabine); ODEFSEY® (tenofovir alafenamide, emtricitabine, and rilpivirine); GENVOYA® (tenofovir alafenamide, emtricitabine, cobicistat, and elvitegravir); adefovir; adefovir dipivoxil; cobicistat; emtricitabine; tenofovir; tenofovir disoproxil; tenofovir disoproxil fumarate; tenofovir alafenamide; tenofovir alafenamide hemifumarate; TRIUMEQ® (dolutegravir, abacavir, and lamivudine); dolutegravir, abacavir sulfate, and lamivudine; raltegravir; raltegravir and lamivudine; maraviroc; enfuvirtide; ALUVIA® (KALETRA®; lopinavir and ritonavir); COMBIVIR® (zidovudine and lamivudine; AZT+3TC); EPZICOM® (LIVEXA®; abacavir sulfate and lamivudine; ABC+3TC); TRIZIVIR® (abacavir sulfate, zidovudine, and lamivudine; ABC+AZT+3TC); rilpivirine; rilpivirine hydrochloride; atazanavir sulfate and cobicistat; atazanavir and cobicistat; darunavir and cobicistat; atazanavir; atazanavir sulfate; dolutegravir; elvitegravir; ritonavir; atazanavir sulfate and ritonavir; darunavir; lamivudine; prolastin; fosamprenavir; fosamprenavir calcium efavirenz; etravirine; nelfinavir; nelfinavir mesylate; interferon; didanosine; stavudine; indinavir; indinavir sulfate; tenofovir and lamivudine; zidovudine; nevirapine; saquinavir; saquinavir mesylate; aldesleukin; zalcitabine; tipranavir; amprenavir; delavirdine; delavirdine mesylate; Radha-108 (receptol); lamivudine and tenofovir disoproxil fumarate; efavirenz, lamivudine, and tenofovir disoproxil fumarate; phosphazid; lamivudine, nevirapine, and zidovudine; abacavir; and abacavir sulfate.

In some embodiments, the additional therapeutic agent is selected from the group consisting of colistin, valrubicin, icatibant, bepotastine, epirubicin, epoprosetnol, vapreotide, aprepitant, caspofungin, perphenazine, atazanavir, efavirenz, ritonavir, acyclovir, ganciclovir, penciclovir, prulifloxacin, bictegravir, nelfinavir, tegobuvi, nelfinavir, praziquantel, pitavastatin, perampanel, eszopiclone, and zopiclone.

In some embodiments, the additional therapeutic agent is an inhibitor of Bruton tyrosine kinase (BTK, AGMX1, AT, ATK, BPK, IGHD3, IMD1, PSCTK1, XLA; NCBI Gene ID: 695). For example, in some embodiments, the additional therapeutic agent is selected from the group consisting of (S)-6-amino-9-(1-(but-2-ynoyl)pyrrolidin-3-yl)-7-(4-phenoxyphenyl)-7H-purin-8(9H)-one, acalabrutinib (ACP-196), BGB-3111, CB988, HM71224, ibrutinib (Imbruvica), M-2951 (evobrutinib), M7583, tirabrutinib (ONO-4059), PRN-1008, spebrutinib (CC-292), TAK-020, vecabrutinib, ARQ-531, SHR-1459, DTRMWXHS-12, TAS-5315, AZD6738, calquence, danvatirsen, and combinations thereof. In some embodiments, the additional therapeutic agent is selected from a group consisting of tirabrutinib, ibrutinib, acalabrutinib, and combinations thereof. In some embodiments, the additional therapeutic agent is selected from a group consisting of tirabrutinib, ibrutinib, and combinations thereof. In some embodiments, the additional therapeutic agent is tyrphostin A9 (A9).

In some embodiments, the additional therapeutic agent is a KRAS inhibitor. For example, in some embodiments, the additional therapeutic agent is selected from the group consisting of AMG-510, COTI-219, MRTX-1257, ARS-3248, ARS-853, WDB-178, BI-3406, BI-1701963, ARS-1620 (G12C), SML-8-73-1 (G12C), Compound 3144 (G12D), Kobe0065/2602 (Ras GTP), room temperature11, MRTX-849 (G12C) and KRAS(G12D)-selective inhibitory peptides, including KRpep-2, KRpep-2d, and combinations thereof.

In some embodiments, the additional therapeutic agent is a proteasome inhibitor. For example, in some embodiments, the additional therapeutic agent is selected from a group consisting of ixazomib, carfilzomib, marizomib, bortezomib, and combinations thereof in some embodiments, the additional therapeutic agent is carfilzomib.

In some embodiments, the additional therapeutic agent is a vaccine. For example, in some embodiments, the additional therapeutic agent is a DNA vaccine, RNA vaccine, live-attenuated vaccine, therapeutic vaccine, prophylactic vaccine, protein-based vaccine, or a combination thereof. In some embodiments, the additional therapeutic agent is mRNA-1273. In some embodiments, the additional therapeutic agent is INO-4800 or INO-4700. In some embodiments, the additional therapeutic agent is live-attenuated RSV vaccine MEDI-559, human monoclonal antibody REGN2222 against RSV, palivizumab, respiratory syncytial virus immune globulin, intravenous [RSV-IGIV], and combinations thereof. In some embodiments, the additional therapeutic agent is a HBV vaccine, for example pediarix, engerix-B, and recombivax HB. In some embodiments, the additional therapeutic agent is a VZV vaccine, for example zostavax and varivax. In some embodiments, the additional therapeutic agent is a HPV vaccine, for example cervarix, gardasil 9, and gardasil. In some embodiments, the additional therapeutic agent is an influenza virus vaccine. For example, a (i) monovalent vaccine for influenza A (e.g., influenza A [H5N1] virus monovalent vaccine and influenza A [H1N1] 2009 virus monovalent vaccines), (ii) trivalent vaccine for influenza A and B viruses (e.g., Afluria, Agriflu, Fluad, Fluarix, Flublok, Flucelvax, FluLaval, Fluvirin, and Fluzone), and (iii) quadrivalent vaccine for influenza A and B viruses (FluMist, Fluarix, Fluzone, and FluLaval). In some embodiments, the additional therapeutic agent is a human adenovirus vaccine (e.g., Adenovirus Type 4 and Type 7 Vaccine, Live, Oral). In some embodiments, the additional therapeutic agent is a rotavirus vaccine (e.g., Rotarix for rotavirus serotype G1, G3, G4, or G9 and RotaTeq for rotavirus serotype G1, G2, G3, or G4). In some embodiments, the additional therapeutic agent is a hepatitis A virus vaccine (e.g., Havrix and Vagta). In some embodiments, the additional therapeutic agent is poliovirus vaccines (e.g., Kinrix, Quadracel, and Ipol). In some embodiments, the additional therapeutic agent is a yellow fever virus vaccine (e.g., YF-Vax). In some embodiments, the additional therapeutic agent is a Japanese encephalitis virus vaccine (e.g., Ixiaro and JE-Vax). In some embodiments, the additional therapeutic agent is a measles vaccine (e.g., M-M-R II and ProQuad). In some embodiments, the additional therapeutic agent is a mumps vaccine (e.g., M-M-R II and ProQuad). In some embodiments, the additional therapeutic agent is a rubella vaccine (e.g., M-M-R II and ProQuad). In some embodiments, the additional therapeutic agent is a varicella vaccine (e.g., ProQuad). In some embodiments, the additional therapeutic agent is a rabies vaccine (e.g., Imovax and RabAvert). In some embodiments, the additional therapeutic agent is a variola virus (smallpox) vaccine (ACAM2000). In some embodiments, the additional therapeutic agent is a and hepatitis E virus (HEV) vaccine (e.g., HEV239). In some embodiments, the additional therapeutic agent is a SARS-COV-2 vaccine.

In some embodiments, the additional therapeutic agent is an antibody, for example a monoclonal antibody. For example, the additional therapeutic agent is an antibody against SARS-COV-2 selected from the group consisting of the Regeneron antibodies, the Wuxi Antibodies, the Vir Biotechnology Antibodies, antibodies that target the SARS-CoV-2 spike protein, antibodies that can neutralize SARS-CoV-2 (SARS-CoV-2 neutralizing antibodies), and combinations thereof. In some embodiments, the additional therapeutic agent is anti-SARS CoV antibody CR-3022. In some embodiments, the additional therapeutic agent is aPD-1 antibody.

In some embodiments, the additional therapeutic agent is recombinant cytokine gene-derived protein injection.

In some embodiments, the additional therapeutic agent is a polymerase inhibitor. In some embodiments, the additional therapeutic agent is a DNA polymerase inhibitor. For example, in some embodiments, the additional therapeutic agent is cidofovir. In some embodiments, the additional therapeutic agent is a RNA polymerase inhibitor. For example, in some embodiments, the additional therapeutic agent is selected from the group consisting of ribavirin, favipiravir, lamivudine, pimodivir and combination thereof.

In some embodiments, the additional therapeutic agent is selected from the group consisting of lopinavir, ritonavir, interferon-alpha-2b, ritonavir, arbidol, hydroxychloroquine, darunavir and cobicistat, abidol hydrochloride, oseltamivir, litonavir, emtricitabine, tenofovir alafenamide fumarate, baloxavir marboxil, ruxolitinib, and combinations thereof.

In some embodiments, the additional therapeutic agent is selected from the group consisting of 6′-fluorinated aristeromycin analogues, acyclovir fleximer analogues, disulfiram, thiopurine analogues, ASC09F, GC376, GC813, phenylisoserine derivatives, neuroiminidase inhibitor analogues, pyrithiobac derivatives, bananins and 5-hydroxychromone derivatives, SSYA10-001, griffithsin, HR2P-M1, HR2P-M2, P21S10, Dihydrotanshinone E-64-C and E-64-D, OC43-HR2P, MERS-5HB, 229E-HR1P, 229E-HR2P, resveratrol, 1-thia-4-azaspiro[4.5]decan-3-one derivatives, gemcitabine hydrochloride, loperamide, recombinant interferons, cyclosporine A, alisporivir, imatinib mesylate, dasatinib, selumetinib, trametinib, rapamycin, saracatinib, chlorpromazine, triflupromazine, fluphenazine, thiethylperazine, promethazine, cyclophilin inhibitors, K11777, camostat, k22, teicoplanin derivatives, benzo-heterocyclic amine derivatives N30, mycophenolic acid, silvestrol, and combinations thereof.

In some embodiments, the additional therapeutic agent is an antibody. In some embodiments, the additional therapeutic agent is an antibody that binds to a coronavirus, for example an antibody that binds to SARS or MERS. In some embodiments, the additional therapeutic agent is a of SARS-COV-2 virus antibody.

Formulations of the disclosure are also used in combination with other active ingredients. For the treatment of SARS-COV-2 virus infections, in some embodiments, the other active therapeutic agent is active against coronavirus infections, for example SARS-COV-2 virus infections. The compounds and formulations of the present disclosure are also intended for use with general care provided subjects with SARS-COV-2 viral infections, including parenteral fluids (including dextrose saline and Ringer's lactate) and nutrition, antibiotic (including metronidazole and cephalosporin antibiotics, such as ceftriaxone and cefuroxime) and/or antifungal prophylaxis, fever and pain medication, antiemetic (such as metoclopramide) and/or antidiarrheal agents, vitamin and mineral supplements (including Vitamin K and zinc sulfate), anti-inflammatory agents (such as ibuprofen or steroids), corticosteroids such as methylprednisolone, immonumodulatory medications (e.g., interferon), other small molecule or biologics antiviral agents targeting SARS-COV-2 (such as but not limited to lopinavir/ritonavir, EIDD-1931, favipiravir, ribavirine, neutralizing antibodies, etc.), vaccines, pain medications, and medications for other common diseases in the subject population, such anti-malarial agents (including artemether and artesunate-lumefantrine combination therapy), typhoid (including quinolone antibiotics, such as ciprofloxacin, macrolide antibiotics, such as azithromycin, cephalosporin antibiotics, such as ceftriaxone, or aminopenicillins, such as ampicillin), or shigellosis. In some embodiments, the additional therapeutic agent is dihydroartemisinin/piperaquine.

In some embodiments, the additional therapeutic agent is an immunomodulator. Examples of immune-based therapies include toll-like receptors modulators such as tlr1, tlr2, tlr3, tlr4, tlr5, tlr6, tlr7, tlr8, tlr9, tlr10, tlr11, tlr12, and tlr13; programmed cell death protein 1 (Pd-1) modulators; programmed death-ligand 1 (Pd-L1) modulators; IL-15 modulators; DermaVir; interleukin-7; plaquenil (hydroxychloroquine); proleukin (aldesleukin, IL-2); interferon alfa; interferon alfa-2b; interferon alfa-n3; pegylated interferon alfa; interferon gamma; hydroxyurea; mycophenolate mofetil (MPA) and its ester derivative mycophenolate mofetil (MMF); ribavirin; polymer polyethyleneimine (PEI); gepon; IL-12; WF-10; VGV-1; MOR-22; BMS-936559; CYT-107, interleukin-15/Fc fusion protein, AM-0015, ALT-803, NIZ-985, NKTR-255, NKTR-262, NKTR-214, normferon, peginterferon alfa-2a, peginterferon alfa-2b, recombinant interleukin-15, Xmab-24306, RPI-MN, STING modulators, RIG-I modulators, NOD2 modulators, SB-9200, and IR-103. In some embodiments, the additional therapeutic agent is fingolimod, leflunomide, or a combination thereof. In some embodiments, the additional therapeutic agent is thalidomide.

In some embodiments, the additional therapeutic agent is an IL-6 inhibitor, for example tocilizumab, sarilumab, or a combination thereof.

In some embodiments, the additional therapeutic agent is an anti-TNF inhibitor. For example, the additional therapeutic agent is adalimumab, etanercept, golimumab, infliximab, or a combination thereof.

In some embodiments, the additional therapeutic agent is a JAK inhibitor, for example the additional therapeutic agent is baricitinib, filgotinib, olumiant, or a combination thereof.

In some embodiments, the additional therapeutic agent is an inflammation inhibitor, for example pirfenidone.

In some embodiments, the additional therapeutic agent is an antibiotic for secondary bacterial pneumonia. For example, the additional therapeutic agent is macrolide antibiotics (e.g., azithromycin, clarithromycin, and Mycoplasma pneumoniae), fluoroquinolones (e.g., ciprofloxacin and levofloxacin), tetracyclines (e.g., doxycycline and tetracycline), or a combination thereof.

In some embodiments, the compounds disclosed herein are used in combination with pneumonia standard of care (see e.g., Pediatric Community Pneumonia Guidelines, CID 2011:53 (1 October)). Treatment for pneumonia generally involves curing the infection and preventing complications. Specific treatment will depend on several factors, including the type and severity of pneumonia, age and overall health of the subjects. The options include: (i) antibiotics, (ii) cough medicine, and (iii) fever reducers/pain relievers (for e.g., aspirin, ibuprofen (Advil, Motrin IB, others) and acetaminophen (Tylenol, others)). In some embodiments, the additional therapeutic agent is bromhexine anti-cough.

In some embodiments, the compounds disclosed herein are used in combination with immunoglobulin from cured COVID-19 subjects. In some embodiments, the compounds disclosed herein are used in combination with plasma transfusion. In some embodiments, the compounds disclosed herein are used in combination with stem cells.

In some embodiments, the additional therapeutic agent is an TLR agonist. Examples of TLR agonists include, but are not limited to, vesatolimod (GS-9620), GS-986, IR-103, lefitolimod, tilsotolimod, rintatolimod, DSP-0509, AL-034, G-100, cobitolimod, AST-008, motolimod, GSK-1795091, GSK-2245035, VTX-1463, GS-9688, LHC-165, BDB-001, RG-7854, telratolimod.RO-7020531.

In some embodiments, the additional therapeutic agent is selected from the group consisting of bortezomid, flurazepam, ponatinib, sorafenib, paramethasone, clocortolone, flucloxacillin, sertindole, clevidipine, atorvastatin, cinolazepam, clofazimine, fosaprepitant, and combinations thereof.

In some embodiments, the additional therapeutic agent is carrimycin, suramin, triazavirin, dipyridamole, bevacizumab, meplazumab, GD31 (rhizobium), NLRP inflammasome inhibitor, or α-ketoamine. In some embodiments, the additional therapeutic agent is recombinant human angiotensin-converting enzyme 2 (rhACE2). In some embodiments, the additional therapeutic agent is viral macrophage inflammatory protein (vMIP).

In some embodiments, the additional therapeutic agent is an anti-viroporin therapeutic. For example, the additional therapeutic agent is BIT-314 or BIT-225. In some embodiments, the additional therapeutic agent is coronavirus E protein inhibitor. For example, the additional therapeutic agent is BIT-009. Further examples of additional therapeutic agents include those described in WO-2004112687, WO-2006135978, WO-2018145148, and WO-2009018609.

It is also possible to combine any compound of the disclosure with one or more additional active therapeutic agents in a unitary dosage form for simultaneous or sequential administration to a subject. The combination therapy can be administered as a simultaneous or sequential regimen. When administered sequentially, the combination can be administered in two or more administrations.

Co-administration of a compound of the disclosure with one or more other active therapeutic agents generally refers to simultaneous or sequential administration of a compound of the disclosure and one or more other active therapeutic agents, such that therapeutically effective amounts of the compound of the disclosure and one or more other active therapeutic agents are both present in the body of the subject.

Co-administration includes administration of unit dosages of the compounds of the disclosure before or after administration of unit dosages of one or more other active therapeutic agents, for example, administration of the compounds of the disclosure within seconds, minutes, or hours of the administration of one or more other active therapeutic agents. For example, a unit dose of a compound of the disclosure can be administered first, followed within seconds or minutes by administration of a unit dose of one or more other active therapeutic agents.

Alternatively, a unit dose of one or more other therapeutic agents can be administered first, followed by administration of a unit dose of a compound of the disclosure within seconds or minutes. In some cases, it can be desirable to administer a unit dose of a compound of the disclosure first, followed, after a period of hours (e.g., 1-12 hours), by administration of a unit dose of one or more other active therapeutic agents. In other cases, it can be desirable to administer a unit dose of one or more other active therapeutic agents first, followed, after a period of hours (e.g., 1-12 hours), by administration of a unit dose of a compound of the disclosure.

The combination therapy can provide “synergy” and “synergistic,” i.e., the effect achieved when the active ingredients used together is greater than the sum of the effects that results from using the compounds separately. A synergistic effect can be attained when the active ingredients are: (1) co-formulated and administered or delivered simultaneously in a combined formulation; (2) delivered by alternation or in parallel as separate formulations; or (3) by some other regimen.

When delivered in alternation therapy, a synergistic effect can be attained when the compounds are administered or delivered sequentially, e.g., in separate tablets, pills or capsules, or by different injections in separate syringes. In general, during alternation therapy, an effective dosage of each active ingredient is administered sequentially, i.e., serially, whereas in combination therapy, effective dosages of two or more active ingredients are administered together. A synergistic anti-viral effect denotes an antiviral effect which is greater than the predicted purely additive effects of the individual compounds of the combination.

### A. Combination Therapy for the Treatment of Pneumoviridae

The compounds and pharmaceutically acceptable salts thereof disclosed herein can be used in combination with any of the active therapeutic agents discussed in Section VIII herein and/or with other active therapeutic agents for the treatment of Pneumoviridae virus infections discussed specifically here in Section VIII.A. In some embodiments, the other active therapeutic agent is active against Pneumoviridae virus infections, particularly respiratory syncytial virus infections and/or metapneumovirus infections. As described more fully herein, compounds of the present disclosure can be administered with one or more additional therapeutic agent(s) to an subject (e.g., a human) infected with RSV. Further, in certain embodiments, when used to treat or prevent RSV, a compound of the present disclosure may be administered with one or more (e.g., one, two, three, four or more) additional therapeutic agent(s) selected from the group consisting of RSV combination drugs, RSV vaccines, RSV RNA polymerase inhibitors, immunomodulators toll-like receptor (TLR) modulators, interferon alpha receptor ligands, hyaluronidase inhibitors, respiratory syncytial surface antigen inhibitors, cytotoxic T-lymphocyte-associated protein 4 (ipi4) inhibitors, cyclophilin inhibitors, RSV viral entry inhibitors, antisense oligonucleotide targeting viral mRNA, short interfering RNAs (siRNA) and ddRNAi endonuclease modulators, ribonucelotide reductase inhibitors, farnesoid X receptor agonists, RSV antibodies, CCR2 chemokine antagonists, thymosin agonists, cytokines, nucleoprotein modulators, retinoic acid-inducible gene 1 stimulators, NOD2 stimulators, phosphatidylinositol 3-kinase (PI3K) inhibitors, indoleamine-2, 3-dioxygenase (IDO) pathway inhibitors, PD-1 inhibitors, PD-L1 inhibitors, recombinant thymosin alpha-1, bruton's tyrosine kinase (BTK) inhibitors, KDM inhibitors, RSV replication inhibitors, arginase inhibitors, and other RSV drugs.

Non-limiting examples of these other active therapeutic agents active against RSV include active monoclonal antibody and nanobody therapeutic agents, agents active against RSV infections, respiratory syncytial virus protein F inhibitors, viral replication inhibitors, RNA polymerase inhibitors, siRNA-based therapies, and combinations thereof. Non-limiting examples of active monoclonal antibody and nanobody therapeutic agents include palivizumab, RSV-IGIV (RESPIGAM®), MEDI-557 (motavizumab), MEDI8897 (nirsevimab), MK-1654, ALX-0171, A-60444 (also known as RSV604), anti-RSV G protein antibodies, and mixtures thereof. Other non-limiting examples of other active therapeutic agents active against respiratory syncytial virus infections include respiratory syncytial virus protein F inhibitors, such as MDT-637, BMS-433771, AK-0529, RV-521 (sisunatovir), JNJ-53718678 (rilematovir), BTA-585, and presatovir; RNA polymerase inhibitors, such as ribavirin, A-60444 (also known as RSV604), JNJ-64417184, ALS-8112 (JNJ-64041575; lumicitabine), and ALS-8112 (the parent nuc of lumicitabine); and viral replication inhibitors, such as EDP-938 and nitazoxanide; siRNA-based therapies, such as ALN-RSVO1; and combinations thereof.

In some embodiments, the other active therapeutic agent can be a vaccine for the treatment or prevention of RSV, including but not limited to MVA-BN RSV, RSV-F, MEDI-8897, JNJ-64400141, DPX-RSV, SynGEM, GSK-3389245A, GSK-300389-1A, RSV-MEDI deltaM2-2 vaccine, VRC-RSVRGP084-00VP, Ad35-RSV-FA2, Ad26-RSV-FA2, and RSV fusion glycoprotein subunit vaccine.

Non-limiting examples of other active therapeutic agents active against metapneumovirus infections include sialidase modulators such as DAS-181; RNA polymerase inhibitors, such as ALS-8112; and antibodies for the treatment of Metapneumovirus infections, such as EV-046113.

In some embodiments, the other active therapeutic agent can be a vaccine for the treatment or prevention of metapneumovirus infections, including but not limited to mRNA-1653 and rHMPV-Pa vaccine.

### B. Combination Therapy for the Treatment of Picornaviridae

The compounds and pharmaceutically acceptable salts thereof disclosed herein can be used in combination with any of the active therapeutic agents discussed in Section VIII herein and/or with other active therapeutic agents for the treatment of Picornaviridae virus infections discussed specifically here in Section VIIIB. In some embodiments, the other active therapeutic agent is active against Picornaviridae virus infections, particularly Enterovirus infections. Non-limiting examples of these other active therapeutic agents are capsid binding inhibitors such as pleconaril, BTA-798 (vapendavir) and other compounds disclosed by Wu, et al. (U.S. Pat. No. 7,078,403) and Watson (U.S. Pat. No. 7,166,604); fusion sialidase protein such as DAS-181; a capsid protein VP1 inhibitor such as VVX-003 and AZN-001; a viral protease inhibitor such as CW-33; a phosphatidylinositol 4 kinase beta inhibitor such as GSK-480 and GSK-533; anti-EV71 antibody.

In some embodiments, the other active therapeutic agent can be a vaccine for the treatment or prevention of Picornaviridae virus infections, including but not limited to EV71 vaccines, TAK-021, and EV-D68 adenovector-based vaccine.

### C. Combination Therapy for the Treatment of Respiratory Infections

The compounds and pharmaceutically acceptable salts thereof disclosed herein can be used in combination with any of the active therapeutic agents discussed in Section VIII herein and/or with other active therapeutic agents discussed specifically here in Section VIIIC. Many of the infections of the Pneumoviridae and Picornaviridae viruses are respiratory infections. Therefore, additional active therapeutics used to treat respiratory symptoms and sequelae of infection can be used in combination with the compounds provided herein. The additional agents can be administered orally or by direct inhalation. For example, other additional therapeutic agents in combination with the compounds provided herein for the treatment of viral respiratory infections include, but are not limited to, bronchodilators and corticosteroids.

### Glucocorticoids

Glucocorticoids, which were first introduced as an asthma therapy in 1950 (Carryer, Journal of Allergy, 21, 282-287, 1950), remain the most potent and consistently effective therapy for this disease, although their mechanism of action is not yet fully understood (Morris, J. ALLERGY CLIN. IMMUNOL., 75 (1 Pt) 1-13, 1985). Unfortunately, oral glucocorticoid therapies are associated with profound undesirable side effects such as truncal obesity, hypertension, glaucoma, glucose intolerance, acceleration of cataract formation, bone mineral loss, and psychological effects, all of which limit their use as long-term therapeutic agents (Goodman and Gilman, 10th edition, 2001). A solution to systemic side effects is to deliver steroid drugs directly to the site of inflammation. Inhaled corticosteroids (ICS) have been developed to mitigate the severe adverse effects of oral steroids. Non-limiting examples of corticosteroids that can be used in combinations with the compounds provided herein are dexamethasone, dexamethasone sodium phosphate, fluorometholone, fluorometholone acetate, loteprednol, loteprednol etabonate, hydrocortisone, prednisolone, fludrocortisones, triamcinolone, triamcinolone acetonide, betamethasone, beclomethasone diproprionate, methylprednisolone, fluocinolone, fluocinolone acetonide, flunisolide, fluocortin-21-butylate, flumethasone, flumetasone pivalate, budesonide, halobetasol propionate, mometasone furoate, fluticasone, AZD-7594, ciclesonide; or a pharmaceutically acceptable salts thereof.

### Anti-Inflammatory Agents

Other anti-inflammatory agents working through anti-inflammatory cascade mechanisms are also useful as additional therapeutic agents in combination with the compounds provided herein for the treatment of viral respiratory infections. Applying “anti-inflammatory signal transduction modulators” (referred to in this text as AIS™), like phosphodiesterase inhibitors (e.g., PDE-4, PDE-5, or PDE-7 specific), transcription factor inhibitors (e.g., blocking NFκB through IKK inhibition), or kinase inhibitors (e.g., blocking P38 MAP, INK, PI3K, EGFR or Syk) is a logical approach to switching off inflammation as these small molecules target a limited number of common intracellular pathways—those signal transduction pathways that are critical points for the anti-inflammatory therapeutic intervention (see review by P. J. Barnes, 2006). These non-limiting additional therapeutic agents include: 5-(2,4-Difluoro-phenoxy)-1-isobutyl-1H-indazole-6-carboxylic acid (2-dimethylamino-ethyl)-amide (P38 Map kinase inhibitor ARRY-797); 3-Cyclopropylmethoxy-N-(3,5-dichloro-pyridin-4-yl)-4-difluorormethoxy-benzamide (PDE-4 inhibitor Roflumilast); 4-[2-(3-cyclopentyloxy-4-methoxyphenyl)-2-phenyl-ethyl]-pyridine (PDE-4 inhibitor CDP-840); N-(3,5-dichloro-4-pyridinyl)-4-(difluoromethoxy)-8-[(methylsulfonyl)amino]-1-dibenzofurancarboxamide (PDE-4 inhibitor Oglemilast); N-(3,5-Dichloro-pyridin-4-yl)-2-[1-(4-fluorobenzyl)-5-hydroxy-1H-indol-3-yl]-2-oxo-acetamide (PDE-4 inhibitor AWD 12-281); 8-Methoxy-2-trifluoromethyl-quinoline-5-carboxylic acid (3,5-dichloro-1-oxy-pyridin-4-yl)-amide (PDE-4 inhibitor Sch 351591); 4-[5-(4-Fluorophenyl)-2-(4-methanesulfinyl-phenyl)-1H-imidazol-4-yl]-pyridine (P38 inhibitor SB-203850); 4-[4-(4-Fluoro-phenyl)-1-(3-phenyl-propyl)-5-pyridin-4-yl-1H-imidazol-2-yl]-but-3-yn-1-ol (P38 inhibitor RWJ-67657); 4-Cyano-4-(3-cyclopentyloxy-4-methoxy-phenyl)-cyclohexanecarboxylic acid 2-diethylamino-ethyl ester (2-diethyl-ethyl ester prodrug of Cilomilast, PDE-4 inhibitor); (3-Chloro-4-fluorophenyl)-[7-methoxy-6-(3-morpholin-4-yl-propoxy)-quinazolin-4-yl]-amine (Gefitinib, EGFR inhibitor); and 4-(4-Methyl-piperazin-1-ylmethyl)-N-[4-methyl-3-(4-pyridin-3-yl-pyrimidin-2-ylamino)-phenyl]-benzamide (Imatinib, EGFR inhibitor).

### β2-Adrenoreceptor Agonist Bronchodilators

Combinations comprising inhaled β2-adrenoreceptor agonist bronchodilators such as formoterol, albuterol or salmeterol with the compounds provided herein are also suitable, but non-limiting, combinations useful for the treatment of respiratory viral infections.

Combinations of inhaled β2-adrenoreceptor agonist bronchodilators such as formoterol or salmeterol with ICS's can be used to treat both the bronchoconstriction and the inflammation (SYMBICORT® and ADVAIR®, respectively). The combinations comprising these ICS and β2-adrenoreceptor agonist combinations along with the compounds provided herein are also suitable, but non-limiting, combinations useful for the treatment of respiratory viral infections.

Other examples of Beta 2 adrenoceptor agonists include, but are not limited to, bedoradrine, vilanterol, indacaterol, olodaterol, tulobuterol, formoterol, abediterol, salbutamol, arformoterol, levalbuterol, fenoterol, and TD-5471.

### Anticholinergics

For the treatment or prophylaxis of pulmonary broncho-constriction, anticholinergics are of potential use and, therefore, useful as an additional therapeutic agent in combination with the compounds provided herein for the treatment of viral respiratory infections. These anticholinergics include, but are not limited to, antagonists of the muscarinic receptor (particularly of the M3 subtype), which have shown therapeutic efficacy in man for the control of cholinergic tone in COPD (Witek, 1999); 1-{4-Hydroxy-1-[3,3,3-tris-(4-fluoro-phenyl)-propionyl]-pyrrolidine-2-carbonyl}-pyrrolidine-2-carboxylic acid (1-methyl-piperidin-4-ylmethyl)-amide; 3-[3-(2-Diethylamino-acetoxy)-2-phenyl-propionyloxy]-8-isopropyl-8-methyl-8-azonia-bicyclo[3.2.1]octane (Ipratropium-N,N-diethylglycinate); 1-Cyclohexyl-3,4-dihydro-1H-isoquinoline-2-carboxylic acid 1-aza-bicyclo[2.2.2]oct-3-yl ester (Solifenacin); 2-Hydroxymethyl-4-methanesulfinyl-2-phenyl-butyric acid 1-aza-bicyclo[2.2.2]oct-3-yl ester (Revatropate); 2-{1-[2-(2,3-Dihydro-benzofuran-5-yl)-ethyl]-pyrrolidin-3-yl}-2,2-diphenyl-acetamide (Darifenacin); 4-Azepan-1-yl-2,2-diphenyl-butyramide (Buzepide); 7-[3-(2-Diethylamino-acetoxy)-2-phenyl-propionyloxy]-9-ethyl-9-methyl-3-oxa-9-azonia-tricyclo[3.3.1.02,4]nonane (Oxitropium-N,N-diethylglycinate); 7-[2-(2-Diethylamino-acetoxy)-2,2-di-thiophen-2-yl-acetoxy]-9,9-dimethyl-3-oxa-9-azonia-tricyclo[3.3.1.02,4]nonane (Tiotropium-N,N-diethylglycinate); Dimethylamino-acetic acid 2-(3-diisopropylamino-1-phenyl-propyl)-4-methyl-phenyl ester (Tolterodine-N,N-dimethylglycinate); 3-[4,4-Bis-(4-fluoro-phenyl)-2-oxo-imidazolidin-1-yl]-1-methyl-1-(2-oxo-2-pyridin-2-yl-ethyl)-pyrrolidinium; 1-[1-(3-Fluoro-benzyl)-piperidin-4-yl]-4,4-bis-(4-fluoro-phenyl)-imidazolidin-2-one; 1-Cyclooctyl-3-(3-methoxy-1-aza-bicyclo[2.2.2]oct-3-yl)-1-phenyl-prop-2-yn-1-ol; 3-[2-(2-Diethylamino-acetoxy)-2,2-di-thiophen-2-yl-acetoxy]-1-(3-phenoxy-propyl)-1-azonia-bicyclo[2.2.2]octane (Aclidinium-N,N-diethylglycinate); or (2-Diethylamino-acetoxy)-di-thiophen-2-yl-acetic acid 1-methyl-1-(2-phenoxy-ethyl)-piperidin-4-yl ester; revefenacin, glycopyrronium bromide, umeclidinium bromide, tiotropium bromide, aclidinium bromide, and bencycloquidium bromide.

### Mucolytic Agents

The compounds provided herein can also be combined with mucolytic agents to treat both the infection and symptoms of respiratory infections. A non-limiting example of a mucolytic agent is ambroxol. Similarly, the compounds can be combined with expectorants to treat both the infection and symptoms of respiratory infections. A non-limiting example of an expectorant is guaifenesin.

Nebulized hypertonic saline is used to improve immediate and long-term clearance of small airways in subjects with lung diseases (Kuzik, J. Pediatrics 2007, 266). Thus, the compounds provided herein can also be combined with nebulized hypertonic saline particularly when the virus infection is complicated with bronchiolitis. The combination of the compound provided herein with hypertonic saline can also comprise any of the additional agents discussed above. In some embodiments, 3% hypertonic saline is used.

### D. Combination Therapy for the Treatment of COPD

The compounds and pharmaceutically acceptable salts thereof disclosed herein can be used in combination with any of the active therapeutic agents discussed in Section VIII herein and/or with other active therapeutic agents for the treatment of respiratory exacerbations of COPD discussed specifically here in Section VIII.D. In some embodiments, the other active therapeutic agents include other active agents against COPD. Non-limiting examples of these other active therapeutic agents include anti-IL5 antibodies, such as benralizumab, mepolizumab; dipeptidyl peptidase I (DPP1) inhibitors, such as AZD-7986 (INS-1007); DNA gyrase inhibitor/topoisomerase IV inhibitors, such as ciprofloxacin hydrochloride; MDR associated protein 4/phosphodiesterase (PDE) 3 and 4 inhibitors, such as RPL-554; CFTR stimulators, such as ivacaftor, QBW-251; MMP-9/MMP-12 inhibitors, such as RBx-10017609; Adenosine A1 receptor antagonists, such as PBF-680; GATA 3 transcription factor inhibitors, such as SB-010; muscarinic receptor modulator/nicotinic acetylcholine receptor agonists, such as ASM-024; MARCKS protein inhibitors, such as BIO-11006; kit tyrosine kinase/PDGF inhibitors such as masitinib; phosphodiesterase (PDE) 4 inhibitors, such as roflumilast, CHF-6001; phosphoinositide-3 kinase delta inhibitors, such as nemiralisib; 5-Lipoxygenase inhibitors, such as TA-270; muscarinic receptor antagonist/beta 2 adrenoceptor agonist, such as batefenterol succinate, AZD-887, ipratropium bromide; TRN-157; elastase inhibitors, such as erdosteine; metalloprotease-12 inhibitors such as FP-025; interleukin 18 ligand inhibitors, such as tadekinig alfa; skeletal muscle troponin activators, such as CK-2127107; p38 MAP kinase inhibitors, such as acumapimod; IL-17 receptor modulators, such as CNTO-6785; CXCR2 chemokine antagonists, such as danirixin; leukocyte elastase inhibitors, such as POL-6014; epoxide hydrolase inhibitors, such as GSK-2256294; HNE inhibitors, such as CHF-6333; VIP agonists, such as aviptadil; phosphoinositide-3 kinase delta/gamma inhibitors, such as RV-1729; complement C₃ inhibitors, such as APL-1; and G-protein coupled receptor-44 antagonists, such as AM-211.

Other non-limiting examples of active therapeutic agents also include, but are not limited to, budesonide, adipocell, nitric oxide, PUR-1800, YLP-001, LT-4001, azithromycin, gamunex, QBKPN, sodium pyruvate, MUL-1867, mannitol, MV-130, MEDI-3506, BI-443651, VR-096, OPK-0018, TEV-48107, doxofylline, TEV-46017, OligoG-COPD-5/20, STEMPEUCEL®, ZP-051, and lysine acetylsalicylate.

In some embodiments, the other active therapeutic agent may be a vaccine that is active against COPD, including but not limited to MV-130 and GSK-2838497A.

### E. Combination Therapy for the Treatment of Flaviviridae Virus Infections

The compounds and pharmaceutically acceptable salts thereof disclosed herein can be used in combination with any of the active therapeutic agents discussed in Section VIII herein and/or with other active therapeutic agents for the treatment of Flaviviridae virus infections discussed specifically here in Section VIII.E. In some embodiments, the other active therapeutic agent is active against Flaviviridae virus infections.

For treatment of the Flaviviridae virus infections, non-limiting examples of the other active therapeutic agents are host cell factor modulators, such as GBV-006; fenretinide ABX-220, BRM-211; alpha-glucosidase 1 inhibitors, such as celgosivir; platelet activating factor receptor (PAFR) antagonists, such as modipafant; cadherin-5/Factor Ia modulators, such as FX-06; NS4B inhibitors, such as JNJ-8359; viral RNA splicing modulators, such as ABX-202; a NS5 polymerase inhibitor; a NS3 protease inhibitor; and a TLR modulator.

In some embodiments, the other active therapeutic agent can be a vaccine for the treatment or prevention of dengue, including but not limited to TETRAVAX-DV, DENGVAXIA®, DPIV-001, TAK-003, live attenuated dengue vaccine, tetravalent dengue fever vaccine, tetravalent DNA vaccine, rDEN2delta30-7169; and DENV-1 PIV.

### F. Combination Therapy for the Treatment of Filoviridae Virus Infections

The compounds and pharmaceutically acceptable salts thereof disclosed herein can be used in combination with any of the active therapeutic agents discussed in Section VIII herein and/or with other active therapeutic agents for the treatment of Filoviridae virus infections discussed specifically here in Section VIIIF. In some embodiments, the other active therapeutic agent is active against Filoviridae virus infections (e.g., marburg virus, ebola virus, Sudan virus, and cueva virus infections). Non-limiting examples of these other active therapeutic agents include: MR186-YTE, remdesivir, ribavirin, palivizumab, motavizumab, RSV-IGIV (RESPIGAM©), MEDI-557, A-60444, MDT-637, BMS-433771, amiodarone, dronedarone, verapamil, Ebola Convalescent Plasma (ECP), TKM-100201, BCX4430 ((2S,3S,4R,5R)-2-(4-amino-5H-pyrrolo[3,2-d]pyrimidin-7-yl)-5-(hydroxymethyl)pyrrolidine-3,4-diol), TKM-Ebola, T-705 monophosphate, T-705 diphosphate, T-705 triphosphate, FGI-106 (1-N,7-N-bis[3-(dimethylamino)propyl]-3,9-dimethylquinolino[8,7-h]quinolone-1,7-diamine), rNAPc2, OS-2966, brincidofovir, remdesivir; RNA polymerase inhibitors, such as galidesivir, favipiravir (also known as T-705 or Avigan), JK-05; host cell factor modulators, such as GMV-006; cadherin-5/factor Ia modulators, such as FX-06; and antibodies for the treatment of Ebola, such as INMAZEB (atoltivimab, maftivimab, and odesivimab), ZMapp, and mAb 114 (EBANGA).

Other non-limiting active therapeutic agents active against Ebola include, but are not limited to, an alpha-glucosidase 1 inhibitor, a cathepsin B inhibitor, a CD29 antagonist, a dendritic ICAM-3 grabbing nonintegrin 1 inhibitor, an estrogen receptor antagonist, a factor VII antagonist HLA class II antigen modulator, a host cell factor modulator, a Interferon alpha ligand, a neutral alpha glucosidase AB inhibitor, a niemann-Pick C1 protein inhibitor, a nucleoprotein inhibitor, a polymerase cofactor VP35 inhibitor, a Serine protease inhibitor, a tissue factor inhibitor, a TLR-3 agonist, a viral envelope glycoprotein inhibitor, and an Ebola virus entry inhibitors (NPC1 inhibitors).

In some embodiments, the other active therapeutic agent can be a vaccine for the treatment or prevention of Ebola, including but not limited to VRC-EBOADC076-00-VP, adenovirus-based Ebola vaccine, rVSV-EBOV, rVSVN4CT1-EBOVGP, MVA-BN Filo+Ad26-ZEBOV regimen, INO-4212, VRC-EBODNA023-00-VP, VRC-EBOADC069-00-VP, GamEvac-combi vaccine, SRC VB Vector, HPIV3/EboGP vaccine, MVA-EBOZ, Ebola recombinant glycoprotein vaccine, Vaxart adenovirus vector 5-based Ebola vaccine, FiloVax vaccine, GOVX-E301, and GOVX-E302.

The compounds provided herein can also be used in combination with phosphoramidate morpholino oligomers (PMOs), which are synthetic antisense oligonucleotide analogs designed to interfere with translational processes by forming base-pair duplexes with specific RNA sequences. Examples of PMOs include but are not limited to AVI-7287, AVI-7288, AVI-7537, AVI-7539, AVI-6002, and AVI-6003.

The compounds provided herein are also intended for use with general care provided to subjects with Filoviridae viral infections, including parenteral fluids (including dextrose saline and Ringer's lactate) and nutrition, antibiotic (including metronidazole and cephalosporin antibiotics, such as ceftriaxone and cefuroxime) and/or antifungal prophylaxis, fever and pain medication, antiemetic (such as metoclopramide) and/or antidiarrheal agents, vitamin and mineral supplements (including Vitamin K and zinc sulfate), anti-inflammatory agents (such as ibuprofen), pain medications, and medications for other common diseases in the subject population, such anti-malarial agents (including artemether and artesunate-lumefantrine combination therapy), typhoid (including quinolone antibiotics, such as ciprofloxacin, macrolide antibiotics, such as azithromycin, cephalosporin antibiotics, such as ceftriaxone, or aminopenicillins, such as ampicillin), or shigellosis.

### G. Combination Therapy for the Treatment of Influenza

The compounds and pharmaceutically acceptable salts thereof disclosed herein can be used in combination with any of the active therapeutic agents discussed in Section VIII herein and/or with other active therapeutic agents for the treatment of influenza virus infections discussed specifically here in Section VIII.G. In some embodiments, the compounds provided herein are also used in combination with other active therapeutic agents for the treatment of influenza virus infections. The compounds and compositions provided herein are also used in combination with other active therapeutic agents. In some embodiments, the compounds provided herein can also be combined with influenza treatments. In some embodiments, the compounds provided herein are used with influenza treatments when treating influenza viruses. In some embodiments, the compounds provided herein are used with influenza treatments to treat a broader spectrum of respiratory viruses, such as those disclosed herein. In some embodiments, the influenza treatment is a neuraminidase (NA) inhibitor. In some embodiments, the influenza treatment is an M2 inhibitor. Examples of influenza treatments include, but are not limited to, AB-5080, ALS-1, amantadine (GOCOVRI®), AV-001, AV-5124, AVM-0703, baloxavir marboxil (XOFLUZA®), CB-012, CC-42344, CD-388, CT-P27, Codivir, DAS-181, DNK-651, ENOB-FL-01, ENOB-FL-11, favipiravir, GP-584, GP-681, H-015, HC-imAb, HEC-116094HCl·3H2O, HNC-042, histamine glutarimide, IFV-PA, Ingavirin, INI-2004, INNA-051, KYAHO1-2019-121, laninamivir, molnupiravir, niclosamide, nitazoxanide, norketotifen, NX-2016, oseltamivir phosphate (TAMIFLU®), peramivir (RAPIVAB®), REVTx-99, rimantadine, S-416, SAB-176, STP-702, T-705IV, TG-1000, TJ-27, TSR-066, 7HP-349, VIR-2482, VIS-410, VIS-FLX, XC-221, zanamivir (RELENZA®), zanamivir-dinitrophenyl conjugate, ZSP-1273, and ZX-7101A.

### IX. Compound Preparation

In some embodiments, the present disclosure provides processes and intermediates useful for preparing the compounds disclosed herein or pharmaceutically acceptable salts thereof.

Compounds disclosed herein can be purified by any of the means known in the art, including chromatographic means, including but not limited to high-performance liquid chromatography (HPLC), preparative thin layer chromatography, flash column chromatography, and ion exchange chromatography. Any suitable stationary phase can be used, including but not limited to, normal and reversed phases as well as ionic resins. In some embodiments, the disclosed compounds are purified via silica gel and/or alumina chromatography.

During any of the processes for preparation of the compounds provided herein, it can be necessary and/or desirable to protect sensitive or reactive groups on any of the molecules concerned. This can be achieved by means of conventional protecting groups as described in standard works, such as T. W. Greene and P. G. M. Wuts, PROTECTIVE GROUPS IN ORGANIC SYNTHESIS, 4^{th} ed., Wiley, New York 2006. The protecting groups can be removed at a convenient subsequent stage using methods known from the art.

Exemplary chemical entities useful in methods of the embodiments will now be described by reference to illustrative synthetic schemes for their general preparation herein and the specific examples that follow. Skilled artisans will recognize that, to obtain the various compounds herein, starting materials can be suitably selected so that the ultimately desired substituents will be carried through the reaction scheme with or without protection as appropriate to yield the desired product.

Alternatively, it can be necessary or desirable to employ, in the place of the ultimately desired substituent, a suitable group that can be carried through the reaction scheme and replaced as appropriate with the desired substituent. Furthermore, one of skill in the art will recognize that the transformations shown in the schemes below can be performed in any order that is compatible with the functionality of the particular pendant groups.

The methods of the present disclosure generally provide a specific enantiomer or diastereomer as the desired product, although the stereochemistry of the enantiomer or diastereomer was not determined in all cases. When the stereochemistry of the specific stereocenter in the enantiomer or diastereomer is not determined, the compound is drawn without showing any stereochemistry at that specific stereocenter even though the compound can be substantially enantiomerically or disatereomerically pure.

Representative syntheses of compounds of the present disclosure are described in the schemes below, and the particular examples that follow.

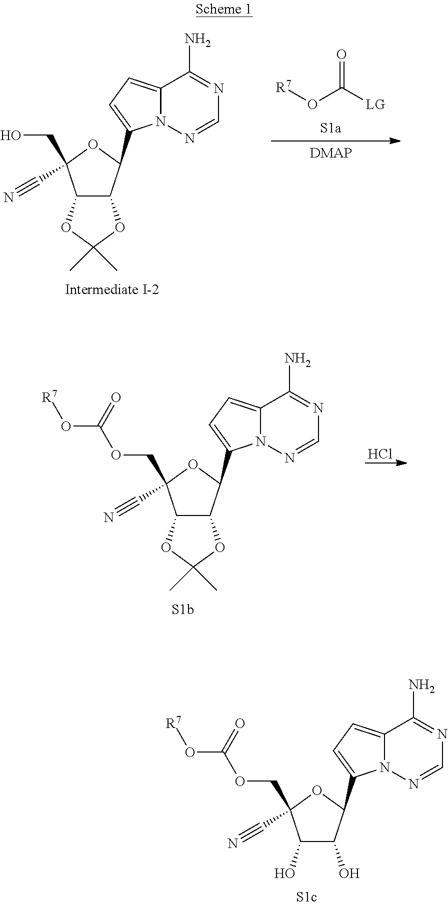

Scheme 1 shows the general synthesis of compounds starting with the reaction of Intermediate I-2 with Sla containing a leaving group (e.g., LG=C₁, or anhydride —OC(═O)OR⁷) under basic condition (e.g., DMAP) to afford intermediate Sib. This is followed by acetonide cleavage under acidic conditions (e.g., HCl) to afford final compounds of the type Sic.

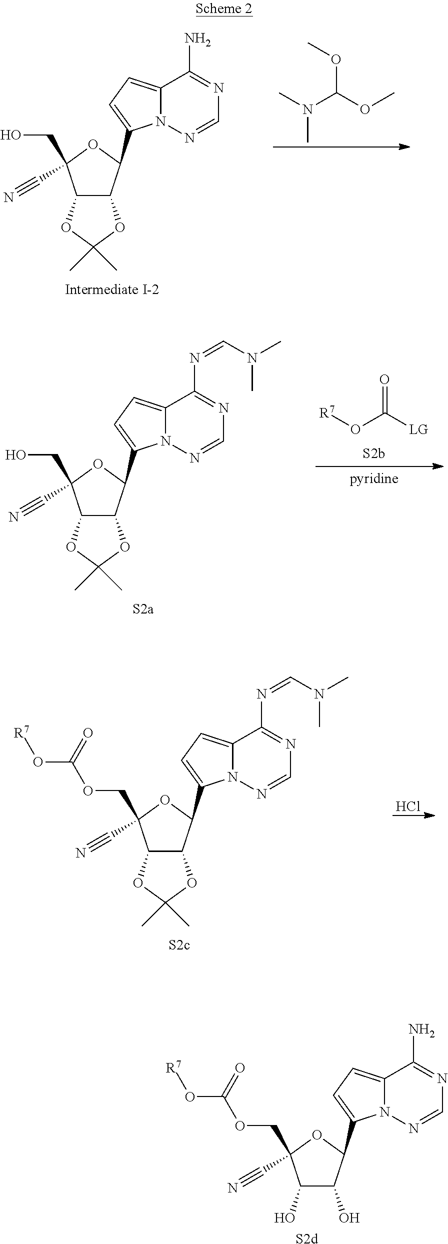

Scheme 2 shows the general synthesis of compounds starting with the reaction of Intermediate I-2 with 1,1-dimethoxy-N,N-dimethylmethanamine to afford the amidine protected base S2a. Coupling of S2a with S2b containing a leaving group (e.g., LG=C1, or anhydride —OC(═O)OR⁷) under basic condition (e.g., pyridine) affords intermediate S2c. Cleavage of the acetonide and amidine protecting groups under acidic conditions (e.g., HCl) affords final compounds of the type S2d.

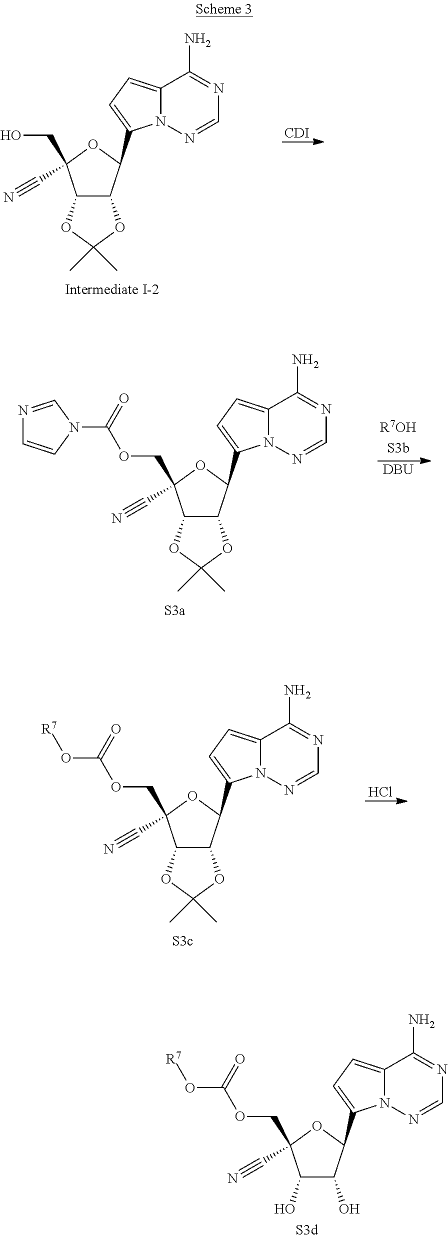

Scheme 3 shows the general synthesis of compounds starting with the reaction of Intermediate I-2 with carbonyldiimidazole (CDI) to afford intermediate S3a. Coupling of S3a with alcohol S3b under basic condition (e.g., DBU) affords intermediate S3c. Cleavage of the acetonide group under acidic conditions (e.g., HCl) affords final compounds of the type S3d.

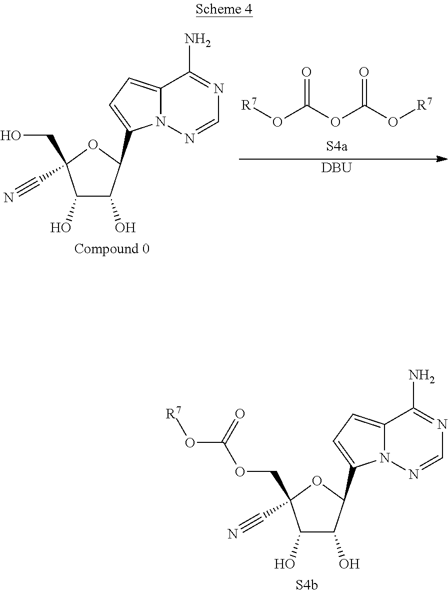

Scheme 4 shows the general synthesis of compounds starting with the reaction of Compound 0 with anhydride S4a under basic condition (e.g., DBU) to affords final compounds of the type S4b.

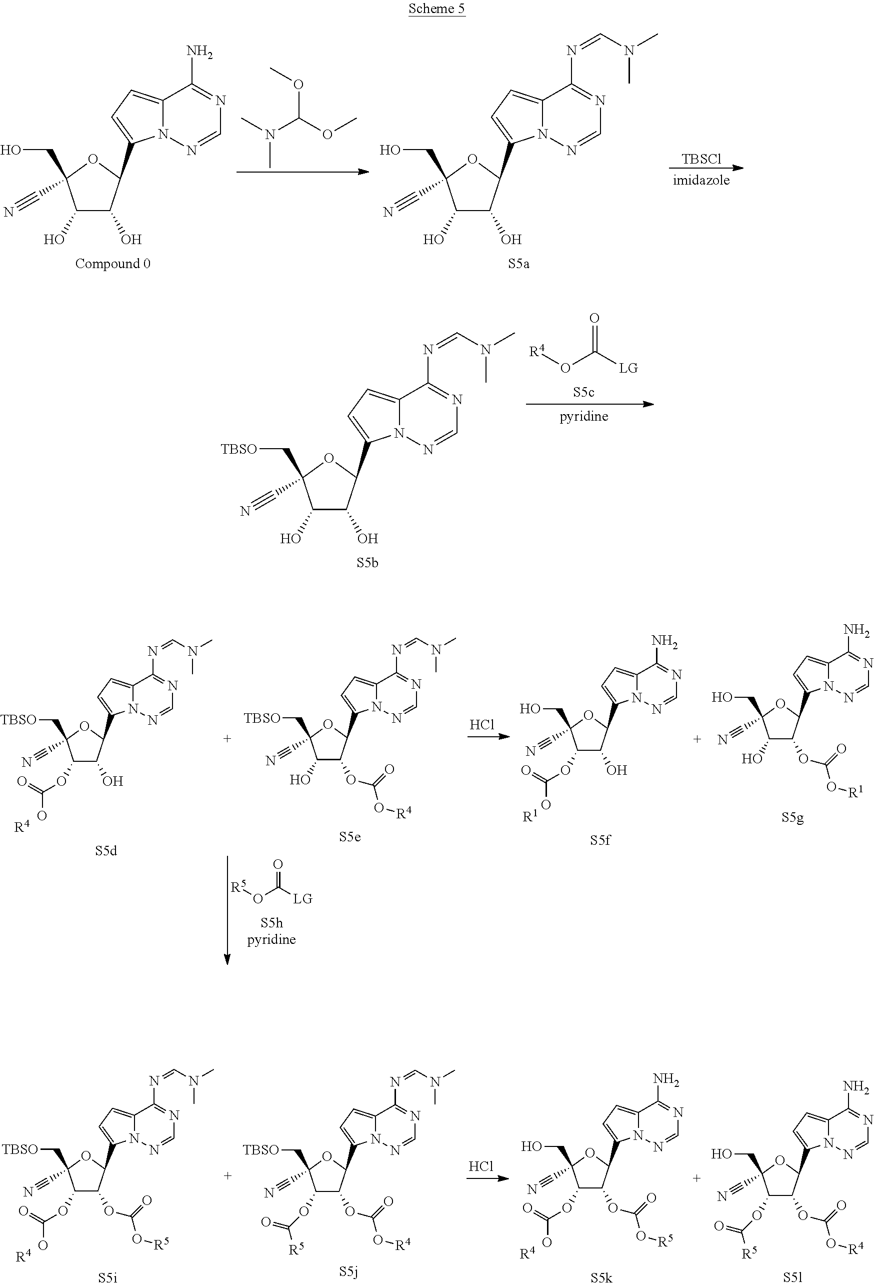

Scheme 5 shows the general synthesis of compounds starting with the reaction of Compound 0 with 1,1-dimethoxy-N,N-dimethylmethanamine to afford the amidine protected base S5a. Protection of the 5′-alcohol with a silyl protecting group reagent (e.g., TBSCl) in the presence of base (e.g., imidazole) affords intermediate S5b. Coupling of S5b with S5c containing a leaving group (e.g., LG=C₁, or anhydride —OC(═O)OR⁴) under basic condition (e.g., pyridine) affords intermediates S5d and S5e. Cleavage of the silyl and amidine protecting groups under acidic conditions (e.g., HCl) affords final compounds of the type S5f and S5g. Alternatively, intermediates S5d and S5e can be coupled to S5h containing a leaving group (e.g., LG=C₁, or anhydride —OC(═O)OR⁵) under basic condition (e.g., pyridine) affords intermediates S5i and S5j.

Cleavage of the silyl and amidine protecting groups under acidic conditions (e.g., HCl) affords final compounds of the type S5k and S5l.

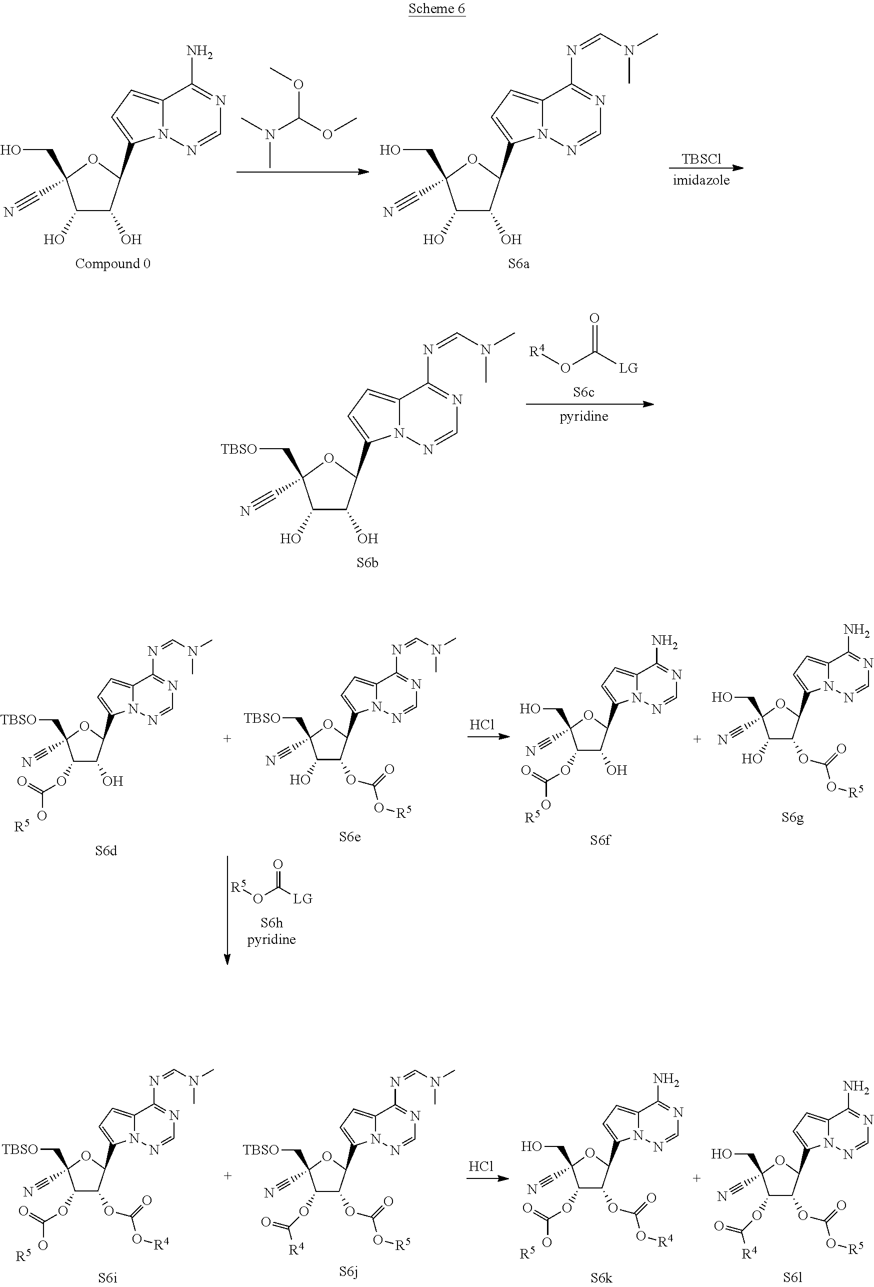

Scheme 6 shows the general synthesis of compounds starting with the reaction of Compound 0 with 1,1-dimethoxy-N,N-dimethylmethanamine to afford the amidine protected base S6a. Protection of the 5′-alcohol with a silyl protecting group reagent (e.g., TBSCl) in the presence of base (e.g., imidazole) affords intermediate S6b. Coupling of S6b with S6c containing a leaving group (e.g., LG=C₁, or anhydride —OC(═O)OR⁵) under basic condition (e.g., pyridine) affords intermediates S6d and S6e. Cleavage of the silyl and amidine protecting groups under acidic conditions (e.g., HCl) affords final compounds of the type S6f and S6g. Alternatively, intermediates S6d and S6e can be coupled to S6h containing a leaving group (e.g., LG=C₁, or anhydride —OC(═O)OR⁴) under basic condition (e.g., pyridine) affords intermediates S6i and S6j.

Cleavage of the silyl and amidine protecting groups under acidic conditions (e.g., HCl) affords final compounds of the type S6k and S6l.

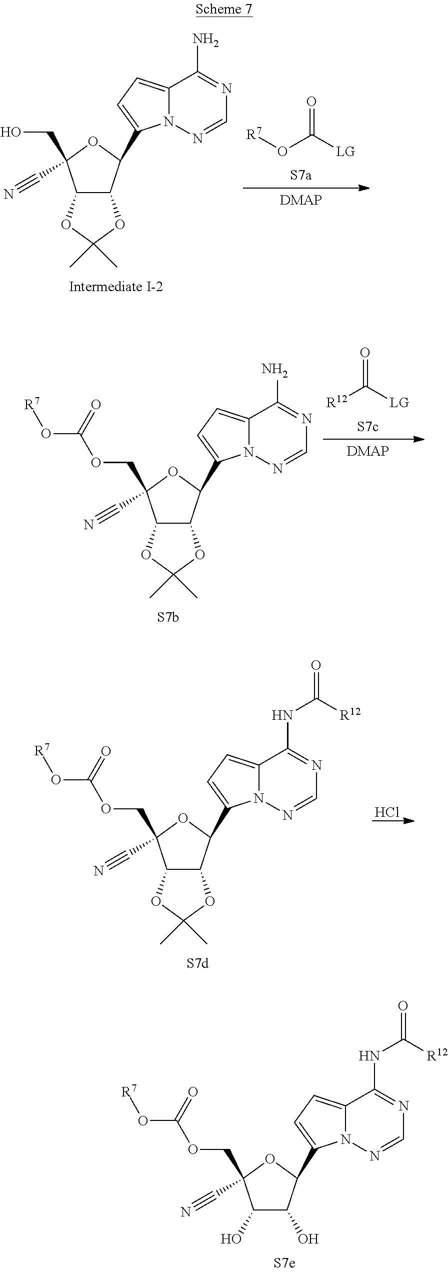

Scheme 7 shows the general synthesis of compounds starting with the reaction of Intermediate 1-2 with S7a containing a leaving group (e.g., LG=C1, or anhydride —OC(═O)OR⁷) under basic condition (e.g., DMAP) to afford intermediate S7b. Coupling of S7b with S7c containing a leaving group (e.g., LG=C1, or anhydride —OC(═O)R¹²) under basic condition (e.g., DMAP) affords intermediate S7d. Cleavage of the silyl and amidine protecting groups under acidic conditions (e.g., HCl) affords final compounds of the type S7e.

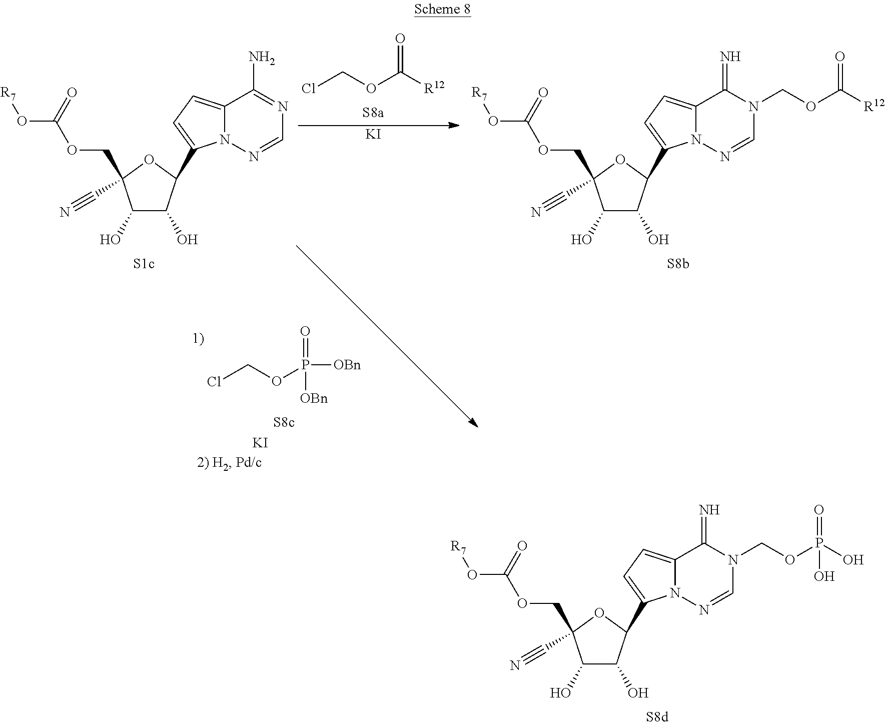

Scheme 8 shows the general synthesis of compounds starting with the reaction of S1c with S8a in the presence of iodide (e.g., KI) to afford compounds of the type S8b. Alternatively, S1c can be coupled with S8c in the presence of iodide (e.g., KI) followed by cleavage of the benzyl groups under hydrogenation conditions (e.g., H₂, Pd/C) to afford compounds of the type S8d.

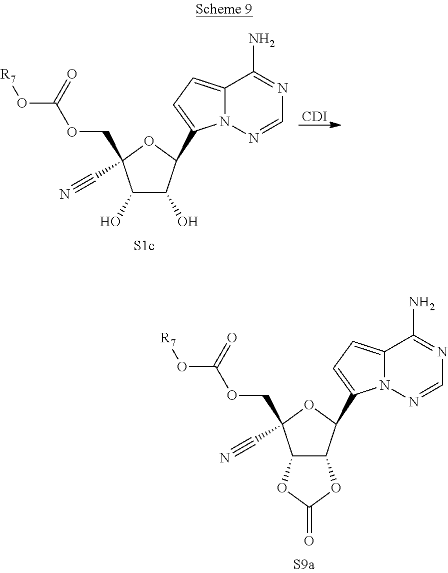

Scheme 9 shows the general synthesis of compounds starting with the reaction of S1c with CDI to afford compounds of the type S9a.

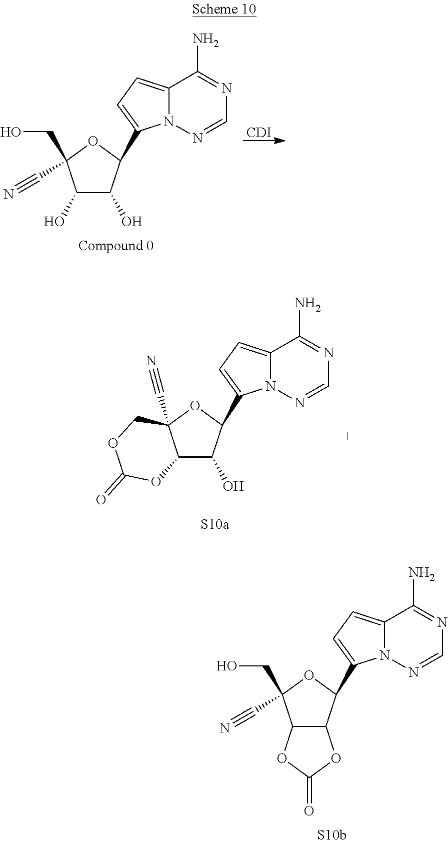

Scheme 10 shows the general synthesis of compounds starting with the reaction of Compound 0 with CDI to afford compounds of the type S10a and S10b.

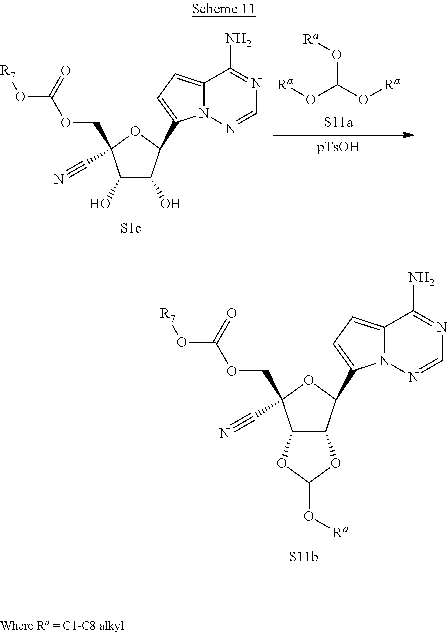

Scheme 11 shows the general synthesis of compounds starting with the reaction of S1c with orthoformate S11a under acidic condition (e.g., para-toluenesulfonic acid) to afford compounds of the type S11b.

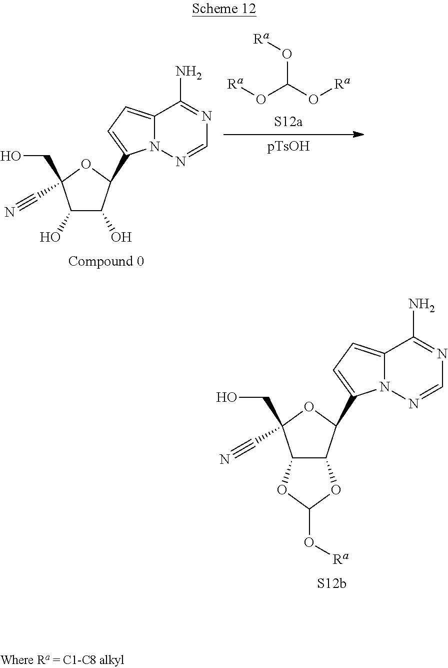

Scheme 12 shows the general synthesis of compounds starting with the reaction of Compound 0 with orthoformate S12a under acidic condition (e.g., para-toluenesulfonic acid) to afford compounds of the type S12b.

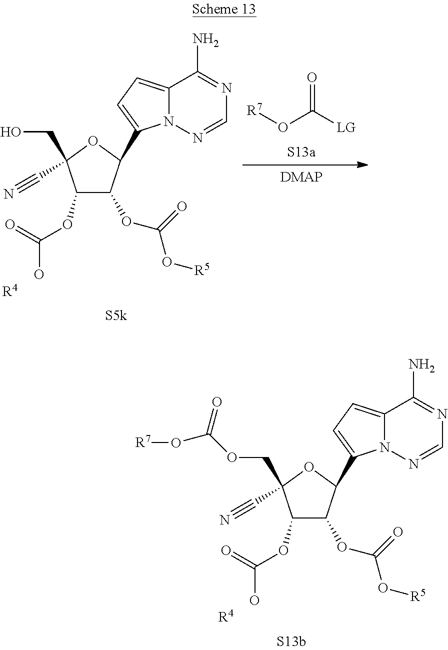

Scheme 13 shows the general synthesis of compounds starting with the reaction of S5k with S13a containing a leaving group (e.g., LG=C₁, or anhydride —OC(═O)OR⁷) under basic condition (e.g., DMAP) to afford compounds of the type S13b.

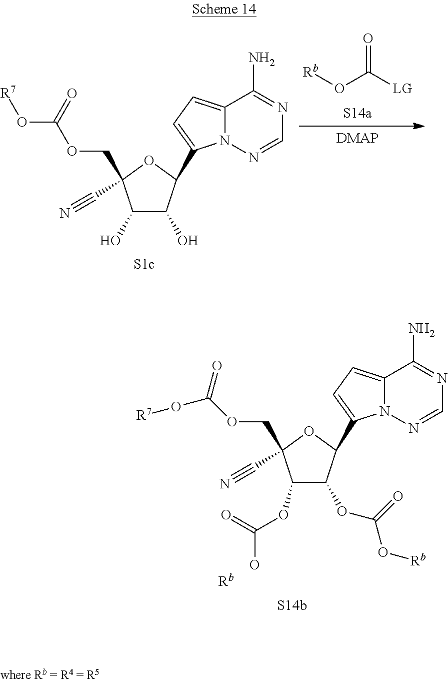

Scheme 14 shows the general synthesis of compounds starting with the reaction of Sic with S14a containing a leaving group (e.g., LG=C₁, or anhydride —OC(═O)OR^{b}) under basic condition (e.g., DMAP) to afford compounds of the type S14b.

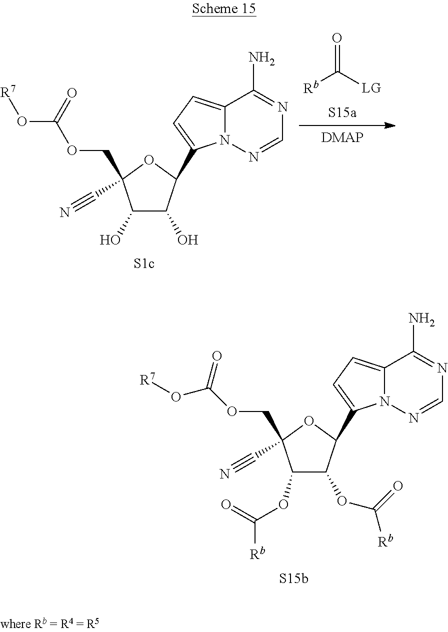

Scheme 15 shows the general synthesis of compounds starting with the reaction of Slc with S15a containing a leaving group (e.g., LG=C1, or anhydride —OC(═O)R^{b}) under basic condition (e.g., DMAP) to afford compounds of the type S15b.

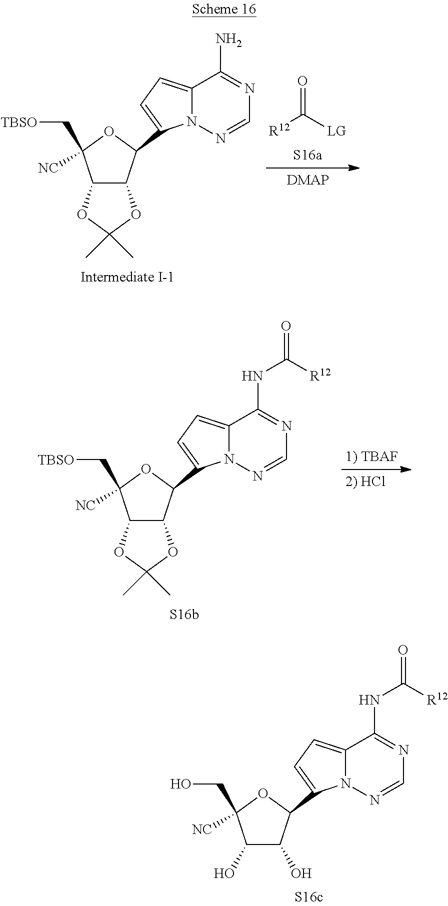

Scheme 16 shows the general synthesis of compounds starting with the reaction of Intermediate I-1 with S16a containing a leaving group (e.g., LG=C1, or anhydride —OC(═O)R^{b}) under basic conditions (e.g., DMAP) to afford intermediate S15b. Removal of the silyl protecting group with fluoride (e.g., TBAF) and cleavage of the acetonide under acidic conditions (e.g., HCl) affords compounds of the type S16c.

### EXAMPLES

### A. Abbreviations

Certain abbreviations and acronyms are used in describing the experimental details. Although most of these would be understood by one skilled in the art, Table 2 contains a list of many of these abbreviations and acronyms.

TABLE 2
List of Abbreviations and Acronyms

| Abbreviation | Meaning |
|---|---|
| Ac | acetate |
| ACN | acetonitrile |
| AIBN | azobisisobutyronitrile |
| Bn | benzyl |
| Bu | butyl |
| Bz | benzoyl |
| BzCl | benzoyl chloride |
| CDI | 1,1′-carbonyldiimidazole |
| DAST | diethylaminosulfur trifluoride |
| DCE | 1,2-dichloroethane |
| DCM | dichloromethane |
| DIPEA | N,N-diisopropylethylamine |
| DMAP | 4-dimethylamiopyridine |
| DMDO | dimethydioxirane |
| DMSO | dimethylsulfoxide |
| DMF | dimethylformamide |
| DMTrCl | 4,4′-dimethoxytritylchloride |
| DMTr | 4,4′-dimethoxytrityl |
| EDCI | N-(3-dimethylaminopropyl)-N′- |
| | ethylcarbodiimide hydrochloride |
| Et | ethyl |
| Imid | imidazole |
| KOtBu | potassium tert-butoxide |
| LC | liquid chromatography |
| MCPBA | meta-chloroperbenzoic acid |
| Me | methyl |
| m/z | mass to charge ratio |
| MS or ms | mass spectrum |
| NIS | N-iodosuccinimide |
| NMP | N-methyl-2-pyrrolidone |
| Ph | phenyl |
| Ph₃P | triphenylphosphine |
| PMB | para-methoxybenzyl |
| PMBCl | para-methoxybenzyl chloride |
| PhOC(S)Cl | phenylchlorothionoformate |
| (PhO)₃PMeI | methyltriphenoxyphosphonium iodide |
| Pyr | pyridine |
| RT | room temperature |
| SFC | supercritical fluid chromatography |
| TBAF | tetrabutylammonium fluoride |
| TBS | tert-butyldimethylsilyl |
| TBSCl | tert-Butyldimethylsilyl chloride |
| TMSN₃ | trimethylsilyl azide |
| TEA | triethylamine |
| TES | triethylsilane |
| TFA | trifluoroacetic acid |
| THF | tetrahydrofuran |
| TMS | trimethylsilyl |
| TMSCl | trimethylsilyl chloride |
| Ts | 4-toluenesulfonyl |
| TsOH | tosylic acid |
| δ | parts per million referenced to residual |
| | non-deuterated solvent peak |

### B. Intermediates

### Intermediate I-1: (3aS,4R,6S,6aS)-6-(4-aminopyrrolo[2,1-f][1,2,4]triazin-7-yl)-4-(((tert-butyldimethylsilyl)oxy)methyl)-2,2-dimethyltetrahydrofuro[3,4-d][1,3]dioxole-4-carbonitrile

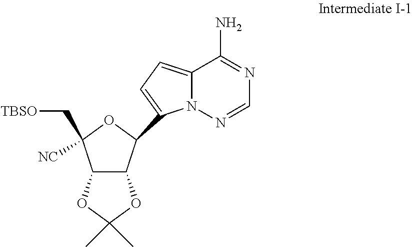 [SMILES: CC1(C)O[C@H]2[C@H](c3ccc4c(N)ncnn34)O[C@](C#N)(CO[Si](C)(C)C(C)(C)C)[C@H]2O1]

Intermediate I-1 was prepared according to WO2015/069939. For example, pages 127-138 of WO2015/069939 provide a process for preparing this compound (identified as compound 14k in WO2015/069939).

### Intermediate 1-2: (3aS,4R,6S,6aS)-6-(4-aminopyrrolo[2,1-f][1,2,4]triazin-7-yl)-4-(hydroxymethyl)-2,2-dimethyltetrahydrofuro[3,4-d][1,3]dioxole-4-carbonitrile

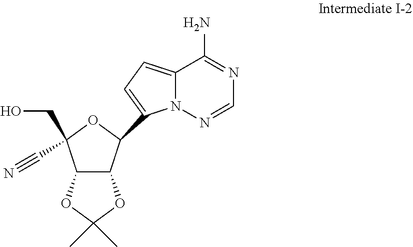 [SMILES: CC1(C)O[C@H]2[C@H](c3ccc4c(N)ncnn34)O[C@](C#N)(CO)[C@H]2O1]

Took up Intermediate I-1 (18.87 mmol) in THE (100 mL). Added TBAF 1.0 M in THE (28.31 mmol) in one portion at ambient temperature. Allowed to stir at ambient temperature for 10 min. The reaction was determined to be complete by LCMS. The reaction mixture was quenched with water and the organics were removed under reduced pressure. The crude was partitioned between EtOAc and Water. The layers were separated and the aqueous was washed with EtOAc. The organics were combined and dried over sodium sulfate. The solids were filtered off and the solvent removed under reduced pressure. The crude was purified by silica gel chromatography 120 g column 0% to 10% CH₃OH in CH₂Cl₂ to afford Intermediate I-2. LC/MS: t_{R}=0.76 min, MS m/z=332.14 [M+1]; LC system: Thermo Accela 1250 UHPLC. MS system: Thermo LCQ Fleet; Column: Kinetex 2.6μ XB-C18 100A, 50×3.00 mm. Solvents: Acetonitrile with 0.1% formic acid, Water with 0.1% formic acid. Gradient: 0 min-2.4 min 2-100% ACN, 2.4 min-2.80 min 100% ACN, 2.8 min-2.85 min 100%-2% ACN, 2.85 min-3.0 min 2% ACN at 1.8 mL/min. ¹H NMR (400 MHz, DMSO-d₆) δ 7.87-7.80 (m, 3H), 6.85 (d, J=4.5 Hz, 1H), 6.82 (d, J=4.5 Hz, 1H), 5.74 (t, J=5.8 Hz, 1H), 5.52 (d, J=4.2 Hz, 1H), 5.24 (dd, J=6.8, 4.2 Hz, 1H), 4.92 (d, J=6.8 Hz, 1H), 3.65 (dd, J=6.1, 1.7 Hz, 2H), 1.61 (s, 3H), 1.33 (s, 3H).

### Intermediate I-3: ((3aS,4R,6S,6aS)-6-(4-aminopyrrolo[2,1-f][1,2,4]triazin-7-yl)-4-cyano-2,2-dimethyltetrahydrofuro[3,4-d][1,3]dioxol-4-yl)methyl isobutyl carbonate

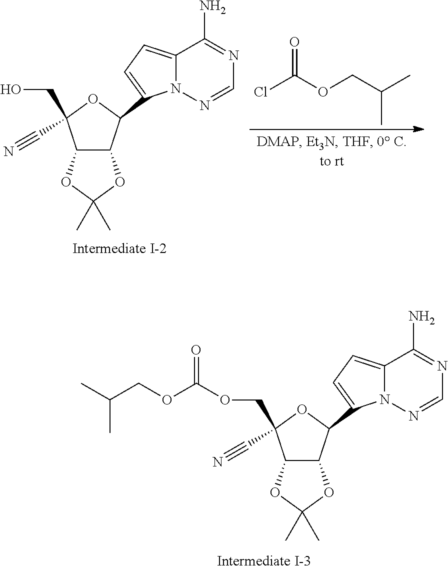

To a solution of Intermediate I-2 (3.0 mmol) and DMAP (0.76 mmol) in THF (10.0 mL) was added triethylamine (1.0 mL, 7.2 mmol). The solution was cooled in an ice bath prior to the addition of isobutyl chloroformate (0.47 mL, 3.6 mmol). The reaction mixture was stirred for 3 h while coming to room temperature in the ice bath. The reaction was quenched with water (100 mL) and extracted with EtOAc (3×50 mL) The organic fractions were combined, washed with 1:1 brine:water, dried over Na₂SO₄ and concentrated in vacuo prior to purification by silica gel chromatography (0-100% EtOAc in hexanes) to afford Intermediate I-3.

¹H NMR (400 MHz, DMSO-d₆) δ 7.94-7.74 (m, 3H), 6.87 (d, J=4.5 Hz, 1H), 6.83 (d, J=4.5 Hz, 1H), 5.62 (d, J=3.6 Hz, 1H), 5.30 (dd, J=6.6, 3.7 Hz, 1H), 5.10 (d, J=6.6 Hz, 1H), 4.51 (d, J=11.5 Hz, 1H), 4.40 (d, J=11.5 Hz, 1H), 3.92-3.87 (m, 2H), 1.95-1.84 (m, 1H), 1.64 (s, 3H), 1.35 (s, 3H), 0.87 (d, J=6.7 Hz, 6H). MS m/z [M+1]=432.1.

### Intermediate I-4: ((3aS,4R,6S,6aS)-6-(4-aminopyrrolo[2,1-f][1,2,4]triazin-7-yl)-4-cyano-2,2-dimethyltetrahydrofuro[3,4-d][1,3]dioxol-4-yl)methyl neopentyl carbonate

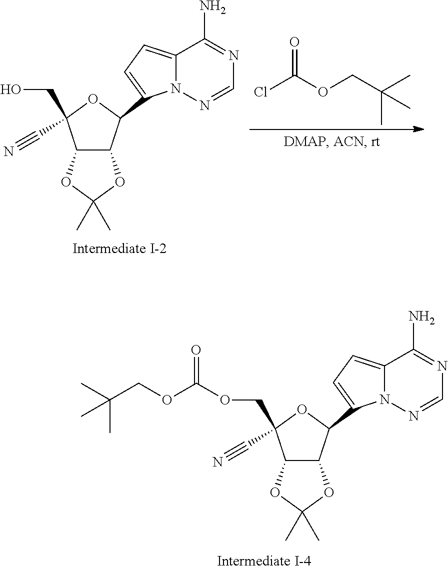

To a solution of Intermediate I-2 (0.45 mmol) and DMAP (0.48 mmol) in ACN (5.0 mL) cooled in an ice bath was added neopentyl chloroformate (68 μL, 0.45 mmol). The solution was stirred for 3 h in the ice bath while gradually coming to room temperature. The reaction was treated with MeOH (0.5 mL) and stirred for 2 h at room temperature prior to concentrating in vacuo. The crude residue was taken up in EtOAc and filtered. The filtrate was concentrated in vacuo and subjected to silica gel chromatography (0-100% EtOAc in hexanes) to afford Intermediate I-4.

¹H NMR (400 MHz, DMSO-d₆) δ 7.91-7.75 (m, 3H), 6.87 (d, J=4.6 Hz, 1H), 6.83 (d, J=4.4 Hz, 1H), 5.62 (d, J=3.6 Hz, 1H), 5.30 (dd, J=6.6, 3.6 Hz, 1H), 5.10 (d, J=6.6 Hz, 1H), 4.53 (d, J=11.6 Hz, 1H), 4.41 (d, J=11.8 Hz, 1H), 3.86-3.78 (m, 2H), 1.64 (s, 3H), 1.35 (s, 3H), 0.89 (s, 9H). MS m/z [M+1]=446.1.

### Intermediate I-5 ((3aS,4R,6S,6aS)-6-(4-aminopyrrolo[2,1-f][1,2,4]triazin-7-yl)-4-cyano-2,2-dimethyltetrahydrofuro[3,4-d][1,3]dioxol-4-yl)methyl methyl carbonate

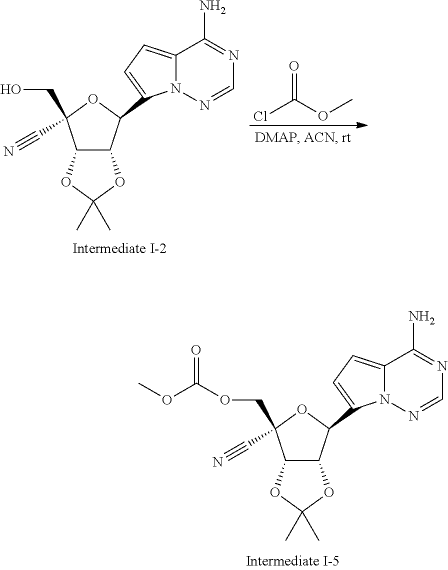

To a solution of Intermediate I-2 (0.45 mmol) and DMAP (0.77 mmol) in CAN (5.0 mL) cooled in an ice bath was added methyl chloroformate (51 μL, 0.66 mmol). The reaction mixture was stirred in the ice bath while coming to room temperature for 2 h and 20 min prior to treating with MeOH (0.5 mL) and concentrating in vacuo. The crude material was taken up in EtOAc and filtered. The filtrate was concentrated in vacuo and the crude residue was subjected to silica gel chromatography (0-100% EtOAc in hexanes) to afford Intermediate I-5.

¹H NMR (400 MHz, DMSO-d6) δ 7.99-7.67 (m, 3H), 6.89-6.81 (m, 2H), 5.62 (d, J=3.6 Hz, 1H), 5.30 (dd, J=6.7, 3.6 Hz, 1H), 5.10 (d, J=6.6 Hz, 1H), 4.50 (d, J=11.5 Hz, 1H), 4.41 (d, J=11.5 Hz, 1H), 3.72 (s, 3H), 1.64 (s, 3H), 1.35 (s, 3H). MS m/z [M+1]=390.1.

### Intermediate I-6: ((3aS,4R,6S,6aS)-6-(4-aminopyrrolo[2,1-f][1,2,4]triazin-7-yl)-4-cyano-2,2-dimethyltetrahydrofuro[3,4-d][1,3]dioxol-4-yl)methyl propyl carbonate

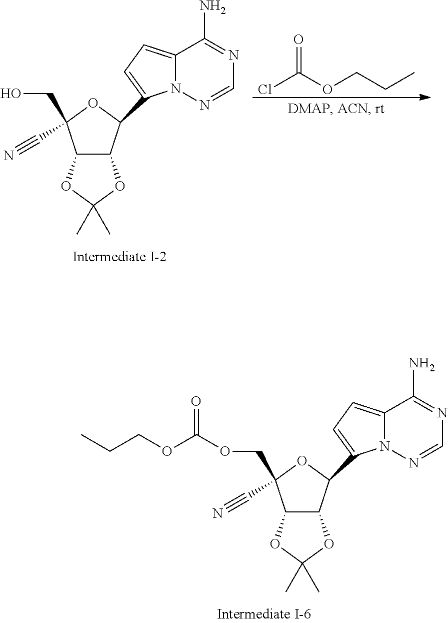

To a solution of Intermediate I-2 (0.45 mmol) and DMAP (0.77 mmol) in ACN (5.0 mL) was added propyl chloroformate (51 μL, 0.45 mmol). The reaction mixture was stirred at room temperature for 1 h prior to treating with MeOH (0.5 mL) and concentrating in vacuo. The crude material was taken up in EtOAc and filtered. After concentrating, the residue was diluted with EtOAc (30 mL) and water (30 mL). The layers were separated and the aqueous layer was extracted once more with EtOAc (30 mL). The organic fractions were combined, washed with 1:1 water:brine (30 mL), dried over Na₂SO₄, filtered, concentrated in vacuo and subjected to silica gel chromatography (0-100% EtOAc in hexanes) to afford Intermediate I-6.

¹H NMR (400 MHz, DMSO-d6) δ 7.94-7.75 (m, 3H), 6.89-6.80 (m, 2H), 5.62 (d, J=3.6 Hz, 1H), 5.30 (dd, J=6.7, 3.7 Hz, 1H), 5.10 (d, J=6.6 Hz, 1H), 4.50 (d, J=11.6 Hz, 1H), 4.40 (d, J=11.5 Hz, 1H), 4.05 (t, J=6.6 Hz, 2H), 1.67-1.55 (m, 5H), 1.35 (s, 3H), 0.87 (t, J=7.4 Hz, 3H). MS m/z [M+1]=418.1.

### Intermediate I-7 ((3aS,4R,6S,6aS)-6-(4-aminopyrrolo[2,1-f][1,2,4]triazin-7-yl)-4-cyano-2,2-dimethyltetrahydrofuro[3,4-d][1,3]dioxol-4-yl)methyl 1H-imidazole-1-carboxylate

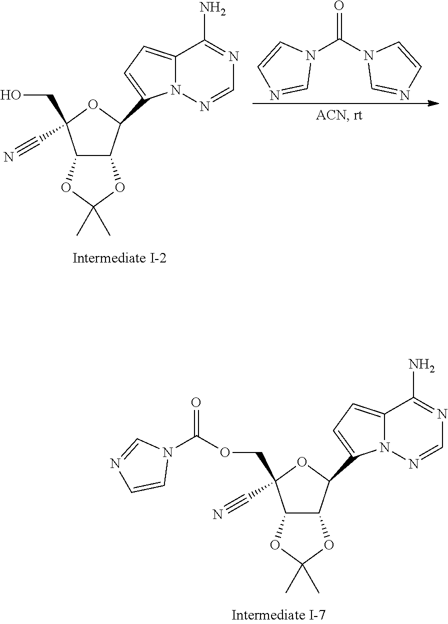

To a solution of Intermediate I-2 (1.8 mmol) in ACN (20.0 mL) was added CDI (2.6 mmol). The solution was stirred at room temperature for 2.5 h. The reaction was quenched with water (100 mL) and extracted with EtOAc (2×100 mL). The organic fractions were combined, washed with 1:1 water:brine (80 mL), dried over Mg₂SO₄, filtered and concentrated in vacuo to afford Intermediate I-7, which was used without further purification.

¹H NMR (400 MHz, DMSO-d6) δ 8.28-8.26 (m, 1H), 7.91-7.74 (m, 3H), 7.60-7.57 (m, 1H), 7.13-7.11 (m, 1H), 6.84 (d, J=4.4 Hz, 1H), 6.80 (d, J=4.5 Hz, 1H), 5.63 (d, J=3.5 Hz, 1H), 5.37-5.28 (m, 2H), 4.86 (d, J=11.6 Hz, 1H), 4.73 (d, J=11.6 Hz, 1H), 1.66 (s, 3H), 1.37 (s, 3H). MS m/z [M+1]=426.1.

Intermediate I-8 ((3aS,4R,6S,6aS)-6-(4-aminopyrrolo[2,1-f][1,2,4]triazin-7-yl)-4-cyano-2,2-dimethyltetrahydrofuro[3,4-d][1,3]dioxol-4-yl)methyl ((R)-sec-butyl) carbonate

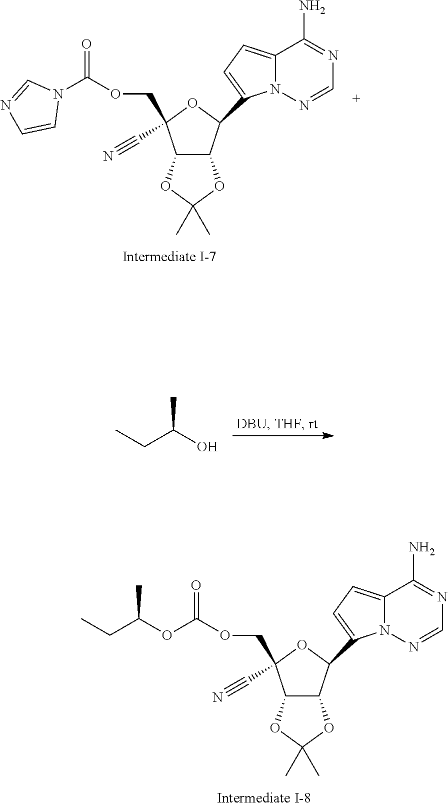

To a solution of Intermediate I-7 (0.47 mmol) in THF (5.0 mL) was added (R)-butan-2-ol (0.17 mL, 1.9 mmol), followed by a catalytic amount of DBU (one drop). The solution was stirred at room temperature for 1 h prior to adding an additional 2 drops of DBU. Additional (R)-butan-2-ol (0.34 mL, 3.7 mmol, 7.9 equiv.) was added and the solution was stirred for 20 min. The reaction mixture was concentrated in vacuo and subjected to silica gel chromatography (0-100% EtOAc in hexanes) to afford Intermediate I-8.

¹H NMR (400 MHz, DMSO-d6) δ 7.92-7.75 (m, 3H), 6.87 (d, J=4.5 Hz, 1H), 6.83 (d, J=4.5 Hz, 1H), 5.62 (d, J=3.6 Hz, 1H), 5.30 (dd, J=6.7, 3.6 Hz, 1H), 5.09 (d, J=6.6 Hz, 1H), 4.65-4.55 (m, 1H), 4.49 (d, J=11.5 Hz, 1H), 4.38 (d, J=11.5 Hz, 1H), 1.64 (s, 3H), 1.60-1.49 (m, 2H), 1.35 (s, 3H), 1.20 (d, J=6.1 Hz, 3H), 0.86-0.80 (m, 3H). MS m/z [M+1]=432.1.

### Intermediate I-9: ((3aS,4R,6S,6aS)-6-(4-aminopyrrolo[2,1-f][1,2,4]triazin-7-yl)-4-cyano-2,2-dimethyltetrahydrofuro[3,4-d][1,3]dioxol-4-yl)methyl ((S)-sec-butyl) carbonate

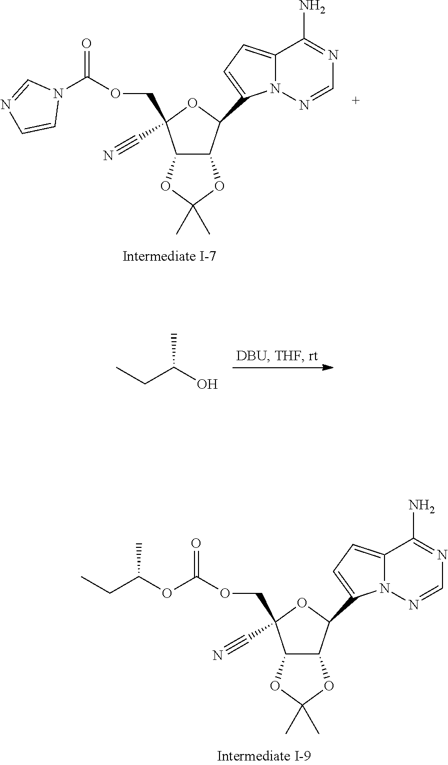

To a solution of Intermediate I-7 (0.34 mmol) in THF (5.0 mL) was added (S)-butan-2-ol (0.16 mL, 1.7 mmol), followed by DBU (0.02 mL, 0.14 mmol). The solution was stirred at room temperature for 3.5 h. The reaction mixture was concentrated in vacuo and subjected to silica gel chromatography (0-100% EtOAc in hexanes) to afford Intermediate I-9.

¹H NMR (400 MHz, DMSO-d6) δ 7.93-7.75 (m, 3H), 6.87 (d, J=4.5 Hz, 1H), 6.83 (d, J=4.5 Hz, 1H), 5.62 (d, J=3.6 Hz, 1H), 5.30 (dd, J=6.6, 3.6 Hz, 1H), 5.09 (d, J=6.6 Hz, 1H), 4.66-4.56 (m, 1H), 4.50 (d, J=11.5 Hz, 1H), 4.38 (d, J=11.5 Hz, 1H), 1.64 (s, 3H), 1.61-1.50 (m, 2H), 1.35 (s, 3H), 1.18 (d, J=6.3 Hz, 3H), 0.88-0.82 (m, 3H). MS m/z [M+1]=432.1.

### Intermediate I-10 ((3aS,4R,6S,6aS)-6-(4-aminopyrrolo[2,1-f][1,2,4]triazin-7-yl)-4-cyano-2,2-dimethyltetrahydrofuro[3,4-d][1,3]dioxol-4-yl)methyl pentan-3-yl carbonate

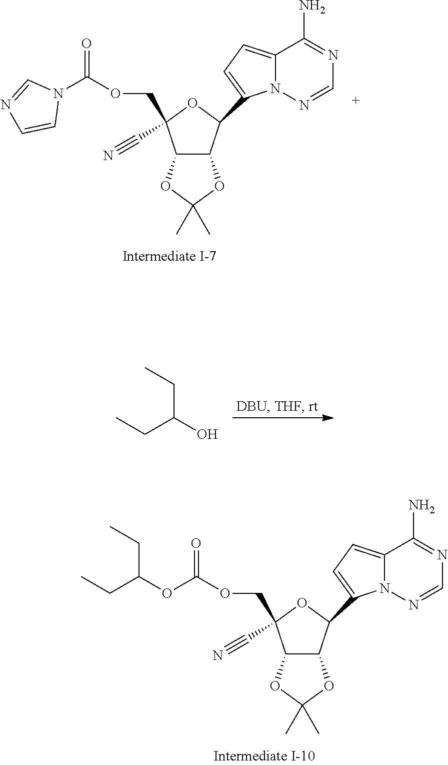

To a solution of Intermediate I-7 (0.35 mmol) in THF (5.0 mL) was added pentan-3-ol (0.15 mL, 1.4 mmol), followed by DBU (0.08 mL, 0.53 mmol) The solution was stirred at room temperature for 40 min prior to concentrating in vacuo and subjecting to silica gel chromatography (0-100% EtOAc in hexanes) to afford Intermediate I-10.

¹H NMR (400 MHz, DMSO-d6) δ 7.94-7.74 (m, 3H), 6.86 (d, J=4.4 Hz, 1H), 6.83 (d, J=4.5 Hz, 1H), 5.62 (d, J=3.6 Hz, 1H), 5.31 (dd, J=6.6, 3.6 Hz, 1H), 5.09 (d, J=6.7 Hz, 1H), 4.56-4.46 (m, 2H), 4.39 (d, J=11.6 Hz, 1H), 1.65-1.44 (m, 7H), 1.35 (s, 3H), 0.86-0.77 (m, 6H). MS m/z [M+1]=446.1.

### Intermediate I-11: ((3aS,4R,6S,6aS)-6-(4-aminopyrrolo[2,1-f][1,2,4]triazin-7-yl)-4-cyano-2,2-dimethyltetrahydrofuro[3,4-d][1,3]dioxol-4-yl)methyl isopropyl carbonate

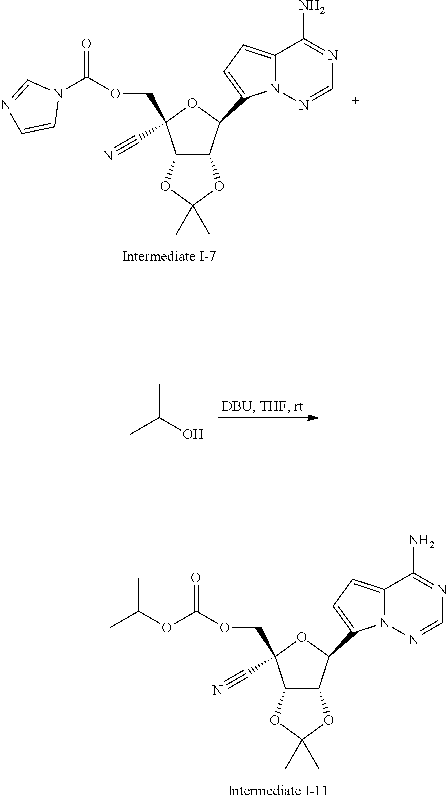

To a solution of Intermediate I-7 (0.47 mmol) in THF (5.0 mL) was added isopropanol (0.14 mL, 1.9 mmol), followed by DBU (0.11 mL, 0.71 mmol). The solution was stirred at room temperature for 2 h and 10 min. The reaction mixture was concentrated in vacuo and subjected to silica gel chromatography (0-100% EtOAc in hexanes then 0-15% MeOH in EtOAc) to afford Intermediate I-11.

¹H NMR (400 MHz, DMSO-d6) δ 7.93-7.76 (m, 3H), 6.87 (d, J=4.6 Hz, 1H), 6.83 (d, J=4.5 Hz, 1H), 5.62 (d, J=3.6 Hz, 1H), 5.30 (dd, J=6.6, 3.7 Hz, 1H), 5.09 (d, J=6.6 Hz, 1H), 4.82-4.71 (m, 1H), 4.47 (d, J=11.5 Hz, 1H), 4.38 (d, J=11.4 Hz, 1H), 1.64 (s, 3H), 1.35 (s, 3H), 1.24-1.20 (m, 6H). MS m/z [M+1]=418.1.

### Intermediate I-12: Isobutyl (7-((3aS,4S,6R,6aS)-6-cyano-6-(hydroxymethyl)-2,2-dimethyltetrahydrofuro[3,4-d][1,3]dioxo-4-yl)pyrrolo[2,1-f][1,2,4]triazin-4-yl)carbamate

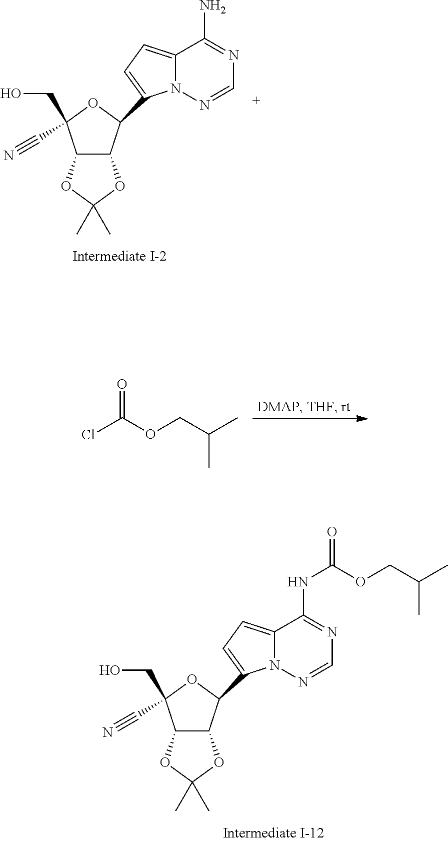

To a solution of Intermediate I-2 (0.45 mmol) and DMAP (0.26 mmol) in THF (5.0 mL) was added isobutyl chloroformate (0.06 mL, 0.45 mmol). The reaction mixture was stirred at room temperature for 5 h and 30 min prior to quenching with a 1:1 solution of water and saturated NaHCO₃ (20 mL) and extracting with EtOAc (2×20 mL). The organic fractions were combined, washed with brine (20 mL), dried over MgSO₄, filtered, concentrated in vacuo and subjected to silica gel chromatography (0-100% EtOAc in hexanes) to afford Intermediate I-12.

¹H NMR (400 MHz, DMSO-d6) δ 10.84 (s, 1H), 8.29 (s, 1H), 7.29-7.20 (m, 1H), 7.10-7.02 (m, 1H), 5.82-5.75 (m, 1H), 5.62 (d, J=3.9 Hz, 1H), 5.29 (dd, J=6.7, 4.0 Hz, 1H), 4.96 (d, J=6.7 Hz, 1H), 3.99-3.93 (m, 2H), 3.73-3.63 (m, 2H), 2.05-1.90 (m, 1H), 1.64 (s, 3H), 1.35 (s, 3H), 0.96 (d, J=6.7 Hz, 6H). MS m/z [M+1]=432.1.

### Intermediate I-13 ((3aS,4R,6S,6aS)-6-(4-aminopyrrolo[2,1-f][1,2,4]triazin-7-yl)-4-cyano-2,2-dimethyltetrahydrofuro[3,4-d][1,3]dioxol-4-yl)methyl isopentyl carbonate

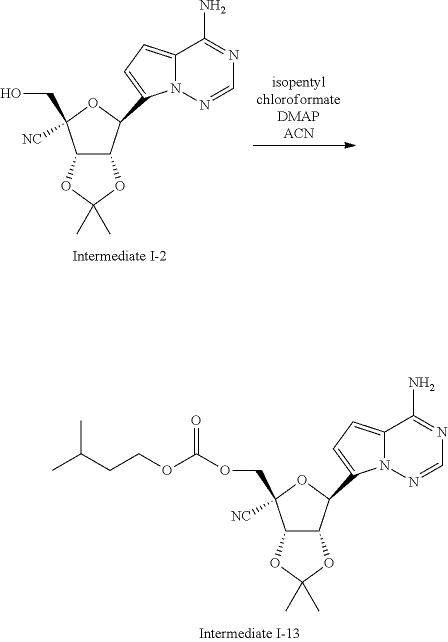 [SMILES: CC1(C)O[C@H]2[C@H](c3ccc4c(N)ncnn34)O[C@](C#N)(CO)[C@H]2O1 ; CC(C)CCOC(=O)OC[C@@]1(C#N)O[C@@H](c2ccc3c(N)ncnn23)[C@@H]2OC(C)(C)O[C@@H]21]

To a mixture of Intermediate I-2 (0.302 mmol) and isopentyl carbonochloridate (0.362 mmol) in ACN (2 mL) was added DMAP (0.604 mmol). The resulting mixture was stirred at rt for 1 h, MeOH added, concentrated in vacuo, and purified by silica gel column chromatography (0 to 5% MeOH in DCM) to give Intermediate I-13.

¹H NMR (400 MHz, Acetonitrile-d3) δ 7.91 (s, 1H), 6.80 (d, J=4.5 Hz, 1H), 6.77 (d, J=4.5 Hz, 1H), 6.36 (s, 2H), 5.67 (d, J=3.3 Hz, 1H), 5.32 (dd, J=6.6, 3.4 Hz, 1H), 5.10 (d, J=6.6 Hz, 1H), 4.50 (d, J=11.3 Hz, 1H), 4.43 (d, J=11.3 Hz, 1H), 4.18 (m, 2H), 1.76-1.63 (m, 4H), 1.55 (q, J=6.82 Hz, 2H), 1.39 (s, 3H), 0.94 (d, J=1.3 Hz, 3H), 0.92 (d, J=1.3 Hz, 3H). MS m/z [M+1]=446.0.

### Intermediate I-14: ((3aS,4R,6S,6aS)-6-(4-aminopyrrolo[2,1-f][1,2,4]triazin-7-yl)-4-cyano-2,2-dimethyltetrahydrofuro[3,4-d][1,3]dioxol-4-yl)methyl cyclopentyl carbonate

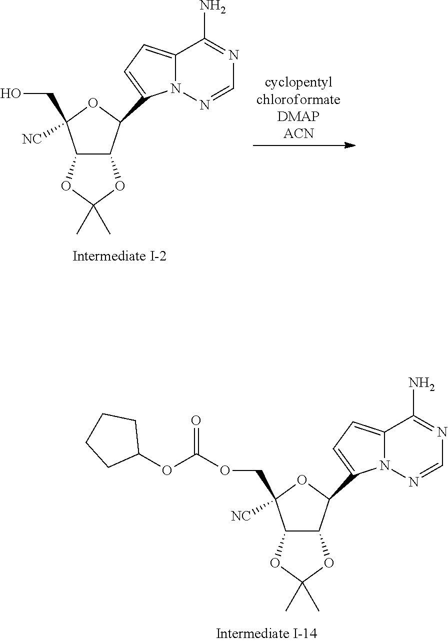 [SMILES: CC1(C)O[C@H]2[C@H](c3ccc4c(N)ncnn34)O[C@](C#N)(CO)[C@H]2O1 ; CC1(C)O[C@H]2[C@H](c3ccc4c(N)ncnn34)O[C@](C#N)(COC(=O)OC3CCCC3)[C@H]2O1]

To a mixture of Intermediate I-2 (0.302 mmol) and cyclopentyl carbonochloridate (0.045 mL, 0.362 mmol) in ACN (2 mL) was added DMAP (0.604 mmol). The resulting mixture was stirred at rt for 1 h, MeOH added, concentrated in vacuo, and purified by silica gel column chromatography (0 to 5% MeOH in DCM) to give Intermediate I-14.

¹H NMR (400 MHz, Acetonitrile-d3) δ 7.91 (s, 1H), 6.80 (d, J=4.5 Hz, 1H), 6.77 (d, J=4.5 Hz, 1H), 6.34 (s, 2H), 5.67 (d, J=3.3 Hz, 1H), 5.32 (dd, J=6.6, 3.3 Hz, 1H), 5.14-5.00 (m, 2H), 4.48 (d, J=11.4 Hz, 1H), 4.41 (d, J=11.3 Hz, 1H), 1.88 (m, 2H), 1.81-1.67 (m, 7H), 1.67-1.53 (m, 2H), 1.39 (s, 3H). MS m/z [M+1]=443.9.

### Intermediate I-15: ((3aS,4R,6S,6aS)-6-(4-aminopyrrolo[2,1-f][1,2,4]triazin-7-yl)-4-cyano-2,2-dimethyltetrahydrofuro[3,4-d][1,3]dioxol-4-yl)methyl phenethyl carbonate

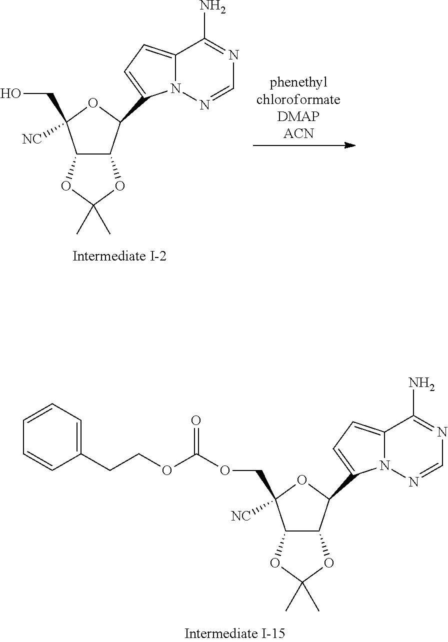 [SMILES: CC1(C)O[C@H]2[C@H](c3ccc4c(N)ncnn34)O[C@](C#N)(CO)[C@H]2O1 ; CC1(C)O[C@H]2[C@H](c3ccc4c(N)ncnn34)O[C@](C#N)(COC(=O)OCCc3ccccc3)[C@H]2O1]

To a mixture of Intermediate I-2 (0.341 mmol) and 2-phenylethyl carbonochloridate (0.409 mmol) in ACN (2 mL) was added DMAP (0.682 mmol). The resulting mixture was stirred at rt for 1h, concentrated in vacuo, and purified by silica gel column chromatography (0 to 5% MeOH in DCM) to give Intermediate I-15.

¹H NMR (400 MHz, Acetonitrile-d3) δ 7.91 (s, 1H), 7.37-7.22 (m, 5H), 6.79 (d, J=4.5 Hz, 1H), 6.77 (d, J=4.5 Hz, 1H), 6.30 (s, 2H), 5.67 (d, J=3.3 Hz, 1H), 5.31 (dd, J=6.7, 3.4 Hz, 1H), 5.08 (d, J=6.6 Hz, 1H), 4.48 (d, J=11.32 Hz, 1H), 4.42 (d, J=11.38 Hz, 1H), 4.37 (t, J=6.8 Hz, 2H), 2.98 (t, J=6.8 Hz, 2H), 1.72 (s, 3H), 1.39 (s, 3H). MS m/z [M+1]=479.9.

### Intermediate I-16: ((3aS,4R,6S,6aS)-6-(4-aminopyrrolo[2,1-f][1,2,4]triazin-7-yl)-4-cyano-2,2-dimethyltetrahydrofuro[3,4-d][1,3]dioxol-4-yl)methyl (1-methylcyclopropyl) carbonate

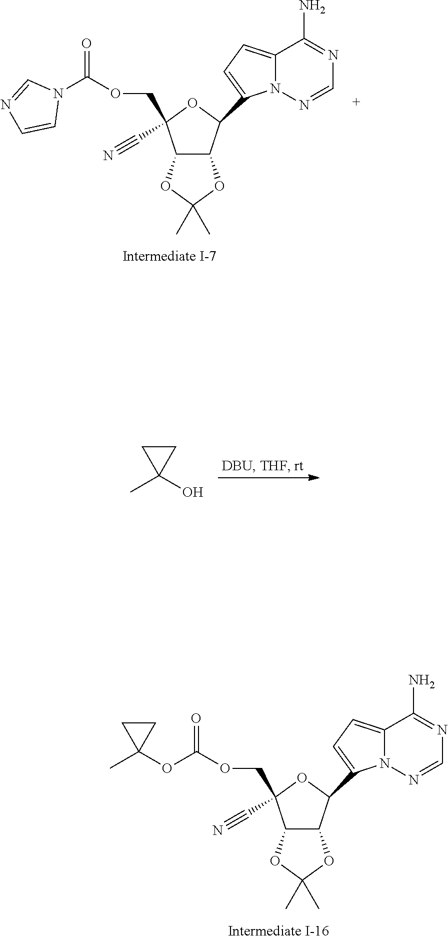

To a solution of 1-methylcyclopropan-1-ol (0.17 mL, 1.9 mmol), in THF (5 mL) was added DBU (0.11 mL, 0.71 mmol) at once and stirred at room temperature for 10 min. To well stirring mixture was added Intermediate I-7 (0.47 mmol) at once and stirred additional 2 h. The reaction mixture was concentrated in vacuo, crude dissolve in DCM, loaded on 40 g gold column, eluted with Hexanes 3 minutes, DCM 1 minutes, and 0-100% EtOAc/DCM to afford the Intermediate I-16.

¹H NMR (400 MHz, Acetonitrile-d₃) δ 7.91 (s, 1H), 6.80 (d, J=4.5 Hz, 1H), 6.77 (d, J=4.5 Hz, 1H), 6.28 (s, 2H), 5.67 (d, J=3.2 Hz, 1H), 5.32 (dd, J=6.6, 3.4 Hz, 1H), 5.09 (d, J=6.5 Hz, 1H), 4.48 (d, J=11.3 Hz, 1H), 4.39 (d, J=11.3 Hz, 1H), 1.72 (s, 3H), 1.53 (s, 3H), 1.39 (s, 3H), 0.95-0.92 (m, 2H), 0.72-0.63 (m, 2H). MS m/z [M+1]=429.9.

### Intermediate I-17: ((3aS,4R,6S,6aS)-6-(4-aminopyrrolo[2,1-f][1,2,4]triazin-7-yl)-4-cyano-2,2-dimethyltetrahydrofuro[3,4-d][1,3]dioxol-4-yl)methyl (tetrahydro-2H-pyran-4-yl) carbonate

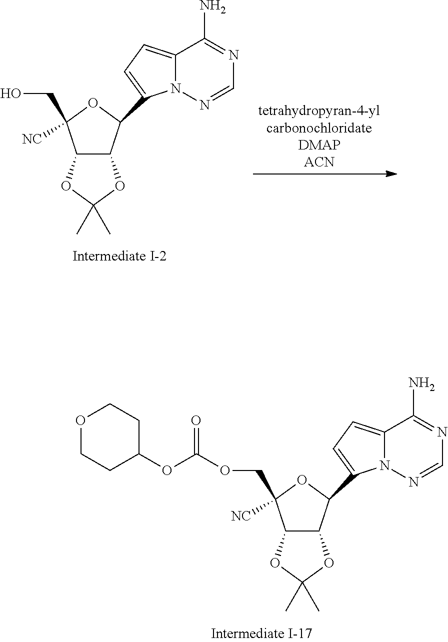 [SMILES: CC1(C)O[C@H]2[C@H](c3ccc4c(N)ncnn34)O[C@](C#N)(CO)[C@H]2O1 ; CC1(C)O[C@H]2[C@H](c3ccc4c(N)ncnn34)O[C@](C#N)(COC(=O)OC3CCOCC3)[C@H]2O1]

To a mixture of Intermediate I-2 (0.302 mmol) and tetrahydropyran-4-yl carbonochloridate (0.362 mmol) in ACN (2 mL) was added DMAP (0.604 mmol). The resulting mixture was stirred at rt for 1h, MeOH added, concentrated in vacuo, and purified by silica gel column chromatography (0 to 5% MeOH in DCM) to give Intermediate I-17.

¹H NMR (400 MHz, Acetonitrile-d3) δ 7.91 (s, 1H), 6.80 (d, J=4.5 Hz, 1H), 6.77 (d, J=4.5 Hz, 1H), 6.34 (s, 2H), 5.68 (d, J=3.3 Hz, 1H), 5.33 (dd, J=6.6, 3.4 Hz, 1H), 5.10 (d, J=6.6 Hz, 1H), 4.79 (m, 1H), 4.50 (d, J=11.3 Hz, 1H), 4.44 (d, J=11.4 Hz, 1H), 3.85 (m, 2H), 3.48 (m, 2H), 1.95 (m, 2H), 1.74-1.61 (m, 5H), 1.39 (s, 3H). MS m/z [M+1]=459.9.

### Intermediate I-18: ((3aS,4R,6S,6aS)-6-(4-aminopyrrolo[2,1-f][1,2,4]triazin-7-yl)-4-cyano-2,2-dimethyltetrahydrofuro[3,4-d][1,3]dioxol-4-yl)methyl cyclobutyl carbonate

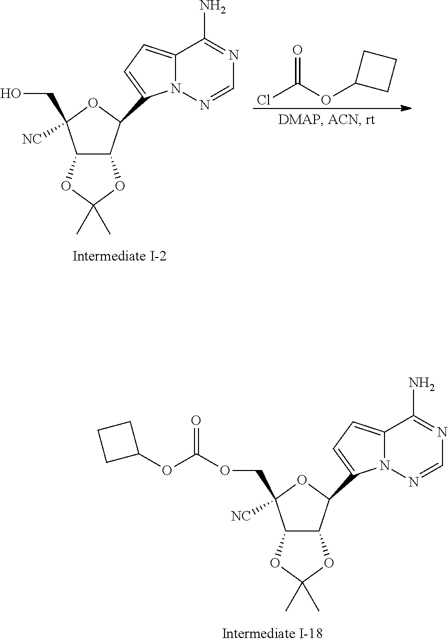

To a solution of Intermediate I-2 (0.2 g, 0.604 mmol) in ACN (3.0 mL) was added DMAP (147 mg, 1.21 mmol) and cooled to 0° C. To well stirred mixture was added cyclobutyl carbonochloridate (99 mg, 0.73 mmol). The solution was warmed to room temperature and stirred 30 min. The reaction was treated with MeOH (0.1 mL) and stirred for 10 min at room temperature prior to concentrating in vacuo. The crude residue was taken up in EtOAc and filtered. The filtrate was concentrated in vacuo and subjected to silica gel chromatography (0-100% EtOAc/DCM) to afford Intermediate I-18. ¹H NMR (400 MHz, DMSO-d₆) δ 7.90 (s, 1H), 7.86 (s, 2H), 6.88 (d, J=4.5 Hz, 1H), 6.84 (d, J=4.5 Hz, 1H), 5.62 (d, J=3.6 Hz, 1H), 5.31 (dd, J=6.6, 3.6 Hz, 1H), 5.10 (d, J=6.7 Hz, 1H), 4.88-4.81 (m, 1H), 4.48 (d, J=11.5 Hz, 1H), 4.39 (d, J=11.5 Hz, 1H), 2.31-2.23 (m, 2H), 2.09-2.00 (m, 2H), 1.82-1.67 (m, 1H), 1.65 (s, 3H), 1.61-1.51 (m, 1H), 1.36 (s, 3H). MS m/z [M+1]=429.96

### Intermediate I-19A: (Z)—N′-(7-((3aS,4S,6R,6aS)-6-cyano-6-(hydroxymethyl)-2,2-dimethyltetrahydrofuro[3,4-d][1,3]dioxol-4-yl)pyrrolo[2,1-f][1,2,4]triazin-4-yl)-N,N-dimethylformimidamide

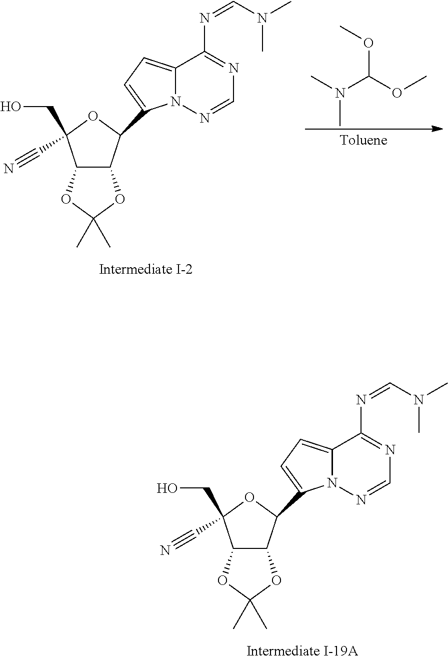

To a suspension of Intermediate I-2 (3000 mg, 9.1 mmol) in toluene (20 mL) N,N-Dimethylformamide dimethyl acetal (2.4 mL, 18 mmol) was added and heated at 50° C. for 1h, during which time the reaction mixture became clear. LC-MS shows the complete conversion to the product. Toluene was distilled off, the residue was dissolved in 10 mL of methanol and 100 mL of water was added and the mixture was stirred overnight. Filtered the precipitate, air dried to get the Intermediate I-19A. MS m/z [M+1]=387.1

### Intermediate I-19: ((3aS,4R,6S,6aS)-4-cyano-6-(4-(((Z)-(dimethylamino)methylene)amino)pyrrolo[2,1-f][1,2,4]triazin-7-yl)-2,2-dimethyltetrahydrofuro[3,4-d][1,3]dioxol-4-yl)methyl cyclooctyl carbonate

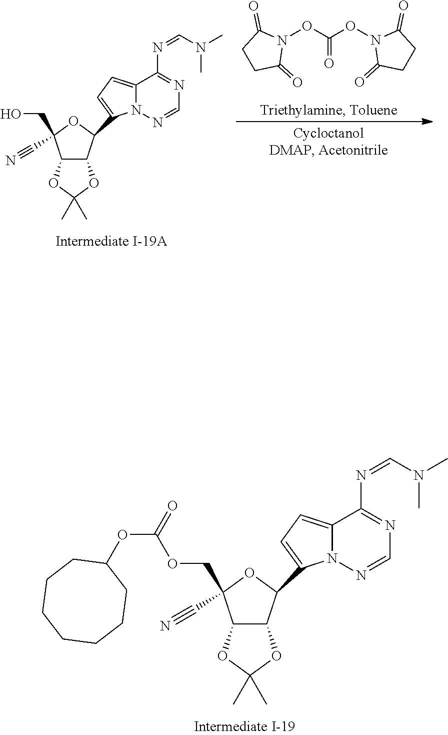

To a solution of Intermediate I-19A (500 mg, 1.3 mmol) in acetonitrile (5 mL), bis(2,5-dioxopyrrolidin-1-yl) carbonate (663 mg, 2.6 mmol) was added followed by triethyl amine (0.72 mL, 5.2 mmol) and stirred at room temperature for 90 min, shows the formation of the intermediate [(3aS,4R,6S,6aS)-4-cyano-6-[4-[(Z)-dimethylaminomethyleneamino]pyrrolo[2,1-f][1,2,4]triazin-7-yl]-2,2-dimethyl-6,6α-dihydro-3aH-furo[3,4-d][1,3]dioxol-4-yl]methyl (2,5-dioxopyrrolidin-1-yl) carbonate by LC-MS (MS m/z [M+1]=528.1). To the reaction mixture cyclooctanol (1 mL, 7.8 mL) followed by dimethyl aminopyridine (158 mg, 1.3 mmol) were added and continued stirring for 3h. After completion of the reaction, the reaction mixture was diluted with ethyl acetate (50 mL), washed with water, brine, dried and concentrated and used for the next step. LC-MS (MS m/z [M+1]=541.2

### Intermediate I-20: ((3aS,4R,6S,6aS)-6-(4-aminopyrrolo[2,1-f][1,2,4]triazin-7-yl)-4-cyano-2,2-dimethyltetrahydrofuro[3,4-d][1,3]dioxol-4-yl)methyl hexyl carbonate

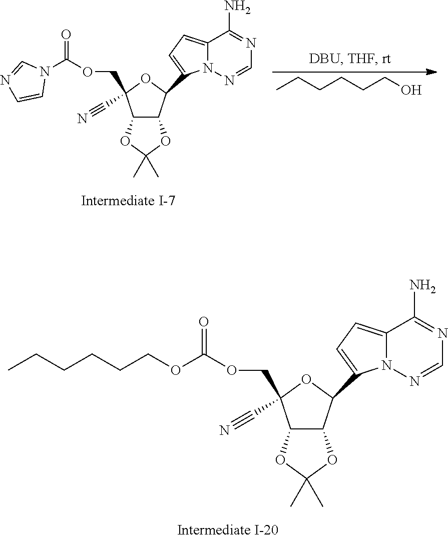

To a solution of ((3aS,4R,6S,6aS)-6-(4-aminopyrrolo[2,1-f][1,2,4]triazin-7-yl)-4-cyano-2,2-dimethyltetrahydrofuro[3,4-d][1,3]dioxol-4-yl)methyl 1H-imidazole-1-carboxylate (157 mg, 0.37 mmol, 1.0 equiv.) in THF (5.0 mL) was added hexan-1-ol (0.23 mL, 1.8 mmol, 5.0 equiv.), followed by DBU (0.08 mL, 0.55 mmol, 1.5 equiv.) The solution was stirred at room temperature for 2 h prior to concentrating in vacuo and subjecting to silica gel chromatography (0-100% EtOAc in hexanes) to afford the title compound Intermediate I-20. ¹H NMR (400 MHz, DMSO-d6) δ 7.93-7.74 (m, 3H), 6.87 (d, J=4.5 Hz, 1H), 6.82 (d, J=4.5 Hz, 1H), 5.62 (d, J=3.6 Hz, 1H), 5.30 (dd, J=6.6, 3.7 Hz, 1H), 5.09 (d, J=6.6 Hz, 1H), 4.50 (d, J=11.5 Hz, 1H), 4.40 (d, J=11.5 Hz, 1H), 4.12-4.06 (m, 2H), 1.67-1.52 (m, 5H), 1.38-1.20 (m, 9H), 0.88-0.81 (m, 3H). MS m/z [M+1]=460.1

### Intermediate I-21: ((3aS,4R,6S,6aS)-6-(4-aminopyrrolo[2,1-f][1,2,4]triazin-7-yl)-4-cyano-2,2-dimethyltetrahydrofuro[3,4-d][1,3]dioxol-4-yl)methyl pentyl carbonate

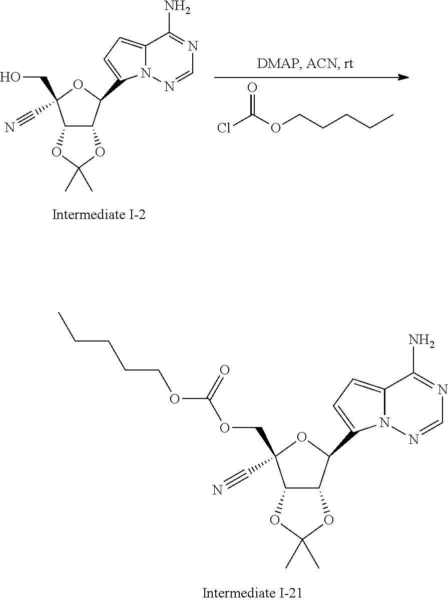

To a solution of (3aS,4R,6S,6aS)-6-(4-aminopyrrolo[2,1-f][1,2,4]triazin-7-yl)-4-(hydroxymethyl)-2,2-dimethyltetrahydrofuro[3,4-d][1,3]dioxole-4-carbonitrile (150 mg, 0.45 mmol, 1.0 equiv.) and DMAP (75 mg, 0.61 mmol, 1.4 equiv.) in ACN (5.0 mL) was added amyl chloroformate (0.07 mL, 0.50 mmol, 1.1 equiv.). The solution was stirred for 1 h and 40 min at room temperature. The reaction was treated with MeOH (0.5 mL) and concentrated in vacuo. The crude residue was taken up in EtOAc and filtered. The filtrate was concentrated in vacuo and subjected to silica gel chromatography (0-100% EtOAc in hexanes) to afford the title compound Intermediate I-21. ¹H NMR (400 MHz, DMSO-d6) δ 7.94-7.75 (m, 3H), 6.87 (d, J=4.5 Hz, 1H), 6.83 (d, J=4.5 Hz, 1H), 5.62 (d, J=3.6 Hz, 1H), 5.30 (dd, J=6.7, 3.7 Hz, 1H), 5.09 (d, J=6.6 Hz, 1H), 4.50 (d, J=11.5 Hz, 1H), 4.40 (d, J=11.5 Hz, 1H), 4.11-4.06 (m, 2H), 1.66-1.53 (m, 5H), 1.35 (s, 3H), 1.32-1.23 (m, 4H), 0.89-0.81 (m, 3H). MS m/z [M+1]=446.1

### Intermediate I-22: ((3aS,4R,6S,6aS)-6-(4-aminopyrrolo[2,1-f][1,2,4]triazin-7-yl)-4-cyano-2,2-dimethyltetrahydrofuro[3,4-d][1,3]dioxol-4-yl)methyl cyclohexyl carbonate

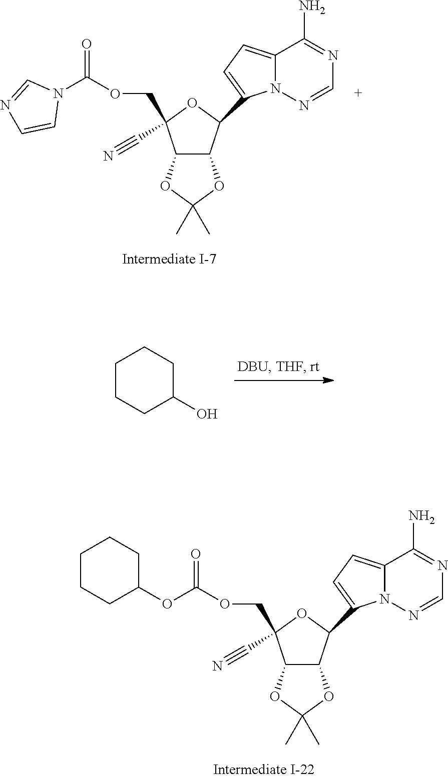 [SMILES: CC1(C)O[C@H]2[C@H](c3ccc4c(N)ncnn34)O[C@](C#N)(COC(=O)n3ccnc3)[C@H]2O1 ; OC1CCCCC1 ; CC1(C)O[C@H]2[C@H](c3ccc4c(N)ncnn34)O[C@](C#N)(COC(=O)OC3CCCCC3)[C@H]2O1]

A round bottom flask was charged with ((3aS,4R,6S,6aS)-6-(4-aminopyrrolo[2,1-f1][1,2,4]triazin-7-yl)-4-cyano-2,2-dimethyltetrahydrofuro[3,4-d][1,3]dioxol-4-yl)methyl 1H-imidazole-1-carboxylate (173 mg, 0.41 mmol, 1.0 equiv.). Cyclohexanol (253 mg, 2.5 mmol, 6.2 equiv.) dissolved in THE (5.0 mL) was transferred to the flask followed by DBU (0.09 mL, 0.61 mmol, 1.5 equiv.) The solution was stirred at room temperature for 2 h and 30 min prior to concentrating in vacuo and subjecting to silica gel chromatography (0-100% EtOAc in hexanes) to afford the title compound Intermediate I-22. ¹H NMR (400 MHz, DMSO-d6) δ 7.96-7.72 (m, 3H), 6.87 (d, J=4.5 Hz, 1H), 6.83 (d, J=4.5 Hz, 1H), 5.62 (d, J=3.6 Hz, 1H), 5.30 (dd, J=6.6, 3.7 Hz, 1H), 5.09 (d, J=6.6 Hz, 1H), 4.57-4.44 (m, 2H), 4.38 (d, J=11.5 Hz, 1H), 1.88-1.76 (m, 2H), 1.70-1.59 (m, 5H), 1.52-1.20 (m, 9H). MS m/z [M+1]=458.1 Intermediate 23: ((3aS,4R,6S,6aS)-6-(4-aminopyrrolo[2,1-f][1,2,4]triazin-7-yl)-4-cyano-2,2-dimethyltetrahydrofuro[3,4-d][1,3]dioxol-4-yl)methyl ethyl carbonate

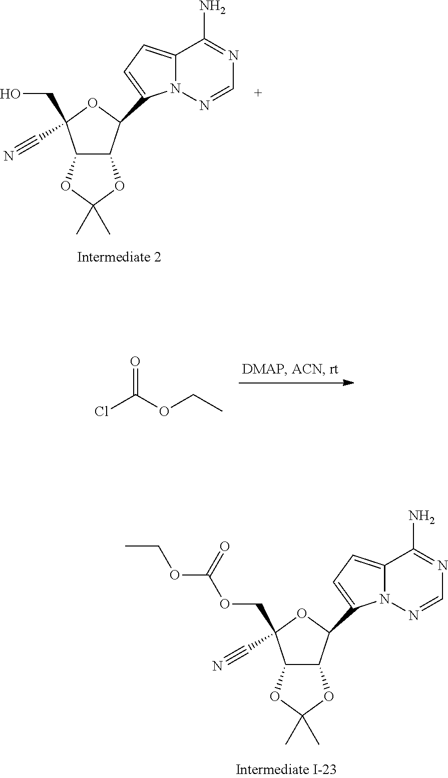

To a solution of (3aS,4R,6S,6aS)-6-(4-aminopyrrolo[2,1-f][1,2,4]triazin-7-yl)-4-(hydroxymethyl)-2,2-dimethyltetrahydrofuro[3,4-d][1,3]dioxole-4-carbonitrile (203 mg, 0.61 mmol, 1.0 equiv.) and DMAP (95 mg, 0.78 mmol, 1.3 equiv.) in ACN (5.0 mL) was added ethyl chloroformate (0.06 mL, 0.67 mmol, 1.1 equiv.). The solution was stirred for 1 h at room temperature. The reaction was treated with MeOH (0.5 mL) and concentrated in vacuo. The crude residue was taken up in EtOAc and filtered. The filtrate was concentrated in vacuo and subjected to silica gel chromatography (0-100% EtOAc in hexanes) to afford the title compound, Intermediate I-23. ¹H NMR (400 MHz, DMSO-d6) δ 7.97-7.73 (m, 3H), 6.87 (d, J=4.5 Hz, 1H), 6.83 (d, J=4.5 Hz, 1H), 5.62 (d, J=3.6 Hz, 1H), 5.30 (dd, J=6.6, 3.7 Hz, 1H), 5.10 (d, J=6.6 Hz, 1H), 4.49 (d, J=11.5 Hz, 1H), 4.40 (d, J=11.4 Hz, 1H), 4.14 (q, J=7.1 Hz, 2H), 1.64 (s, 3H), 1.35 (s, 3H), 1.21 (t, J=7.1 Hz, 3H). MS m/z [M+1]=404.1

### Intermediate 24: ((3aS,4R,6S,6aS)-6-(4-aminopyrrolo[2,1-f][1,2,4]triazin-7-yl)-4-cyano-2,2-dimethyltetrahydrofuro[3,4-d][1,3]dioxol-4-yl)methyl cyclopropyl carbonate

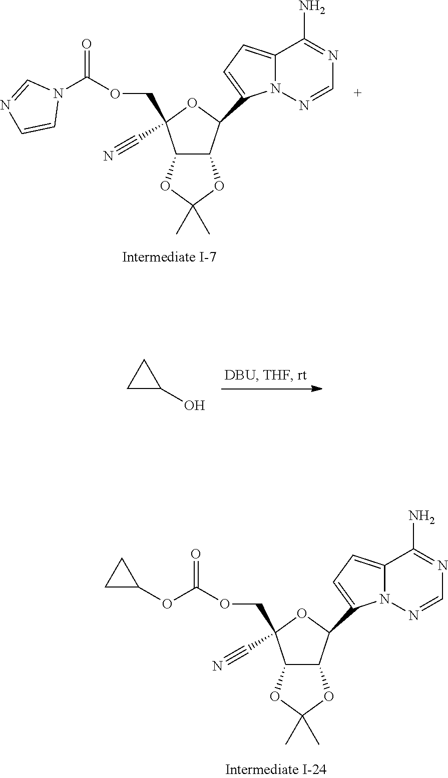

To a solution of ((3aS,4R,6S,6aS)-6-(4-aminopyrrolo[2,1-f][1,2,4]triazin-7-yl)-4-cyano-2,2-dimethyltetrahydrofuro[3,4-d][1,3]dioxol-4-yl)methyl 1H-imidazole-1-carboxylate (150 mg, 0.35 mmol, 1.0 equiv.) in THF (5.0 mL) was added cyclopropanol (0.05 mL, 0.78 mmol, 2.2 equiv.) followed by DBU (0.08 mL, 0.53 mmol, 1.5 equiv.) The solution was stirred at room temperature for 45 min prior to concentrating in vacuo and subjecting to silica gel chromatography (0-100% EtOAc in hexanes) to afford the Intermediate I-24. ¹H NMR (400 MHz, DMSO-d6) δ 7.94-7.74 (m, 3H), 6.87 (d, J=4.5 Hz, 1H), 6.82 (d, J=4.5 Hz, 1H), 5.61 (d, J=3.6 Hz, 1H), 5.30 (dd, J=6.6, 3.7 Hz, 1H), 5.09 (d, J=6.6 Hz, 1H), 4.51 (d, J=11.5 Hz, 1H), 4.41 (d, J=11.5 Hz, 1H), 4.14-4.07 (m, 1H), 1.64 (s, 3H), 1.35 (s, 3H), 0.73-0.66 (m, 4H). MS m/z [M+1]=416.1

### Intermediate I-25: ((3aS,4R,6S,6aS)-6-(4-aminopyrrolo[2,1-f][1,2,4]triazin-7-yl)-4-cyano-2,2-dimethyltetrahydrofuro[3,4-d][1,3]dioxol-4-yl)methyl ((R)-tetrahydrofuran-3-yl) carbonate

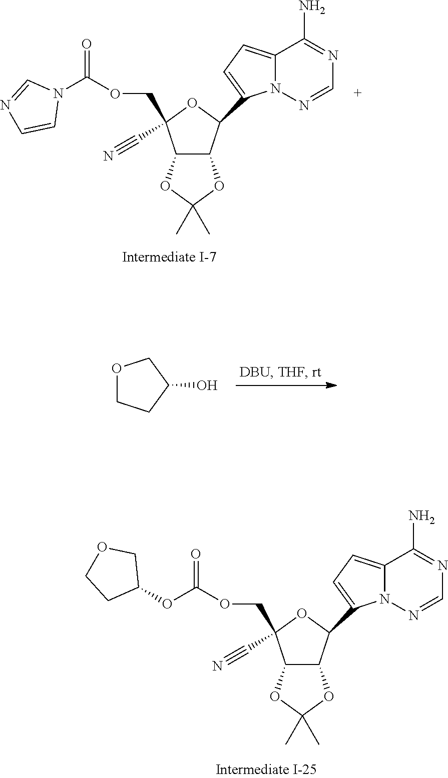 [SMILES: CC1(C)O[C@H]2[C@H](c3ccc4c(N)ncnn34)O[C@](C#N)(COC(=O)n3ccnc3)[C@H]2O1 ; O[C@@H]1CCOC1 ; CC1(C)O[C@H]2[C@H](c3ccc4c(N)ncnn34)O[C@](C#N)(COC(=O)O[C@@H]3CCOC3)[C@H]2O1]

To a solution of ((3aS,4R,6S,6aS)-6-(4-aminopyrrolo[2,1-f][1,2,4]triazin-7-yl)-4-cyano-2,2-dimethyltetrahydrofuro[3,4-d][1,3]dioxol-4-yl)methyl 1H-imidazole-1-carboxylate (109 mg, 0.26 mmol, 1.0 equiv.) in THF (5.0 mL) was added (R)-tetrahydrofuran-3-ol (94 mg, 1.1 mmol, 4.2 equiv.) followed by DBU (0.06 mL, 0.38 mmol, 1.5 equiv.) The solution was stirred at room temperature for 15 min prior to concentrating in vacuo and subjecting to silica gel chromatography twice (0-100% EtOAc in hexanes) to afford the Intermediate I-25. ¹H NMR (400 MHz, DMSO-d6) δ 7.98-7.69 (m, 3H), 6.87 (d, J=4.5 Hz, 1H), 6.83 (d, J=4.5 Hz, 1H), 5.62 (d, J=3.6 Hz, 1H), 5.31 (dd, J=6.6, 3.6 Hz, 1H), 5.19-5.14 (m, 1H), 5.10 (d, J=6.6 Hz, 1H), 4.50 (d, J=11.5 Hz, 1H), 4.40 (d, J=11.5 Hz, 1H), 3.83-3.61 (m, 4H), 2.21-2.09 (m, 1H), 2.02-1.85 (m, 1H), 1.63 (s, 3H), 1.35 (s, 3H). MS m/z [M+1]=446.1

### Intermediate I-26: 1,1-difluoro-2-methylpropan-2-yl 1H-imidazole-1-carboxylate

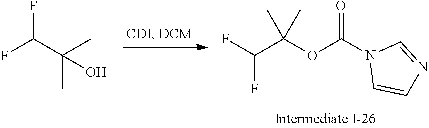

To a solution of CDI (1.18 g, 7.28 mmol, 1.27 equiv.) in DCM (15.0 mL) cooled in an ice bath was added 1,1-difluoro-2-methylpropan-2-ol (632 mg, 5.74 mmol, 1.0 equiv.) dissolved in DCM (3.0 mL). The reaction mixture was stirred at room temperature for 2 days prior to diluting with DCM (75 mL) and water (75 mL). The layers were separated and the organic phase was dried over Na₂SO₄, filtered and concentrated in vacuo without further purification. ¹H NMR (400 MHz, Chloroform-d) δ 8.09-8.06 (m, 1H), 7.38-7.35 (m, 1H), 7.08-7.05 (m, 1H), 6.07 (t, J=56.2 Hz, 1H), 1.70-1.66 (m, 6H). ¹⁹F NMR (376 MHz, Chloroform-d) 6-132.72-−132.96 (m).

### Intermediate 1-27: ((3aS,4R,6S,6aS)-6-(4-aminopyrrolo[2,1-f][1,2,4]triazin-7-yl)-4-cyano-2,2-dimethyltetrahydrofuro[3,4-d][1,3]dioxol-4-yl)methyl (1,1-difluoro-2-methylpropan-2-yl) carbonate

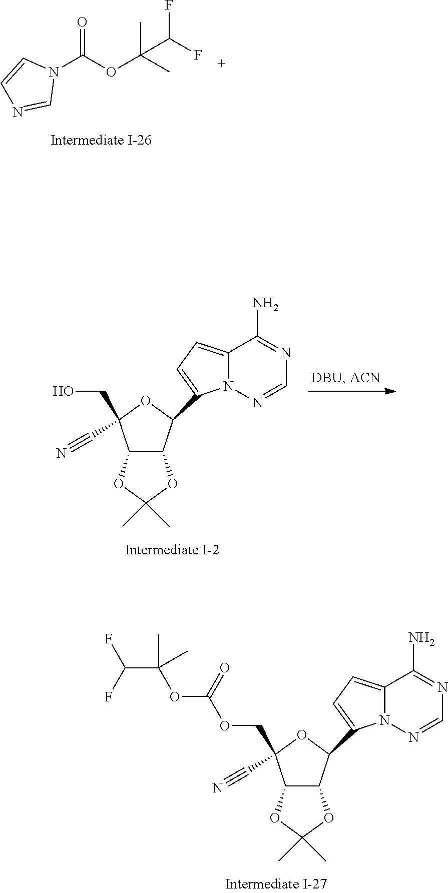 [SMILES: CC(C)(OC(=O)n1ccnc1)C(F)F ; CC1(C)O[C@H]2[C@H](c3ccc4c(N)ncnn34)O[C@](C#N)(CO)[C@H]2O1 ; CC1(C)O[C@H]2[C@H](c3ccc4c(N)ncnn34)O[C@](C#N)(COC(=O)OC(C)(C)C(F)F)[C@H]2O1]

To a solution of (3aS,4R,6S,6aS)-6-(4-aminopyrrolo[2,1-f][1,2,4]triazin-7-yl)-4-(hydroxymethyl)-2,2-dimethyltetrahydrofuro[3,4-d][1,3]dioxole-4-carbonitrile (150 mg, 0.453 mmol, 1.0 equiv.) and 1,1-difluoro-2-methylpropan-2-yl 1H-imidazole-1-carboxylate (111 mg, 0.543 mmol, 1.2 equiv.) in ACN (3.0 mL) was added DBU (0.03 mL, 0.226 mmol, 0.50 equiv.). The reaction mixture was stirred at room temperature overnight. The solution was diluted with EtOAc (100 mL) and water (100 mL). The organic layer was washed with brine (50 mL), dried over sodium sulfate, filtered, concentrated in vacuo and purified by silica gel chromatography (0-100% EtOAc in hexanes) to afford the Intermediate 1-27. ¹H NMR (400 MHz, DMSO-d6) δ 7.93-7.76 (m, 3H), 6.87 (d, J=4.5 Hz, 1H), 6.83 (d, J=4.5 Hz, 1H), 6.17 (t, J=55.4 Hz, 1H), 5.62 (d, J=3.6 Hz, 1H), 5.31 (dd, J=6.6, 3.7 Hz, 1H), 5.10 (d, J=6.7 Hz, 1H), 4.50 (d, J=11.5 Hz, 1H), 4.38 (d, J=11.5 Hz, 1H), 1.64 (s, 3H), 1.48-1.43 (m, 6H), 1.35 (s, 3H). ¹⁹F NMR (376 MHz, DMSO-d6) δ −132.96-−133.25 (m). MS m/z [M+1]=467.9

### Intermediate I-28: ((3aS,4R,6S,6aS)-6-(4-aminopyrrolo[2,1-f][1,2,4]triazin-7-yl)-4-cyano-2,2-dimethyltetrahydrofuro[3,4-d][1,3]dioxol-4-yl)methyl ((S)-2-methylbutyl) carbonate

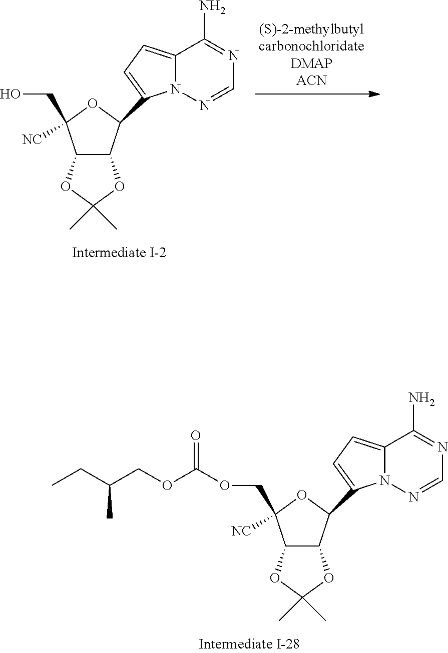 [SMILES: CC1(C)O[C@H]2[C@H](c3ccc4c(N)ncnn34)O[C@](C#N)(CO)[C@H]2O1 ; CC[C@H](C)COC(=O)OC[C@@]1(C#N)O[C@@H](c2ccc3c(N)ncnn23)[C@@H]2OC(C)(C)O[C@@H]21]

To a mixture of Intermediate I-2 (100 mg, 0.302 mmol) and (S)-2-methylbutyl carbonochloridate (0.0545 g, 0.362 mmol) in ACN (2 mL) was added DMAP (74 mg, 0.604 mmol). The resulting mixture was stirred at rt for 1h, MeOH added, concentrated in vacuo, and purified by silica gel column chromatography (0 to 5% MeOH in DCM) to give Intermediate I-28. ¹H NMR (400 MHz, Acetonitrile-d3) δ 7.91 (s, 1H), 6.80 (d, J=4.5 Hz, 1H), 6.77 (d, J=4.5 Hz, 1H), 6.36 (s, 2H), 5.68 (d, J=3.3 Hz, 1H), 5.32 (dd, J=6.6, 3.4 Hz, 1H), 5.10 (d, J=6.6 Hz, 1H), 4.51 (d, J=11.4 Hz, 1H), 4.43 (d, J=11.3 Hz, 1H), 4.05 (ddd, J=10.5, 6.0, 1.9 Hz, 1H), 3.96 (ddd, J=10.5, 6.6, 2.1 Hz, 1H), 1.84-1.65 (m, 4H), 1.52-1.34 (m, 4H), 1.29-1.13 (m, 1H), 0.93 (d, J=0.7 Hz, 3H), 0.92 (d, J=1.1 Hz, 3H). MS m/z [M+1]=446.0

### Intermediate I-29: ((3aS,4R,6S,6aS)-6-(4-aminopyrrolo[2,1-f][1,2,4]triazin-7-yl)-4-cyano-2,2-dimethyltetrahydrofuro[3,4-d][1,3]dioxol-4-yl)methyl benzyl carbonate

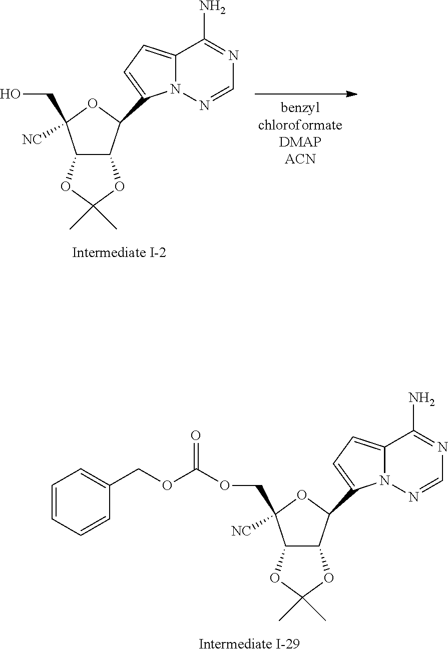 [SMILES: CC1(C)O[C@H]2[C@H](c3ccc4c(N)ncnn34)O[C@](C#N)(CO)[C@H]2O1 ; CC1(C)O[C@H]2[C@H](c3ccc4c(N)ncnn34)O[C@](C#N)(COC(=O)OCc3ccccc3)[C@H]2O1]

To a mixture of Intermediate I-2 (100 mg, 0.302 mmol) and benzyl chloroformate (61.8 mg, 0.362 mmol) in ACN (2 mL) was added DMAP (74 mg, 0.604 mmol). The resulting mixture was stirred at rt for 1h, MeOH added, concentrated in vacuo, and purified by silica gel column chromatography (0 to 5% MeOH in DCM) to give Intermediate I-29. ¹H NMR (400 MHz, Acetonitrile-d3) δ 7.90 (s, 1H), 7.40 (d, J=3.4 Hz, 5H), 6.79 (d, J=4.5 Hz, 1H), 6.76 (d, J=4.6 Hz, 1H), 6.39 (s, 2H), 5.68 (d, J=3.4 Hz, 1H), 5.31 (dd, J=6.6, 3.4 Hz, 1H), 5.18 (s, 2H), 5.10 (d, J=6.6 Hz, 1H), 4.54 (d, J=11.4 Hz, 1H), 4.47 (d, J=11.3 Hz, 1H), 1.72 (s, 3H), 1.38 (s, 3H). MS m/z [M+1]=465.7

### Intermediate I-30: ((3aS,4R,6S,6aS)-6-(4-aminopyrrolo[2,1-f][1,2,4]triazin-7-yl)-4-cyano-2,2-dimethyltetrahydrofuro[3,4-d][1,3]dioxol-4-yl)methyl (cyclopentylmethyl) carbonate

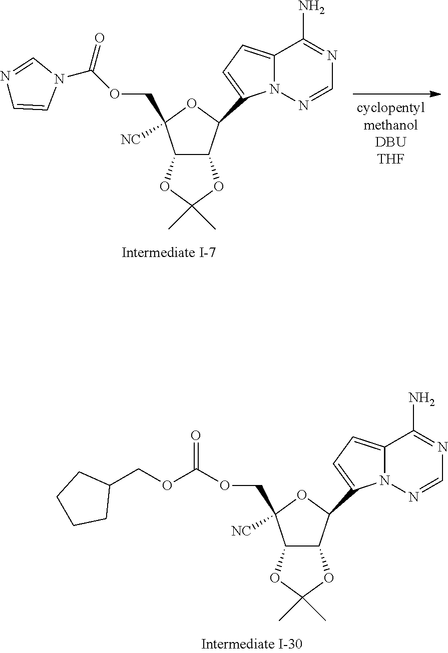 [SMILES: CC1(C)O[C@H]2[C@H](c3ccc4c(N)ncnn34)O[C@](C#N)(COC(=O)n3ccnc3)[C@H]2O1 ; CC1(C)O[C@H]2[C@H](c3ccc4c(N)ncnn34)O[C@](C#N)(COC(=O)OCC3CCCC3)[C@H]2O1]

To a solution of Intermediate I-7 (150 mg, 1.40 mmol) in THE (2 mL) were added cyclopentyl methanol (0.15 mL, 1.4 mmol) and then DBU (0.08 mL, 0.53 mmol). The resulting mixture was stirred at rt for 2 h. The reaction mixture was concentrated in vacuo and purified by silica gel chromatography (0 to 5% MeOH in DCM) to give Intermediate I-30. ¹H NMR (400 MHz, Acetonitrile-d3) δ 7.91 (s, 1H), 6.80 (d, J=4.5 Hz, 1H), 6.77 (d, J=4.5 Hz, 1H), 6.28 (s, 2H), 5.68 (d, J=3.3 Hz, 1H), 5.32 (dd, J=6.7, 3.4 Hz, 1H), 5.10 (d, J=6.6 Hz, 1H), 4.50 (d, J=11.4 Hz, 1H), 4.43 (d, J=11.3 Hz, 1H), 4.05 (d, J=7.2 Hz, 2H), 2.33-2.17 (m, 1H), 1.84-1.67 (m, 5H), 1.67-1.53 (m, 4H), 1.40 (s, 3H), 1.36-1.20 (m, 2H); MS m/z [M+1]=458.0

### Intermediate I-31: ((3aS,4R,6S,6aS)-6-(4-aminopyrrolo[2,1-f][1,2,4]triazin-7-yl)-4-cyano-2,2-dimethyltetrahydrofuro[3,4-d][1,3]dioxol-4-yl)methyl (2-ethylbutyl) carbonate

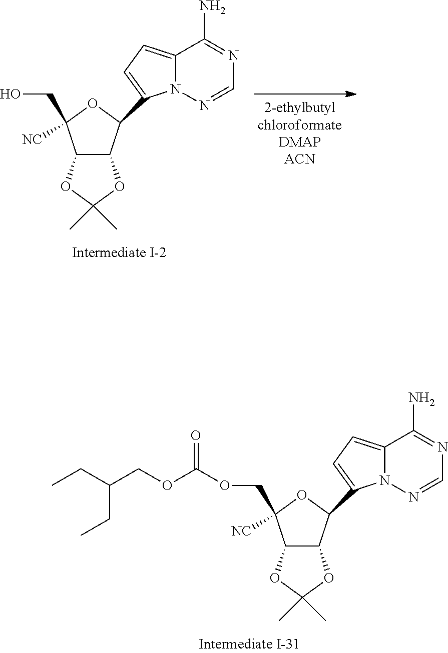 [SMILES: CC1(C)O[C@H]2[C@H](c3ccc4c(N)ncnn34)O[C@](C#N)(CO)[C@H]2O1 ; CCC(CC)COC(=O)OC[C@@]1(C#N)O[C@@H](c2ccc3c(N)ncnn23)[C@@H]2OC(C)(C)O[C@@H]21]

To a mixture of Intermediate I-2 (100 mg, 0.302 mmol) and 2-ethylbutyl chloroformate (0.058 mL, 0.362 mmol) in ACN (2 mL) was added DMAP (74 mg, 0.604 mmol). The resulting mixture was stirred at rt for 1h, MeOH added, concentrated in vacuo, and purified by silica gel column chromatography (0 to 5% MeOH in DCM) to give Intermediate I-31. ¹H NMR (400 MHz, Acetonitrile-d3) δ 7.91 (s, 1H), 6.80 (d, J=4.5 Hz, 1H), 6.77 (d, J=4.4 Hz, 1H), 6.32 (s, 2H), 5.68 (d, J=3.3 Hz, 1H), 5.32 (dd, J=6.7, 3.3 Hz, 1H), 5.10 (d, J=6.6 Hz, 1H), 4.51 (d, J=11.4 Hz, 1H), 4.43 (d, J=11.4 Hz, 1H), 4.10 (d, J=5.8 Hz, 2H), 1.72 (s, 3H), 1.56 (p, J=6.2 Hz, 1H), 1.43-1.30 (m, 7H), 0.91 (t, J=7.5 Hz, 6H); MS m/z [M+1]=460.0

### Intermediate I-32: ((3aS,4R,6S,6aS)-6-(4-aminopyrrolo[2,1-f][1,2,4]triazin-7-yl)-4-cyano-2,2-dimethyltetrahydrofuro[3,4-d][1,3]dioxol-4-yl)methyl (cyclopropylmethyl) carbonate

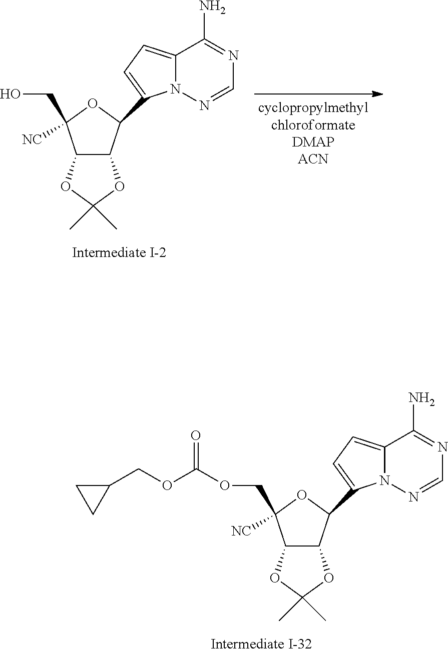 [SMILES: CC1(C)O[C@H]2[C@H](c3ccc4c(N)ncnn34)O[C@](C#N)(CO)[C@H]2O1 ; CC1(C)O[C@H]2[C@H](c3ccc4c(N)ncnn34)O[C@](C#N)(COC(=O)OCC3CC3)[C@H]2O1]

To a mixture of Intermediate I-2 (100 mg, 0.302 mmol) and cyclopropylmethyl chloroformate (48.7 mg, 0.362 mmol) in ACN (2 mL) was added DMAP (74 mg, 0.604 mmol). The resulting mixture was stirred at rt for 1h, MeOH added, concentrated in vacuo, and purified by silica gel column chromatography (0 to 5% MeOH in DCM) to give Intermediate I-32. ¹H NMR (400 MHz, Acetonitrile-d3) δ 7.92 (s, 1H), 6.80 (d, J=4.5 Hz, 1H), 6.77 (d, J=4.5 Hz, 1H), 6.29 (s, 2H), 5.68 (d, J=3.4 Hz, 1H), 5.32 (dd, J=6.6, 3.4 Hz, 1H), 5.11 (d, J=6.6 Hz, 1H), 4.49 (d, J=11.3 Hz, 1H), 4.44 (d, J=11.3 Hz, 1H), 3.98 (d, J=7.4 Hz, 2H), 1.72 (s, 3H), 1.40 (s, 3H), 1.22-1.04 (m, 1H), 0.63-0.54 (m, 2H), 0.38-0.26 (m, 2H); MS m/z [M+1]=430.0

### Intermediate I-33A: (S)-tetrahydrofuran-3-yl carbonochloridate

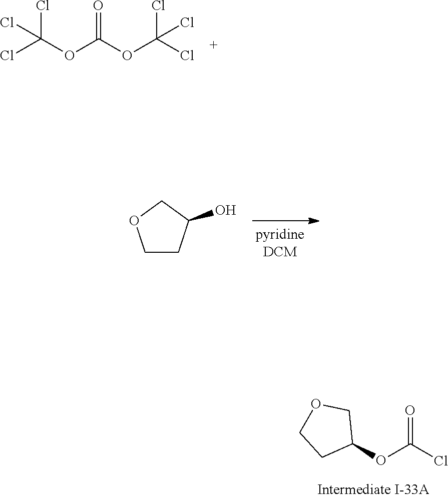 [SMILES: O=C(OC(Cl)(Cl)Cl)OC(Cl)(Cl)Cl ; O[C@H]1CCOC1 ; O=C(Cl)O[C@H]1CCOC1]

To a solution of (3 S)-tetrahydrofuran-3-ol (0.67 mL, 8.4 mmol) and pyridine (0.68 mL, 8.4 mmol) in DCM (5.0 mL) was added triphosgene (1000 mg, 3.4 mmol) at 0° C. portion wise (gas generated). The resulting mixture was stirred at rt for 15h, hexanes (50 mL) added, filtered, and concentrated in vacuo at 17° C. The resulting residue was treated with hexanes (20 mL), filtered, and concentrated in vacuo at 17° C. to give Intermediate I-33A which was used in next reaction without further purification (ca 500 mg). ¹H NMR (400 MHz, Chloroform-d) δ 5.45-5.39 (m, 1H), 4.04-3.88 (m, 4H), 2.35-2.16 (m, 2H).

### Intermediate I-33: ((3aS,4R,6S,6aS)-6-(4-aminopyrrolo[2,1-f][1,2,4]triazin-7-yl)-4-cyano-2,2-dimethyltetrahydrofuro[3,4-d][1,3]dioxol-4-yl)methyl ((S)-tetrahydrofuran-3-yl) carbonate

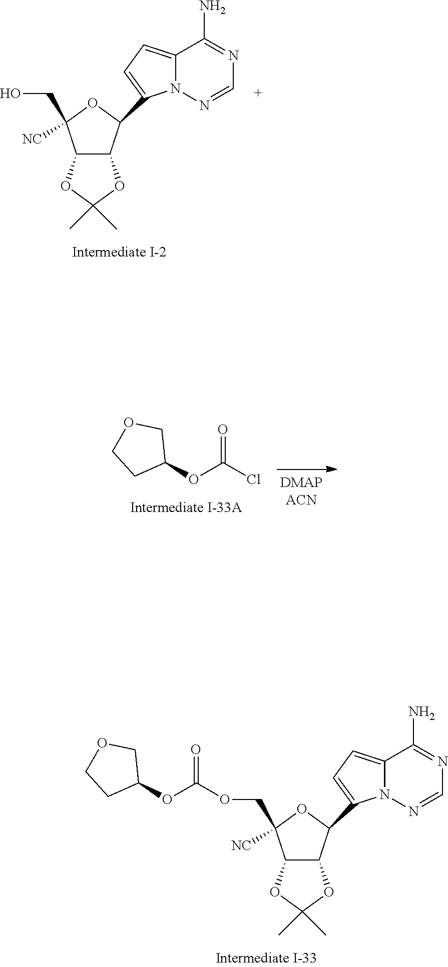 [SMILES: CC1(C)O[C@H]2[C@H](c3ccc4c(N)ncnn34)O[C@](C#N)(CO)[C@H]2O1 ; O=C(Cl)O[C@H]1CCOC1 ; CC1(C)O[C@H]2[C@H](c3ccc4c(N)ncnn34)O[C@](C#N)(COC(=O)O[C@H]3CCOC3)[C@H]2O1]

To a mixture of Intermediate I-2 (200 mg, 0.604 mmol) and DMAP (147 mg, 1.21 mmol) in ACN (4 mL) was added Intermediate I-33A (109 mg, 0.724 mmol). The resulting mixture was stirred at rt for 1h, MeOH added, concentrated in vacuo, and purified by silica gel column chromatography (0 to 5% MeOH in DCM) to give Intermediate I-33. ¹H NMR (400 MHz, Acetonitrile-d3) δ 7.91 (s, 1H), 6.81 (d, J=4.5 Hz, 1H), 6.77 (d, J=4.5 Hz, 1H), 6.27 (s, 2H), 5.68 (d, J=3.3 Hz, 1H), 5.33 (dd, J=6.6, 3.4 Hz, 1H), 5.22-5.14 (m, 1H), 5.11 (d, J=6.7 Hz, 1H), 4.50 (d, J=11.3 Hz, 1H), 4.46 (d, J=11.3 Hz, 1H), 3.99-3.63 (m, 4H), 2.124-1.93 (m, 2H), 1.72 (s, 3H), 1.42-1.35 (m, 3H); MS m/z [M+1]=445.9

### Intermediate I-34: ((3aS,4R,6S,6aS)-6-(4-aminopyrrolo[2,1-f][1,2,4]triazin-7-yl)-4-cyano-2,2-dimethyltetrahydrofuro[3,4-d][1,3]dioxol-4-yl)methyl (2-propoxyethyl) carbonate

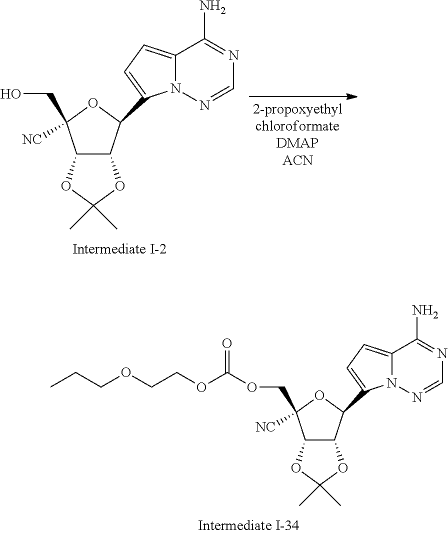 [SMILES: CC1(C)O[C@H]2[C@H](c3ccc4c(N)ncnn34)O[C@](C#N)(CO)[C@H]2O1 ; CCCOCCOC(=O)OC[C@@]1(C#N)O[C@@H](c2ccc3c(N)ncnn23)[C@@H]2OC(C)(C)O[C@@H]21]

To a mixture of Intermediate I-2 (100 mg, 0.604 mmol) and DMAP (74 mg, 0.604 mmol) in ACN (2 mL) was added 2-propoxyethyl chloroformate (60.3 mg, 0.362 mmol). The resulting mixture was stirred at rt for 1h, MeOH added, concentrated in vacuo, and purified by silica gel column chromatography (0 to 5% MeOH in DCM) to give Intermediate I-34. ¹H NMR (400 MHz, Acetonitrile-d3) δ 7.92 (s, 1H), 6.80 (d, J=4.5 Hz, 1H), 6.77 (dd, J=4.5, 0.8 Hz, 1H), 6.37 (s, 2H), 5.68 (d, J=3.3 Hz, 1H), 5.35-5.25 (m, 1H), 5.11 (d, J=6.6 Hz, 1H), 4.50 (d, J=11.3 Hz, 1H), 4.46 (d, J=11.6 Hz, 1H), 4.29-4.23 (m, 2H), 3.68-3.60 (m, 2H), 3.44-3.37 (m, 2H), 1.72 (s, 3H), 1.62-1.51 (m, 2H), 1.39 (s, 3H), 0.90 (t, J=7.8 Hz, 3H). MS m/z [M+1]=462.0

### Intermediate I-35A: 3,3-dimethylbutyl carbonochloridate

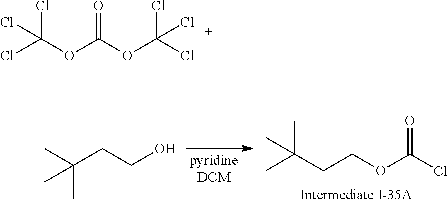

To a solution of 3,3-dimethylbutan-1-ol (1.2 mL, 4.0 mmol) and pyridine (0.81 mL, 10 mmol) in DCM (10 mL) was added triphosgene (1200 mg, 4.0 mmol) at 0° C. portion wise. The resulting mixture was stirred at rt for 18h, hexanes (50 mL) added, filtered, and concentrated in vacuo at 17° C. The resulting residue was treated with hexanes (20 mL), filtered, and concentrated in vacuo at 17° C. to give Intermediate I-35A (995 mg). ¹H NMR (400 MHz, Chloroform-d) δ 4.40 (t, J=7.5 Hz, 2H), 1.69 (t, J=7.5 Hz, 2H), 0.98 (s, 9H).

### Intermediate I-35: ((3aS,4R,6S,6aS)-6-(4-aminopyrrolo[2,1-f][1,2,4]triazin-7-yl)-4-cyano-2,2-dimethyltetrahydrofuro[3,4-d][1,3]dioxol-4-yl)methyl (3,3-dimethylbutyl) carbonate

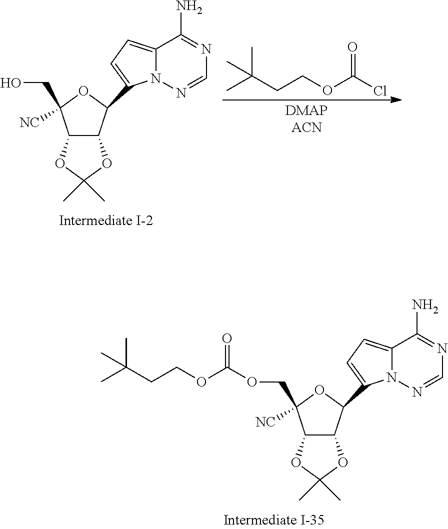

To a mixture of Intermediate I-2 (100 mg, 0.604 mmol) and DMAP (147 mg, 1.21 mmol) in ACN (4 mL) was added BQ6784-1 (119 mg, 0.724 mmol). The resulting mixture was stirred at rt for 1h, MeOH added, concentrated in vacuo, and purified by silica gel column chromatography (0 to 5% MeOH in DCM) to give Intermediate I-35. ¹H NMR (400 MHz, Acetonitrile-d3) δ 7.91 (s, 1H), 6.80 (d, J=4.5 Hz, 1H), 6.77 (d, J=4.5 Hz, 1H), 6.31 (s, 2H), 5.67 (d, J=3.3 Hz, 1H), 5.32 (dd, J=6.6, 3.4 Hz, 1H), 5.10 (d, J=6.6 Hz, 1H), 4.50 (d, J=11.4 Hz, 1H), 4.43 (d, J=11.4 Hz, 1H), 4.21 (t, J=7.5 Hz, 2H), 1.72 (s, 3H), 1.60 (t, J=7.5 Hz, 2H), 1.39 (s, 3H), 0.95 (s, 9H). MS m/z [M+1]=460.0

### Intermediate I-36: ((3aS,4R,6S,6aS)-6-(4-aminopyrrolo[2,1-f][1,2,4]triazin-7-yl)-4-cyano-2,2-dimethyltetrahydrofuro[3,4-d][1,3]dioxol-4-yl)methyl spiro[3.3]heptan-2-yl carbonate

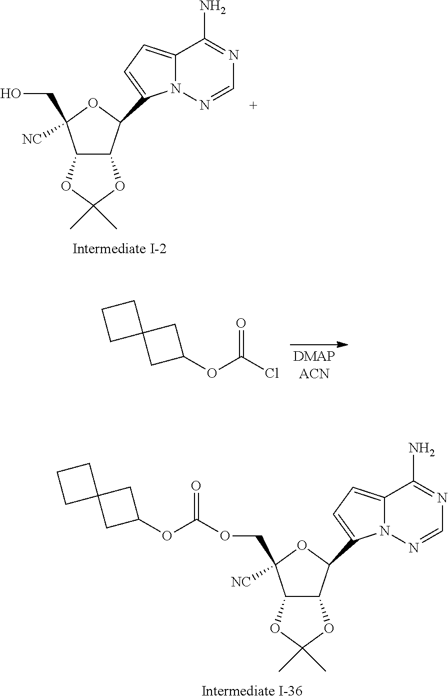

To a mixture of Intermediate I-2 (100 mg, 0.604 mmol) and DMAP (147 mg, 1.21 mmol) in ACN (4 mL) was added spiro[3.3]heptan-2-yl carbonochloridate (126 mg, 0.724 mmol). The resulting mixture was stirred at rt for 1h, MeOH added, concentrated in vacuo, and purified by silica gel column chromatography (0 to 5% MeOH in DCM) to give Intermediate I-36. ¹H NMR (400 MHz, Acetonitrile-d3) δ 7.91 (s, 1H), 6.80 (d, J=4.5 Hz, 1H), 6.77 (d, J=4.5 Hz, 1H), 6.31 (s, 2H), 5.67 (d, J=3.3 Hz, 1H), 5.32 (dd, J=6.6, 3.3 Hz, 1H), 5.09 (d, J=6.6 Hz, 1H), 4.78 (p, J=7.2 Hz, 1H), 4.47 (d, J=11.4 Hz, 1H), 4.40 (d, J=11.3 Hz, 1H), 2.57-2.30 (m, 2H), 2.10-1.94 (m, 6H), 1.89-1.79 (m, 2H), 1.72 (s, 3H), 1.39 (s, 3H). MS m/z [M+1]=469.9

### Intermediate I-37A: 3,3-dimethylcyclobutyl carbonochloridate

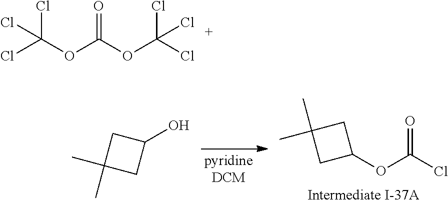

To a solution of 3,3-dimethylcyclobutan-1-ol (0.67 mL, 8.40 mmol) and pyridine (0.68 mL, 8.40 mmol) in DCM (10 mL) was added triphosgene (1000 mg, 3.40 mmol) at 0° C. portion wise. The resulting mixture was stirred at rt for 15h, hexanes (50 mL) added, filtered, concentrated in vacuo at 17° C. The resulting residue was treated with hexanes (20 mL), filtered, and concentrated in vacuo at 17° C. to give Intermediate I-37A (645 mg). ¹H NMR (400 MHz, Chloroform-d) δ 5.14 (p, J=7.2 Hz, 1H), 2.35-2.28 (m, 2H), 2.09-1.99 (m, 2H), 1.21 (s, 3H), 1.16 (s, 3H).

### Intermediate 37: ((3aS,4R,6S,6aS)-6-(4-aminopyrrolo[2,1-f][1,2,4]triazin-7-yl)-4-cyano-2,2-dimethyltetrahydrofuro[3,4-d][1,3]dioxol-4-yl)methyl (3,3-dimethylcyclobutyl) carbonate

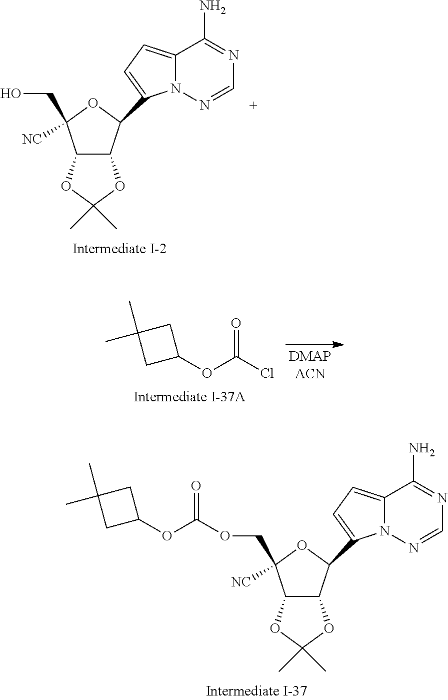

To a mixture of Intermediate I-2 (150 mg, 0.453 mmol) and DMAP (111 mg, 0.905 mmol) in ACN (3 mL) was added Intermediate I-37A (88.3 mg, 0.543 mmol). The resulting mixture was stirred at rt for 1h, MeOH added, concentrated in vacuo, and purified by silica gel column chromatography (0 to 5% MeOH in DCM) to give Intermediate I-37. ¹H NMR (400 MHz, Acetonitrile-d3) δ 7.91 (s, 1H), 6.80 (d, J=4.5 Hz, 1H), 6.77 (d, J=4.5 Hz, 1H), 6.29 (s, 2H), 5.67 (d, J=3.4 Hz, 1H), 5.32 (dd, J=6.6, 3.4 Hz, 1H), 5.09 (d, J=6.6 Hz, 1H), 4.93 (p, J=7.2 Hz, 1H), 4.48 (d, J=11.3 Hz, 1H), 4.41 (d, J=11.3 Hz, 1H), 2.30-2.17 (m, 2H), 1.96-1.84 (m, 2H), 1.72 (s, 3H), 1.39 (s, 3H), 1.17 (s, 3H), 1.14 (s, 3H). MS m/z [M+1]=458.0

### Intermediate 38A: (R)-1-methoxypropan-2-yl carbonochloridate

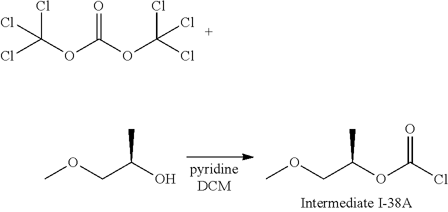

To a solution of (2R)-1-methoxypropan-2-ol (0.84 mL, 8.4 mmol) and pyridine (0.68 mL, 8.4 mmol) in DCM (10 mL) was added triphosgene (1000 mg, 3.4 mmol) at 0° C. portion wise (gas generated). The resulting mixture was stirred at rt for 15h, hexanes (50 mL) added, filtered, and concentrated in vacuo at 17° C. The resulting residue was treated with hexanes (20 mL), re-filtered, and concentrated in vacuo at 17° C. to give Intermediate I-38A. ¹H NMR (400 MHz, Chloroform-d) δ 5.15 (td, J=6.5, 3.9 Hz, 1H), 3.59-3.46 (m, 2H), 3.41 (s, 3H), 1.38 (d, J=6.5 Hz, 3H).

### Intermediate 38: ((3aS,4R,6S,6aS)-6-(4-aminopyrrolo[2,1-f][1,2,4]triazin-7-yl)-4-cyano-2,2-dimethyltetrahydrofuro[3,4-d][1,3]dioxol-4-yl)methyl ((R)-1-methoxypropan-2-yl) carbonate

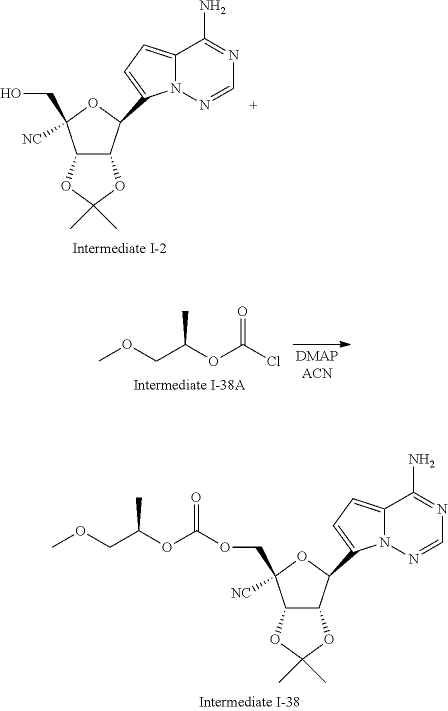

To a mixture of Intermediate I-2 (200 mg, 0.604 mmol) and DMAP (147 mg, 1.21 mmol) in ACN (4 mL) was added Intermediate I-38A (111.0 mg, 0.724 mmol). The resulting mixture was stirred at rt for 1 h, MeOH added, concentrated in vacuo, and purified by silica gel column chromatography (0 to 5% MeOH in DCM) to give Intermediate I-38. ¹H NMR (400 MHz, Acetonitrile-d3) δ 7.92 (s, 1H), 6.81 (d, J=4.5 Hz, 1H), 6.77 (d, J=4.5 Hz, 1H), 6.28 (s, 2H), 5.67 (d, J=3.4 Hz, 1H), 5.32 (dd, J=6.6, 3.4 Hz, 1H), 5.10 (d, J=6.6 Hz, 1H), 4.94-4.81 (m, 1H), 4.49 (d, J=11.3 Hz, 1H), 4.44 (d, J=11.3 Hz, 1H), 3.50-3.37 (m, 2H), 3.31 (s, 3H), 1.72 (s, 3H), 1.40 (s, 3H), 1.25 (d, J=6.5 Hz, 3H). MS m/z [M+1]=448.0

### Intermediate I-39A: (1R,3r,5S)-bicyclo[3.1.0]hexan-3-yl carbonochloridate

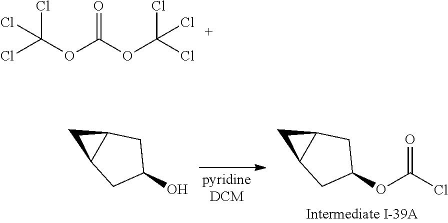

To a solution of (1S,5R)-bicyclo[3.1.0]hexan-3-ol (0.67 mL, 8.4 mmol) and pyridine (0.81 mL, 10.00 mmol) in DCM (10 mL) was added triphosgene (1200 mg, 4.0 mmol) at 0° C. portion wise (gas generated). The resulting mixture was stirred at rt for 15h, hexanes (50 mL) added, filtered, and concentrated in vacuo at 17° C. The resulting residue was treated with hexanes (20 mL), re-filtered, and concentrated in vacuo at 17° C. to give Intermediate I-39A. ¹H NMR (400 MHz, Chloroform-d) δ 5.32 (t, J=6.7 Hz, 1H), 2.31-2.21 (m, 2H), 2.07 (s, 1H), 2.03 (s, 1H), 1.43-1.34 (m, 2H), 0.68-0.53 (m, 1H), 0.44-0.37 (m, 1H).

### Intermediate 39: ((3aS,4R,6S,6aS)-6-(4-aminopyrrolo[2,1-f][1,2,4]triazin-7-yl)-4-cyano-2,2-dimethyltetrahydrofuro[3,4-d][1,3]dioxol-4-yl)methyl ((1R,3r,5S)-bicyclo[3.1.0]hexan-3-yl) carbonate

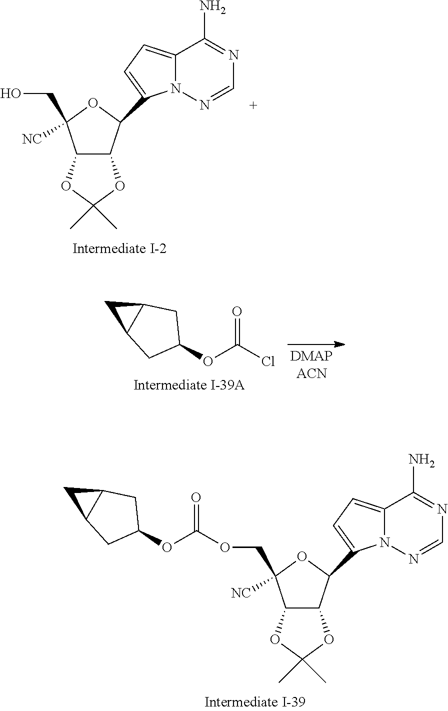

To a mixture of Intermediate I-2 (200 mg, 0.604 mmol) and DMAP (147 mg, 1.21 mmol) in ACN (4 mL) was added Intermediate I-39A (116.0 mg, 0.724 mmol). The resulting mixture was stirred at rt for 1 h, MeOH added, concentrated in vacuo, and purified by silica gel column chromatography (0 to 5% MeOH in DCM) to give Intermediate I-39. ¹H NMR (400 MHz, Acetonitrile-d3) δ 7.91 (s, 1H), 6.80 (d, J=4.5 Hz, 1H), 6.77 (d, J=4.5 Hz, 1H), 6.28 (s, 2H), 5.67 (d, J=3.4 Hz, 1H), 5.31 (dd, J=6.6, 3.4 Hz, 1H), 5.09 (t, J=7.1 Hz, 2H), 4.47 (d, J=11.4 Hz, 1H), 4.38 (d, J=11.4 Hz, 1H), 2.29-2.16 (m, 2H), 1.93-1.81 (m, 2H), 1.72 (s, 3H), 1.42-1.28 (m, 5H), 0.57-0.44 (m, 1H), 0.29 (q, J=4.1 Hz, 1H). MS m/z [M+1]=456.0

### Intermediate I-40A: (1r,4r)-4-methylcyclohexyl carbonochloridate

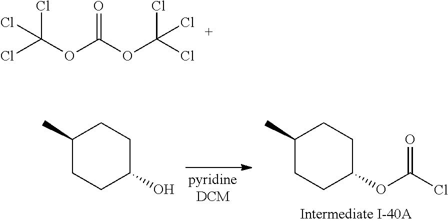

To a solution of trans-4-methylcyclohexanol (1.3 mL, 10 mmol) and pyridine (0.91 mL, 11 mmol) in ACN (10 mL) was added triphosgene (1200 mg, 4.0 mmol) at 0° C. portion wise (gas generated). The resulting mixture was stirred at rt for 2 h and concentrated in vacuo at 21° C. The resulting residue was treated with hexanes (50 mL)-DCM (5 mL), filtered, and concentrated in vacuo at 21° C. The resulting residue was treated with hexanes (5 mL), re-filtered, and concentrated in vacuo at 21° C. to give Intermediate I-40A. ¹H NMR (400 MHz, Chloroform-d) δ 4.82-4.70 (m, 1H), 2.17-1.97 (m, 2H), 1.88-1.70 (m, 2H), 1.65-1.27 (m, 3H), 1.17-0.97 (m, 2H), 0.92 (d, J=6.5 Hz, 3H).

### Intermediate I-40: ((3aS,4R,6S,6aS)-6-(4-aminopyrrolo[2,1-f][1,2,4]triazin-7-yl)-4-cyano-2,2-dimethyltetrahydrofuro[3,4-d][1,3]dioxol-4-yl)methyl ((1r,4R)-4-methylcyclohexyl) carbonate

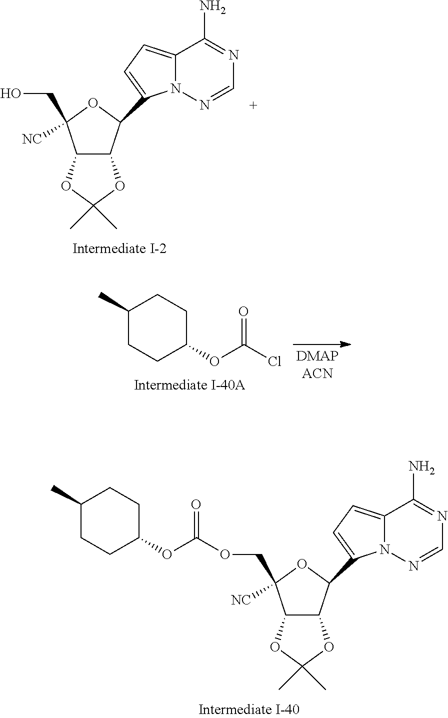

To a mixture of Intermediate I-2 (100 mg, 0.302 mmol) and DMAP (74 mg, 0.604 mmol) in ACN (2 mL) was added Intermediate I-40A (64.0 mg, 0.362 mmol). The resulting mixture was stirred at rt for 1 h, MeOH added, concentrated in vacuo, and purified by silica gel column chromatography (0 to 5% MeOH in DCM) to give Intermediate I-40. ¹H NMR (400 MHz, Acetonitrile-d3) δ 7.91 (s, 1H), 6.80 (d, J=4.5 Hz, 1H), 6.77 (d, J=4.5 Hz, 1H), 6.27 (s, 2H), 5.67 (d, J=3.4 Hz, 1H), 5.32 (dd, J=6.6, 3.4 Hz, 1H), 5.09 (d, J=6.6 Hz, 1H), 4.54-4.35 (m, 3H), 2.06-1.85 (m, 2H), 1.80-1.67 (m, 5H), 1.47-1.27 (m, 6H), 1.15-0.96 (m, 2H), 0.91 (d, J=6.6 Hz, 3H). MS m/z [M+1]=472.0

### Intermediate I-42B: ((2R,3S,4R,5S)-2-cyano-5-(4-(((Z)-(dimethylamino)methylene)amino)pyrrolo[2,1-f][1,2,4]triazin-7-yl)-3,4-dihydroxytetrahydrofuran-2-yl)methyl isopropyl carbonate

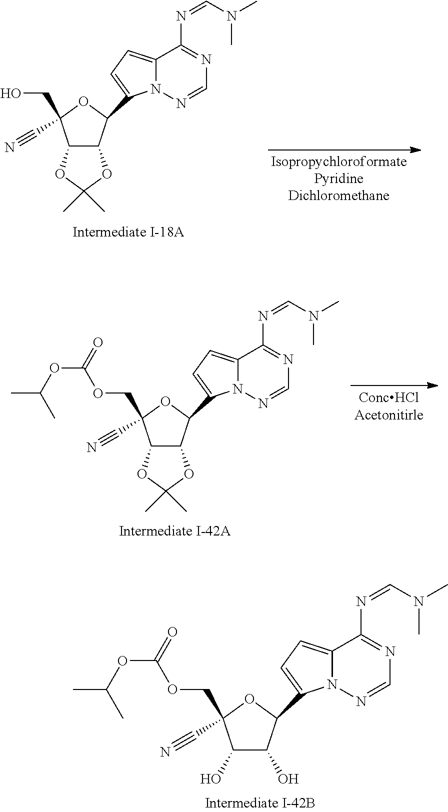

To a solution of Intermediate I-18A (500 mg, 1.3 mmol) in dichloromethane (15 mL) isopropyl chloroformate (1.3 mL, 2.6 mmol) was added followed by pyridine (0.2 mL, 2 mmol) and stirred at room temperature for 1h. After completion of the rection, diluted the reaction mixture with dichloromethane, washed with water, brine, dried and concentrated. The residue was purified by flash chromatography using silica gel column chromatography (0 to 10% MeOH in DCM) to provide Intermediate I-42A. MS m/z [M+1]=473.2

To a solution of Intermediate I-42A (600 mg, 1.3 mmol) in acetonitrile (10 mL), Conc. HCl (0.6 mL, 7.5 mmol) was added and stirred at room temperature for 30 min. After completion of the reaction, diluted with ethyl acetate (50 mL), neutralized with aqueous sodium bicarbonate, separated the organic layer, washed with water, brine, dried and concentrated. The residue is purified by flash chromatography using 0-20% MeOH in DCM as eluent to afford Intermediate I-42B. MS m/z [M+1]=433.2

### Intermediate I-42: (2R,3S,4S,5S)-2-cyano-5-(4-(((Z)-(dimethylamino)methylene)amino)pyrrolo[2,1-f][1,2,4]triazin-7-yl)-2-(((isopropoxycarbonyl)oxy)methyl)tetrahydrofuran-3,4-diyl diisopropyl bis(carbonate)

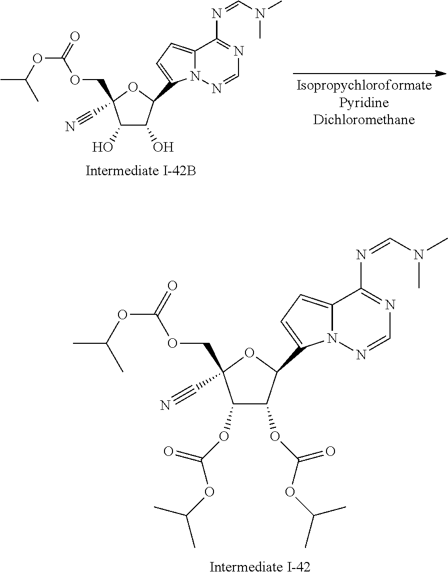

To a solution of Intermediate I-42B (200 mg, 0.32 mmol) in dichloromethane (15 mL) isopropyl chloroformate (0.8 mL, 1.6 mmol) was added followed by pyridine (0.1 mL, 1 mmol) and stirred at room temperature for 1h. After completion of the reaction, diluted the reaction mixture with dichloromethane, washed with water, brine, dried and concentrated. The residue was purified by flash chromatography using silica gel column chromatography (0 to 10% MeOH in DCM) to provide Intermediate I-42. MS m/z [M+1]=605.2

### Intermediate I-43B: ((2R,3S,4R,5S)-2-cyano-5-(4-(((Z)-(dimethylamino)methylene)amino)pyrrolo[2,1-f][1,2,4]triazin-7-yl)-3,4-dihydroxytetrahydrofuran-2-yl)methyl methyl carbonate

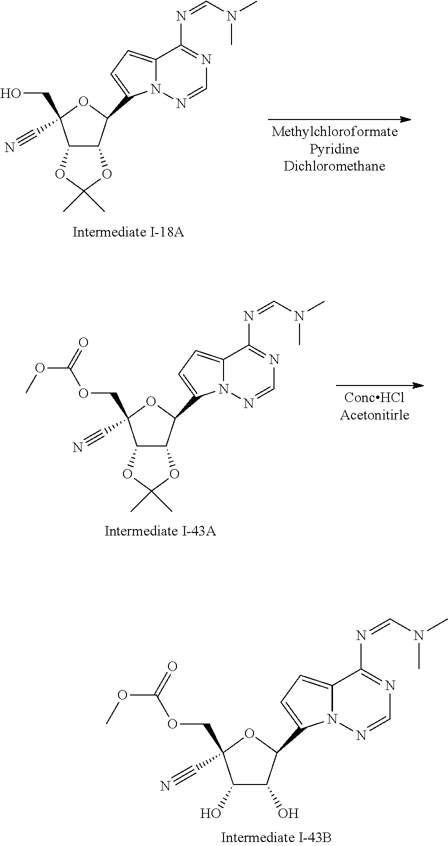

To a solution of Intermediate I-18A (400 mg, 1 mmol) in dichloromethane (15 mL) methyl chloroformate (1 mL, 2 mmol) was added followed by pyridine (0.2 mL, 2 mmol) and stirred at room temperature for 1h. After completion of the reaction, diluted the reaction mixture with dichloromethane, washed with water, brine, dried and concentrated. The residue was purified by flash chromatography using silica gel column chromatography (0 to 10% MeOH in DCM) to provide Intermediate I-43A. MS m/z [M+1]=445.1

To a solution of Intermediate I-43A (300 mg, 0.7 mmol) in acetonitrile (10 mL), Conc. HCl (0.6 mL, 7.5 mmol) was added and stirred at room temperature for 30 min. After completion of the reaction, diluted with ethyl acetate (50 mL), neutralized with aqueous sodium bicarbonate, separated the organic layer, washed with water, brine, dried and concentrated. The residue is purified by flash chromatography using 0-20% MeOH in DCM as eluent to afford Intermediate I-43B. MS m/z [M+1]=405.1

### Intermediate I-43: (2R,3S,4S,5S)-2-cyano-5-(4-(((Z)-(dimethylamino)methylene)amino)pyrrolo[2,1-f][1,2,4]triazin-7-yl)-2-(((methoxycarbonyl)oxy)methyl)tetrahydrofuran-3,4-diyl dimethyl bis(carbonate)

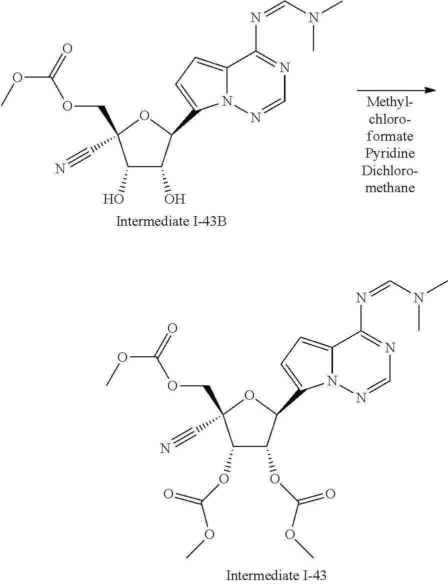

To a solution of Intermediate I-43B (130 mg, 0.32 mmol) in dichloromethane (15 mL) methyl chloroformate (1 mL, 2 mmol) was added followed by pyridine (0.2 mL, 2 mmol) and stirred at room temperature for 1h. After completion of the reaction, diluted the reaction mixture with dichloromethane, washed with water, brine, dried and concentrated. The residue was purified by flash chromatography using silica gel column chromatography (0 to 10% MeOH in DCM) to provide Intermediate I-43. MS m/z [M+1]=521.2

### C. Compounds

### Example 0. Compound 0 (2R,3S,4R,5S)-5-(4-aminopyrrolo[1,2-f][1,2,4]triazin-7-yl)-3,4-dihydroxy-2-(hydroxymethyl)tetrahydrofuran-2-carbonitrile

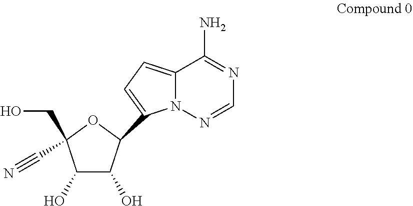 [SMILES: N#C[C@]1(CO)O[C@@H](c2ccc3c(N)ncnn23)[C@H](O)[C@@H]1O]

Compound 0 was prepared according to WO2015/069939. For example, pages 43-55 of WO2015/069939 provide a process for preparing this compound (identified as compound 1 in WO2015/069939).

### Example 1: Compound 1 ((2R,3S,4R,5S)-5-(4-aminopyrrolo[2,1-f][1,2,4]triazin-7-yl)-2-cyano-3,4-dihydroxytetrahydrofuran-2-yl)methyl cyclopentyl carbonate

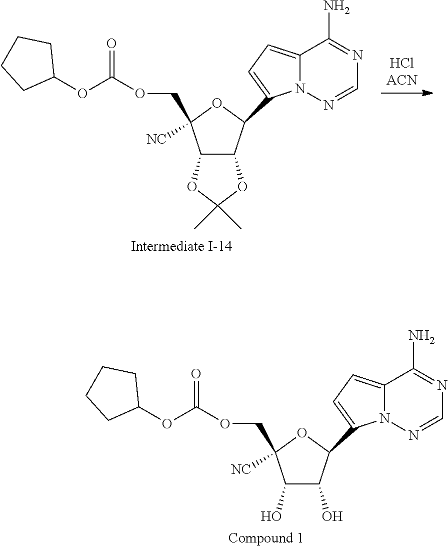 [SMILES: CC1(C)O[C@H]2[C@H](c3ccc4c(N)ncnn34)O[C@](C#N)(COC(=O)OC3CCCC3)[C@H]2O1 ; N#C[C@]1(COC(=O)OC2CCCC2)O[C@@H](c2ccc3c(N)ncnn23)[C@H](O)[C@@H]1O]

To a solution of Intermediate I-14 (0.293 mmol) in ACN (1 mL) was added 25% HCl (0.2 mL) at rt. The mixture was stirred for 2 h, diluted with EtOAc (20 mL), and aqNaHCO₃ (10 mL) added slowly. The mixture was stirred for 10 min, the phases separated, and the aqueous phase extracted with EtOAc (10 mL×3). The combined organic phase was dried under sodium sulfate, concentrated in vacuo, and purified by silica gel column chromatography (0 to 5% MeOH in DCM) to give Compound 1. ¹H NMR (400 MHz, Methanol-d4) δ 7.82 (s, 1H), 6.87 (d, J=4.5 Hz, 1H), 6.74 (d, J=4.5 Hz, 1H), 5.53 (d, J=4.7 Hz, 1H), 5.09-5.00 (m, 1H), 4.64-4.56 (m, 2H), 4.49 (d, J=5.5 Hz, 1H), 4.41 (d, J=11.7 Hz, 1H), 1.94-1.84 (m, 2H), 1.80-1.69 (m, 4H), 1.62 (m, 2H). MS m/z [M+1]=404.0.

### Example 2: Compound 2 ((2R,3S,4R,5S)-5-(4-aminopyrrolo[2,1-f][1,2,4]triazin-7-yl)-2-cyano-3,4-dihydroxytetrahydrofuran-2-yl)methyl neopentyl carbonate

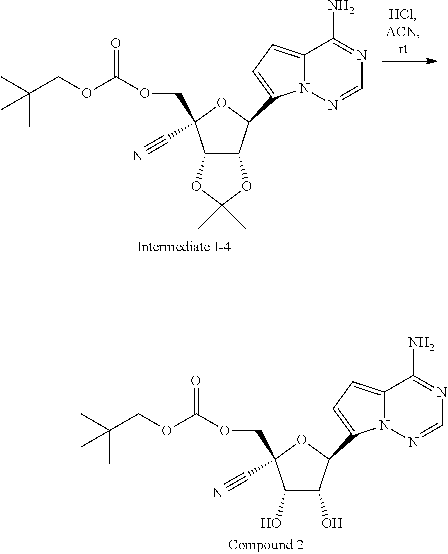 [SMILES: CC(C)(C)COC(=O)OC[C@@]1(C#N)O[C@@H](c2ccc3c(N)ncnn23)[C@@H]2OC(C)(C)O[C@@H]21 ; CC(C)(C)COC(=O)OC[C@@]1(C#N)O[C@@H](c2ccc3c(N)ncnn23)[C@H](O)[C@@H]1O]

To a solution of Intermediate I-4 (0.33 mmol) in ACN (5.0 mL) was added concentrated HCl (0.14 mL) at room temperature. The reaction mixture was stirred for 1 h and 45 min prior to quenching with a 1:1 solution of water:saturated NaHCO₃ (30 mL) and diluting with EtOAc (30 mL). The layers were separated and the aqueous layer was extracted once more with EtOAc (30 mL). The organic fractions were combined, washed with 1:1 water:brine (30 mL), dried over Na₂SO₄, filtered and concentrated in vacuo prior to purification by silica gel chromatography (0-20% MeOH in DCM) to afford Compound 2. ¹H NMR (400 MHz, DMSO-d₆) δ 7.91-7.64 (m, 3H), 6.86 (d, J=4.5 Hz, 1H), 6.72 (d, J=4.5 Hz, 1H), 6.15 (d, J=5.9 Hz, 1H), 5.52 (d, J=5.5 Hz, 1H), 5.40 (d, J=5.5 Hz, 1H), 4.54 (d, J=11.6 Hz, 1H), 4.49-4.42 (m, 1H), 4.37-4.25 (m, 2H), 3.86-3.78 (m, 2H), 0.90 (s, 9H). MS m/z [M+1]=406.1.

### Example 3: Compound 3 ((2R,3S,4R,5S)-5-(4-aminopyrrolo[2,1-f][1,2,4]triazin-7-yl)-2-cyano-3,4-dihydroxytetrahydrofuran-2-yl)methyl (1-methylcyclopropyl) carbonate

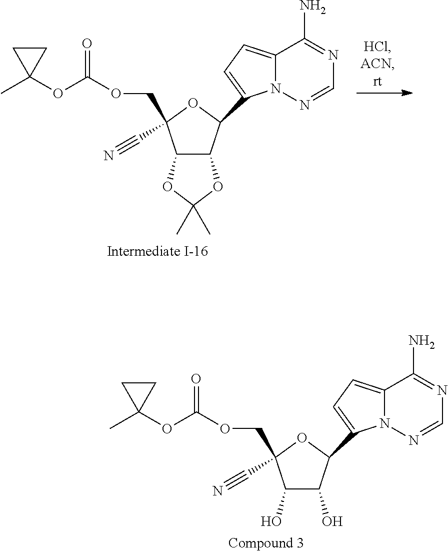 [SMILES: CC1(OC(=O)OC[C@@]2(C#N)O[C@@H](c3ccc4c(N)ncnn34)[C@@H]3OC(C)(C)O[C@@H]32)CC1 ; CC1(OC(=O)OC[C@@]2(C#N)O[C@@H](c3ccc4c(N)ncnn34)[C@H](O)[C@@H]2O)CC1]

To a solution of Intermediate I-16 (0.19 mmol) in CH₃CN (4.0 mL) was added concentrated HCl (0.12 mL) at room temperature drop wise. The reaction mixture was stirred for 60 min prior to quenching with a 1:1 solution of water:saturated NaHCO₃ (30 mL) and diluting with EtOAc (30 mL). The layers were separated and the aqueous layer was extracted once more with EtOAc (30 mL). The organic fractions were combined, washed with 1:1 water:brine (30 mL), dried over Na₂SO₄, filtered and concentrated in vacuo prior to purification by silica gel column chromatography (0-15% MeOH in DCM) to afford Compound 3. ¹H NMR (400 MHz, Acetonitrile-d₃) δ 7.90 (s, 1H), 6.77 (d, J=4.5 Hz, 1H), 6.74 (d, J=4.5 Hz, 1H), 6.25 (s, 2H), 5.47 (d, J=4.9 Hz, 1H), 4.59 (d, J=7.1 Hz, 1H), 4.54 (d, J=11.7 Hz, 1H), 4.47 (d, J=4.4 Hz, 1H), 4.36 (d, J=11.7 Hz, 1H), 4.24-4.16 (m, 2H), 3.99 (s, 1H), 1.52 (s, 3H), 0.95-0.84 (m, 2H), 0.71-0.60 (m, 2H). MS m/z [M+1]=390.0.

### Example 4: Compound 4 ((2R,3S,4R,5S)-5-(4-aminopyrrolo[2,1-f][1,2,4]triazin-7-yl)-2-cyano-3,4-dihydroxytetrahydrofuran-2-yl)methyl ((S)-sec-butyl) carbonate

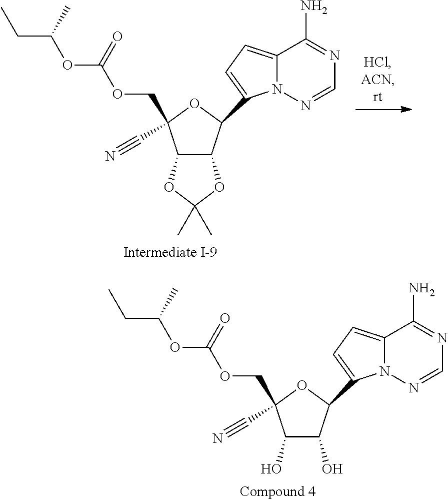 [SMILES: CC[C@H](C)OC(=O)OC[C@@]1(C#N)O[C@@H](c2ccc3c(N)ncnn23)[C@@H]2OC(C)(C)O[C@@H]21 ; CC[C@H](C)OC(=O)OC[C@@]1(C#N)O[C@@H](c2ccc3c(N)ncnn23)[C@H](O)[C@@H]1O]

To a solution of Intermediate I-9 (0.20 mmol) in ACN (5.0 mL) was added concentrated HCl (0.08 mL, 12 M) at room temperature. The reaction mixture was stirred for 1 h and 25 min prior to quenching with a 1:1 solution of water:saturated NaHCO₃ (30 mL) and diluting with EtOAc (30 mL). The layers were separated and the aqueous layer was extracted once more with EtOAc (30 mL). The organic fractions were combined, washed with 1:1 water:brine (40 mL), dried over MgSO₄, filtered and concentrated in vacuo prior to purification by silica gel chromatography (0-15% MeOH in DCM) to afford Compound 4. ¹H NMR (400 MHz, DMSO-d6) δ 7.89-7.67 (m, 3H), 6.86 (d, J=4.5 Hz, 1H), 6.72 (d, J=4.5 Hz, 1H), 6.14 (d, J=5.9 Hz, 1H), 5.50 (d, J=5.6 Hz, 1H), 5.39 (d, J=5.7 Hz, 1H), 4.67-4.56 (m, 1H), 4.55-4.43 (m, 2H), 4.35-4.24 (m, 2H), 1.64-1.46 (m, 2H), 1.19 (d, J=6.2 Hz, 3H), 0.88-0.82 (m, 3H). MS m/z [M+1]=392.1.

### Example 5: Compound 5 ((2R,3S,4R,5S)-5-(4-aminopyrrolo[2,1-f][1,2,4]triazin-7-yl)-2-cyano-3,4-dihydroxytetrahydrofuran-2-yl)methyl phenethyl carbonate

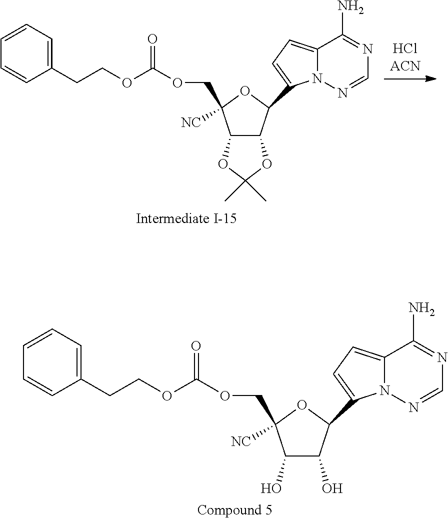 [SMILES: CC1(C)O[C@H]2[C@H](c3ccc4c(N)ncnn34)O[C@](C#N)(COC(=O)OCCc3ccccc3)[C@H]2O1 ; N#C[C@]1(COC(=O)OCCc2ccccc2)O[C@@H](c2ccc3c(N)ncnn23)[C@H](O)[C@@H]1O]

To a solution of Intermediate I-15 (0.257 mmol) in ACN (1 mL) was added 25% HCl (0.2 mL) at rt. The mixture was stirred for 2 h, diluted with EtOAc (20 mL), and aqNaHCO₃ (10 mL) added slowly. The mixture was stirred for 10 min, the phases separated, and the aqueous phase extracted with EtOAc (10 mL×3). The combined organic phase was dried under sodium sulfate, concentrated in vacuo, and purified by silica gel column chromatography (0 to 5% MeOH in DCM) to give Compound 5. ¹H NMR (400 MHz, Methanol-d4) δ 7.81 (s, 1H), 7.31-7.17 (m, 5H), 6.87 (d, J=4.5 Hz, 1H), 6.73 (d, J=4.5 Hz, 1H), 5.53 (d, J=4.8 Hz, 1H), 4.64-4.53 (m, 2H), 4.48 (d, J=5.5 Hz, 1H), 4.42 (d, J=11.6 Hz, 1H), 4.38-4.26 (m, 2H), 2.96 (t, J=7.0 Hz, 2H). MS m/z [M+1]=439.9.

### Example 6: Compound 6 ((2R,3S,4R,5S)-5-(4-aminopyrrolo[2,1-f][1,2,4]triazin-7-yl)-2-cyano-3,4-dihydroxytetrahydrofuran-2-yl)methyl methyl carbonate

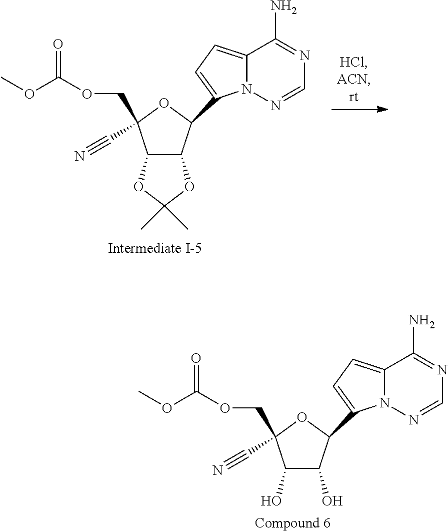 [SMILES: COC(=O)OC[C@@]1(C#N)O[C@@H](c2ccc3c(N)ncnn23)[C@@H]2OC(C)(C)O[C@@H]21 ; COC(=O)OC[C@@]1(C#N)O[C@@H](c2ccc3c(N)ncnn23)[C@H](O)[C@@H]1O]

To a solution of Intermediate I-5 (0.28 mmol) in ACN (5.0 mL) was added concentrated HCl (0.12 mL) at room temperature. The reaction mixture was stirred for 3.5 h prior to quenching with a 1:1 solution of water:saturated NaHCO₃ (30 mL) and diluting with EtOAc (30 mL). The layers were separated and the aqueous layer was extracted once more with EtOAc (30 mL). The organic fractions were combined, washed with 1:1 water:brine (30 mL), dried over Na₂SO₄, filtered and concentrated in vacuo prior to purification by silica gel chromatography (0-20% MeOH in DCM) to afford Compound 6. ¹H NMR (400 MHz, DMSO-d6) δ 7.91-7.65 (m, 3H), 6.86 (d, J=4.5 Hz, 1H), 6.72 (d, J=4.5 Hz, 1H), 6.14 (d, J=5.9 Hz, 1H), 5.51 (d, J=5.6 Hz, 1H), 5.39 (d, J=5.7 Hz, 1H), 4.55-4.43 (m, 2H), 4.38-4.25 (m, 2H), 3.72 (s, 3H). MS m/z [M+1]=350.0.

### Example 7: Compound 7 ((2R,3S,4R,5S)-5-(4-aminopyrrolo[2,1-f][1,2,4]triazin-7-yl)-2-cyano-3,4-dihydroxytetrahydrofuran-2-yl)methyl isopentyl carbonate

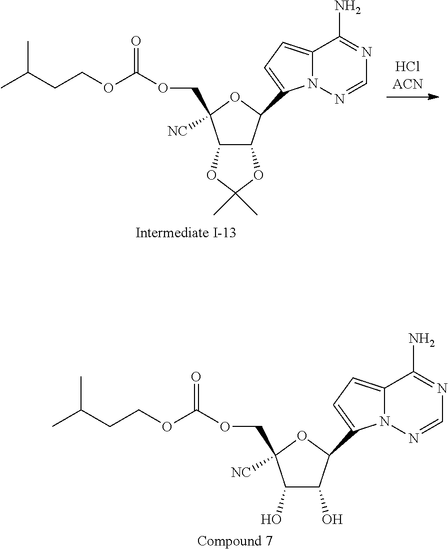 [SMILES: CC(C)CCOC(=O)OC[C@@]1(C#N)O[C@@H](c2ccc3c(N)ncnn23)[C@@H]2OC(C)(C)O[C@@H]21 ; CC(C)CCOC(=O)OC[C@@]1(C#N)O[C@@H](c2ccc3c(N)ncnn23)[C@H](O)[C@@H]1O]

To a solution of Intermediate I-13 (0.247 mmol) in ACN (1 mL) was added 25% HCl (0.2 mL) at rt. The mixture was stirred for 2 h, diluted with EtOAc (20 mL), and aqNaHCO₃ (10 mL) added slowly. The mixture was stirred for 10 min, the phases separated, and the aqueous phase extracted with EtOAc (10 mL×3). The combined organic phase was dried under sodium sulfate, concentrated in vacuo, and purified by silica gel column chromatography (0 to 5% MeOH in DCM) to give Compound 7. ¹H NMR (400 MHz, Methanol-d4) δ 7.82 (s, 1H), 6.87 (d, J=4.5 Hz, 1H), 6.75 (d, J=4.5 Hz, 1H), 5.53 (d, J=4.8 Hz, 1H), 4.66-4.57 (m, 2H), 4.49 (d, J=5.5 Hz, 1H), 4.43 (d, J=11.7 Hz, 1H), 4.19 (t, J=6.8, 2H), 1.71 (m, 1H), 1.55 (q, J=6.8 Hz, 2H), 0.95 (d, J=1.8 Hz, 3H), 0.93 (d, J=1.9 Hz, 3H). MS m/z [M+1]=406.0.

### Example 8: Compound 8 ((2R,3S,4R,5S)-5-(4-aminopyrrolo[2,1-f][1,2,4]triazin-7-yl)-2-cyano-3,4-dihydroxytetrahydrofuran-2-yl)methyl isobutyl carbonate

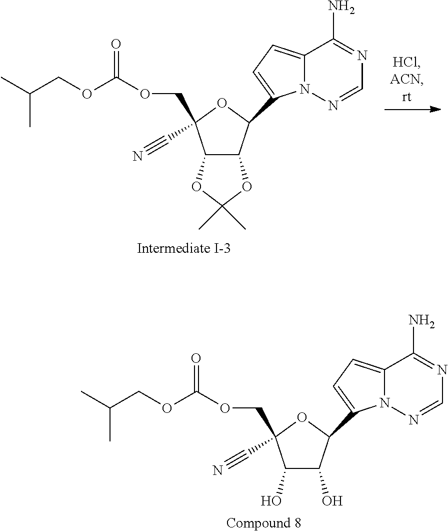 [SMILES: CC(C)COC(=O)OC[C@@]1(C#N)O[C@@H](c2ccc3c(N)ncnn23)[C@@H]2OC(C)(C)O[C@@H]21 ; CC(C)COC(=O)OC[C@@]1(C#N)O[C@@H](c2ccc3c(N)ncnn23)[C@H](O)[C@@H]1O]

To a solution of Intermediate I-3 (5.9 mmol) in ACN (25.0 mL) was added concentrated HCl (2.4 mL) at room temperature. The reaction mixture was stirred for 1 h prior to cooling in an ice bath and quenching with a 1:1 solution of water and saturated NaHCO₃ (150 mL). The aqueous layer was extracted with EtOAc (3×100 mL). The organic fractions were combined, washed with 1:1 water:brine (100 mL), dried over Na₂SO₄, filtered and concentrated in vacuo prior to purification by silica gel chromatography (0-20% MeOH in DCM) to afford Compound 8. ¹H NMR (400 MHz, DMSO-d₆) δ 7.91-7.65 (m, 3H), 6.86 (d, J=4.5 Hz, 1H), 6.73 (d, J=4.5 Hz, 1H), 6.15 (d, J=5.9 Hz, 1H), 5.52 (d, J=5.6 Hz, 1H), 5.40 (d, J=5.6 Hz, 1H), 4.57-4.42 (m, 2H), 4.39-4.24 (m, 2H), 3.93-3.84 (m, 2H), 1.96-1.82 (m, 1H), 0.88 (d, J=6.8 Hz, 6H). MS m/z [M+1]=392.1.

### Example 9: Compound 9 ((2R,3S,4R,5S)-5-(4-aminopyrrolo[2,1-f][1,2,4]triazin-7-yl)-2-cyano-3,4-dihydroxytetrahydrofuran-2-yl)methyl propyl carbonate

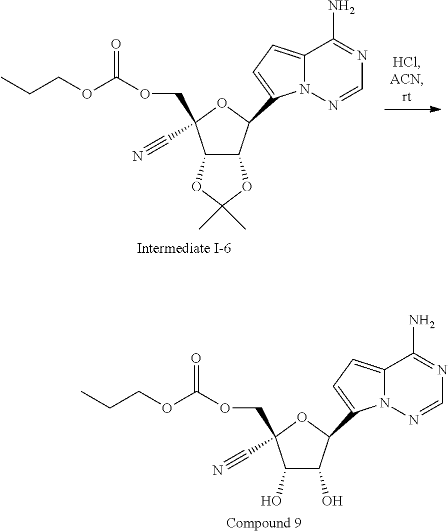 [SMILES: CCCOC(=O)OC[C@@]1(C#N)O[C@@H](c2ccc3c(N)ncnn23)[C@@H]2OC(C)(C)O[C@@H]21 ; CCCOC(=O)OC[C@@]1(C#N)O[C@@H](c2ccc3c(N)ncnn23)[C@H](O)[C@@H]1O]

To a solution of Intermediate I-6 (0.34 mmol) in ACN (5.0 mL) was added concentrated HCl (0.14 mL) at room temperature. The reaction mixture was stirred for 4 h and 25 min prior to quenching with a 1:1 solution of water:saturated NaHCO₃ (30 mL) and diluting with EtOAc (30 mL). The layers were separated, and the aqueous layer was extracted once more with EtOAc (30 mL). The organic fractions were combined, washed with 1:1 water:brine (30 mL), dried over Na₂SO₄, filtered and concentrated in vacuo prior to purification by silica gel chromatography (0-20% MeOH in DCM) to afford Compound 9. ¹H NMR (400 MHz, DMSO-d6) δ 7.88-7.65 (m, 3H), 6.86 (d, J=4.5 Hz, 1H), 6.72 (d, J=4.5 Hz, 1H), 6.14 (d, J=5.9 Hz, 1H), 5.51 (d, J=5.6 Hz, 1H), 5.39 (d, J=5.7 Hz, 1H), 4.55-4.43 (m, 2H), 4.36-4.25 (m, 2H), 4.05 (t, J=6.6 Hz, 2H), 1.66-1.54 (m, 2H), 0.87 (t, J=7.4 Hz, 3H). MS m/z [M+1]=378.1.

### Example 10: Compound 10 ((2R,3S,4R,5S)-5-(4-aminopyrrolo[2,1-f][1,2,4]triazin-7-yl)-2-cyano-3,4-dihydroxytetrahydrofuran-2-yl)methyl ((R)-sec-butyl) carbonate

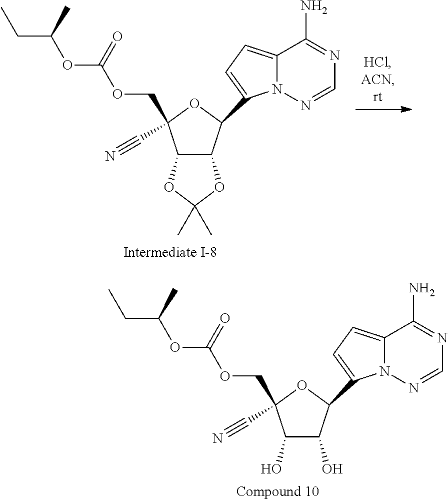 [SMILES: CC[C@@H](C)OC(=O)OC[C@@]1(C#N)O[C@@H](c2ccc3c(N)ncnn23)[C@@H]2OC(C)(C)O[C@@H]21 ; CC[C@@H](C)OC(=O)OC[C@@]1(C#N)O[C@@H](c2ccc3c(N)ncnn23)[C@H](O)[C@@H]1O]

To a solution of Intermediate I-8 (0.24 mmol) in ACN (5.0 mL) was added concentrated HCl (0.10 mL) at room temperature. The reaction mixture was stirred for 50 min prior to quenching with a 1:1 solution of water:saturated NaHCO₃ (30 mL) and diluting with EtOAc (30 mL). The layers were separated and the aqueous layer was extracted once more with EtOAc (30 mL). The organic fractions were combined, washed with 1:1 water:brine (20 mL), dried over MgSO₄, filtered and concentrated in vacuo prior to purification by silica gel chromatography (0-15% MeOH in DCM) to afford Compound 10. ¹H NMR (400 MHz, DMSO-d6) δ 7.90-7.67 (m, 3H), 6.86 (d, J=4.5 Hz, 1H), 6.72 (d, J=4.5 Hz, 1H), 6.14 (d, J=5.9 Hz, 1H), 5.50 (d, J=5.6 Hz, 1H), 5.39 (d, J=5.7 Hz, 1H), 4.65-4.56 (m, 1H), 4.54-4.43 (m, 2H), 4.36-4.23 (m, 2H), 1.61-1.47 (m, 2H), 1.19 (d, J=6.2 Hz, 3H), 0.86-0.78 (m, 3H). MS m/z [M+1]=392.1.

### Example 11: Compound 11 ((2R,3S,4R,5S)-5-(4-aminopyrrolo[2,1-f][1,2,4]triazin-7-yl)-2-cyano-3,4-dihydroxytetrahydrofuran-2-yl)methyl isopropyl carbonate

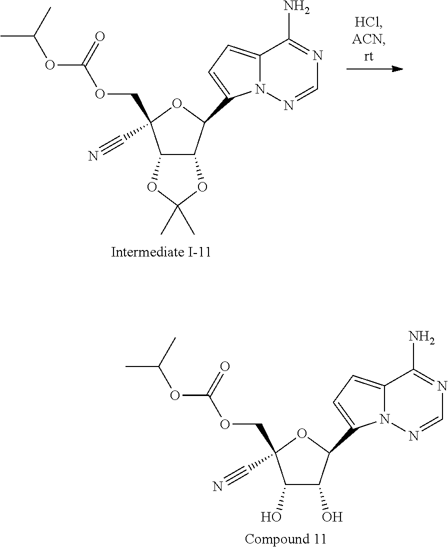 [SMILES: CC(C)OC(=O)OC[C@@]1(C#N)O[C@@H](c2ccc3c(N)ncnn23)[C@@H]2OC(C)(C)O[C@@H]21 ; CC(C)OC(=O)OC[C@@]1(C#N)O[C@@H](c2ccc3c(N)ncnn23)[C@H](O)[C@@H]1O]

To a solution of Intermediate I-11 (0.29 mmol) in ACN (5.0 mL) was added concentrated HCl (0.12 mL) at room temperature. The reaction mixture was stirred for 40 min prior to quenching with a 1:1 solution of water:saturated NaHCO₃ (30 mL) and diluting with EtOAc (30 mL). The layers were separated and the aqueous layer was extracted once more with EtOAc (30 mL). The organic fractions were combined, washed with 1:1 water:brine (40 mL), dried over MgSO₄, filtered and concentrated in vacuo prior to purification by silica gel chromatography (0-15% MeOH in DCM) to afford Compound 11. ¹H NMR (400 MHz, DMSO-d6) δ 7.90-7.64 (m, 3H), 6.86 (d, J=4.4 Hz, 1H), 6.72 (d, J=4.5 Hz, 1H), 6.14 (d, J=5.9 Hz, 1H), 5.50 (d, J=5.6 Hz, 1H), 5.38 (d, J=5.7 Hz, 1H), 4.82-4.71 (m, 1H), 4.55-4.42 (m, 2H), 4.35-4.24 (m, 2H), 1.25-1.19 (m, 6H). MS m/z [M+1]=378.1.

### Example 12: Compound 12 ((2R,3S,4R,5S)-5-(4-aminopyrrolo[2,1-f][1,2,4]triazin-7-yl)-2-cyano-3,4-dihydroxytetrahydrofuran-2-yl)methyl (tetrahydro-2H-pyran-4-yl) carbonate

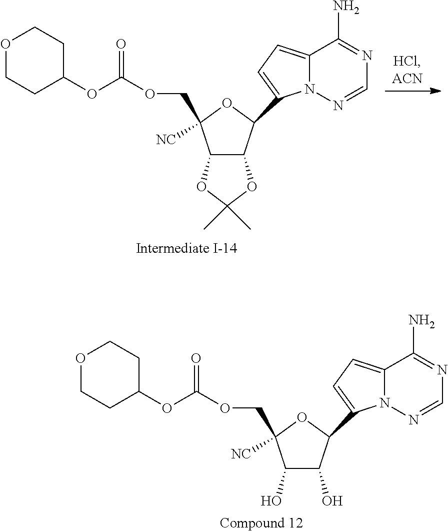 [SMILES: CC1(C)O[C@H]2[C@H](c3ccc4c(N)ncnn34)O[C@](C#N)(COC(=O)OC3CCOCC3)[C@H]2O1 ; N#C[C@]1(COC(=O)OC2CCOCC2)O[C@@H](c2ccc3c(N)ncnn23)[C@H](O)[C@@H]1O]

To a solution of Intermediate 1-17 (0.266 mmol) in ACN (1 mL) was added 25% HCl (0.2 mL) at rt. The mixture was stirred for 2 h, diluted with EtOAc (20 mL), and aqNaHCO₃ (10 mL) added slowly. The mixture was stirred for 10 min, the phases separated, and the aqueous phase extracted with EtOAc (10 mL×3). The combined organic phase was dried under sodium sulfate, concentrated in vacuo, and purified by silica gel column chromatography (0 to 5% MeOH in DCM) to give Compound 12. ¹H NMR (400 MHz, Methanol-d4) δ 7.82 (s, 1H), 6.87 (d, J=4.5 Hz, 1H), 6.75 (d, J=4.5 Hz, 1H), 5.53 (d, J=4.6 Hz, 1H), 4.80 (m, 1H), 4.67-4.56 (m, 2H), 4.50 (d, J=5.5 Hz, 1H), 4.44 (d, J=11.7 Hz, 1H), 3.88 (m, 2H), 3.54 (m, 2H), 1.95 (m, 2H), 1.68 (m, 2H). MS m/z [M+1]=419.9.

### Example 13: Compound 13 ((2R,3S,4R,5S)-5-(4-aminopyrrolo[2,1-f][1,2,4]triazin-7-yl)-2-cyano-3,4-dihydroxytetrahydrofuran-2-yl)methyl tert-butyl carbonate

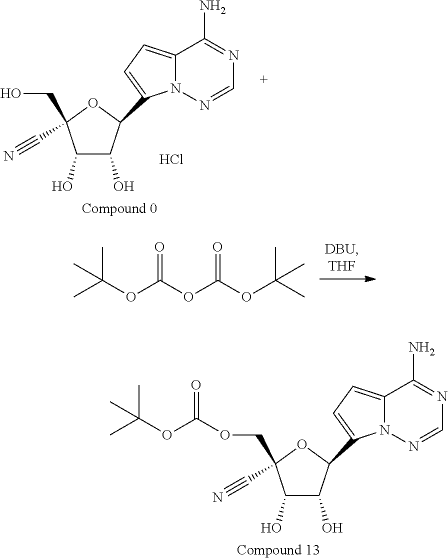 [SMILES: N#C[C@]1(CO)O[C@@H](c2ccc3c(N)ncnn23)[C@H](O)[C@@H]1O ; Cl ; CC(C)(C)OC(=O)OC(=O)OC(C)(C)C ; CC(C)(C)OC(=O)OC[C@@]1(C#N)O[C@@H](c2ccc3c(N)ncnn23)[C@H](O)[C@@H]1O]

A 2.0-5.0 mL microwave vial was charged with (2R,3S,4R,5S)-5-(4-aminopyrrolo[2,1-f][1,2,4]triazin-7-yl)-3,4-dihydroxy-2-(hydroxymethyl)tetrahydrofuran-2-carbonitrile hydrogen chloride (0.25 mmol). A solution of di-tert-butyl dicarbonate (0.39 mmol) in THE (4.0 mL) was transferred to the vial followed by DBU (0.11 mL, 0.76 mmol). The reaction mixture was stirred at room temperature overnight. Additional di-tert-butyl dicarbonate (0.17 mmol) dissolved in THE (1.0 mL) was added and the reaction mixture was stirred at room temperature for an additional 3 h and then heated to 50° C. and stirred overnight. The solution was diluted with water (30 mL) and extracted with EtOAc (2×30 mL). The organic fractions were combined, washed with 1:1 brine:water (20 mL), dried over MgSO₄, filtered, concentrated in vacuo and purified by silica gel chromatography (0-20% MeOH in DCM) to afford Compound 13. ¹H NMR (400 MHz, DMSO-d6) δ 7.89-7.68 (m, 3H), 6.86 (d, J=4.5 Hz, 1H), 6.73 (d, J=4.5 Hz, 1H), 6.13 (d, J=5.9 Hz, 1H), 5.49 (d, J=5.7 Hz, 1H), 5.38 (d, J=5.8 Hz, 1H), 4.51-4.41 (m, 2H), 4.29-4.20 (m, 2H), 1.40 (s, 9H). MS m/z [M+1]=392.1.

### Example 14: Compound 14 ((2R,3S,4R,5S)-5-(4-aminopyrrolo[2,1-f][1,2,4]triazin-7-yl)-2-cyano-3,4-dihydroxytetrahydrofuran-2-yl)methyl pentan-3-yl carbonate

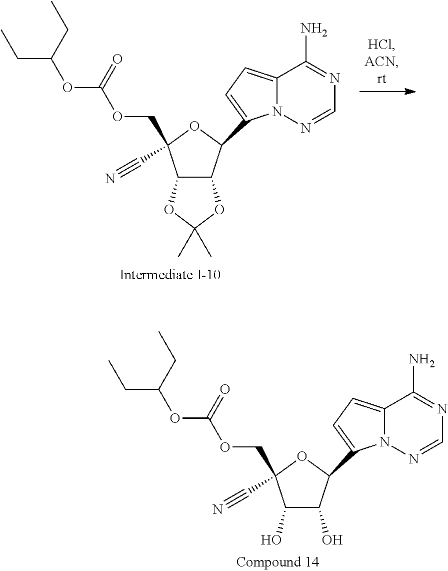 [SMILES: CCC(CC)OC(=O)OC[C@@]1(C#N)O[C@@H](c2ccc3c(N)ncnn23)[C@@H]2OC(C)(C)O[C@@H]21 ; CCC(CC)OC(=O)OC[C@@]1(C#N)O[C@@H](c2ccc3c(N)ncnn23)[C@H](O)[C@@H]1O]

To a solution of Intermediate 1-10 (0.17 mmol) in ACN (5.0 mL) was added concentrated HCl acid (0.07 mL, 12 M) at room temperature. The reaction mixture was stirred for 3 h prior to quenching with a 1:1 solution of water:saturated NaHCO₃ (30 mL) and diluting with EtOAc (30 mL). The layers were separated and the aqueous layer was extracted once more with EtOAc (30 mL). The organic fractions were combined, washed with 1:1 water:brine (20 mL), dried over MgSO₄, filtered and concentrated in vacuo prior to purification by silica gel chromatography (0-15% MeOH in DCM) to afford Compound 14. ¹H NMR (400 MHz, DMSO-d6) δ 7.90-7.63 (m, 3H), 6.86 (d, J=4.4 Hz, 1H), 6.72 (d, J=4.5 Hz, 1H), 6.14 (d, J=5.9 Hz, 1H), 5.50 (d, J=5.6 Hz, 1H), 5.39 (d, J=5.7 Hz, 1H), 4.57-4.44 (m, 3H), 4.36-4.23 (m, 2H), 1.66-1.43 (m, 4H), 0.88-0.76 (m, 6H). MS m/z [M+1]=406.1.

### Example 15: Compound 15 ((3aS,4R,6S,6aS)-6-(4-aminopyrrolo[2,1-f][1,2,4]triazin-7-yl)-4-cyano-2-oxotetrahydrofuro[3,4-d][1,3]dioxol-4-yl)methyl isobutyl carbonate

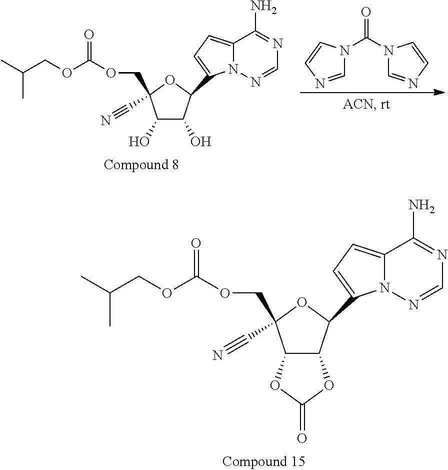

To a solution of Compound 8 (0.13 mmol) in ACN (7.5 mL) was added CDI (0.27 mmol). The reaction mixture was stirred at room temperature for 55 min prior to quenching with water (30 mL). The solution was stirred for 25 min before extracting with EtOAc (2×30 mL). The organic fractions were combined, washed with 1:1 brine:water (40 mL), dried over MgSO₄, filtered and concentrated in vacuo. The crude residue was subjected to purification by silica gel chromatography (0-100% EtOAc in hexanes) to afford Compound 15. ¹H NMR (400 MHz, DMSO-d6) δ 8.06-7.78 (m, 3H), 6.90-6.86 (m, 2H), 5.85-5.79 (m, 2H), 5.77-5.71 (m, 1H), 4.70 (d, J=11.6 Hz, 1H), 4.56 (d, J=11.6 Hz, 1H), 3.94-3.87 (m, 2H), 1.97-1.82 (m, 1H), 0.87 (d, J=6.7 Hz, 6H). MS m/z [M+1]=418.1.

### Example 16: Compound 16 Isobutyl (7-((2S,3R,4S,5R)-5-cyano-3,4-dihydroxy-5-(hydroxymethyl)tetrahydrofuran-2-yl)pyrrolo[2,1-f][1,2,4]triazin-4-yl)carbamate

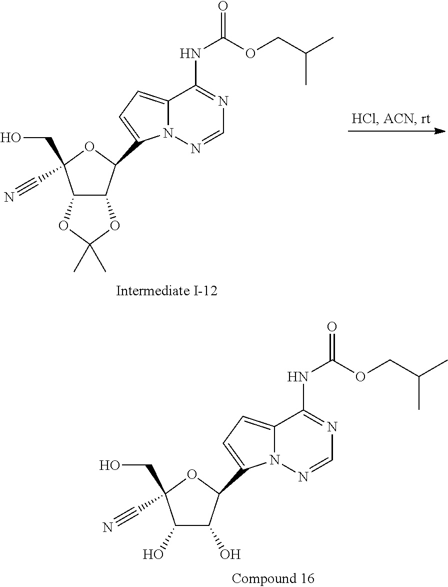 [SMILES: CC(C)COC(=O)Nc1ncnn2c([C@@H]3O[C@](C#N)(CO)[C@H]4OC(C)(C)O[C@@H]34)ccc12 ; CC(C)COC(=O)Nc1ncnn2c([C@@H]3O[C@](C#N)(CO)[C@@H](O)[C@H]3O)ccc12]

To a solution of Intermediate 1-12 (0.11 mmol) in ACN (5.0 mL) was added concentrated HCl acid (0.05 mL) at room temperature. The reaction mixture was stirred for 3.5h prior to quenching with a 1:1 solution of water:saturated NaHCO₃ (20 mL) and diluting with EtOAc (20 mL). The layers were separated and the aqueous layer was extracted once more with EtOAc (20 mL). The organic fractions were combined, washed with 1:1 water:brine (20 mL), dried over MgSO₄, filtered and concentrated in vacuo prior to purification by silica gel chromatography (0-20% MeOH in DCM) to afford Compound 16. ¹H NMR (400 MHz, DMSO-d6) δ 10.76 (s, 1H), 8.29 (s, 1H), 7.26 (d, J=4.7 Hz, 1H), 7.00 (d, J=4.7 Hz, 1H), 5.98-5.93 (m, 1H), 5.68-5.62 (m, 1H), 5.46-5.39 (m, 2H), 4.48-4.40 (m, 1H), 4.25-4.18 (m, 1H), 4.01-3.93 (m, 2H), 3.74-3.65 (m, 1H), 3.64-3.55 (m, 1H), 2.05-1.90 (m, 1H), 0.96 (d, J=6.7 Hz, 6H). MS m/z [M+1]=392.1.

### Example 17: ((2R,3S,4R,5S)-5-(4-aminopyrrolo[2,1-f][1,2,4]triazin-7-yl)-2-cyano-3,4-dihydroxytetrahydrofuran-2-yl)methyl cyclobutyl carbonate

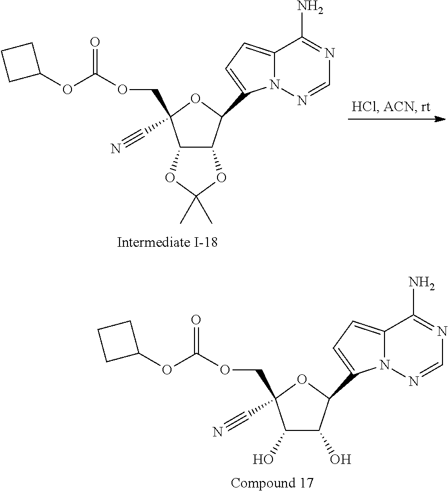 [SMILES: CC1(C)O[C@H]2[C@H](c3ccc4c(N)ncnn34)O[C@](C#N)(COC(=O)OC3CCC3)[C@H]2O1 ; N#C[C@]1(COC(=O)OC2CCC2)O[C@@H](c2ccc3c(N)ncnn23)[C@H](O)[C@@H]1O]

To a solution of Intermediate I-18 (250 mg, 0.58 mmol) in ACN (4.0 mL) was added concentrated HCl (0.24 mL) drop wise at room temperature. The reaction mixture was stirred for 1 h prior to quenching with a 1:1 saturated NaHCO₃ solution (10 mL) and EtOAc (10 mL). The layers were separated, and the aqueous layer was extracted once more with EtOAc (30 mL). The organic fractions were combined, washed with 1:1 water:brine (40 mL), dried over Na₂SO₄, filtered and concentrated in vacuo prior to purification by silica gel chromatography (0-20% MeOH in DCM) to afford Compound 17. ¹H NMR (400 MHz, DMSO-d₆) δ 7.86 (s, 1H), 7.78 (s, 2H), 6.87 (d, J=4.5 Hz, 1H), 6.73 (d, J=4.5 Hz, 1H), 6.16 (d, J=5.9 Hz, 1H), 5.52 (d, J=5.6 Hz, 1H), 5.39 (d, J=5.7 Hz, 1H), 4.95-4.74 (m, 1H), 4.55-4.40 (m, 2H), 4.38-4.16 (m, 2H), 2.36-2.17 (m, 2H), 2.11-1.89 (m, 2H), 1.85-1.44 (m, 2H). MS m/z [M+1]=389.97.

### Example 18: ((2R,3S,4R,5S)-5-(4-aminopyrrolo[2,1-f][1,2,4]triazin-7-yl)-2-cyano-3,4-dihydroxytetrahydrofuran-2-yl)methyl cyclooctyl carbonate

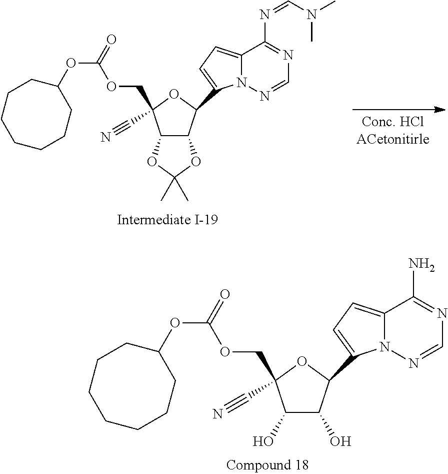 [SMILES: CN(C)/C=N\c1ncnn2c([C@@H]3O[C@](C#N)(COC(=O)OC4CCCCCCC4)[C@H]4OC(C)(C)O[C@@H]34)ccc12 ; N#C[C@]1(COC(=O)OC2CCCCCCC2)O[C@@H](c2ccc3c(N)ncnn23)[C@H](O)[C@@H]1O]

To a solution of Intermediate I-19 (200 mg, 0.37 mmol) in acetonitrile (10 mL), Conc. HCl (0.6 mL, 7.5 mmol) was added and stirred at room temperature for 2 h. After completion of the reaction, diluted with ethyl acetate (50 mL), neutralized with aqueous sodium bicarbonate, separated the organic layer, washed with water, brine, dried and concentrated. The residue is purified by flash chromatography using 0-20% MeOH in DCM as eluent to afford Compound 18. ¹H NMR (400 MHz, Methanol-d4) δ 7.82 (s, 1H), 6.87 (d, J=4.5 Hz, 1H), 6.74 (d, J=4.5 Hz, 1H), 5.52 (d, J=4.7 Hz, 1H), 4.77 (tt, J=8.2, 4.0 Hz, 1H), 4.66-4.57 (m, 2H), 4.50 (d, J=5.5 Hz, 1H), 4.40 (d, J=11.7 Hz, 1H), 1.94-1.65 (m, 4H), 1.63-1.44 (m, 8H), 1.31 (dt, J=3.9, 2.4 Hz, 1H), 0.96-0.85 (m, 1H). MS m/z [M+1]=446.1

### Example 19: ((2R,3S,4R,5S)-5-(4-aminopyrrolo[2,1-f][1,2,4]triazin-7-yl)-2-cyano-3,4-dihydroxytetrahydrofuran-2-yl)methyl hexyl carbonate

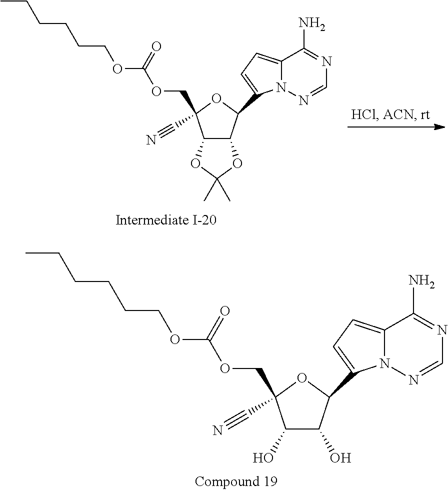 [SMILES: CCCCCCOC(=O)OC[C@@]1(C#N)O[C@@H](c2ccc3c(N)ncnn23)[C@@H]2OC(C)(C)O[C@@H]21 ; CCCCCCOC(=O)OC[C@@]1(C#N)O[C@@H](c2ccc3c(N)ncnn23)[C@H](O)[C@@H]1O]

To a solution of ((3aS,4R,6S,6aS)-6-(4-aminopyrrolo[2,1-f][1,2,4]triazin-7-yl)-4-cyano-2,2-dimethyltetrahydrofuro[3,4-d][1,3]dioxol-4-yl)methyl hexyl carbonate (79 mg, 0.17 mmol, 1.0 equiv.) in ACN (5.0 mL) was added concentrated HCl acid (0.07 mL, 12 M, 5.0 equiv.) at room temperature. The reaction mixture was stirred for 3 h prior to quenching with a 1:1 solution of water:saturated NaHCO₃ (30 mL) and diluting with EtOAc (30 mL). The layers were separated and the aqueous layer was extracted once more with EtOAc (30 mL). The organic fractions were combined, washed with 1:1 water:brine (40 mL), dried over MgSO₄, filtered and concentrated in vacuo prior to purification by silica gel chromatography (0-15% MeOH in DCM) to afford the title Compound 19. ¹H NMR (400 MHz, DMSO-d6) δ 7.89-7.67 (m, 3H), 6.86 (d, J=4.5 Hz, 1H), 6.72 (d, J=4.5 Hz, 1H), 6.14 (d, J=5.9 Hz, 1H), 5.51 (d, J=5.6 Hz, 1H), 5.39 (d, J=5.7 Hz, 1H), 4.54-4.42 (m, 2H), 4.37-4.24 (m, 2H), 4.12-4.05 (m, 2H), 1.63-1.52 (m, 2H), 1.35-1.18 (m, 6H), 0.89-0.81 (m, 3H). MS m/z [M+1]=420.1

### Example 20: ((2R,3S,4R,5S)-5-(4-aminopyrrolo[2,1-f][1,2,4]triazin-7-yl)-2-cyano-3,4-dihydroxytetrahydrofuran-2-yl)methyl pentyl carbonate

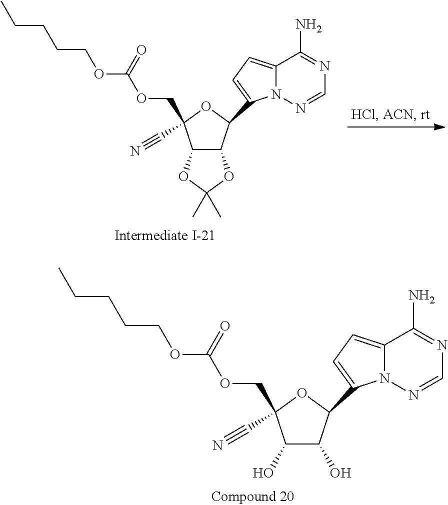 [SMILES: CCCCCOC(=O)OC[C@@]1(C#N)O[C@@H](c2ccc3c(N)ncnn23)[C@@H]2OC(C)(C)O[C@@H]21 ; CCCCCOC(=O)OC[C@@]1(C#N)O[C@@H](c2ccc3c(N)ncnn23)[C@H](O)[C@@H]1O]

To a solution of ((3aS,4R,6S,6aS)-6-(4-aminopyrrolo[2,1-f][1,2,4]triazin-7-yl)-4-cyano-2,2-dimethyltetrahydrofuro[3,4-d][1,3]dioxol-4-yl)methyl pentyl carbonate (161 mg, 0.36 mmol, 1.0 equiv.) in ACN (5.0 mL) was added concentrated HCl acid (0.15 mL, 12 M, 5.0 equiv.) at room temperature. The reaction mixture was stirred for 4 h and 35 min prior to quenching with a 1:1 solution of water:saturated NaHCO₃ (30 mL) and diluting with EtOAc (30 mL). The layers were separated and the aqueous layer was extracted once more with EtOAc (30 mL). The organic fractions were combined, washed with 1:1 water:brine (30 mL), dried over MgSO₄, filtered and concentrated in vacuo prior to purification by silica gel chromatography (0-15% MeOH in DCM) to afford the title Compound 20. ¹H NMR (400 MHz, DMSO-d6) δ 7.97-7.70 (m, 3H), 6.88 (d, J=4.5 Hz, 1H), 6.73 (d, J=4.4 Hz, 1H), 6.20-6.09 (m, 1H), 5.51 (s, 1H), 5.39 (d, J=5.7 Hz, 1H), 4.56-4.42 (m, 2H), 4.36-4.23 (m, 2H), 4.12-4.05 (m, 2H), 1.65-1.52 (m, 2H), 1.34-1.21 (m, 4H), 0.90-0.81 (m, 3H). MS m/z [M+1]=406.1

### Example 21: ((2R,3S,4R,5S)-5-(4-aminopyrrolo[2,1-f][1,2,4]triazin-7-yl)-2-cyano-3,4-dihydroxytetrahydrofuran-2-yl)methyl cyclohexyl carbonate

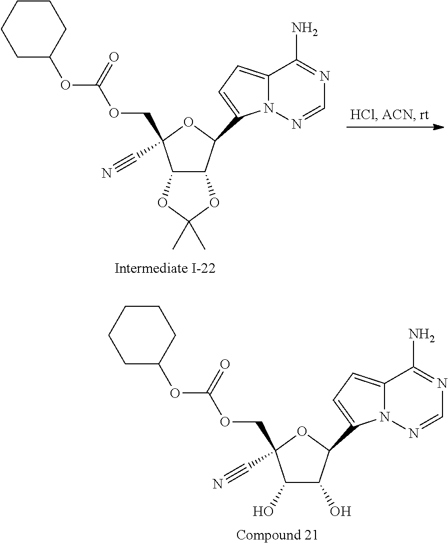 [SMILES: CC1(C)O[C@H]2[C@H](c3ccc4c(N)ncnn34)O[C@](C#N)(COC(=O)OC3CCCCC3)[C@H]2O1 ; N#C[C@]1(COC(=O)OC2CCCCC2)O[C@@H](c2ccc3c(N)ncnn23)[C@H](O)[C@@H]1O]

To a solution of ((3aS,4R,6S,6aS)-6-(4-aminopyrrolo[2,1-f][1,2,4]triazin-7-yl)-4-cyano-2,2-dimethyltetrahydrofuro[3,4-d][1,3]dioxol-4-yl)methyl cyclohexyl carbonate (108 mg, 0.24 mmol, 1.0 equiv.) in ACN (5.0 mL) was added concentrated HCl acid (0.10 mL, 12 M, 5.0 equiv.) at room temperature. The reaction mixture was stirred for 3 h and 45 min prior to filtering and washing with a minimal amount of ACN. The filtrate was filtered once more and both filter cakes were combined. The filter cakes were dissolved in methanol and concentrated in vacuo prior to lyophilization to afford the final Compound 21 as the HCl salt. ¹H NMR (400 MHz, DMSO-d6) δ 9.79 (s, 1H), 9.10 (s, 1H), 8.17 (s, 1H), 7.41 (d, J=4.6 Hz, 1H), 6.92 (d, J=4.6 Hz, 1H), 5.39 (d, J=5.5 Hz, 1H), 4.59-4.48 (m, 2H), 4.47-4.41 (m, 1H), 4.34 (d, J=11.7 Hz, 1H), 4.25 (d, J=5.1 Hz, 1H), 1.89-1.76 (m, 2H), 1.70-1.59 (m, 2H), 1.53-1.15 (m, 6H). MS m/z [M+1]=418.1

### Example 22: ((2R,3S,4R,5S)-5-(4-aminopyrrolo[2,1-f][1,2,4]triazin-7-yl)-2-cyano-3,4-dihydroxytetrahydrofuran-2-yl)methyl ethyl carbonate

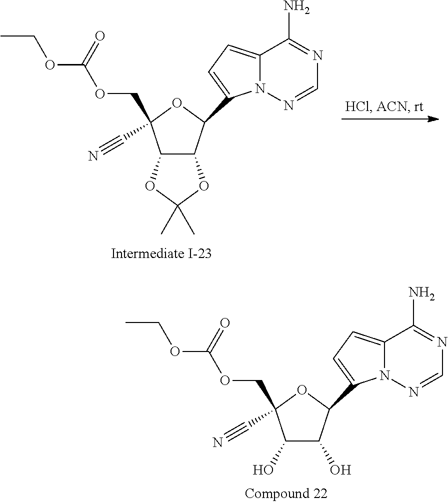 [SMILES: CCOC(=O)OC[C@@]1(C#N)O[C@@H](c2ccc3c(N)ncnn23)[C@@H]2OC(C)(C)O[C@@H]21 ; CCOC(=O)OC[C@@]1(C#N)O[C@@H](c2ccc3c(N)ncnn23)[C@H](O)[C@@H]1O]

To a solution of ((3aS,4R,6S,6aS)-6-(4-aminopyrrolo[2,1-f][1,2,4]triazin-7-yl)-4-cyano-2,2-dimethyltetrahydrofuro[3,4-d][1,3]dioxol-4-yl)methyl ethyl carbonate (193 mg, 0.48 mmol, 1.0 equiv.) in ACN (5.0 mL) was added concentrated HCl acid (0.20 mL, 12 M, 5.0 equiv.) at room temperature. The reaction mixture was stirred for 2 h and 50 min prior to diluting with EtOAc (50 mL) and quenching with a 1:1 solution of water:saturated NaHCO₃ (50 mL). The layers were separated and the aqueous layer was extracted once more with EtOAc (50 mL). The organic fractions were combined, washed with 1:1 water:brine (40 mL), dried over MgSO₄, filtered and concentrated in vacuo prior to purification by silica gel chromatography (0-15% MeOH in DCM) to afford the title Compound 22. ¹H NMR (400 MHz, DMSO-d6) δ 7.95-7.62 (m, 3H), 6.86 (d, J=4.5 Hz, 1H), 6.72 (d, J=4.5 Hz, 1H), 6.14 (d, J=5.9 Hz, 1H), 5.51 (d, J=5.6 Hz, 1H), 5.39 (d, J=5.7 Hz, 1H), 4.56-4.42 (m, 2H), 4.38-4.23 (m, 2H), 4.14 (q, J=7.1 Hz, 2H), 1.21 (t, J=7.1 Hz, 3H). MS m/z [M+1]=364.1

### Example 23: ((2R,3S,4R,5S)-5-(4-aminopyrrolo[2,1-f][1,2,4]triazin-7-yl)-2-cyano-3,4-dihydroxytetrahydrofuran-2-yl)methyl cyclopropyl carbonate

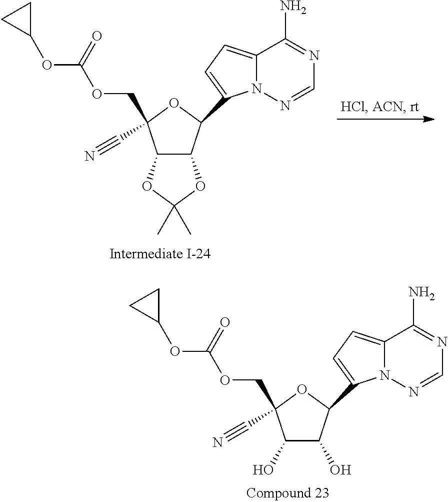 [SMILES: CC1(C)O[C@H]2[C@H](c3ccc4c(N)ncnn34)O[C@](C#N)(COC(=O)OC3CC3)[C@H]2O1 ; N#C[C@]1(COC(=O)OC2CC2)O[C@@H](c2ccc3c(N)ncnn23)[C@H](O)[C@@H]1O]

To a solution of ((3aS,4R,6S,6aS)-6-(4-aminopyrrolo[2,1-f][1,2,4]triazin-7-yl)-4-cyano-2,2-dimethyltetrahydrofuro[3,4-d][1,3]dioxol-4-yl)methyl cyclopropyl carbonate (44 mg, 0.11 mmol, 1.0 equiv.) in ACN (5.0 mL) was added concentrated HCl acid (0.04 mL, 12 M, 5.0 equiv.) at room temperature. The reaction mixture was stirred for 4 h and 45 min prior to neutralizing with triethylamine (0.15 mL, 1.1 mmol, 10 equiv.), concentrating in vacuo, filtering through a PTFE membrane and subjecting to RP prep-HPLC (10-90% ACN in water) to afford the Compound 23. ¹H NMR (400 MHz, DMSO-d6) δ 7.92-7.65 (m, 3H), 6.86 (d, J=4.5 Hz, 1H), 6.72 (d, J=4.5 Hz, 1H), 6.14 (d, J=5.9 Hz, 1H), 5.50 (d, J=5.6 Hz, 1H), 5.38 (d, J=5.7 Hz, 1H), 4.55-4.43 (m, 2H), 4.34 (d, J=11.6 Hz, 1H), 4.30-4.25 (m, 1H), 4.13-4.06 (m, 1H), 0.72-0.65 (m, 4H). MS m/z [M+1]=376.0

### Example 24: ((2R,3S,4R,5S)-5-(4-aminopyrrolo[2,1-f][1,2,4]triazin-7-yl)-2-cyano-3,4-dihydroxytetrahydrofuran-2-yl)methyl ((R)-tetrahydrofuran-3-yl) carbonate

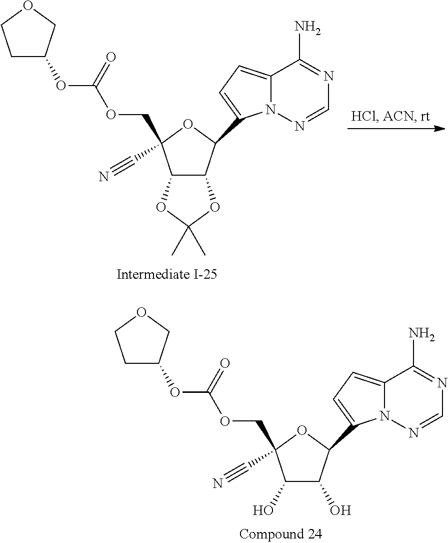 [SMILES: CC1(C)O[C@H]2[C@H](c3ccc4c(N)ncnn34)O[C@](C#N)(COC(=O)O[C@@H]3CCOC3)[C@H]2O1 ; N#C[C@]1(COC(=O)O[C@@H]2CCOC2)O[C@@H](c2ccc3c(N)ncnn23)[C@H](O)[C@@H]1O]

To a solution of ((3aS,4R,6S,6aS)-6-(4-aminopyrrolo[2,1-f][1,2,4]triazin-7-yl)-4-cyano-2,2-dimethyltetrahydrofuro[3,4-d][1,3]dioxol-4-yl)methyl ((R)-tetrahydrofuran-3-yl) carbonate (44 mg, 0.098 mmol, 1.0 equiv.) in ACN (5.0 mL) was added concentrated HCl acid (0.04 mL, 12 M, 5.0 equiv.) at room temperature. The reaction mixture was stirred for 1 h and 25 min before adding additional concentrated HCl acid (0.04 mL, 12 M, 5.0 equiv.). The solution was quenched with triethylamine (0.20 mL, 1.5 mmol, 15 equiv.) after stirring for an additional 1 h and 20 min.

The reaction mixture was concentrated in vacuo, filtered through a PTFE membrane and subjected to RP prep-HPLC (10-90% ACN in water) to afford the Compound 24. ¹H NMR (400 MHz, DMSO-d6) δ 7.91-7.68 (m, 3H), 6.86 (d, J=4.5 Hz, 1H), 6.72 (d, J=4.5 Hz, 1H), 6.15 (d, J=5.9 Hz, 1H), 5.51 (d, J=5.6 Hz, 1H), 5.39 (d, J=5.7 Hz, 1H), 5.19-5.12 (m, 1H), 4.56-4.43 (m, 2H), 4.34 (d, J=11.6 Hz, 1H), 4.30-4.25 (m, 1H), 3.82-3.67 (m, 4H), 2.21-2.09 (m, 1H), 1.99-1.89 (m, 1H). MS m/z [M+1]=406.0

### Example 25 and 26: ((2R,3aS,4R,6S,6aS)-6-(4-aminopyrrolo[2,1-f][1,2,4]triazin-7-yl)-4-cyano-2-methoxytetrahydrofuro[3,4-d][1,3]dioxol-4-yl)methyl isobutyl carbonate and ((2S,3aS,4R,6S,6aS)-6-(4-aminopyrrolo[2,1-f][1,2,4]triazin-7-yl)-4-cyano-2-methoxytetrahydrofuro[3,4-d][1,3]dioxol-4-yl)methyl isobutyl carbonate

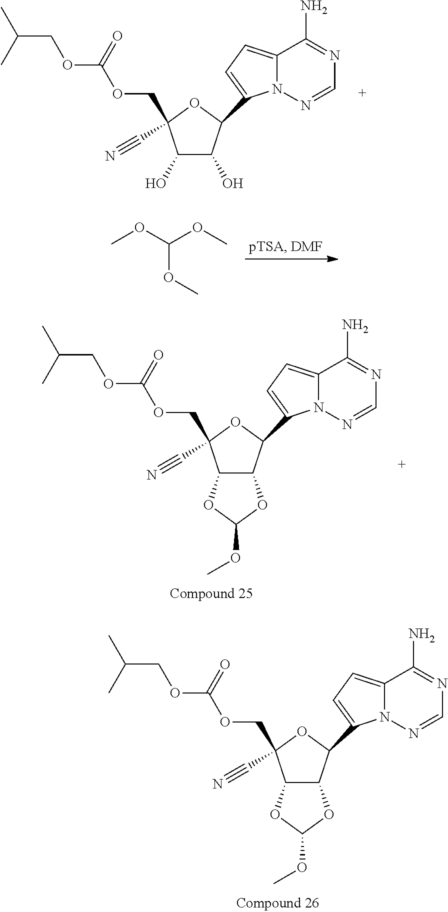

To a solution of ((2R,3S,4R,5S)-5-(4-aminopyrrolo[2,1-f][1,2,4]triazin-7-yl)-2-cyano-3,4-dihydroxytetrahydrofuran-2-yl)methyl isobutyl carbonate (100 mg, 0.26 mmol, 1.0 equiv.) in DMF (5.0 mL) was added PTSA monohydrate (100 mg, 0.53 mmol, 2.1 equiv.) followed by trimethyl orthoformate (0.14 mL, 1.3 mmol, 5.0 equiv.). The reaction mixture was stirred at room temperature for 2 days. The reaction was quenched with triethylamine (0.20 mL, 1.4 mmol, 5.6 equiv.), concentrated in vacuo, filtered through a PTFE membrane and purified by RP prep-HPLC (10-90% ACN in water) to afford the title compounds. Stereochemistry is arbitrarily assigned.

### Example 25: ((2R,3aS,4R,6S,6aS)-6-(4-aminopyrrolo[2,1-f][1,2,4]triazin-7-yl)-4-cyano-2-methoxytetrahydrofuro[3,4-d][1,3]dioxol-4-yl)methyl isobutyl carbonate

¹H NMR (400 MHz, DMSO-d6) δ 7.97-7.75 (m, 3H), 6.91-6.84 (m, 2H), 6.17 (s, 1H), 5.79 (d, J=5.7 Hz, 1H), 5.49-5.42 (m, 1H), 5.09 (d, J=7.3 Hz, 1H), 4.53 (d, J=11.3 Hz, 1H), 4.42 (d, J=11.3 Hz, 1H), 3.89 (d, J=6.6 Hz, 2H), 3.43 (s, 3H), 1.97-1.81 (m, 1H), 0.87 (d, J=6.7 Hz, 6H). MS m/z [M+1]=434.1

### Example 26: ((2S,3aS,4R,6S,6aS)-6-(4-aminopyrrolo[2,1-f][1,2,4]triazin-7-yl)-4-cyano-2-methoxytetrahydrofuro[3,4-d][1,3]dioxol-4-yl)methyl isobutyl carbonate

¹H NMR (400 MHz, DMSO-d6) δ 7.98-7.75 (m, 3H), 6.89-6.85 (m, 2H), 6.31 (s, 1H), 5.62 (d, J=3.5 Hz, 1H), 5.39-5.30 (m, 2H), 4.56 (d, J=11.6 Hz, 1H), 4.44 (d, J=11.6 Hz, 1H), 3.89 (d, J=6.6 Hz, 2H), 3.27 (s, 3H), 1.97-1.81 (m, 1H), 0.87 (d, J=6.7 Hz, 6H). MS m/z [M+1]=434.1

### Example 27: ((2R,3S,4R,5S)-5-(4-aminopyrrolo[2,1-f][1,2,4]triazin-7-yl)-2-cyano-3,4-dihydroxytetrahydrofuran-2-yl)methyl (1,1-difluoro-2-methylpropan-2-yl) carbonate

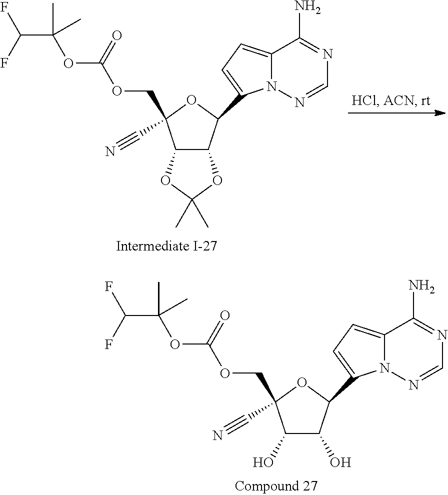 [SMILES: CC1(C)O[C@H]2[C@H](c3ccc4c(N)ncnn34)O[C@](C#N)(COC(=O)OC(C)(C)C(F)F)[C@H]2O1 ; CC(C)(OC(=O)OC[C@@]1(C#N)O[C@@H](c2ccc3c(N)ncnn23)[C@H](O)[C@@H]1O)C(F)F]

To a solution of ((3aS,4R,6S,6aS)-6-(4-aminopyrrolo[2,1-f][1,2,4]triazin-7-yl)-4-cyano-2,2-dimethyltetrahydrofuro[3,4-d][1,3]dioxol-4-yl)methyl (1,1-difluoro-2-methylpropan-2-yl) carbonate (59 mg, 0.126 mmol, 1.0 equiv.) in THE (5.0 mL) was added conc. HCl (0.1 mL, 1.19 mmol, 9.46 equiv.). The reaction mixture was stirred at room temperature for 6 h prior to adding additional HCl (0.1 mL, 1.19 mmol, 9.46 equiv.). The solution was stirred overnight prior to diluting with EtOAc (75 mL) and quenching with 3:2 saturated sodium bicarbonate:water (50 mL). The aqueous layer was extracted once more with EtOAc (1×75 mL). The organic layers were washed with brine (50 mL), dried over sodium sulfate, filtered, concentrated in vacuo and purified by silica gel chromatography (0-20% MeOH in DCM) to afford the Compound 27.

¹H NMR (400 MHz, DMSO-d6) δ 7.89-7.68 (m, 3H), 6.86 (d, J=4.5 Hz, 1H), 6.72 (d, J=4.5 Hz, 1H), 6.32-6.00 (m, 2H), 5.52 (d, J=5.7 Hz, 1H), 5.39 (d, J=5.8 Hz, 1H), 4.55-4.43 (m, 2H), 4.36-4.24 (m, 2H), 1.47-1.43 (m, 6H). ¹⁹F NMR (376 MHz, DMSO-d6) δ −132.98-−133.27 (m). MS m/z [M+1]=427.9

### Example 28: ((2R,3S,4R,5S)-5-(4-aminopyrrolo[2,1-f][1,2,4]triazin-7-yl)-2-cyano-3,4-dihydroxytetrahydrofuran-2-yl)methyl ((S)-2-methylbutyl) carbonate

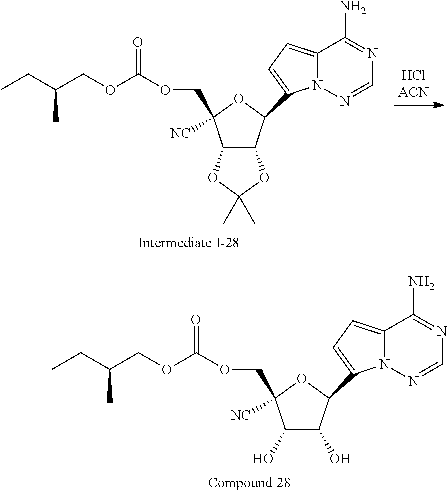

To a solution of Intermediate I-28 (122 mg, 0.274 mmol) in ACN (1 mL) was added 25% HCl (0.2 mL) at rt. The mixture was stirred for 2 h, diluted with EtOAc (20 mL), and aqNaHCO₃ (10 mL) added slowly. The mixture was stirred for 10 min, the phases separated, and the aqueous phase extracted with EtOAc (10 mL×3). The combined organic phase was dried under sodium sulfate, concentrated in vacuo, and purified by silica gel column chromatography (0 to 5% MeOH in DCM) to give Compound 28. ¹H NMR (400 MHz, Methanol-d4) δ 7.82 (s, 1H), 6.87 (d, J=4.5 Hz, 1H), 6.75 (d, J=4.6 Hz, 1H), 5.53 (d, J=4.8 Hz, 1H), 4.65-4.57 (m, 2H), 4.49 (d, J=5.5 Hz, 1H), 4.44 (d, J=11.7 Hz, 1H), 4.10-4.01 (m, 1H), 4.00-3.92 (m, 1H), 1.81-1.66 (m, 1H), 1.53-1.39 (m, 1H), 1.29-1.14 (m, 1H), 1.04-0.77 (m, 6H). MS m/z [M+1]=406.0

### Example 29: ((2R,3S,4R,5S)-5-(4-aminopyrrolo[2,1-f][1,2,4]triazin-7-yl)-2-cyano-3,4-dihydroxytetrahydrofuran-2-yl)methyl benzyl carbonate

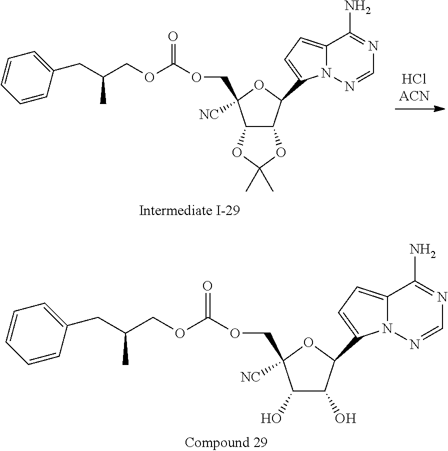

To a solution of Intermediate I-29 (116 mg, 0.249 mmol) in ACN (1 mL) was added 25% HCl (0.2 mL) at rt. The mixture was stirred for 2 h, diluted with EtOAc (20 mL), and aqNaHCO3 (10 mL) added slowly. The mixture was stirred for 10 min, the phases separated, and the aqueous phase extracted with EtOAc (10 mL×3). The combined organic phase was dried under sodium sulfate, concentrated in vacuo, and purified by silica gel column chromatography (0 to 5% MeOH in DCM) to give Compound 29. ¹H NMR (400 MHz, Methanol-d4) δ 7.80 (s, 1H), 7.43-7.25 (m, 5H), 6.85 (d, J=4.5 Hz, 1H), 6.73 (d, J=4.5 Hz, 1H), 5.53 (d, J=4.9 Hz, 1H), 5.18 (d, J=1.2 Hz, 2H), 4.67-4.58 (m, 2H), 4.50-4.43 (m, 2H). MS m/z [M+1]=426.0

### Example 30: ((2R,3S,4R,5S)-5-(4-aminopyrrolo[2,1-f][1,2,4]triazin-7-yl)-2-cyano-3,4-dihydroxytetrahydrofuran-2-yl)methyl (cyclopentylmethyl) carbonate

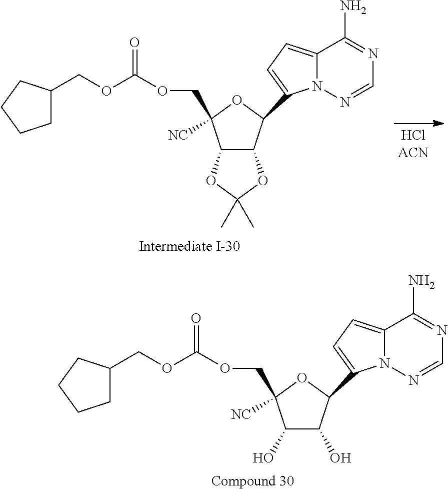

To a solution of Intermediate I-30 (51 mg, 0.111 mmol) in ACN (1 mL) was added 25% HCl (0.1 mL) at rt. The mixture was stirred for 2 h, diluted with EtOAc (20 mL), and aqNaHCO3 (10 mL) added slowly. The mixture was stirred for 10 min, the phases separated, and the aqueous phase extracted with EtOAc (10 mL×3). The combined organic phase was dried under sodium sulfate, concentrated in vacuo, and purified by silica gel column chromatography (0 to 5% MeOH in DCM) to give Compound 30. ¹H NMR (400 MHz, Methanol-d4) δ 7.82 (s, 1H), 6.87 (d, J=4.5 Hz, 1H), 6.75 (d, J=4.5 Hz, 1H), 5.53 (d, J=4.8 Hz, 1H), 4.64-4.57 (m, 2H), 4.49 (d, J=5.5 Hz, 1H), 4.43 (d, J=11.7 Hz, 1H), 4.05 (d, J=2.0 Hz, 1H), 4.03 (d, J=2.0 Hz, 1H), 2.24 (m, 1H), 1.86-1.71 (m, 2H), 1.71-1.52 (m, 4H), 1.40-1.20 (m, 2H); MS m/z [M+1]=418.0

### Example 31: ((2R,3S,4R,5S)-5-(4-aminopyrrolo[2,1-f][1,2,4]triazin-7-yl)-2-cyano-3,4-dihydroxytetrahydrofuran-2-yl)methyl (2-ethylbutyl) carbonate

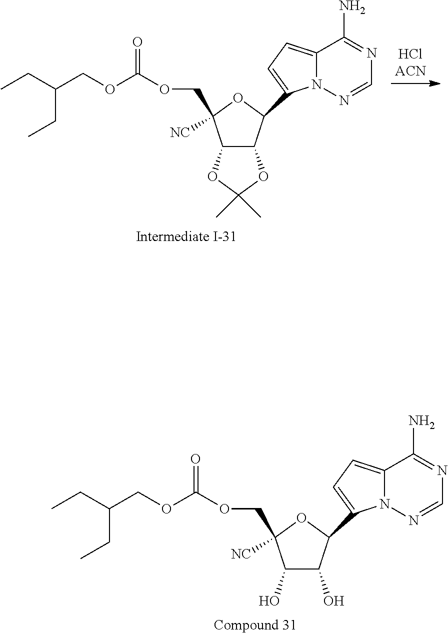 [SMILES: CCC(CC)COC(=O)OC[C@@]1(C#N)O[C@@H](c2ccc3c(N)ncnn23)[C@@H]2OC(C)(C)O[C@@H]21 ; CCC(CC)COC(=O)OC[C@@]1(C#N)O[C@@H](c2ccc3c(N)ncnn23)[C@H](O)[C@@H]1O]

To a solution of Intermediate I-31 (123 mg, 0.268 mmol) in ACN (1 mL) was added 25% HCl (0.2 mL) at rt. The mixture was stirred for 2 h, diluted with EtOAc (20 mL), and aqNaHCO3 (10 mL) added slowly. The mixture was stirred for 10 min, the phases separated, and the aqueous phase extracted with EtOAc (10 mL×3). The combined organic phase was dried under sodium sulfate, concentrated in vacuo, and purified by silica gel column chromatography (0 to 5% MeOH in DCM) to give Compound 31. ¹H NMR (400 MHz, Methanol-d4) δ 7.82 (s, 1H), 6.87 (d, J=4.5 Hz, 1H), 6.75 (d, J=4.5 Hz, 1H), 5.53 (d, J=4.8 Hz, 1H), 4.64-4.57 (m, 2H), 4.49 (d, J=5.5 Hz, 1H), 4.44 (d, J=11.7 Hz, 1H), 4.10 (dd, J=5.7, 1.1 Hz, 2H), 1.60-1.47 (m, 1H), 1.44-1.30 (m, 4H), 0.92 (td, J=7.4, 1.2 Hz, 6H); MS m/z [M+1]=420.0

### Example 32: ((2R,3S,4R,5S)-5-(4-aminopyrrolo[2,1-f][1,2,4]triazin-7-yl)-2-cyano-3,4-dihydroxytetrahydrofuran-2-yl)methyl (cyclopropylmethyl) carbonate

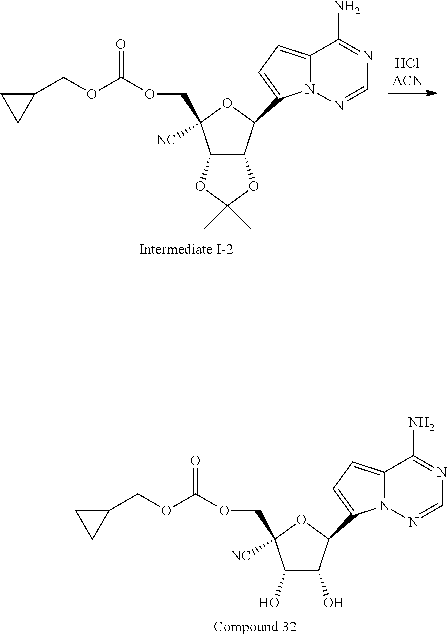 [SMILES: CC1(C)O[C@H]2[C@H](c3ccc4c(N)ncnn34)O[C@](C#N)(COC(=O)OCC3CC3)[C@H]2O1 ; N#C[C@]1(COC(=O)OCC2CC2)O[C@@H](c2ccc3c(N)ncnn23)[C@H](O)[C@@H]1O]

To a solution of Intermediate I-32 (112 mg, 0.261 mmol) in ACN (1 mL) was added 25% HCl (0.2 mL) at rt. The mixture was stirred for 2 h, diluted with EtOAc (20 mL), and aqNaHCO3 (10 mL) added slowly. The mixture was stirred for 10 min, the phases separated, and the aqueous phase extracted with EtOAc (10 mL×3). The combined organic phase was dried under sodium sulfate, concentrated in vacuo, and purified by silica gel column chromatography (0 to 5% MeOH in DCM) to give Compound 32. ¹H NMR (400 MHz, Methanol-d4) δ 7.82 (s, 1H), 6.87 (d, J=4.5 Hz, 1H), 6.75 (d, J=4.5 Hz, 1H), 5.53 (d, J=4.8 Hz, 1H), 4.65-4.56 (m, 2H), 4.49 (d, J=5.5 Hz, 1H), 4.43 (d, J=11.7 Hz, 1H), 3.98 (d, J=7.3 Hz, 2H), 1.25-1.08 (m, 1H), 0.65-0.48 (m, 2H), 0.37-0.21 (m, 2H); MS m/z [M+1]=390.0

### Example 33: ((2R,3S,4R,5S)-5-(4-aminopyrrolo[2,1-f][1,2,4]triazin-7-yl)-2-cyano-3,4-dihydroxytetrahydrofuran-2-yl)methyl ((S)-tetrahydrofuran-3-yl) carbonate

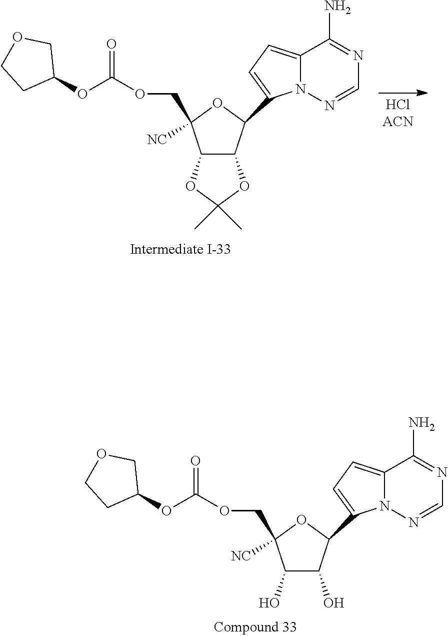 [SMILES: CC1(C)O[C@H]2[C@H](c3ccc4c(N)ncnn34)O[C@](C#N)(COC(=O)O[C@H]3CCOC3)[C@H]2O1 ; N#C[C@]1(COC(=O)O[C@H]2CCOC2)O[C@@H](c2ccc3c(N)ncnn23)[C@H](O)[C@@H]1O]

To a solution of Intermediate I-33 (189 mg, 0.424 mmol) in ACN (2 mL) was added 25% HCl (0.3 mL) at rt. The mixture was stirred for 2 h, diluted with EtOAc (20 mL), and aqNaHCO3 (10 mL) added slowly. The mixture was stirred for 10 min, the phases separated, and the aqueous phase extracted with EtOAc (10 mL×3). The combined organic phase was dried under sodium sulfate, concentrated in vacuo, and purified by silica gel column chromatography (0 to 5% MeOH in DCM) to give Compound 33. ¹H NMR (400 MHz, Methanol-d4) δ 7.82 (s, 1H), 6.87 (d, J=4.5 Hz, 1H), 6.74 (d, J=4.5 Hz, 1H), 5.52 (d, J=4.7 Hz, 1H), 5.25-5.17 (m, 1H), 4.68-4.56 (m, 2H), 4.50 (d, J=5.5 Hz, 1H), 4.45 (d, J=11.7 Hz, 1H), 3.99-3.75 (m, 4H), 2.26-2.14 (m, 1H), 2.14-1.95 (m, 1H); MS m/z [M+1]=405.9

### Example 34: ((2R,3S,4R,5S)-5-(4-aminopyrrolo[2,1-f][1,2,4]triazin-7-yl)-2-cyano-3,4-dihydroxytetrahydrofuran-2-yl)methyl (2-propoxyethyl) carbonate

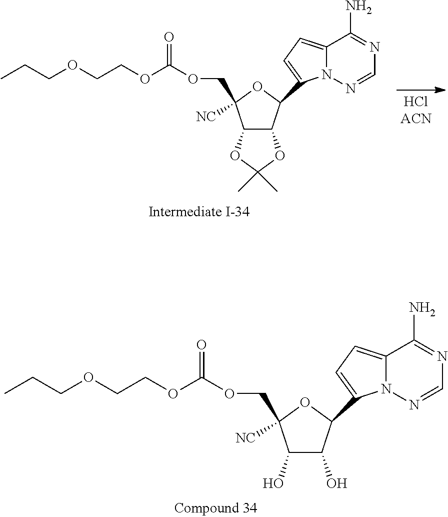 [SMILES: CCCOCCOC(=O)OC[C@@]1(C#N)O[C@@H](c2ccc3c(N)ncnn23)[C@@H]2OC(C)(C)O[C@@H]21 ; CCCOCCOC(=O)OC[C@@]1(C#N)O[C@@H](c2ccc3c(N)ncnn23)[C@H](O)[C@@H]1O]

To a solution of Intermediate I-34 (115 mg, 0.249 mmol) in ACN (1 mL) was added 25% HCl (0.2 mL) at rt. The mixture was stirred for 2 h, diluted with EtOAc (20 mL), and aqNaHCO3 (10 mL) added slowly. The mixture was stirred for 10 min, the phases separated, and the aqueous phase extracted with EtOAc (10 mL×3). The combined organic phase was dried under sodium sulfate, concentrated in vacuo, and purified by silica gel column chromatography (0 to 5% MeOH in DCM) to give Compound 34. ¹H NMR (400 MHz, Methanol-d4) δ 7.82 (s, 1H), 6.87 (d, J=4.5 Hz, 1H), 6.75 (d, J=4.5 Hz, 1H), 5.53 (d, J=4.9 Hz, 1H), 4.64-4.55 (m, 2H), 4.50-4.43 (m, 2H), 4.34-4.21 (m, 2H), 3.66 (t, J=4.7 Hz, 2H), 3.45 (t, J=6.6 Hz, 2H), 165-1.46 (m, 2H), 0.92 (t, J=7.4 Hz, 3H); MS m/z [M+1]=421.9

### Example 35: ((2R,3S,4R,5S)-5-(4-aminopyrrolo[2,1-f][1,2,4]triazin-7-yl)-2-cyano-3,4-dihydroxytetrahydrofuran-2-yl)methyl (3,3-dimethylbutyl) carbonate

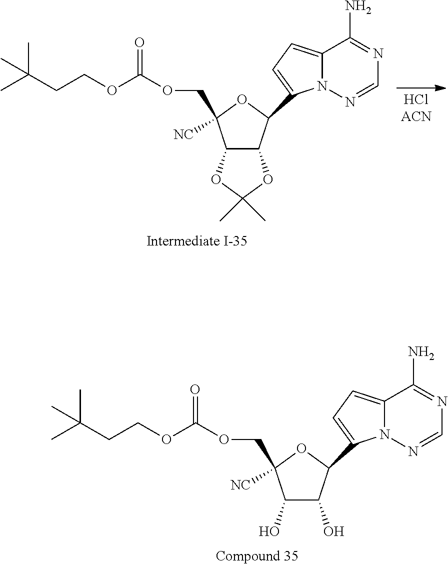 [SMILES: CC(C)(C)CCOC(=O)OC[C@@]1(C#N)O[C@@H](c2ccc3c(N)ncnn23)[C@@H]2OC(C)(C)O[C@@H]21 ; CC(C)(C)CCOC(=O)OC[C@@]1(C#N)O[C@@H](c2ccc3c(N)ncnn23)[C@H](O)[C@@H]1O]

To a solution of Intermediate I-35 (217 mg, 0.472 mmol) in ACN (2 mL) was added 25% HCl (0.4 mL) at rt. The mixture was stirred for 2 h, diluted with EtOAc (20 mL), and aqNaHCO3 (10 mL) added slowly. The mixture was stirred for 10 min, the phases separated, and the aqueous phase extracted with EtOAc (10 mL×3). The combined organic phase was dried under sodium sulfate, concentrated in vacuo, and purified by silica gel column chromatography (0 to 5% MeOH in DCM) to give Compound 35. ¹H NMR (400 MHz, Methanol-d4) δ 7.82 (s, 1H), 6.87 (d, J=4.5 Hz, 1H), 6.75 (d, J=4.5 Hz, 1H), 5.53 (d, J=4.7 Hz, 1H), 4.65-4.53 (m, 2H), 4.50 (d, J=5.5 Hz, 1H), 4.43 (d, J=11.6 Hz, 1H), 4.26-4.10 (m, 2H), 1.68-1.50 (m, 2H), 0.96 (s, 9H). MS m/z [M+1]=420.0

### Example 36: ((2R,3S,4R,5S)-5-(4-aminopyrrolo[2,1-f][1,2,4]triazin-7-yl)-2-cyano-3,4-dihydroxytetrahydrofuran-2-yl)methyl spiro[3.3]heptan-2-yl carbonate

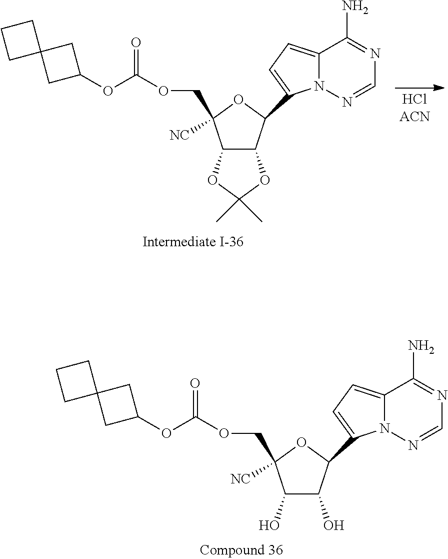 [SMILES: CC1(C)O[C@H]2[C@H](c3ccc4c(N)ncnn34)O[C@](C#N)(COC(=O)OC3CC4(CCC4)C3)[C@H]2O1 ; N#C[C@]1(COC(=O)OC2CC3(CCC3)C2)O[C@@H](c2ccc3c(N)ncnn23)[C@H](O)[C@@H]1O]

To a solution of Intermediate I-36 (267 mg, 0.567 mmol) in ACN (2 mL) was added 25% HCl (0.4 mL) at rt. The mixture was stirred for 2 h, diluted with EtOAc (20 mL), and aqNaHCO3 (10 mL) added slowly. The mixture was stirred for 10 min, the phases separated, and the aqueous phase extracted with EtOAc (10 mL×3). The combined organic phase was dried under sodium sulfate, concentrated in vacuo, and purified by silica gel column chromatography (0 to 5% MeOH in DCM) to give Compound 36. ¹H NMR (400 MHz, Methanol-d4) δ 7.82 (s, 1H), 6.87 (d, J=4.5 Hz, 1H), 6.74 (d, J=4.5 Hz, 1H), 5.51 (d, J=4.8 Hz, 1H), 4.75 (p, J=7.2 Hz, 1H), 4.67-4.55 (m, 2H), 4.50 (d, J=5.5 Hz, 1H), 4.39 (d, J=11.6 Hz, 1H), 2.49-2.35 (m, 2H), 2.09-1.95 (m, 6H), 1.91-1.80 (m, 2H). MS m/z [M+1]=430.0

### Example 37: ((2R,3S,4R,5S)-5-(4-aminopyrrolo[2,1-f][1,2,4]triazin-7-yl)-2-cyano-3,4-dihydroxytetrahydrofuran-2-yl)methyl (3,3-dimethylcyclobutyl) carbonate

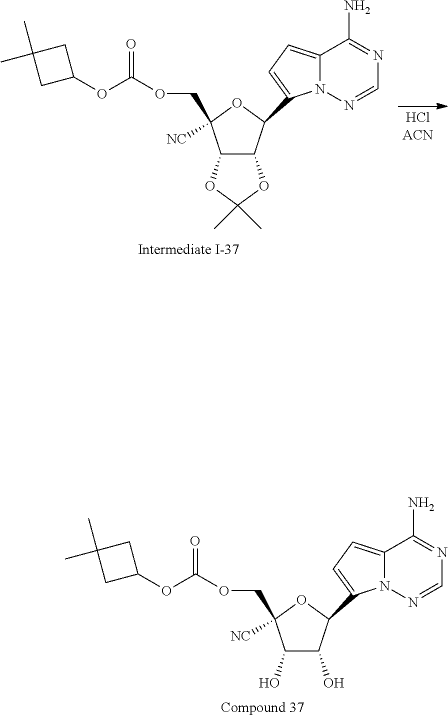 [SMILES: CC1(C)CC(OC(=O)OC[C@@]2(C#N)O[C@@H](c3ccc4c(N)ncnn34)[C@@H]3OC(C)(C)O[C@@H]32)C1 ; CC1(C)CC(OC(=O)OC[C@@]2(C#N)O[C@@H](c3ccc4c(N)ncnn34)[C@H](O)[C@@H]2O)C1]

To a solution of Intermediate I-37 (159 mg, 0.348 mmol) in ACN (1 mL) was added 25% HCl (0.2 mL) at rt. The mixture was stirred for 2 h, diluted with EtOAc (20 mL), and aqNaHCO3 (10 mL) added slowly. The mixture was stirred for 10 min, the phases separated, and the aqueous phase extracted with EtOAc (10 mL×3). The combined organic phase was dried under sodium sulfate, concentrated in vacuo, and purified by silica gel column chromatography (0 to 5% MeOH in DCM) to give Compound 37. ¹H NMR (400 MHz, Methanol-d4) δ 7.82 (s, 1H), 6.87 (d, J=4.4 Hz, 1H), 6.74 (d, J=4.5 Hz, 1H), 5.52 (d, J=4.8 Hz, 1H), 4.97-4.87 (m, 1H), 4.66-4.57 (m, 2H), 4.49 (d, J=5.5 Hz, 1H), 4.40 (d, J=11.7 Hz, 1H), 2.28-2.17 (m, 2H), 1.93-1.80 (m, 2H), 1.17 (s, 3H), 1.15 (s, 3H). MS m/z [M+1]=417.9

### Example 38: ((2R,3S,4R,5S)-5-(4-aminopyrrolo[2,1-f][1,2,4]triazin-7-yl)-2-cyano-3,4-dihydroxytetrahydrofuran-2-yl)methyl ((R)-1-methoxypropan-2-yl) carbonate

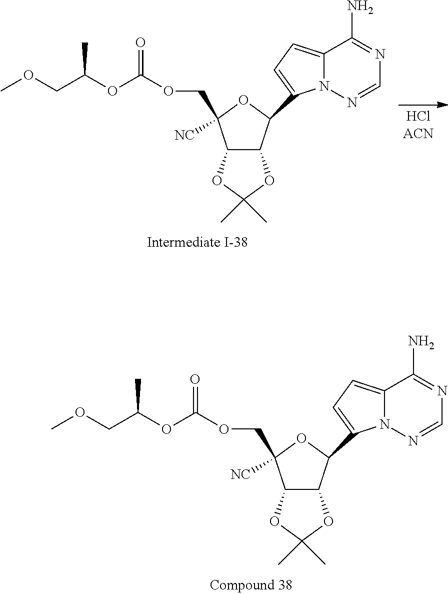 [SMILES: COC[C@@H](C)OC(=O)OC[C@@]1(C#N)O[C@@H](c2ccc3c(N)ncnn23)[C@@H]2OC(C)(C)O[C@@H]21 ; COC[C@@H](C)OC(=O)OC[C@@]1(C#N)O[C@@H](c2ccc3c(N)ncnn23)[C@@H]2OC(C)(C)O[C@@H]21]

To a solution of Intermediate I-38 (267 mg, 0.597 mmol) in ACN (2 mL) was added 25% HCl (0.4 mL) at rt. The mixture was stirred for 2 h, diluted with EtOAc (20 mL), and aqNaHCO3 (10 mL) added slowly. The mixture was stirred for 10 min, the phases separated, and the aqueous phase extracted with EtOAc (10 mL×3). The combined organic phase was dried under sodium sulfate, concentrated in vacuo, and purified by silica gel column chromatography (0 to 5% MeOH in DCM) to give Compound 38. ¹H NMR (400 MHz, Methanol-d4) δ 7.82 (s, 1H), 6.87 (d, J=4.5 Hz, 1H), 6.75 (d, J=4.6 Hz, 1H), 5.53 (d, J=4.8 Hz, 1H), 4.96-4.86 (m, 1H), 4.68-4.57 (m, 2H), 4.50 (d, J=5.5 Hz, 1H), 4.43 (d, J=11.7 Hz, 1H), 3.53-3.38 (m, 2H), 3.35 (s, 3H), 1.24 (d, J=6.5 Hz, 3H). MS m/z [M+1]=407.9

### Example 39: ((2R,3S,4R,5S)-5-(4-aminopyrrolo[2,1-f][1,2,4]triazin-7-yl)-2-cyano-3,4-dihydroxytetrahydrofuran-2-yl)methyl ((1R,3r,5S)-bicyclo[3.1.0]hexan-3-yl) carbonate

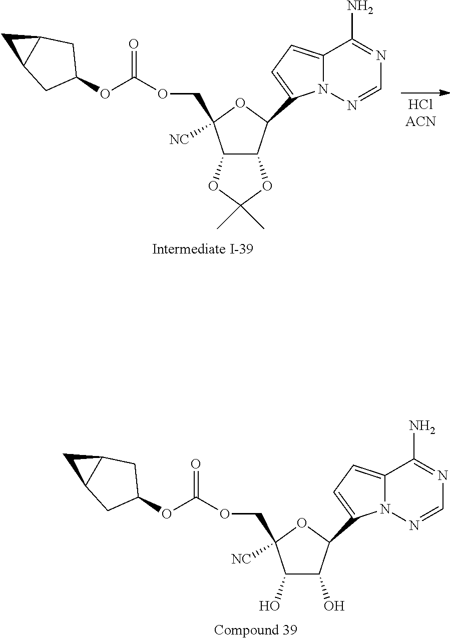 [SMILES: CC1(C)O[C@H]2[C@H](c3ccc4c(N)ncnn34)O[C@](C#N)(COC(=O)O[C@@H]3C[C@@H]4C[C@@H]4C3)[C@H]2O1 ; N#C[C@]1(COC(=O)O[C@@H]2C[C@@H]3C[C@@H]3C2)O[C@@H](c2ccc3c(N)ncnn23)[C@H](O)[C@@H]1O]

To a solution of Intermediate I-39 (221 mg, 0.485 mmol) in ACN (2 mL) was added 25% HCl (0.4 mL) at rt. The mixture was stirred for 2 h, diluted with EtOAc (20 mL), and aqNaHCO3 (10 mL) added slowly. The mixture was stirred for 10 min, the phases separated, and the aqueous phase extracted with EtOAc (10 mL×3). The combined organic phase was dried under sodium sulfate, concentrated in vacuo, and purified by silica gel column chromatography (0 to 5% MeOH in DCM) to give Compound 39. ¹H NMR (400 MHz, Methanol-d4) δ 7.82 (s, 1H), 6.88 (d, J=4.5 Hz, 1H), 6.74 (d, J=4.5 Hz, 1H), 5.53 (d, J=4.7 Hz, 1H), 5.10 (t, J=6.8 Hz, 1H), 4.64-4.56 (m, 2H), 4.47 (d, J=5.5 Hz, 1H), 4.39 (d, J=11.7 Hz, 1H), 2.30-2.12 (m, 2H), 1.91 (d, J=1.8 Hz, 1H), 1.87 (d, J=1.8 Hz, 1H), 1.41-1.26 (m, 2H), 0.56-0.47 (m, 1H), 0.39 (q, J=4.2 Hz, 1H). MS m/z [M+1]=416.0

### Example 40: ((2R,3S,4R,5S)-5-(4-aminopyrrolo[2,1-f][1,2,4]triazin-7-yl)-2-cyano-3,4-dihydroxytetrahydrofuran-2-yl)methyl ((1r,4R)-4-methylcyclohexyl) carbonate

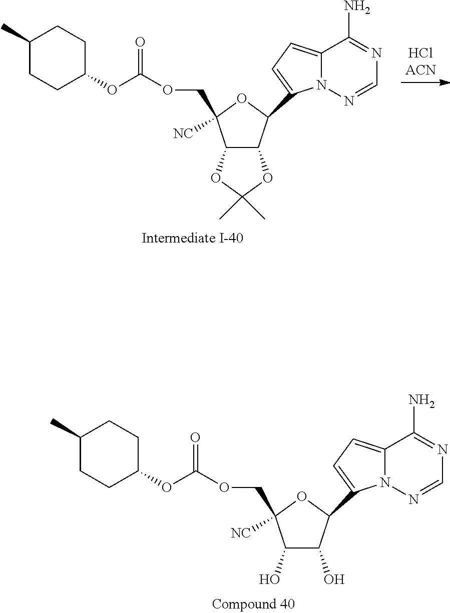 [SMILES: CC1(C)O[C@H]2[C@H](c3ccc4c(N)ncnn34)O[C@](C#N)(COC(=O)O[C@H]3CC[C@H](C)CC3)[C@H]2O1 ; C[C@H]1CC[C@H](OC(=O)OC[C@@]2(C#N)O[C@@H](c3ccc4c(N)ncnn34)[C@H](O)[C@@H]2O)CC1]

To a solution of Intermediate I-40 (136 mg, 0.288 mmol) in ACN (1 mL) was added 25% HCl (0.2 mL) at rt. The mixture was stirred for 2 h, diluted with EtOAc (20 mL), and aqNaHCO3 (10 mL) added slowly. The mixture was stirred for 10 min, the phases separated, and the aqueous phase extracted with EtOAc (10 mL×3). The combined organic phase was dried under sodium sulfate, concentrated in vacuo, and purified by silica gel column chromatography (0 to 5% MeOH in DCM) to give Compound 40. ¹H NMR (400 MHz, DMSO-d6) δ 7.86 (d, J=0.8 Hz, 1H), 7.77 (s, 2H), 6.87 (d, J=4.6 Hz, 1H), 6.72 (d, J=4.7 Hz, 1H), 6.14 (dd, J=5.9, 0.9 Hz, 1H), 5.51 (dd, J=5.7, 0.9 Hz, 1H), 5.39 (d, J=5.7 Hz, 1H), 4.55-4.39 (m, 3H), 4.37-4.20 (m, 2H), 1.99-1.83 (m, 2H), 1.73-1.62 (m, 2H), 1.40-1.23 (m, 3H), 1.07-0.92 (m, 2H), 0.86 (d, J=6.5 Hz, 3H). MS m/z [M+1]=432.0

### Example 42: Isopropyl (7-((2S,3S,4S,5R)-5-cyano-3,4-bis((isopropoxycarbonyl)oxy)-5-(((isopropoxycarbonyl)oxy)methyl)tetrahydrofuran-2-yl)pyrrolo[2,1-f][1,2,4]triazin-4-yl)carbamate

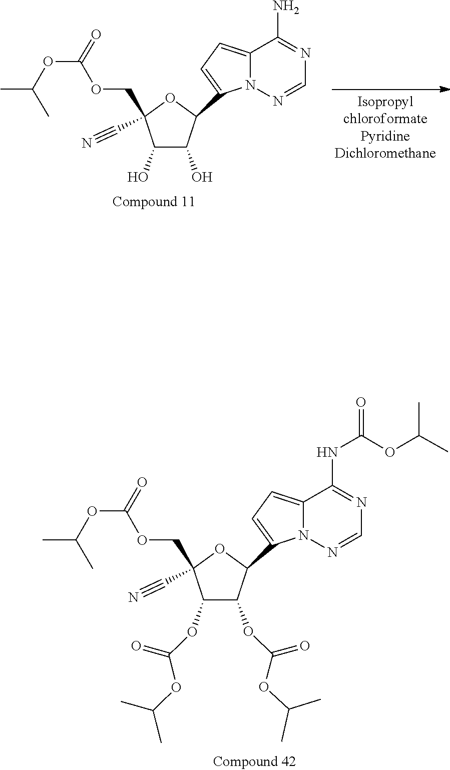 [SMILES: CC(C)OC(=O)OC[C@@]1(C#N)O[C@@H](c2ccc3c(N)ncnn23)[C@H](O)[C@@H]1O ; CC(C)OC(=O)Nc1ncnn2c([C@@H]3O[C@](C#N)(COC(=O)OC(C)C)[C@@H](OC(=O)OC(C)C)[C@H]3OC(=O)OC(C)C)ccc12]

To a solution of Compound 11 (100 mg, 0.27 mmol) in dichloromethane (15 mL) isopropyl chloroformate (130 mg, 1 mmol) was added followed by pyridine (84 mg, 1 mmol) and stirred at room temperature for 1h. After completion of the reaction, diluted the reaction mixture with dichloromethane, washed with water, brine, dried and concentrated. The residue was purified by flash chromatography using silica gel column chromatography (0 to 10% MeOH in DCM) to give Compound 42. ¹H NMR (400 MHz, Methanol-d4) δ 8.21 (s, 1H), 7.24 (d, J=4.7 Hz, 1H), 7.01 (d, J=4.7 Hz, 1H), 5.76 (h, J=5.0 Hz, 4H), 5.11 (p, J=6.3 Hz, 1H), 4.99-4.89 (m, 2H), 4.83 (dd, J=6.3, 1.6 Hz, 1H), 4.68 (d, J=11.9 Hz, 1H), 4.52 (d, J=11.9 Hz, 1H), 1.37 (dd, J=7.8, 6.2 Hz, 12H), 1.31-1.25 (m, 12H). MS m/z [M+1]=636.2

### Example 43: (2R,3S,4S,5S)-5-(4-aminopyrrolo[2,1-f][1,2,4]triazin-7-yl)-2-cyano-2-(((isopropoxycarbonyl)oxy)methyl)tetrahydrofuran-3,4-diyl diisopropyl bis(carbonate)

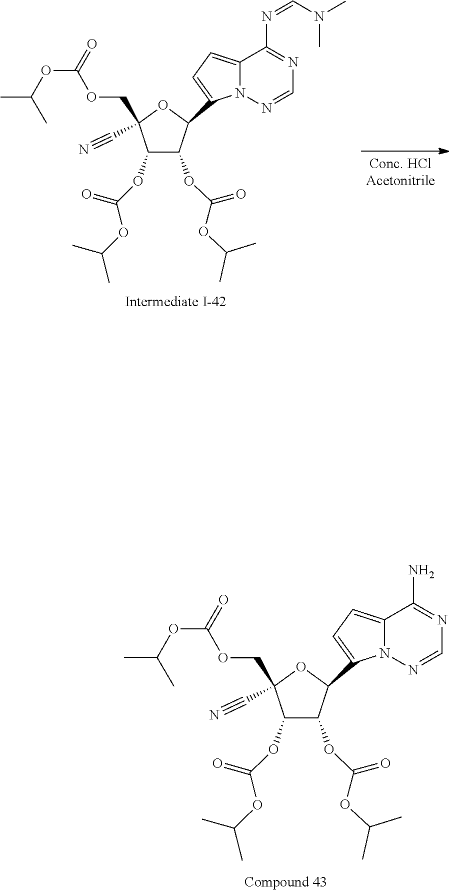

To a solution of Intermediate I-42 (200 mg, 0.33 mmol) in acetonitrile (10 mL), Conc. HCl (0.6 mL, 7.5 mmol) was added and stirred at room temperature for 2 h. After completion of the reaction, diluted with ethyl acetate (50 mL), neutralized with aqueous sodium bicarbonate, separated the organic layer, washed with water, brine, dried and concentrated. The residue is purified by flash chromatography using 0-20% MeOH in DCM as eluent to afford Compound 43. ¹H NMR (400 MHz, Methanol-d4) δ 7.88 (s, 1H), 6.90 (d, J=4.6 Hz, 1H), 6.82 (d, J=4.6 Hz, 1H), 5.82 (d, J=5.9 Hz, 1H), 5.75 (dd, J=5.9, 4.7 Hz, 1H), 5.68 (d, J=4.7 Hz, 1H), 4.94 (p, J=6.2 Hz, 1H), 4.85-4.80 (m, 2H), 4.68 (d, J=11.8 Hz, 1H), 4.50 (d, J=11.8 Hz, 1H), 1.36 (t, J=5.9 Hz, 6H), 1.33-1.24 (m, 12H). MS m/z [M+1]=550.1

### Example 44: Methyl (7-((2S,3S,4S,5R)-5-cyano-3,4-bis((methoxycarbonyl)oxy)-5-(((methoxycarbonyl)oxy)methyl)tetrahydrofuran-2-yl)pyrrolo[2,1-f][1,2,4]triazin-4-yl)carbamate

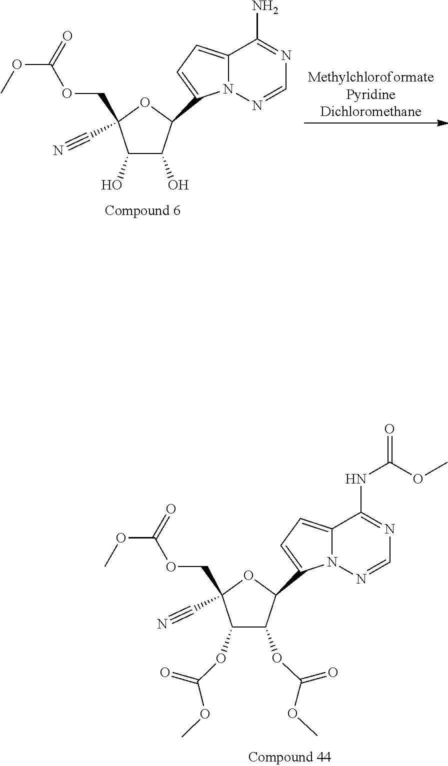

To a solution of Compound 6B (75 mg, 0.2 mmol) in dichloromethane (15 mL) methyl chloroformate (104 mg, 0.8 mmol) was added followed by pyridine (64 mg, 0.8 mmol) and stirred at room temperature for 1h. After completion of the reaction, diluted the reaction mixture with dichloromethane, washed with water, brine, dried and concentrated. The residue was purified by flash chromatography using silica gel column chromatography (0 to 10% MeOH in DCM) to provide Compound 44. ¹H NMR (400 MHz, Methanol-d4) δ 8.18 (s, 1H), 7.20 (d, J=4.7 Hz, 1H), 7.05-6.93 (m, 1H), 5.83 (dt, J=4.4, 2.2 Hz, 1H), 5.78 (d, J=1.5 Hz, 2H), 4.72 (d, J=11.9 Hz, 1H), 4.55 (d, J=11.9 Hz, 1H), 3.89 (d, J=9.1 Hz, 6H), 3.80 (d, J=8.7 Hz, 6H). MS m/z [M+1]=447.9

### Example 45: (2R,3S,4S,5S)-5-(4-aminopyrrolo[2,1-f][1,2,4]triazin-7-yl)-2-cyano-2-(((methoxycarbonyl)oxy)methyl)tetrahydrofuran-3,4-diyl dimethyl bis(carbonate)

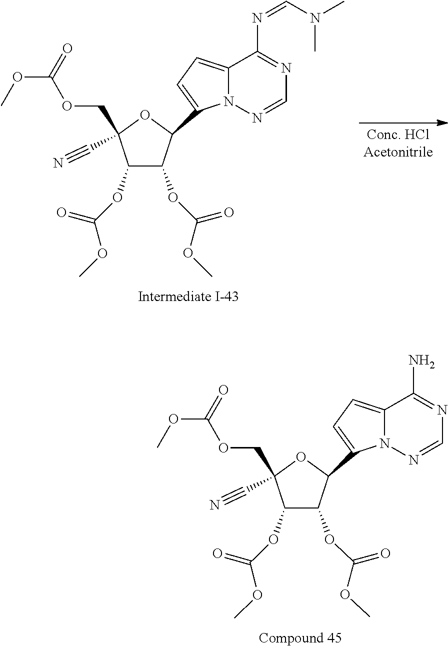

To a solution of Intermediate I-43 (100 mg, 0.2 mmol) in acetonitrile (10 mL), Conc. HCl (0.6 mL, 7.5 mmol) was added and stirred at room temperature for 2 h. After completion of the reaction, diluted with ethyl acetate (50 mL), neutralized with aqueous sodium bicarbonate, separated the organic layer, washed with water, brine, dried and concentrated. The residue is purified by flash chromatography using 0-20% MeOH in DCM as eluent to afford Compound 45. ¹H NMR (400 MHz, Methanol-d4) δ 7.87 (s, 1H), 6.88 (d, J=4.5 Hz, 1H), 6.81 (d, J=4.5 Hz, 1H), 5.88 (d, J=5.9 Hz, 1H), 5.78 (dd, J=5.9, 4.5 Hz, 1H), 5.70 (d, J=4.5 Hz, 1H), 4.71 (d, J=11.8 Hz, 1H), 4.53 (d, J=11.8 Hz, 1H), 3.89 (s, 3H), 3.80 (s, 3H), 3.78 (s, 3H). MS m/z [M+1]=466.1

### D. Biological Examples

### Example A. DENV-2 moDC EC₅₀

Human monocyte-derived dendritic cells (moDCs) were derived from CD14+ monocytes (AllCells) cultured in Human Mo-DC Differentiation medium containing GM-CSF and IL-4 (Miltenyi Biotec). On day 7, moDCs were harvested by mechanical disruption, washed and suspended in serum-free RPMI. moDCs were infected with Vero-derived Dengue 2, New Guinea strain (NGC) at a MOI=0.1 for two hours in serum-free RPMI with gentle agitation at 37° C. Cells were washed and resuspended in 10% serum-containing RPMI (Gibco, supplemented with sodium pyruvate, NEAA, Penicillin-Streptomycin). 10≡cells were plated in triplicate in 96-well plates with compounds dispensed at graded doses (Hewlett-Packard D300 Digital Dispenser). All wells were normalized to 0.25% DMSO. At 48 hours, cells were washed with 1× PBS and all supernatants removed. Total RNA was extracted using RNEasy 96 plates (Qiagen) and used to generate first-strand cDNA using XLT cDNA 5× Supermix (QuantaBio). cDNA was used as a template in a Taqman qPCR duplex reaction specific to DENV2 viral and GAPDH gene expression. EC₅₀ values were determined using Prism Graphpad software, with normalization to a positive control and no compound negative control wells.

### Example B. moDC CC₅₀

Human monocyte-derived dendritic cells (moDCs) were derived from CD14+ monocytes (AllCells) cultured in Human Mo-DC Differentiation medium containing GM-CSF and IL-4 (Miltenyi Biotec). On day 7, moDCs were harvested by mechanical disruption, washed and cultured in triplicate at 1×10\hat{ }5-5×10\hat{ }4 cells/well in 96-well plates with compounds dispensed at graded doses (Hewlett-Packard D300 Digital Dispenser). All wells were normalized to 0.25% DMSO. After 48 hours, CellTiter Glo (Promega) was added and incubated for 10 minutes at room temp before reading on a luminometer. % viability curves were calculated against no compound and no cell control wells. CC₅₀ values were determined using Prism Graphpad software.

### Example C. DENV-2 Huh-7 EC₅₀

Huh7 (Human hepatocarcinoma 7) cells were maintained in 10% FCS-containing DMEM complete media. On the day of the assay, cells were trypsinized (0.1% Trypsin-EDTA), washed and infected for 2 hours in serum-free DMEM with Dengue serotype 2 New Guinea C (NGC) strain at MOI=0.1 with gentle agitation at 37° C. After 2 hours, cells were washed with serum-free media and suspended in 10% FCS-containing DMEM (Gibco, supplemented with sodium pyruvate, NEAA, Penicillin-Streptomycin). 10\hat{ }5 cells were plated in triplicate in 96-well plates with compounds dispensed at graded doses (Hewlett-Packard D300 Digital Dispenser). All wells were normalized to 0.25% DMSO. At 48 hours, cells were washed with 1× PBS and all supernatants removed. Total RNA was extracted using RNEasy 96 plates (Qiagen) and used to generate first-strand cDNA using XLT cDNA 5× Supermix (QuantaBio). cDNA was used as a template in a Taqman qPCR duplex reaction specific to DENV2 viral and GAPDH gene expression. EC₅₀ values were determined using Prism Graphpad software, with normalization to a positive control and no compound negative control wells.

### Example D. Huh-7 CC₅₀

Human hepatocarcinoma 7 (Huh7) cells were maintained in 10% FCS-containing complete DMEM. On day of assay, cells were trypsinized with 0.1% Trypsin-EDTA, washed and cultured in triplicate at 1-2×10\hat{ }4 cells/well in 96-well plates with compounds dispensed at graded doses (Hewlett-Packard D300 Digital Dispenser). All wells were normalized to 0.25% DMSO. After 48 hours, CellTiter Glo (Promega) was added and incubated for 10 minutes at room temp before reading on a luminometer. % viability curves were calculated against no compound and no cell control wells. CC₅₀ values were determined using Prism Graphpad software.

### Example E. RSV HEp-2 EC₅₀

Antiviral activity against RSV is determined using an infectious cytopathic cell protection assay in HEp-2 cells. In this assay, compounds inhibiting viral infection and/or replication produce a cytoprotective effect against the virus-induced cell killing that can be quantified using a cell viability reagent. The techniques used here are novel adaptations of methods described in published literature (Chapman et al., ANTIMICROB AGENTS CHEMOTHER. 2007, 51(9):3346-53).

HEp-2 cells are obtained from ATCC (Manassas, VI) and maintained in MEM media supplemented with 10% fetal bovine serum and penicillin/streptomycin. Cells are passaged twice a week and kept at subconfluent stage. Commercial stock of RSV strain A2 (Advanced Biotechnologies, Columbia, MD) is titered before compound testing to determine the appropriate dilution of the virus stock that generates desirable cytopathic effect in HEp-2 cells.

For antiviral tests, HEp-2 cells are grown in large cell culture flasks to near confluency but not fully so. The compounds to be tested are prediluted in DMSO in 384-well compound dilution plates, either in an 8 or 40 sample per plate standardized dose response format. 3-fold serial dilution increments of each test compound are prepared in the plates and test samples are transferred via acoustic transfer apparatus (Echo, Labcyte) at 100 nL per well into cell culture assay 384-well plates. Each compound dilution is transferred in single or quadruplicate samples into dry assay plates, which are stored until assay is ready to go. The positive and negative controls are laid out in opposite on ends of the plate in vertical blocks (1 column).

Subsequently, an infectious mixture is prepared using an appropriate dilution of virus stock previously determined by titration with cells at a density of 50,000/ml and 20 μL/well is added to test plates w/compounds via automation (uFlow, Biotek). Each plate includes negative and positive controls (16 replicates each) to create 0% and 100% virus inhibition standards, respectively. Following the infection with RSV, testing plates are incubated for 4 days in a 37° C. cell culture incubator. After the incubation, a cell viability reagent, Cell TiterGlo (Promega, Madison, WI) is added to the assay plates, which are incubated briefly, and a luminescent readout is measured (Envision, Perkin Elmer) in all the assay plates. The RSV-induced cytopathic effect, percentage inhibition, is determined from the levels of remaining cell viability. These numbers are calculated for each tested concentration relative to the 0% and 100% inhibition controls, and the EC₅₀ value for each compound is determined by non-linear regression as a concentration inhibiting the RSV-induced cytopathic effect by 50%. Various potent anti-RSV tool compounds are used as positive controls for antiviral activity.

### Example F. HEp-2 CC₅₀

Cytotoxicity of tested compounds is determined in uninfected HEp-2 cells in parallel with the antiviral activity using the cell viability reagent in a similar fashion as described before for other cell types (Cihlar et al., ANTIMICROB AGENTS CHEMOTHER. 2008, 52(2):655-65). The same protocol as for the determination of antiviral activity is used for the measurement of compound cytotoxicity except that the cells are not infected with RSV. Instead, an uninfected cell mixture at the same density is added at 20 ul/well to plates containing prediluted compounds, also at 100 nL/sample. Assay plates are then incubated for 4 days followed by a cell viability test using the same CellTiter Glo reagent addition and measurement of luminescent readouts. Untreated cell and cells treated at 2 μM puromycin (Sigma, St. Louis, MO) serve as 100% and 0% cell viability control, respectively. The percent of cell viability is calculated for each tested compound concentration relative to the 0% and 100% controls and the CC₅₀ value is determined by non-linear regression as a compound concentration reducing the cell viability by 50%.

### Example G. HEp-2 and MT4 CC₅₀

Cytotoxicity of the compounds was determined in uninfected cells using the cell viability reagent in a similar fashion as described before for other cell types (Cihlar et al., ANTIMICROB AGENTS CHEMOTHER. 2008, 52(2):655-65). HEp-2 (1.5×103 cells/well) and MT-4 (2×103 cells/well) cells were plated in 384-well plates and incubated with the appropriate medium containing 3-fold serially diluted compound ranging from 15 nM to 100,000 nM. Cells were cultured for 4-5 days at 37° C. Following the incubation, the cells were allowed to equilibrate to 25° C., and cell viability was determined by adding Cell-Titer Glo viability reagent. The mixture was incubated for 10 min, and the luminescence signal was quantified using an Envision plate reader. Untreated cell and cells treated at 2 μM puromycin (Sigma, St. Louis, MO) serve as 100% and 0% cell viability control, respectively. The percent of cell viability was calculated for each tested compound concentration relative to the 0% and 100% controls and the CC₅₀ value was determined by non-linear regression as a compound concentration reducing the cell viability by 50%.

### Example H. RSV NHBE EC₅₀

Normal human bronchial epithelial (NHBE) cells were purchased from Lonza (Walkersville, MD, Cat #CC-2540) and cultured in Bronchial Epithelial Growth Media (BEGM) (Lonza, Walkersville, MD, Cat #CC-3170). The cells were passaged 1-2 times per week to maintain <80% confluency. The NHBE cells were discarded after 6 passages in culture.

To conduct the RSV A2 antiviral assay, NHBE cells were plated in 96-well plates at a density of 7,500 cells per well in BEGM and allowed to attach overnight at 37° C. Following attachment, 100 μL of cell culture media was removed and 3-fold serially diluted compound was added using a Hewlett-Packard D300 Digital Dispenser. The final concentration of DMSO was normalized to 0.05%. Following compound addition, the NHBE cells were infected by the addition of 100 μL of RSV A2 at a titer of 1×10^{4.5} tissue culture infectious doses/mL in BEGM and then incubated at 37° C. for 4 days. The NHBE cells were then allowed to equilibrate to 25° C. and cell viability was determined by removing 100 μL of culture medium and adding 100 μL of Cell-Titer Glo viability reagent. The mixtures were incubated for 10 minutes at 25° C., and the luminescence signal was quantified on an Envision luminescence plate reader.

### Example L RSV NHBE FLuc EC₅₀

Normal human bronchial epithelial (NHBE) cells are purchased from Lonza (Walkersville, MD Cat #CC-2540) and maintained in Bronchial Epithelial Cell Growth Medium (BEGM) (Lonza, Walkersville, MD, Cat #CC-3170) with all provided supplements in the BulletKit. Cells are passaged 2-3 times per week to maintain sub-confluent densities and are used for experiments at passages 2-4.

Recombinant Respiratory Syncytial virus strain A2 containing the firefly luciferase reporter between the P and M genes (RSV-Fluc, 6.3×10⁶ TCID₅₀/mL) is purchased from Viratree (Durham, NC, Cat #R145).

NHBE cells (5×10³/well) are seeded in 100 μL white wall/clear bottom 96-well plates (Corning) with culture medium and are incubated for 24 hours at 37° C. with 5% CO₂. On the following day, three-fold serial dilutions of compounds prepared in DMSO are added to the wells using the HP D300e digital dispenser with normalization to the highest concentration of DMSO in all wells. The cells are then infected with RSV-Fluc diluted with BEGM media at an MOI of 0.1 for a final volume of 200 μL media/well. Uninfected and untreated wells are included as controls to determine compound efficacy against RSV-Fluc. Following incubation with compound and virus for three days at 37° C. with 5% CO₂, 100 μL of culture supernatant is removed from each well and replaced with 100 μL of ONE-Glo luciferase reagent (Promega, Madison, WI, Cat #E6110). The plates are gently mixed by rocking for 10 minutes at 21° C. and luminescence signal is measured using an Envision plate reader (PerkinElmer). Values are normalized to the uninfected and infected DMSO controls (0% and 100% infection, respectively). Non-linear regression analysis is applied to determine the compound concentration at which 50% luminescence signal is reduced (EC₅₀) using the XLfit4 add-in for MICROSOFT® EXCEL®. All experiments are performed in duplicate with two technical repeats each.

### Example J. RSV NHBE CC₅₀

NHBE cells were seeded in black 384-TC-treated plates (Corning) at 2×10³ cells/well in a final volume of 20 μL BEBM+supplements (Lonza). The next day, add 0.1 μL of compound was added to the assay plates using an Echo acoustic dispenser. Plates were incubated for 3 additional days at 37° C. and 5% CO₂. On day 3 of treatment, 20 μL of CellTiter Glo (Promega) was added to each well using a Biotek dispenser. After a 10-minute incubation, luminescence signal was measured with 0.1 sec integration time using an EnVision (Perkin-Elmer) plate reader. Values were normalized to the DMSO- and puromycin-treated controls (0% and 100% cell death, respectively). Data was fit using non-linear regression analysis and CC₅₀ values were then determined as the concentration reducing the luciferase signal by 50%. The compiled data was generated based on least two independent experimental replicates, each containing technical quadruplicates for each concentration.

### Example K. RSV HAE EC₅₀

HAE cells are cultured at the air-liquid interface and have an apical side that is exposed to the air and a basal side that is in contact with the medium. Prior to experimentation, HAE were removed from their agar-based shipping packaging and were acclimated to 37° C./5% CO₂ overnight in lml of HAE Assay medium (AIR-100-MM, Mattek Corp). HAE were prepared for infection by washing the apical surface twice with 400 μL of PBS (either utilizing direct pipetting methods or by running each transwell through a trough containing PBS) to remove the mucus layer. Apical chambers were drained of PBS and tapped gently onto absorbent material to remove as much PBS as possible. After washing, the cells were transferred to fresh HAE maintenance media containing 4-fold serially diluted compound, delivered to the basal side of the cell monolayer, and apically infected with 100 μL of a 1:600 dilution of RSV A strain A2 1000× stock (ABI, Columbia, MD, cat #10-124-000) in HAE assay medium for 3 hours at 37° C. in 5% CO₂. The virus inoculum was removed and the apical surface of the cells was washed 3 times with PBS using either method previously described. The cells were then cultured in the presence of compound for 3 days at 37° C. Following the incubation, total RNA was extracted from the HAE cells using a MagMAX-96 Viral RNA isolation kit (Applied Biosystems, Foster City, CA, Cat #AM1836) and intracellular RSV RNA was quantified by real-time PCR. Approximately 25 ng of purified RNA was added to a PCR reaction mixture that contained 0.9 μM RSV N Forward and RSV N Reverse primers, 0.2 μM RSV N Probe and 1× Taqman RNA-to-Ct 1-Step Kit (Applied Biosystems, Foster City, CA, Cat #4392938). RNA levels were normalized using a Tagman GAPDH control primer set (Applied Biosystems, Foster City, CA, Cat #402869). Real-Time PCR Primers and Probe Used in the RSV A2 HAE Antiviral Assay: RSV N Forward CATCCAGCAAATACACCATCCA (SEQ ID NO: 1), RSV N Reverse TTCTGCACATCATAATTAGGAGTATCAA (SEQ ID NO: 2), RSV N Probe FAM-CGGAGCACAGGAGAT-BHQ (SEQ ID NO: 3).

### Example L. HRV16 HELA EC₅₀

H1-HeLa cells, cultured in complete DMEM medium containing 10% heat-inactivated FBS and 1% Penicillin/Streptomycin, were seeded in 96 well plates at 3000 cells/well one day prior to compound dosing and infection. The antiviral activity of each compound was measured in triplicate. Compounds were added directly to the cell cultures in serial 3-fold dilutions using the HP300 digital dispenser (Hewlett Packard, Palo Alto, CA) immediately prior to infection. The plates were transferred to BSL-2 containment and the appropriate dilution of virus stock, previously determined by titration and prepared in cell culture media, was added to test plates containing cells and serially diluted compounds. Each plate included 6 wells of infected untreated cells and 6 wells of uninfected cells that served as 0% and 100% virus inhibition control, respectively. Following the infection, test plates were incubated for 96 h in a tissue culture incubator set to 33° C./5% CO₂. Following incubation, the H1-HeLa cells were removed from incubation and allowed to equilibrate to 25° C. Cell viability was determined by removing 100 μL of culture medium and adding 100 μL of Cell-Titer Glo viability reagent. The mixtures were incubated on a shaker for 10 minutes at 25° C., and the luminescence signal was quantified on an Envision luminescence plate reader. The percentage inhibition of virus infection was calculated for each tested concentration relative to the 0% and 100% inhibition controls and the EC₅₀ value for each compound was determined by 4-parametric non-linear regression as the effective concentration of compound that inhibited cytopathic effect by 50%.

### Example M. HRV1A HELA EC₅₀

H1-HeLa cells, cultured in complete RPMI 1640 medium containing 10% heat-inactivated FBS and 1% Penicillin/Streptomycin, were seeded in 96 well plates at 5000 cells/well one day prior to compound dosing and infection. The antiviral activity of each compound was measured in triplicate. Compounds were added directly to the cell cultures in serial 3-fold dilutions using the HP300 digital dispenser (Hewlett Packard, Palo Alto, CA) immediately prior to infection. The plates were transferred to BSL-2 containment and 100 μL of 1/4000 dilution of HRV1a virus stock was added to each well containing cells and serially diluted compounds. Each plate included 6 wells of infected untreated cells and 6 wells of cells containing 5 μM Rupintrivir that served as 0% and 100% virus inhibition control, respectively. Following the infection, test plates were incubated for 96 h in a tissue culture incubator set to 37° C./5% CO₂. Following incubation, the H1-HeLa cells were removed from incubation and allowed to equilibrate to 25° C. Cell viability was determined by removing 100 μL of culture medium and adding 100 μL of Cell-Titer Glo viability reagent. The mixtures were incubated on a shaker for 10 minutes at 25° C., and the luminescence signal was quantified on an Envision luminescence plate reader. The percentage inhibition of virus infection was calculated for each tested concentration relative to the 0% and 100% inhibition controls and the EC₅₀ value for each compound was determined by 4-parametric non-linear regression as the effective concentration of compound that inhibited cytopathic effect by 50%.

### Example N. HRV14 HELA EC₅₀

H1-HeLa cells, cultured in complete RPMI 1640 medium containing 10% heat-inactivated FBS and 1% Penicillin/Streptomycin, were seeded in 96 well plates at 5000 cells/well one day prior to compound dosing and infection. The antiviral activity of each compound was measured in triplicate. Compounds were added directly to the cell cultures in serial 3-fold dilutions using the HP300 digital dispenser (Hewlett Packard, Palo Alto, CA) immediately prior to infection. The plates were transferred to BSL-2 containment and 100 μL of 1/4000 dilution of HRV14 virus stock was added to each well containing cells and serially diluted compounds. Each plate included 6 wells of infected untreated cells and 6 wells of cells containing 5 μM Rupintrivir that served as 0% and 100% virus inhibition control, respectively. Following the infection, test plates were incubated for 96 h in a tissue culture incubator set to 37° C./5% CO₂. Following incubation, the H1-HeLa cells were removed from incubation and allowed to equilibrate to 25° C. Cell viability was determined by removing 100 μL of culture medium and adding 100 μL of Cell-Titer Glo viability reagent. The mixtures were incubated on a shaker for 10 minutes at 25° C., and the luminescence signal was quantified on an Envision luminescence plate reader. The percentage inhibition of virus infection was calculated for each tested concentration relative to the 0% and 100% inhibition controls and the EC₅₀ value for each compound was determined by 4-parametric non-linear regression as the effective concentration of compound that inhibited cytopathic effect by 50%.

### Example O. HRVc15 and HRVc25 EC₅₀

First, HRV replicon RNA is prepared. Sug of DNA Template (HRVc15 or HRVc25) is linearized with 2 μL of MluI enzyme in NEB buffer-3 in a final volume of 25 μL for 3 hours at 37° C. Following incubation, linearized DNA is purified on a PCR purification column and the following in vitro transcription is performed using the following conditions: 10 μL of RiboMAX Express T7 2× buffer, 1-8 μL of linear DNA template (1 μg), 0-7 μL nuclease free water, 2 μL enzyme mix T7 express. The final volume of 20 μL is mixed and incubated at 37° C. for 30 min. Following incubation, 1 μL of RQ1 RNase free DNase is added and the mixture is incubated at 37° C. for 15 min. The resulting RNA is then purified with the MegaClear Kit (Gibco Life Technologies cat #11835-030) and is eluted two times with 50 μL of elution buffer at 95° C. H1-HeLa cells cultured in complete RPMI 1640 media containing 10% heat-inactivated FBS and 1% Penicillin/Streptomycin are seeded into T-225 flasks at a concentration of 2E6 cells/flask a day prior to transfection and are incubated at 37° C./5% CO₂ overnight. On the day of transfection, cells are trypsinized following standard cell culture protocols and are washed two times with PBS. Following washes, cells are resuspended at a concentration of 1E7 cells/mL in PBS and the suspension is stored on wet ice. Electroporation is used to introduce replicon RNA into the H1-HeLa cells. A final volume of 10 μL containing 10 μg of replicon c15 or 1 μg of c25 replicon RNA, respectively, are pipetted into a 4 mm electroporation cuvette. The H1-HeLa cell stock is mixed by gently swirling and 0.5 mL of the cell stock previously prepared is transferred into the cuvette containing the replicon RNA. The combined solution is flicked to mix. Following mixing, cells are immediately electroporated using the following settings: 900V, 25 uF, infinite resistance, 1 pulse. Cuvettes are rested on ice for 10 min. Following the 10 min incubation, add 19 mL of ambient temperature, phenol red-free and antiobiotic-free RPMI 1640 containing 10% heat-inactivated FBS per electroporation. 150 μL (4E4 cells) of the electroporated cell suspension are seeded per well into a 96well clear-bottom, white cell culture plate, and are incubated at 25° C. for 30 min. Compounds were added directly to the cell cultures in serial 3-fold dilutions using the HP300 digital dispenser (Hewlett Packard, Palo Alto, CA) and were tested in triplicate. Following the addition of compounds, plates are incubated at 33° C. for 48 hrs. Replicon activity is then measured by a Renilla-Glo Luciferase Assay system. Prior to signal quantification, plates are removed from incubators and are allowed to equilibrate to 25° C. after 50 uL is removed from each well. Following manufacturer's protocol, a 1:100 dilution of Renilla-Glo substrate to buffer is prepared and 100 uL of the Renill-Glo luciferase mix is added to each well. Plates are then incubated for 20 min at 25° C. under gentle agitation and luciferase signal are determined with a 0.1 second detection setting using an EnVision luciferase quantification reader. The percentage inhibition of replicon inhibition was calculated for each tested concentration relative to the 0% and 100% inhibition controls included in the experiments and the EC₅₀ value for each compound was determined by 4-parametric non-linear regression as the effective concentration of compound that inhibited luciferase signal by 50%.

### Example P. DENV-2 Huh-7 Rep EC₅₀

In 384 well plates (Greiner, Cat #781091), compounds were acoustically transferred at 200 nl per well in a 8 compound (4 replicates) or 40 compound dose response format (3 replicates). For all plates tested, Balapiravir, GS-5734 and NITD008 were included as positive inhibition controls alongside 0% inhibition, DMSO-only negative control wells. Following compound addition, Huh-7 cells containing the DENV2 replicon construct were harvested following standard cell culture procedures and were adjusted to a concentration of 1.25E5 cells/mL in cell culture media composed of cDMEM without genticin. 40 μL of the cell stock was then added to each well for a final cell density of 5,000 cells/well. Cell and compound mixtures were incubated at 37° C./5% CO₂ for 48 hours. Prior to harvesting cells, EnduRen Live Cell Substrate (Promega, Cat #E6481) was prepared by suspending 3.4 mg into 100 uL of DMSO to generate a 60 mM stock solution. The stock solution was then diluted 1:200 in pre-warmed cDMEM and 10 uL of this diluted solution was added to each well of the 384 well plates. Plates were then centrifuged at 500 rpm briefly and were placed on a plate shaker for 2 min. Following mixing, plates were incubated at 7° C./5% CO₂ for 1.5 hours prior to measuring luminescence on an Envision luminometer. The percentage inhibition of replicon signal was calculated for each tested concentration relative to the 0% and 100% inhibition controls and the EC₅₀ value for each compound was determined by 4-parametric non-linear regression as the effective concentration of compound that inhibited replicon signal by 50%.

### Example Q. HCV Rep 1B and 2A EC₅₀

Compounds were serially diluted in ten steps of 1:3 dilutions in 384-well plates. All serial dilutions were performed in four replicates per compound within the same 384-well plate. An HCV protease inhibitor ITMN-191 at 100 μM was added as a control of 100% inhibition of HCV replication while puromycin at 10 mM was included as a control of 100% cytotoxicity. To each well of a black polystyrene 384-well plate (Greiner Bio-one, Monroe, NC), 90 μL of cell culture medium (without Geneticin) containing 2000 suspended HCV replicon cells was added with a Biotek Flow workstation. For compound transfer into cell culture plates, 0.4 μL of compound solution from the compound serial dilution plate was transferred to the cell culture plate on a Biomek FX workstation. The DMSO concentration in the final assay wells was 0.44%. The plates were incubated for 3 days at 37° C. with 5% CO₂ and 85% humidity. The HCV replicon assay was a multiplex assay, able to assess both cytotoxicity and antireplicon activity from the same well. The CC₅₀ assay was performed first. The media in the 384-well cell culture plate was aspirated, and the wells were washed four times with 100 μL of PBS each, using a Biotek ELX405 plate washer. A volume of 50 μL of a solution containing 400 nM calcein AM (Anaspec, Fremont, CA) in 1× PBS was added to each well of the plate with a Biotek Flow workstation. The plate was incubated for 30 min at room temperature before the fluorescence signal (excitation 490 nm, emission 520 nm) was measured with a Perkin-Elmer Envision plate reader. The EC₅₀ assay was performed in the same wells as the CC₅₀ assay. The calcein-PBS solution in the 384-well cell culture plate was aspirated with a Biotek ELX405 plate washer. A volume of 20 μL of Dual-Glo luciferase buffer (Promega, Madison, WI) was added to each well of the plate with a Biotek Flow Workstation. The plate was incubated for 10 min at room temperature. A volume of 20 μL of a solution containing a 1:100 mixture of Dual-Glo Stop & Glo substrate (Promega, Madison, WI) and Dual-Glo Stop & Glo buffer (Promega, Madison, WI) was added to each well of the plate with a Biotek Flow Workstation. The plate was then incubated at room temperature for 10 min before the luminescence signal was measured with a Perkin-Elmer Envision Plate Reader.

### Example R. HEp-2 RSV-Luc5 384-Well Assay (EC50 RSVFLUC Hep2-384)

HEp-2 cell line was purchased from ATCC (Manassas, VA Cat #CCL-23) and maintained in Dulbecco's Minimum Essential Medium (DMEM) (Corning, New York, NY, Cat #15-018CM) supplemented with 10% fetal bovine serum (FBS) (Hyclone, Logan, UT, Cat #SH30071-03) and 1× Penicillin-Streptomycin-L-Glutamine (Corning, New York, NY, Cat #30-009-C1). Cells were passaged 2 times per week to maintain sub-confluent densities and were used for experiments at passage 5-20. Respiratory syncytial virus recombinant with luciferase (RSV-Luc5) direct pelleted virus (≥1×107 TCID50/ml) was purchased from Microbiologics (Saint Cloud, MN). Viral replication was determined in HEp-2 cells in the following manner.

Compounds are prepared in 384-well polypropylene plates (Greiner, Monroe, NC, Cat #784201) with 8 compounds per plate in grouped replicates of 4 at 10 serially diluted concentrations (1:3).

HEp-2 cells were suspended in DMEM (supplemented with 10% FBS and 1× Penicillin-Streptomycin-L-Glutamine) and 60 uL of 4,000 cells per well were seeded into 384-well plates (Greiner, Monroe, NC, Cat #781080) using Biotek MultiFlo dispenser. After overnight incubation at 37° C. and 5% CO₂, 0.4 uL of three-fold serial dilutions of compound was added to each well using a Biomek FX pipette station. RSV-Luc5 viruses were diluted in DMEM (supplemented with 10% FBS and 1× Penicillin-Streptomycin-L-Gluitamine) at an MOI=0.5. Virus suspension was added to each 384-well compound plate at 20 uL per well using a Biotek MultiFlo dispenser. The assay plates were incubated for 3 days at 37° C. and 5% CO₂. At the end of incubation, One-Glo reagent (Promega, Madison, WI, Cat #E6120) was prepared. The assay plate and the reagent were equilibrated to room temperature for 30 minutes. 50 uL per well of medium was removed from assay plate and 40 uL per well of One-Glo reagent was added to each plate by Biomek FX. The plates were sat at room temp for 15 minutes. Viral replication was then assessed by measuring luminescence signal using and Envision plate reader. Remdesivir was used as positive control and DMSO was used as negative control. Values were normalized to the positive and negative controls (as 0% and 100% replication, respectively) and data was fitted using non-linear regression analysis by Gilead's dose response tool. The EC₅₀ value for each compound was then determined as the concentration reducing the viral replication by 50%.

### Example S. HEp-2 and MT4 CC₅₀

Cytotoxicity of the compounds was determined in uninfected cells using the cell viability reagent in a similar fashion as described before for other cell types (Cihlar et al., ANTIMICROB AGENTS CHEMOTHER. 2008, 52(2):655-65). HEp-2 (1.5×103 cells/well) and MT-4 (2×103 cells/well) cells were plated in 384-well plates and incubated with the appropriate medium containing 3-fold serially diluted compound ranging from 15 nM to 100,000 nM. Cells were cultured for 4-5 days at 37° C. Following the incubation, the cells were allowed to equilibrate to 25° C., and cell viability was determined by adding Cell-Titer Glo viability reagent. The mixture was incubated for 10 min, and the luminescence signal was quantified using an Envision plate reader. Untreated cell and cells treated at 2 μM puromycin (Sigma, St. Louis, MO) serve as 100% and 0% cell viability control, respectively. The percent of cell viability was calculated for each tested compound concentration relative to the 0% and 100% controls and the CC₅₀ value was determined by non-linear regression as a compound concentration reducing the cell viability by 50%.

### Example T. H1-Hela Anti-HRV Assay

Both H1-HeLa cells and human rhinovirus 16 (HRV-16) are purchased from ATCC.

H1-HeLa maintenance media is composed of DMEM supplemented with 10% FBS and 1% Penn/Strep. Virus infection medium (VIM) is composed of DMEM+2% FBS.

H1-HeLa cells are seeded into 96-well black/clear bottom plates with 5,000 cells/well in 100 μL/well in H1-HeLa maintenance medium and incubated for 24 hours at 37° C. and 5% CO₂.

On the following day medium is aspirated and replaced with 100 μL VIM, next three-fold serial dilutions of compounds prepared in DMSO are added to the wells using the HP D300e digital dispenser with normalization to the highest concentration of DMSO in all wells. HRV-16 is diluted with the VIM to an MOI=0.05 and added to the cells in 100 μL/well. On each plate, uninfected and infected DMSO controls are included to determine compound efficacy against HRV. When extensive cytopathic effect is observed in the positive control (usually 3-6 days post infection) following the incubation at 37° C. and 5% CO₂, the culture plates are cooled to room temperature. The culture medium is removed and 200 μL of CellTiter Glo (1:2 dilution in PBS) is added to each well. The plates are agitated for 10 minutes on a shaker at room temperature and luminescence signal is measured using an EnVision plate reader (PerkinElmer). Values are normalized to the uninfected and infected DMSO controls (0% and 100% infection, respectively). Non-linear regression analysis is applied to determine the compound concentration at which 50% luminescence signal is reduced (EC₅₀) using the XLfit4 add-in for MICROSOFT® EXCEL®. All experiments are performed in duplicate with two technical repeats.

### Example U. NHBE RSV-Luc5 384-well Assay (EC50 RSVFLUC NHBE-384)

Normal Human Bronchial Epithelial (NHBE) cells were purchased from Lonza (Walkersville, MD Cat #CC2540) and maintained in BEGM Bronchial Epithelial Cell Growth Medium BulletKit (Lonza CC-3170).

Cells were thawed, expanded, and were used for experiments at passage 2. Respiratory syncytial virus recombinant with luciferase (RSV-Luc5) (≥1×10⁷ Infectious Units/ml (IU/ml) determined by TCID₅₀) was purchased from Microbiologics (Saint Cloud, MN). Viral replication was determined in NHBE cells in the following manner.

Compounds are prepared in 100% DMSO in 384-well polypropylene plates (Greiner, Monroe, NC, Cat #784201) with 8 compounds per plate in grouped replicates of 4 at 10 serially diluted concentrations (1:3). The serially diluted compounds were transferred to low dead volume Echo plates (Labcyte, Sunnyvale, CA, Cat #LP-0200).

The test compounds were spotted to 384-well assay plates (Greiner, Monroe, NC, Cat #781091) at 200 nL per well. NHBE cells were harvested and suspended in BEGM Bronchial Epithelial Cell Growth Medium BulletKit and seeded to the pre-spotted assay plates at 5,000 cells per well in 30 μL. RSV-Luc5 virus was diluted in BEGM Bronchial Epithelial Cell Growth Medium BulletKit at 500,000 Infectious Units (IU) per mL and 10 μL per well was added to the assay plates containing cells and compounds, for an MOI of 1. The assay plates were incubated for 3 days at 37° C. and 5% CO₂. At the end of incubation, One-Glo reagent (Promega, Madison, WI, Cat #E6120) was prepared. The assay plates and One-Glo reagent were equilibrated to room temperature for at least 15 minutes. 40 μL per well of One-Glo reagent was added and the plates were incubated at room temperature for 15 minutes before reading the luminescence signal on an EnVision multimode plate reader (Perkin Elmer, Waltham, MA). Remdesivir was used as positive control and DMSO was used as negative control. Values were normalized to the positive and negative controls (as 0% and 100% replication, respectively) and data was fitted using non-linear regression analysis by Gilead's dose response tool. The EC₅₀ value for each compound was defined as the concentration reducing the viral replication by 50%.

### Example V: HEp-2 RSV-Luc5 384-Well Assay (EC50 RSVFLUC Hep2-384 v2)

HEp-2 cell line was purchased from ATCC (Manassas, VA Cat #CCL-23) and maintained in Dulbecco's Minimum Essential Medium (DMEM) (Corning, New York, NY, Cat #15-018CM) supplemented with 10% fetal bovine serum (FBS) (Hyclone, Logan, UT, Cat #SH30071-03) and 1× Penicillin-Streptomycin-L-Glutamine (Corning, New York, NY, Cat #30-009-C1). Cells were passaged 2 times per week to maintain sub-confluent densities and were used for experiments at passage 5-20. Respiratory syncytial virus recombinant with luciferase (RSV-Luc5) (≥1×107 TCID50/ml) was purchased from Microbiologics (Saint Cloud, MN). Viral replication was determined in HEp-2 cells in the following manner.

Compounds are prepared in 100% DMSO in 384-well polypropylene plates (Greiner, Monroe, NC, Cat #784201) with 8 compounds per plate in grouped replicates of 4 at 10 serially diluted concentrations (1:3). The serially diluted compounds were transferred to low dead volume Echo plates (Labcyte, Sunnyvale, CA, Cat #LP-0200).

The test compounds were spotted to 384-well assay plates (Greiner, Monroe, NC, Cat #781091) at 200 nL per well. HEp-2 cells were harvested and suspended in DMEM (supplemented with 10% FBS and 1× Penicillin-Streptomycin-L-Glutamine) and seeded to the pre-spotted assay plates at 4,000 cells per well in 30 μL. RSV-Luc5 viruses were diluted in DMEM (supplemented with 10% FBS and 1× Penicillin-Streptomycin-L-(Glutamine) at 200,000 Infectious Units (IU) per mL and 10 μL per well was added to the assay plates containing cells and compounds, for an MOI=0.5. The assay plates were incubated for 3 days at 37° C. and 5% CO₂. At the end of incubation, One-Glo reagent (Promega, Madison, WI, Cat #E6120) was prepared. The assay plates and One-Glo reagent were equilibrated to room temperature for at least 15 minutes. 40 μL per well of One-Glo reagent was added and the plates were incubated at room temp for 15 minutes before reading the luminescence signal on an EnVision multimode plate reader (Perkin Elmer, Waltham, MA). Remdesivir was used as positive control and DMSO was used as negative control. Values were normalized to the positive and negative controls (as 0% and 100% replication, respectively) and data was fitted using non-linear regression analysis by Gilead's dose response tool. The EC₅₀ value for each compound was then determined as the concentration reducing the viral replication by 50%.

### Example W: NHBE RSV-Luc5 384-Well Assay (EC50 RSVFLUC NHBE-384 v2)

Normal Human Bronchial Epithelial (NHBE) cells were purchased from Lonza (Walkersville, MD Cat #CC2540) and maintained in BEGM Bronchial Epithelial Cell Growth Medium BulletKit (Lonza CC-3170).

Cells were thawed, expanded, and were used for experiments at passage 2. Respiratory syncytial virus recombinant with luciferase (RSV-Luc5) (≥1×10⁷ Infectious Units/mL (IU/mL) determined by TCID₅₀) was purchased from Microbiologics (Saint Cloud, MN). Viral replication was determined in NHBE cells in the following manner.

Compounds are prepared in 100% DMSO in 384-well polypropylene plates (Greiner, Monroe, NC, Cat #784201) with 8 compounds per plate in grouped replicates of 4 at 10 serially diluted concentrations (1:3). The serially diluted compounds were transferred to low dead volume Echo plates (Labcyte, Sunnyvale, CA, Cat #LP-0200).

The test compounds were spotted to 384-well assay plates (Greiner, Monroe, NC, Cat #781091) at 200 nL per well using an Echo acoustic dispenser (Labcyte, Sunnyvale, CA). NHBE cells were harvested and suspended in BEGM Bronchial Epithelial Cell Growth Medium BulletKit and seeded to the pre-spotted assay plates at 5,000 cells per well in 30 μL. RSV-Luc5 virus was diluted in BEGM Bronchial Epithelial Cell Growth Medium BulletKit at 500,000 Infectious Units (IU) per mL and 10 μL per well was added to the assay plates containing cells and compounds, for an MOI of 1. The assay plates were incubated for 3 days at 37° C. and 5% CO₂. At the end of incubation, One-Glo reagent (Promega, Madison, WI, Cat #E6120) was prepared. The assay plates and One-Glo reagent were equilibrated to room temperature for at least 15 minutes. 40 μL per well of One-Glo reagent was added and the plates were incubated at room temperature for 15 minutes before reading the luminescence signal on an EnVision multimode plate reader (Perkin Elmer, Waltham, MA). Remdesivir was used as positive control and DMSO was used as negative control. Values were normalized to the positive and negative controls (as 0% and 100% replication, respectively) and data was fitted using non-linear regression analysis by Gilead's dose response tool. The EC₅₀ value for each compound was defined as the concentration reducing the viral replication by 50%.

### Example X: H1-HeLa HRV-CTG 384-Well Assay

H1-Hela cell line (ATCC, Manassas, VA, Cat #CRL-1958) was maintained in Dulbecco's Minimum Essential Medium (DMEM) (Corning, New York, NY, Cat #15-018CM) supplemented with 10% fetal bovine serum (FBS) (Hyclone, Logan, UT, Cat #SH30071-03), and 1× Penicillin-Streptomycin-L-Glutamine (Corning, New York, NY, Cat #30-009-CI). Cells were passaged 2 times per week to maintain sub-confluent densities and were used for experiments at passage 5-30. The Human Rhinovirus 1B (HRV1B) (ATCC, Manassas, VA, Cat #VR-1645), Human Rhinovirus 14 (HRV14) (ATCC, Manassas, VA, Cat #VR-284), and Human Rhinovirus 16 (HRV16) (ATCC, Manassas, VA, Cat #BR-283) was obtained through ATCC. Viral infection was monitored by determining viability of H1-HeLa cells as described below.

Test molecules are prepared in 100% DMSO in 384-well polypropylene plates (Greiner, Monroe, NC, Cat #784201) with 8 compounds per plate in grouped replicates of 4 at 10 serially diluted concentrations (1:3). The serially diluted compounds were transferred to low dead volume Echo plates (Labcyte, Sunnyvale, CA, Cat #LP-0200).

The test compounds were spotted to 384-well assay plates (Greiner, Monroe, NC, Cat #781091) at 200 nL per well using an Echo acoustic dispenser (Labcyte, Sunnyvale, CA). H1-HeLa cells were harvested and suspended in DMEM (supplemented with 2% FBS and 1× Penicillin-Streptomycin-L-Glutamine) and seeded to the pre-spotted assay plates at 5,000 cells per well in 30 μL. HRV1B, HRV14, and HRV16 was diluted in DMEM (supplemented with 2% FBS and 1× Penicillin-Streptomycin-L-Glutamine) at 97.1 million Infectious Units (IU) per mL, 151 million IU per mL and 221 million IU per mL respectively. 10 μL of virus per well was added to the assay plates containing cells and compounds, for an MOI of 0.5, 1.0, and 0.25 respectively. The assay plates were incubated for 4 days at 37° C. and 5% CO2. At the end of incubation, Celltiter-Glo (Promega, Madison, WI, Cat #G7573) was prepared. The assay plates and Celltiter-Glo reagent were equilibrated to room temperature for at least 15 minutes. 40 μL per well of Celltiter-Glo reagent was added and the plates were incubated at room temperature for 15 minutes before reading the luminescence signal on an EnVision multimode plate reader (Perkin Elmer, Waltham, MA). Rupintrivir was used as positive control and DMSO was used as negative control. Values were normalized to the positive and negative controls (as 0% and 100% replication, respectively) and data was fitted using non-linear regression analysis by Gilead's dose response tool. The EC50 value for each compound was defined as the concentration reducing viral replication by 50%.

### Biological Data

Provided below in Table 3 is data related to some compounds disclosed herein.

TABLE 3
Biological Data for Some Compounds Disclosed Herein

| | | | EC50 H1- | EC50 H1- | EC50 H1- |
|---|---|---|---|---|---|
| | EC50 RSV | EC50 RSV | HeLa_HRV | HeLa_HRV | HeLa_HRV |
| | FLUC NHBE | FLuc Hep-2 | 14_HRV- | 16_HRV- | 1B_HRV- |
| | 384 | 384 | CTG 384- | CTG 384- | CTG 384- |
| | V2 | V2 | well Assay | well Assay | well Assay |
| | (per | (per | (per | (per | (per |
| Compound | Ex. W) | Ex. V) | Ex. X) | Ex. X) | Ex. X) |
| # | nM | nM | nM | nM | nM |
| 1 | 12793 | 21237 | 19077 | 31175 | 8650 |
| 2 | 11424 | 9487 | 7663 | 9744 | 7000 |
| 3 | 7926 | 20896 | 24362 | 34494 | 16012 |
| 4 | 9852 | 24118 | 22151 | 38586 | 11150 |
| 5 | 12532 | 17913 | 9745 | 11217 | 3347 |
| 6 | 36577 | 17847 | 15184 | 13671 | 8913 |
| 7 | 13859 | 16785 | 7311 | 9134 | 2815 |
| 8 | 12489 | 11531 | 7599 | 9819 | 5164 |
| 9 | 15939 | 13272 | 14709 | 10931 | 7566 |
| 10 | 11366 | 24654 | 35701 | 36724 | 18138 |
| 11 | 12020 | 31206 | 31074 | 37399 | 12190 |
| 12 | 8879 | >50000 | 27236 | 30337 | 10036 |
| 13 | 10776 | 12088 | 4669 | 7675 | 3194 |
| 14 | 14908 | 31620 | 44543 | 50000 | 22871 |
| 15 | 11409 | 14383 | 7555 | 9146 | 7049 |
| 16 | 50000 | 50000 | 50000 | 50000 | 50000 |
| 17 | 5960 | 24930 | 18431 | 15129 | 6079 |
| 18 | 5770 | | | | |
| 19 | 7026 | 13610 | 6999 | 8392 | 3574 |
| 20 | 7509 | 13669 | 12302 | 9577 | 4693 |
| 21 | 6997 | 21925 | 23507 | 26710 | 7869 |
| 22 | 6624 | 26328 | 19940 | 26256 | 9801 |
| 23 | 7789 | 15431 | 11369 | 9644 | 5316 |
| 24 | 7325 | 29473 | 10167 | 14136 | 6750 |
| 25 | 50000 | 50000 | 50000 | 50000 | 50000 |
| 26 | 50000 | 50000 | 50000 | 50000 | 50000 |
| 27 | 8027 | | | | |
| 28 | 9402 | 18198 | 8728 | 9510 | 3532 |
| 29 | 10936 | 19576 | 8063 | 9856 | 3263 |
| 30 | 7535 | 23048 | 5585 | 8883 | 3118 |
| 31 | 8650 | 20340 | 7622 | 9073 | 3162 |
| 32 | 7125 | 20636 | 11496 | 12068 | 5685 |
| 33 | 8013 | 25106 | 15574 | 18457 | 8698 |
| 34 | 6386 | 17610 | 13987 | 12057 | 7310 |
| 35 | 5782 | 18941 | 12223 | 15103 | 8630 |
| 36 | 8420 | 14275 | 10621 | 11974 | 5933 |
| 37 | 7442 | 21325 | 20504 | 12556 | 6742 |
| 38 | 7607 | 50000 | 24367 | 39995 | 23796 |
| 39 | 7838 | 28127 | 11699 | 22008 | 8261 |
| 40 | 7424 | 26686 | 15353 | 29337 | 9484 |
| 42 | 26104 | | | | |
| 43 | 6990 | | | | |
| 44 | 15787 | | | | |
| 45 | 29418 | | | | |

### Example Y: GI S9 Stability Data

### Reagents

Stock solutions of test compounds in dimethyl sulfoxide (DMSO) having a final concentration of 10 mM were prepared and used in all experiments. Sekisui XenoTech (Kansas City, KS) provided pooled intestinal S9 fractions. All other chemicals were purchased from Sigma-Aldrich (St. Louis, MO) or VWR (West Chester, PA). Internal Standard/Quench (IS/Q) used to stop reactions was 100 nM labetalol in (by volume) methanol (10%) and acetonitrile (90%).

### Assay

Stability in Intestinal S9 Fractions: Duplicate aliquots of test compound or positive control substrate were added to S9 stock diluted with phosphate buffered saline (137 mM NaCl, 2.7 mM KCl, 10 mM phosphate buffer), pH 7.4, to obtain a protein concentration of 1.0 mg/mL. The metabolic reactions were initiated by the addition of the substrates to the S9 reaction mixture to a final concentration of 2 μM. At 0, 10, 20, 30, 60 and 120 min, 25 μL aliquots of the reaction mixture were transferred to plates containing 225 μl of IS/Q solution. After quenching, the plates were centrifuged at 3000×g for 30 minutes. 150 μL aliquots of each supernatant were dried down then reconstituted with 300 μL water. Aliquots (10 μL) of the prepared samples were analyzed on a Thermo Q-Exactive mass spectrometer as described below.

Liquid Chromatography—Mass Spectrometry: Quantification of test compounds and control substrate was performed by analyte/internal standard peak area ratio (PAR) values measured on a Thermo Q-Exactive mass spectrometer coupled to a Dionex UltiMate 3000 HPLC with a Leap Technologies HTC PAL autosampler. The column used for analysis was a Waters Acquity BEH C₁₈ (1.7 μm particle size, 2.1×50 mm). Mobile phase A consisted of 0.1% (v/v) formic acid in water. Mobile phase B consisted of 0.1% (v/v) formic acid in acetonitrile. Elution of analytes was achieved by a series of linear gradients varying the proportions of A and B. The mass spectrometer was calibrated on a weekly basis and mass tolerance of 5 ppm was used.

### Data Analysis

Metabolic stabilities in S9 fractions were determined by measuring the rates of disappearance of test compound and positive control substrate.

Data (% of substrate remaining) were plotted on a semi-log scale and fitted using an exponential decline model:

Cₜ=C₀×e^{-ln}2/T1/2×t
Where Cₜ—% of substrate remaining at time=t; C₀—% of substrate remaining at time=0; t—time; T1/2—half-life.
The Half-life (T1/2) is determined by the following equation:

T1/2=−ln0.5/k=0.693/k

Assuming a first-order reaction, the slope (k) is extrapolated from the aforementioned plotted data.

TABLE 4
T½ (half-life) data of some compounds disclosed herein

| | GIS9 Rat | GIS9 DOG | GIS9 Cyno | GIS9 Human |
|---|---|---|---|---|
| | T½ | T½ | T½ | T½ |
| | (per | (per | (per | (per |
| | Ex. Y) | Ex. Y) | Ex. Y) | Ex. Y) |
| Compound | min | min | min | min |
| 1 | 6 | 256 | 11 | 31 |
| 2 | 6 | 162 | 4 | 23 |
| 4 | 10 | 462 | 22 | 65 |
| 5 | 4 | | | 44 |
| 6 | 37 | 789 | 38 | 201 |
| 7 | 3 | | | 20 |
| 8 | 7 | 311 | 8 | 29 |
| 9 | 8 | 156 | 19 | 29 |
| 10 | 13 | 789 | 9 | 104 |
| 11 | 14 | 789 | 35 | 104 |
| 12 | 58 | 789 | 274 | 789 |
| 13 | 74 | 96 | 72 | 97 |
| 14 | 16 | 534 | 8 | 104 |
| 15 | 2 | 12 | 2 | 1 |
| 16 | 789 | | | 789 |
| 17 | 7 | | | 29 |
| 19 | 2 | | | 11 |
| 20 | 3 | | | 16 |
| 21 | 8 | | | 44 |
| 22 | 14 | | | 55 |
| 23 | 9 | | | 19 |
| 24 | 47 | 789 | 147 | 178 |
| 28 | 4 | | | 18 |
| 29 | 3 | | | 15 |
| 30 | 3 | | | 15 |
| 31 | 3 | | | 18 |
| 32 | 8 | | | 33 |
| 33 | 58 | 789 | 163 | 250 |
| 34 | 11 | | | 108 |
| 35 | 4 | | | 19 |
| 36 | 3 | | | 14 |
| 37 | 4 | | | 29 |
| 38 | 38 | | | 292 |
| 39 | 7 | | | 27 |
| 40 | 6 | | | 36 |
| 41 | 8 | | | 1 |

### Example A1: Rat Pharmacokinetics Assay

Reference Compound 0, Compound 1, and all compounds were dosed orally by gavage to male Sprague-Dawley rats (n=3/group); Compound 0 was dosed at 10 mg/kg in 500 Ethanol; 55% Polyethylene glycol 400 and 4000 water+1 equiv HCl, pH 3.4; All other compounds were dosed at 10 mg/kg in 2.5% Dimethyl sulfoxide; 10% Kolliphor HS-15; 10% Labrasol; 2.5% Propylene glycol and 75% water. Blood samples were collected into pre-chilled collection tubes containing K₂EDTA and processed to plasma at 10-11 timepoints over a span of predose to 24 h post-administration. Plasma samples were subject to protein precipitation with 8-fold volume of acetonitrile, vortexed and centrifuged. Supernatants were transferred and diluted by water. Separation was achieved on a Phenomenex Polar-RP column, a mobile phase A of 0.10% formic acid in acetonitrile: water (1:99) and a mobile phase B of 0.1% formic acid in acetonitrile: water (95:5) with a stepwise linear gradient from 1 to 99% mobile phase B. An LCMS/MS method was used to measure the concentrations of the Reference compound 0 and the corresponding compound in plasma. Data for Reference Compound 0 following oral administration of Compound 0, and all other compounds is tabulated below (Table 5).

TABLE 5
Rat pharmacokinetics of some compounds

| | | | Reference | Reference |
|---|---|---|---|---|
| | Oral | Reference | compound 0 | Compound Oral |
| | dose | compound 0 | AUCinf. | Bioavailability |
| Compound | (mg/kg) | Cmax (nM) | (nM · h) | F % |
| 0 | 10 | 5870 | 39200 | 46 |
| 1 | 10 | 7870 | 37600 | 62 |
| 2 | 10 | 7090 | 35200 | 58 |
| 4 | 10 | 8840 | 40300 | 66 |
| 6 | 10 | 7680 | 34700 | 49 |
| 8 | 10 | 10200 | 57600 | 68 |
| 9 | 10 | 8820 | 39800 | 61 |
| 10 | 10 | 9560 | 45000 | 76 |
| 11 | 10 | 6910 | 36500 | 56 |
| 12 | 10 | 4390 | 28400 | 49 |
| 13 | 10 | 8040 | 50800 | 81 |
| 14 | 10 | 9870 | 44400 | 73 |
| 15 | 10 | 10200 | 47500 | 81 |
| 32 | 10 | 4550 | 26100 | 43 |

### Example A2: Dog Pharmacokinetics Assay

Reference Compound 0, and all other test compounds were dosed orally by gavage to male beagle dogs (n=3/group); All Compounds were dosed at 5 mg/kg in 2.5% Dimethyl sulfoxide; 10% Kolliphor HS-15; 10% Labrasol; 2.5% Propylene glycol and 75% water. Blood samples were collected into pre-chilled collection tubes containing K₂EDTA and processed to plasma at 10-11 timepoints over a span of predose to 24 h post-administration. Plasma samples were subject to protein precipitation with 8-fold volume of acetonitrile, vortexed and centrifuged.

Supernatants were transferred and diluted by water. Separation was achieved on a Phenomenex Polar-RP column, a mobile phase A of 0.1% formic acid in acetonitrile: water (1:99) and a mobile phase B of 0.1% formic acid in acetonitrile: water (95:5) with a stepwise linear gradient from 1 to 99% mobile phase B. An LCMS/MS method was used to measure the concentrations of the Reference compound 0 and either any test compound in plasma. Data for Reference Compound 0 following oral administration of Compound 0, and all other test compounds is tabulated below (Table 6).

TABLE 6
Dog pharmacokinetics of some compounds

| | | | Reference | Reference |
|---|---|---|---|---|
| | Oral | Reference | compound 0 | Compound Oral |
| | dose | compound 0 | AUCinf. | Bioavailability |
| Compound | (mg/kg) | Cmax (nM) | (nM · h) | F % |
| 0 | 5 | 8360 | 40600 | 46 |
| 8 | 5 | 9540 | 60000 | 90 |
| 10 | 5 | 6230 | 33200 | 50 |
| 13 | 5 | 9230 | 44500 | 67 |
| 14 | 5 | 6850 | 39400 | 61 |

### Example A3 Monkey Pharmacokinetics Assay

Reference Compound 0, and all other test compounds were dosed orally by gavage to male cynomolgus monkeys (n=3/group) using the following formulations. All compounds were dosed at 5 mg/kg in 2.5% Dimethyl sulfoxide; 10% Kolliphor HS-15; 10% Labrasol; 2.5% Propylene glycol and 75% water. Blood samples were collected into pre-chilled collection tubes containing K₂EDTA and processed to plasma at 10-11 timepoints over a span of predose to 24 h post-administration. Plasma samples were subject to protein precipitation with 8-fold volume of acetonitrile, vortexed and centrifuged. Supernatants were transferred and diluted by water. Separation was achieved on a Phenomenex Polar-RP column, a mobile phase A of 0.1% formic acid in acetonitrile: water (1:99) and a mobile phase B of 0.1% formic acid in acetonitrile: water (95:5) with a stepwise linear gradient from 1 to 99% mobile phase B. An LCMS/MS method was used to measure the concentrations of the Reference compound 0 and test compounds plasma.

Data for Reference Compound 0 following oral administration of Compound 0, and all other test compounds is tabulated below (Table 7).

TABLE 7
Monkey pharmacokinetics of some compounds

| | | | Reference | Reference |
|---|---|---|---|---|
| | Oral | Reference | compound 0 | Compound Oral |
| | dose | compound 0 | AUCinf. | Bioavailability |
| Compound | (mg/kg) | Cmax (nM) | (nM · h) | F % |
| 0 | 5 | 1270 | 7900 | 30 |
| 8 | 5 | 2500 | 10100 | 52 |
| 10 | 5 | 6930 | 33200 | 42 |
| 13 | 5 | 3470 | 11000 | 56 |
| 14 | 5 | 1640 | 7970 | 42 |

The present disclosure provides reference to various embodiments and techniques. However, it should be understood that many variations and modifications can be made while remaining within the spirit and scope of the present disclosure. The description is made with the understanding that it is to be considered an exemplification of the claimed subject matter, and is not intended to limit the appended claims to the specific embodiments illustrated.

1. A compound of Formula I: 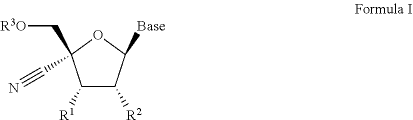
or a pharmaceutically acceptable salt thereof, wherein:
R¹ is OH, OC(O)R⁴, or OC(O)OR⁴, and
R² is OH, OC(O)R⁵, or OC(O)OR⁵, or
R¹ and R² are taken together to form —OC(O)O— or —OCHR⁶O—, wherein
R⁶ is H, C₁-C₆ alkyl, C₁-C₆ alkoxy, or C₆-C₁₀ aryl;
R³ is H or C(O)OR⁷, or
R¹ and R³ are taken together to form —OC(O)—;
R⁴, R⁵, and R⁷ are each independently C₁-C₈ alkyl, C₂-C₈ alkenyl, C₂-C₈ alkynyl, C₃-C₈ carbocyclyl, C₆-C₁₀ aryl, 4 to 6 membered heterocyclyl containing 1, 2, or 3 O, or 5 to 6 membered heteroaryl containing 1, 2, or 3 heteroatoms selected from N, O, and S;
wherein the alkyl, alkenyl, alkynyl, carbocyclyl, aryl, heterocyclyl or heteroaryl of R⁴, R⁵, and R⁷ are each, independently, optionally substituted with one, two or three substituents independently selected from the group consisting of halo, oxo, cyano, N₃, OR⁸, C₁-C₈ alkyl, NR⁹R¹⁰, C₃-C₈ carbocyclyl, and phenyl optionally substituted with one, two, or three substituents independently selected from halo, cyano, and C₁-C₆ alkyl; and
each R⁸ is independently H, C₁-C₆ alkyl, C₁-C₆ haloalkyl, and C₃-C₆ cycloalkyl;
each R⁹ is independently H, C₁-C₆ alkyl, C₁-C₆ haloalkyl, and C₃-C₆ cycloalkyl;
each R¹⁰ is independently H, C₁-C₆ alkyl, C₁-C₆ haloalkyl, and C₃-C₆ cycloalkyl; and
Base is 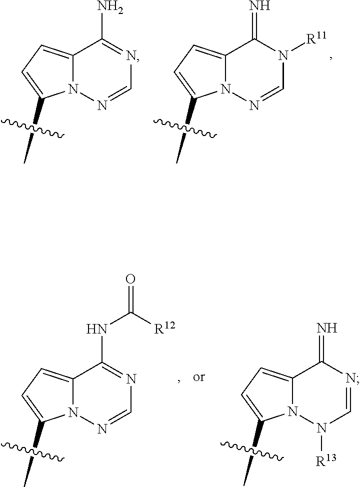
wherein
R¹¹ is C₁-C₆ alkyl substituted with OP(O)(OH)₂ or OC(O)R¹²;
R¹² is C₁-C₈ alkyl or C₁-C₈ alkoxy; and
R¹³ is C₁-C₆ alkyl substituted with OP(O)(OH)₂ or OC(O)R¹²;
wherein the compound is not

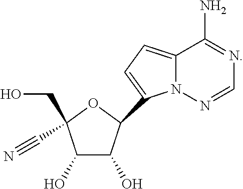 [SMILES: N#C[C@]1(CO)O[C@@H](c2ccc3c(N)ncnn23)[C@H](O)[C@@H]1O]

2. The compound of claim 1, or the pharmaceutically acceptable salt thereof, wherein the compound is of Formula II

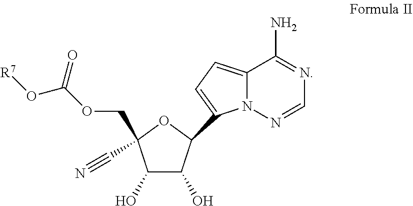

3. The compound according to a claim 1, or a pharmaceutically acceptable salt thereof, wherein
R¹ is OH, and
R² is OH, or
R¹ and R² are taken together to form —OC(O)O—;
R³ is H or C(O)OR⁷;
R⁷ is C₁-C₈ alkyl, C₃-C₈ carbocyclyl, 4 to 6 membered heterocyclyl containing 1, 2, or 3 O;
wherein the alkyl, carbocyclyl, or heterocyclyl of R⁷ is optionally substituted with one, two, or three substituents independently selected from the group consisting of C₁-C₈ alkyl and phenyl; and
Base is 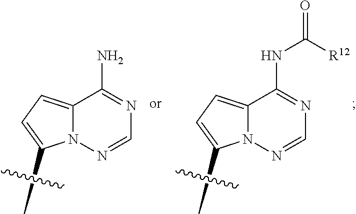
wherein
R¹² is C₁-C₈ alkoxy.

4.-10. (canceled)

11. The compound according to claim 1, or a pharmaceutically acceptable salt thereof, wherein R¹ and R² are taken together to form —OC(O)O—, —OCHR⁶O—; R⁶ is H or C₁-C₆ alkyl.

12.-13. (canceled)

14. The compound according to claim 1, or a pharmaceutically acceptable salt thereof, wherein R³ is C(O)OR⁷.

15. The compound according to claim 1, or a pharmaceutically acceptable salt thereof, wherein R³ is

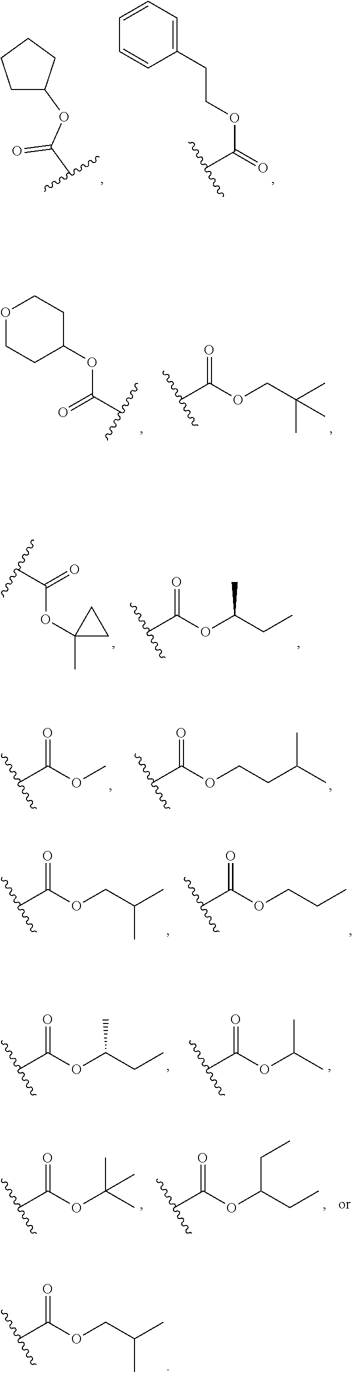

16.-62. (canceled)

63. The compound of claim 1, or the pharmaceutically acceptable salt thereof, wherein R⁷ is C₁-C₆ alkyl.

64. The compound of claim 1, or the pharmaceutically acceptable salt thereof, wherein R⁷ is isobutyl or tert-butyl.

65.-66. (canceled)

67. The compound of claim 1, or the pharmaceutically acceptable salt thereof, wherein R⁷ is C₃-C₈ carbocyclyl, 4 to 6 membered heterocyclyl containing 1, 2, or 3 O.

68.-95. (canceled)

96. The compound of claim 1, or or the pharmaceutically acceptable salt thereof, wherein Base is

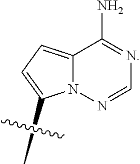

97. (canceled)

98. The compound of claim 1, or the pharmaceutically acceptable salt thereof, wherein Base is

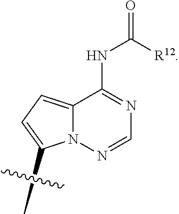

99.-106. (canceled)

107. The compound of claim 1, or the pharmaceutically acceptable salt thereof, wherein the compound is a compound of Table 1.

108. The compound of claim 1, or the pharmaceutically acceptable salt thereof, wherein the compound is

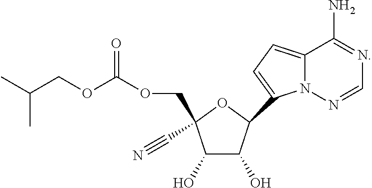 [SMILES: CC(C)COC(=O)OC[C@@]1(C#N)O[C@@H](c2ccc3c(N)ncnn23)[C@H](O)[C@@H]1O]

109. The compound of claim 1, or the pharmaceutically acceptable salt thereof, wherein the compound is

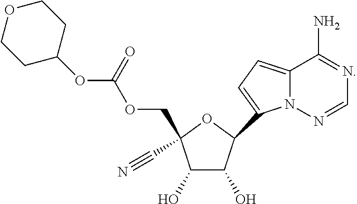 [SMILES: N#C[C@]1(COC(=O)OC2CCOCC2)O[C@@H](c2ccc3c(N)ncnn23)[C@H](O)[C@@H]1O]

110. The compound of claim 1, or the pharmaceutically acceptable salt thereof, wherein the compound is

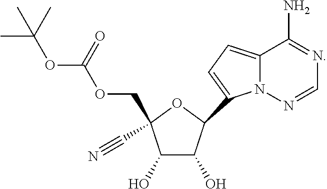 [SMILES: CC(C)(C)OC(=O)OC[C@@]1(C#N)O[C@@H](c2ccc3c(N)ncnn23)[C@H](O)[C@@H]1O]

111. The compound of claim 1, or the pharmaceutically acceptable salt thereof, wherein the compound is

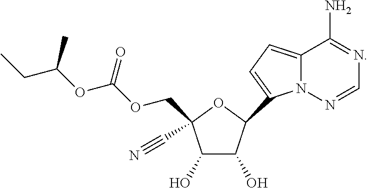 [SMILES: CC[C@@H](C)OC(=O)OC[C@@]1(C#N)O[C@@H](c2ccc3c(N)ncnn23)[C@H](O)[C@@H]1O]

112. The compound of claim 1, or the pharmaceutically acceptable salt thereof, wherein the compound is

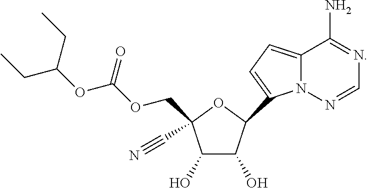 [SMILES: CCC(CC)OC(=O)OC[C@@]1(C#N)O[C@@H](c2ccc3c(N)ncnn23)[C@H](O)[C@@H]1O]

113. A pharmaceutical composition comprising a pharmaceutically effective amount of a compound of claim 1, or a pharmaceutically acceptable salt thereof, and a pharmaceutically acceptable carrier or excipient.

114.-117. (canceled)

118. A method of treating or preventing a viral infection in a subject in need thereof, wherein the method comprises administering to the subject a therapeutically effective amount of a pharmaceutical composition of claim 113.

119.-128. (canceled)

129. The method of claim 118, wherein the viral infection is a pneumoviridae virus infection, or a picornaviridae virus infection.

130.-156. (canceled)
